SEQUENCE LISTING (276 sequences)

SEQ ID NO: 1 - protein, 18 aa, synthetic construct
AARGDVYYGGDDATRYKY

SEQ ID NO: 2 - protein, 15 aa, synthetic construct
NLVVPYGMIAGDHDY

SEQ ID NO: 3 - protein, 15 aa, synthetic construct
AAGLGIRVVSQTPAY

SEQ ID NO: 4 - protein, 15 aa, synthetic construct
AAGRYSRARGWEYDY

SEQ ID NO: 5 - protein, 15 aa, synthetic construct
AAGRYSRVRGWEYDY

SEQ ID NO: 6 - protein, 15 aa, synthetic construct
AAISWTGGSTGYADS

SEQ ID NO: 7 - protein, 14 aa, synthetic construct
AVATSGGTIHYADS

SEQ ID NO: 8 - protein, 14 aa, synthetic construct
AAIGWSGVIEYADS

SEQ ID NO: 9 - protein, 15 aa, synthetic construct
ARKTWDGSSTRYDDS

SEQ ID NO: 10 - protein, 15 aa, synthetic construct
ARRTWDGSSTRYDDS

SEQ ID NO: 11 - protein, 8 aa, synthetic construct
GRTFLSMG

SEQ ID NO: 12 - protein, 10 aa, synthetic construct
GSIFSVNTMG

SEQ ID NO: 13 - protein, 10 aa, synthetic construct
RRSFSEYAMG

SEQ ID NO: 14 - protein, 10 aa, synthetic construct
GRALSDYNIG

SEQ ID NO: 15 - protein, 10 aa, synthetic construct
GRAFGDYNIG

SEQ ID NO: 16 - protein, 126 aa, synthetic construct
QVQLVESGGGLVQAGASLTLSCAASGFTFNTRSIVWFRQAPGKEREAVSCISKVAGSTYYGDSVQGRFSSSTDNAKNTVSLQMNSLKPEDTAVYTCAASFAPNCYGWSVAANMEYWGEGTMVTVSS

SEQ ID NO: 17 - protein, 123 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSS

SEQ ID NO: 18 - protein, 117 aa, synthetic construct
QVQLVESGGGLVQAGESLRLACAASGTIFSGNAVGWYRQAPEKQREWVSSITDYGDTDYAEAVKGRFTISRDNAKNTVYLQMNSLKPEDTAVYYCYVYQRGESEEYWGQGTKVTVSS

SEQ ID NO: 19 - protein, 123 aa, synthetic construct
QVHLVECGGGSVQAGGFLRLSCAASGPSLSNYAMGRFRQAPGKEREFVAGISWGGGRTSYTDSVKGRFTISRDNAKNTVYLQMNNLEPEDTGVYYCAGRYGRGRNVEYEYDYWGQGTKVTVSS

SEQ ID NO: 20 - protein, 122 aa, synthetic construct
QVQLVESGGGLVQAGGSLRLSCEVTRSPMPIDTMAWYRQAPGRQRELVAIISRSGSTNYADSVKGRFTISRNDTKSTMYKSTMYLKMNTLELEDTGVYYCNAVGLWNGAEYWGQGTQVTVSS

SEQ ID NO: 21 - protein, 123 aa, synthetic construct
QVQLVESGGGSVQAGGSLRLSCAASGPSLSHYAMGWFRQAPGKEREFVAGISWGGGRTSYTDSVKGRFTISRDNAKNTVYLQMNNLKPEDTGVYYCAGRYGRGRNVEYEYDYWGQGTQVTVSS

SEQ ID NO: 22 - protein, 123 aa, synthetic construct
QVHLVESGGGSVQAGGSLRLSCAASGPSLSNYAMGWFRQAPGKEREFVAGISWGGGRTSYTDSVKGRFTISRDNAKNTVYLQMNNLKPEDTGVYYCAGRYGRGRNVEYEYDYWGQGTQVTVSS

SEQ ID NO: 23 - protein, 121 aa, synthetic construct
QVQLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSS

SEQ ID NO: 24 - protein, 123 aa, synthetic construct
QVQLVESGGGLVQPGGSLRVSCAASGPSLSNYAMGWFRQAPGKEREFVAGISWGGGRTSYTDSVKGRFTISRDNAKNTVYLQMNNLKPEDTGVYYCAGRYGRGRNVEYEYDYWGQGTQVTVSS

SEQ ID NO: 25 - protein, 117 aa, synthetic construct
QVQLVESGGGLVQAGESLRLACAASGTIFSGNAVGWYRQAPEKQREWVSSITDYGDTDYAEAVKGRFTISRDNAKNTVYLQMNSLKPEDTAVYYCYVYQRGESEEYWGQGTQVTVSS

SEQ ID NO: 26 - protein, 120 aa, synthetic construct
QVQLVESGGGLVQPGGSLRLSCAASESTFSINVMGWYRQAPGKQRELVATITSGDITNYADSVKGRFTISRDNAENTVYLRTNSLKPEDTAVYYCNARGEYHSGNEYDYWGQGTQVTVSS

SEQ ID NO: 27 - protein, 123 aa, synthetic construct
QVHLVESGGGSVQAGGSLRLSCAASGPSLSNYAMGWFRQAPGKEREFVAGISWGGGRTSYTDSVKGRFTISRDNAKNTVYLQMNNLKPEDTGVYYCAGRYGRGRNVEYEYDYWGQGTKVTVSS

SEQ ID NO: 28 - protein, 121 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSS

SEQ ID NO: 29 - protein, 123 aa, synthetic construct
QVQLVESGGGSVQAGGSLRLSCAASGPSLSNYAMGWFRQAPGKEREFVAGISWGGGRTSYTDSVKGRFTISRDNAKNTVYLQMNNLKPEDTGVYYCAGRYGRGRNVEYEYDYWGQGTMVTVSS

SEQ ID NO: 30 - protein, 121 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSS

SEQ ID NO: 31 - protein, 122 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSS

SEQ ID NO: 32 - protein, 123 aa, synthetic construct
QVQLVESGGGSVQAGGSLRLSCAASGPSLSNFAMGWFRQPPGKEREFVAGISWGGGRTSYTDSVKGRFTISRDNAKNTVYLQMNNLKPEDTGVYYCAGRYGRGRNVEYEYDYWGQGTQVTVSS

SEQ ID NO: 33 - protein, 123 aa, synthetic construct
QVQLVESGGGSVQAGGSLRLSCAASGPSISNFAMGWFRQAPGKEREFVAGISWGGGRTNYLDSVKGRFTISRDNAKNMVHLQMNSLKPEDTTVYYCAGRYGRGRDVEYEYDYWGQGTQVTVSS

SEQ ID NO: 34 - protein, 122 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSS

SEQ ID NO: 35 - protein, 123 aa, synthetic construct
QVQLVESGGGSVQAGGSLRLSCAASGPSLSNYAMGWFRQAPGKEREFVAGISWGGGRTSYTDSVKGRFTISRDNAKNTVYLQMNNLKPEDTGVYYCAGRYGRGRNVEYEYDYWGQGTQVTVSS

SEQ ID NO: 36 - protein, 123 aa, synthetic construct
QVQLVESGGGSVQAGGSLRLSCAASGPSLSNYAMGWFRQAPGKEREFVAGISWGGGRTSCTDSVKGRFTISRDNAKNTVYLQMNNLKPEDTGVYYCAGRYGRGRNVEYEYDYWGQGTQVTVSS

SEQ ID NO: 37 - protein, 122 aa, synthetic construct
QVQLVESGGGLVEAGGSLRLSCAVSGSALSDYNIGWFRQAPGKEREFVARKTWDGSSTKYDDSVKGRFTVSIDNAKNTAYLQMNSLKTEDTAVYYCAAGRYSRVRGWEYDYWGQRTQVTVSS

SEQ ID NO: 38 - protein, 122 aa, synthetic construct
QVQLVESGGGTAQAGGSLRLACAASGRSVTMGWLGWVPRAERDDLEFITWFNWGTYYANSVNGRFTISTDNADNTVYPQMNRLKPENPAVYYCASFTGYRPLINRLNDVSDCGHGALVAVSS

SEQ ID NO: 39 - protein, 123 aa, synthetic construct
QVQLVESGGGSVQTGGSLRLSCAASGPSLSNYAMGWFRQAPGKEREFVAGISWGGGRTNYLDSVKGRFTISRDNAKNTMYLQMNSLKPEDTAVYYCAGRYGRGRDVEYEYDYWGQGTQVTVSS

SEQ ID NO: 40 - protein, 123 aa, synthetic construct
QVQLVESGGGSVQTGGSLRLSCAASGPSLSNYAMGWFRQAPGKEREFVAGISWGGGRTNYLDSVKGRFTISRDNAKNTMYLQMNSLKPEDAAVYYCAGRYGRGRDVEYEYDYWGQGTQVTVSS

SEQ ID NO: 41 - protein, 25 aa, synthetic construct
QVQLVESGGGLVQAGASLTLSCAAS

SEQ ID NO: 42 - protein, 25 aa, synthetic construct
QVQLVESGGGLVQAGESLRLACAAS

SEQ ID NO: 43 - protein, 25 aa, synthetic construct
QVHLVECGGGSVQAGGFLRLSCAAS

SEQ ID NO: 44 - protein, 25 aa, synthetic construct
QVQLVESGGGLVQAGGSLRLSCEVT

SEQ ID NO: 45 - protein, 25 aa, synthetic construct
QVQLVESGGGSVQAGGSLRLSCAAS

SEQ ID NO: 46 - protein, 25 aa, synthetic construct
QVHLVESGGGSVQAGGSLRLSCAAS

SEQ ID NO: 47 - protein, 25 aa, synthetic construct
QVQLVESGGGLVQAGGSLRLSCAAS

SEQ ID NO: 48 - protein, 25 aa, synthetic construct
QVQLVESGGGLVQPGGSLRVSCAAS

SEQ ID NO: 49 - protein, 25 aa, synthetic construct
QVQLVESGGGLVQPGGSLRLSCAAS

SEQ ID NO: 50 - protein, 25 aa, synthetic construct
QVQLVESGGGLVEAGGSLRLSCAVS

SEQ ID NO: 51 - protein, 25 aa, synthetic construct
QVQLVESGGGTAQAGGSLRLACAAS

SEQ ID NO: 52 - protein, 25 aa, synthetic construct
QVQLVESGGGSVQTGGSLRLSCAAS

SEQ ID NO: 53 - protein, 25 aa, synthetic construct
QVHLVESGGGLVQAGGSLRLSCAAS

SEQ ID NO: 54 - skipped

SEQ ID NO: 55 - protein, 25 aa, synthetic construct
QVQLVESGGGLVQAGGSLRLSCAVS

SEQ ID NO: 56 - skipped

SEQ ID NO: 57 - protein, 13 aa, synthetic construct
WFRQAPGKEREAV

SEQ ID NO: 58 - protein, 13 aa, synthetic construct
WYRQAPEKQREWV

SEQ ID NO: 59 - protein, 13 aa, synthetic construct
RFRQAPGKEREFV

SEQ ID NO: 60 - protein, 13 aa, synthetic construct
WYRQAPGRQRELV

SEQ ID NO: 61 - protein, 13 aa, synthetic construct
WYRQAPGKQRELV

SEQ ID NO: 62 - protein, 13 aa, synthetic construct
WFRQPPGKEREFV

SEQ ID NO: 63 - protein, 13 aa, synthetic construct
WVPRAERDDLEFI

SEQ ID NO: 64 - protein, 13 aa, synthetic construct
WFRQAPGKEREFV

SEQ ID NO: 65 - protein, 13 aa, synthetic construct
WYRQAPGKQRDLV

SEQ ID NO: 66 - protein, 66 aa, synthetic construct
VQGRFSSSTDNAKNTVQGRFSSSTDNAKNTVSLQMNSLKPEDTAVYTCVSLQMNSLKPEDTAVYTC

SEQ ID NO: 67 - protein, 33 aa, synthetic construct
VKGRFTISRDNAKNTVYLQMNSLKPEDTAVYYC

SEQ ID NO: 68 - protein, 33 aa, synthetic construct
VKGRFTISRDNAKNTVYLQMNNLEPEDTGVYYC

SEQ ID NO: 69 - protein, 38 aa, synthetic construct
VKGRFTISRNDTKSTMYKSTMYLKMNTLELEDTGVYYC

SEQ ID NO: 70 - protein, 33 aa, synthetic construct
VKGRFTISRDNAKNTVYLQMNNLKPEDTGVYYC

SEQ ID NO: 71 - protein, 33 aa, synthetic construct
VKGRFTISRDNAENTVYLRTNSLKPEDTAVYYC

SEQ ID NO: 72 - protein, 33 aa, synthetic construct
VKGRFTISRDNAKNMVHLQMNSLKPEDTTVYYC

SEQ ID NO: 73 - protein, 33 aa, synthetic construct
VNGRFTISTDNADNTVYPQMNRLKPENPAVYYC

SEQ ID NO: 74 - protein, 33 aa, synthetic construct
VKGRFTISRDNAKNTMYLQMNSLKPEDTAVYYC

SEQ ID NO: 75 - protein, 33 aa, synthetic construct
VKGRFTISRDNAKNTMYLQMNSLKPEDAAVYYC

SEQ ID NO: 76 - protein, 33 aa, synthetic construct
VKGRFTISRESDKETMYLQMNSLKPEDTAVYYC

SEQ ID NO: 77 - skipped

SEQ ID NO: 78 - protein, 33 aa, synthetic construct
VKGRFTISRDNAKDTVYLQMNSLKPEDTAVYYC

SEQ ID NO: 79 - protein, 33 aa, synthetic construct
VKGRFTVSIEDAKNTAYLQMSSLKTEDTAVYYC

SEQ ID NO: 80 - protein, 33 aa, synthetic construct
VKGRFTVSIDNAKNTAYLQMNSLKTEDTAVYYC

SEQ ID NO: 81 - protein, 11 aa, synthetic construct
WGEGTMVTVSS

SEQ ID NO: 82 - protein, 11 aa, synthetic construct
WGQRTQVTVSS

SEQ ID NO: 83 - protein, 11 aa, synthetic construct
CGHGALVAVSS

SEQ ID NO: 84 - protein, 11 aa, synthetic construct
WGQGTQVTVSS

SEQ ID NO: 85 - protein, 11 aa, synthetic construct
WGQGTKVTVSS

SEQ ID NO: 86 - protein, 11 aa, synthetic construct
WGQGTMVTVSS

SEQ ID NO: 87 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 90 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 92 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 93 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 94 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 95 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 96 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 97 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 98 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 99 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 100 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 101 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 102 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 103 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 107 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 108 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 109 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 110 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 111 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 112 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 113 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 114 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 115 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 116 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 117 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 118 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 119 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 120 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 121 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 122 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 123 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 124 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 125 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 126 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 127 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 128 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 129 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 130 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 131 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 132 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 133 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 134 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 135 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 136 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 137 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 141 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 142 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 148 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 149 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 150 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 151 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 152 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 153 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 154 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 155 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 156 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 157 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 158 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 159 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 160 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 161 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 162 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 163 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 164 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 165 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 166 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 167 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 168 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 169 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 170 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 171 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 172 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 173 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 174 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 175 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 176 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLCLSPGK

SEQ ID NO: 177 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 178 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 179 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 180 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 181 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 182 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 183 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 184 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 185 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 186 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLCLSPGK

SEQ ID NO: 187 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 188 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 189 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 190 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 191 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 192 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 193 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 194 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 195 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 196 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 197 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLTSKLTVDKSRWQQGNVFSCSVMHEALHNHYTQKSLSLSPGK

SEQ ID NO: 198 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 370 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 200 - protein, 368 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 201 - protein, 368 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 202 - protein, 369 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 203 - protein, 369 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 204 - protein, 360 aa, synthetic construct
QVHLVESGGGLVQAGGSLRLSCAASGRTFLSMGWFRQAPGKEREFVAAISWTGGSTGYADSVKGRFTISRESDKETMYLQMNSLKPEDTAVYYCAARGDVYYGGDDATRYKYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 205 - protein, 358 aa, synthetic construct
QVQLVESGGGLVQPGGSLRLSCAASGSIFSVNTMGWYRQAPGKQRDLVAVATSGGTIHYADSVKGRFTISRDNAKNTVYLQMNSLKPEDTAVYYCNLVVPYGMIAGDHDYWGQGTKVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 358 aa, synthetic construct
QVHLVESGGGLVQAGGSLRLSCAASRRSFSEYAMGWFRQAPGKEREFVAAIGWSGVIEYADSVKGRFTISRDNAKDTVYLQMNSLKPEDTAVYYCAAGLGIRVVSQTPAYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 359 aa, synthetic construct
QVQLVESGGGLVQAGGSLRLSCAVSGRALSDYNIGWFRQAPGKEREFVARKTWDGSSTRYDDSVKGRFTVSIEDAKNTAYLQMSSLKTEDTAVYYCAAGRYSRARGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 359 aa, synthetic construct
QVQLVESGGGLVQAGGSLRLSCAASGRAFGDYNIGWFRQAPGKEREFVARRTWDGSSTRYDDSVKGRFTVSIDNAKNTAYLQMNSLKTEDTAVYYCAAGRYSRVRGWEYDYWGQGTQVTVSSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLYCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 10 aa, synthetic construct
GFTFNTRSIV

SEQ ID NO: 211 - protein, 10 aa, synthetic construct
GTIFSGNAVG

SEQ ID NO: 212 - protein, 10 aa, synthetic construct
GPSLSNYAMG

SEQ ID NO: 213 - protein, 10 aa, synthetic construct
RSPMPIDTMA

SEQ ID NO: 214 - protein, 10 aa, synthetic construct
GPSLSHYAMG

SEQ ID NO: 215 - protein, 10 aa, synthetic construct
ESTFSINVMG

SEQ ID NO: 216 - protein, 10 aa, synthetic construct
GPSLSNFAMG

SEQ ID NO: 217 - protein, 10 aa, synthetic construct
GPSISNFAMG

SEQ ID NO: 218 - protein, 10 aa, synthetic construct
GSALSDYNIG

SEQ ID NO: 219 - protein, 10 aa, synthetic construct
GRSVTMGWLG

SEQ ID NO: 220 - protein, 15 aa, synthetic construct
SCISKVAGSTYYGDS

SEQ ID NO: 221 - protein, 14 aa, synthetic construct
SSITDYGDTDYAEA

SEQ ID NO: 222 - protein, 15 aa, synthetic construct
AGISWGGGRTSYTDS

SEQ ID NO: 223 - protein, 14 aa, synthetic construct
AIISRSGSTNYADS

SEQ ID NO: 224 - protein, 14 aa, synthetic construct
ATITSGDITNYADS

SEQ ID NO: 225 - protein, 15 aa, synthetic construct
AGISWGGGRTNYLDS

SEQ ID NO: 226 - protein, 15 aa, synthetic construct
AGISWGGGRTSCTDS

SEQ ID NO: 227 - protein, 15 aa, synthetic construct
ARKTWDGSSTKYDDS

SEQ ID NO: 228 - protein, 12 aa, synthetic construct
TWFNWGTYYANS

SEQ ID NO: 229 - protein, 19 aa, synthetic construct
AASFAPNCYGWSVAANMEY

SEQ ID NO: 230 - protein, 11 aa, synthetic construct
YVYQRGESEEY

SEQ ID NO: 231 - protein, 16 aa, synthetic construct
AGRYGRGRNVEYEYDY

SEQ ID NO: 232 - protein, 11 aa, synthetic construct
NAVGLWNGAEY

SEQ ID NO: 233 - protein, 14 aa, synthetic construct
NARGEYHSGNEYDY

SEQ ID NO: 234 - protein, 16 aa, synthetic construct
AGRYGRGRDVEYEYDY

SEQ ID NO: 235 - protein, 18 aa, synthetic construct
ASFTGYRPLINRLNDVSD

SEQ ID NO: 236 - protein, 5 aa, synthetic construct
GGGGS

SEQ ID NO: 237 - protein, 15 aa, synthetic construct
GGGGSGGGGSGGGGS

SEQ ID NO: 238-240 - skipped (3 entries)

SEQ ID NO: 241 - protein, 25 aa, synthetic construct
QACKAHHHHHHGAEFEQKLISEEDL

SEQ ID NO: 242 - protein, 25 aa, synthetic construct
GGGGSGGGGSGGGGSGGGGSGGGGS

SEQ ID NO: 243 - protein, 35 aa, synthetic construct
GGGGSGGGGSGGGGSGGGGSGGGGSGGGGSGGGGS

SEQ ID NO: 244 - skipped

SEQ ID NO: 245 - protein, 10 aa, synthetic construct
EQKLISEEDL

SEQ ID NO: 246-248 - skipped (3 entries)

SEQ ID NO: 249 - protein, 23 aa, synthetic construct
AAAHHHHHHGAEFEQKLISEEDL

SEQ ID NO: 250 - protein, 23 aa, synthetic construct
ACAHHHHHHGAEFEQKLISEEDL

SEQ ID NO: 251-253 - skipped (3 entries)

SEQ ID NO: 254 - protein, 9 aa, synthetic construct
AAAHHHHHH

SEQ ID NO: 255 - protein, 9 aa, synthetic construct
ACAHHHHHH

SEQ ID NO: 256-260 - skipped (5 entries)

SEQ ID NO: 261 - protein, 243 aa, synthetic construct
QVQLVQSGAEVKKPGSSVKVSCKASGYTFTRYTMHWVRQAPGQGLEWMGYINPSRGYTNYNQKFKDRVTITADKSTSTAYMELSSLRSEDTAVYYCARYYDDHYCLDYWGQGTMVTVSSVEGGSGGSGGSGGSGGVDDIQMTQSPSSLSASVGDRVTITCSASSSVSYMNWYQQKPGKAPKRLIYDTSKLASGVPSRFSGSGSGTEFTLTISSLQPEDFATYYCQQWSSNPFTFGQGTKVEIK

SEQ ID NO: 262 - protein, 243 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 263 - protein, 495 aa, synthetic construct
IIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPACTTDGGGGSHHHHHH

SEQ ID NO: 264 - protein, 495 aa, synthetic construct
VIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDGGGGSHHHHHH

SEQ ID NO: 265 - protein, 543 aa, synthetic construct
IIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDGGGGSCGGLTDTLQAETDQLEDEKSALQTEIANLLKEKEKLEFILAAHGGCGGHHHHHH

SEQ ID NO: 266 - protein, 543 aa, synthetic construct
IIPVEEENPDFWNRQAAEALGAAKKLQPAQTAAKNLIIFLGDGMGVSTVTAARILKGQKKDKLGPETFLAMDRFPYVALSKTYSVDKHVPDSGATATAYLCGVKGNFQTIGLSAAARFNQCNTTRGNEVISVMNRAKKAGKSVGVVTTTRVQHASPAGAYAHTVNRNWYSDADVPASARQEGCQDIATQLISNMDIDVILGGGRKYMFPMGTPDPEYPDDYSQGGTRLDGKNLVQEWLAKHQGARYVWNRTELLQASLDPSVTHLMGLFEPGDMKYEIHRDSTLDPSLMEMTEAALLLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDGETHAGEDVAVFARGPQAHLVHGVQEQTFIAHVMAFAACLEPYTACDLAPRAGTTDGGGGSCGGLTDTLQAETDQLEDEKSALQTEIANLLKEKEKLEFILAAHGGCGGHHHHHH

SEQ ID NO: 267 - protein, 543 aa, synthetic construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASGGGGSCGGLTDTLQAETDQLEDEKSALQTEIANLLKEKEKLEFILAAHGGCGGHHHHHH

SEQ ID NO: 268 - protein, 232 aa, synthetic construct
EPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 269 - protein, 232 aa, synthetic construct
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 270 - protein, 232 aa, synthetic construct
EPKSSDKTHTCPPCPAPELLGGPCVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLCLSPGK

SEQ ID NO: 271 - protein, 543 aa, synthetic construct
VIPVEEENPAFWNRKAAEALDAAKKLKPIQTSAKNLVILMGDGMGVSTVTATRILKGQQQGHLGPETQLAMDRFPHMALSKTYNTDKQIPDSAGTGTAFLCGVKTNMKVIGLSAAARFNQCNTTWGNEVVSVMHRAKKAGKSVGVVTTTSVQHASPAGTYAHTVNRGWYSDAQMPASALQDGCKDISTQLISNMDIDVILGGGRKFMFPKGTPDQEYPTDTKQAGTRLDGRNLVQEWLAKHQGARYVWNRSELIQASLNRSVTHLMGLFEPNDMKYEIHRDPAQDPSLAEMTEVAVRMLSRNPKGFYLFVEGGRIDHGHHETVAYRALTEAVMFDSAVDKADKLTSEQDTMILVTADHSHVFSFGGYTQRGASIFGLAPFKAEDGKSFTSILYGNGPGYKLHNGARADVTEEESSNPTYQQQAAVPLSSETHSGEDVAIFARGPQAHLVHGVQEQNYIAHVMAFAACLEPYTDCGLASPAGQSSGGGGSCGGLTDTLQAETDQLEDEKSALQTEIANLLKEKEKLEFILAAHGGCGGHHHHHH

SEQ ID NO: 272 - protein, 543 aa, synthetic construct
IIPVEEENPDFWNRQAAEALGAAKKLQPIQTAAKNLIIFLGDGMGVSTVTAARILKGQKEDKLGPETPLAMDHFPYVALSKTYSVDKHVPDSAATATAYLCGVKGNFQTIGLSAAARYNQCNTTRGNEVVSVMNRAKKAGKSVGVVTTTRVQHASPAGAYAHTVNRNWYSDANMPGSARREGCKDIATQLISNMDIDVILGGGRKYMFRMGAPDPEYPHDYSQDGTRMDGKNLVQEWLAKQQGARYVWNRTELMQASLDPSVTHLMGLFEPGDMKYEIHRDPTLDPSLMEMTEAALRLLSRNPRGFFLFVEGGRIDHGHHESRAYRALTEAVMFDDAIERAGQLTSEEDTLTLVTADHSHVFSFGAYPLRGSSIFGLAPGKAQDRKAYTALLYGNGPGYVLKDGARPDVTESESGSPEYRQQAAVPLDEETHAGEDVAVFARGPQAHLVHGVQEQSFIAHVMAFAACLEPYTACDLAPPAGTTDGGGGSCGGLTDTLQAETDQLEDEKSALQTEIANLLKEKEKLEFILAAHGGCGGHHHHHH

SEQ ID NO: 273 - protein, 543 aa, synthetic construct
IIPVEEENPDFWNRQAAEALGAAKKLQPIQTAAKNLIIFLGDGMGVSTVTAARILKGQKEEKLGPETPLAMDHFPYVALSKTYSVDKHVPDSAATATAYLCGVKGNFQTIGLSAAARYNQCNTTRGNEVVSVMNRAKKAGKSVGVVTTTRVQHASPAGAYAHTVNRNWYSDANMPGSARREGCKDIATQLISNMDIDVILGGGRKYMFRMGAPDPEYPHDYSQDGTRMDGKNLVQEWLAKHQGARYVWNRTELMQASLDPSVTHLMGLFEPGDMKYEIHRDPTLDPSLMEMTEAALRLLSRNPRGFFLFVEGGRIDHGHHESRAYRALTEAVMFDDAIERAGQLTSEEDTLTLVTADHSHVFSFGAYPLRGSSIFGLAPGKAQDRKAYTALLYGNGPGYVLKDGARPDVTESESGSPEYRQQAAVPLDEETHAGEDVAVFARGPQAHLVHGVQEQSFVAHVMAFAACLEPYTACDLAPPAGTTDGGGGSCGGLTDTLQAETDQLEDEKSALQTEIANLLKEKEKLEFILAAHGGCGGHHHHHH

SEQ ID NO: 274 - protein, 15 aa, synthetic construct
GCGGSGGGGSGGGGS

SEQ ID NO: 275 - protein, 535 aa, synthetic construct
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 276 - protein, 232 aa, synthetic construct; identical to SEQ ID NO: 198

(19) United States
(12) Patent Application Publication
(10) Pub. No.: US 2026/0216242 A1
(43) Pub. Date: Jul. 30, 2026

(54) ALPP-SPECIFIC VARIANT ANTIGEN BINDING MOLECULES

(71) Applicant: ALMAC DISCOVERY LIMITED, Craigavon (GB)

(72) Inventors: Graham John COTTON, Craigavon (GB); Paul Richard TRUMPER, Craigavon (GB); Estelle MCLEAN, Craigavon (GB); Mark WAPPETT, Craigavon (GB); Aaron CRANSTON, Craigavon (GB)

(21) Appl. No.: 19/141,146

(22) PCT Filed: Dec. 21, 2023
(86) PCT No.: PCT/EP2023/087391

§ 371 Date: Jun. 19, 2025

(30) Foreign Application Priority Data
Dec. 21, 2022 (GB) 2219467.4
Apr. 13, 2023 (GB) 2305455.4

(51) Int. Cl. A61K 35/17; A61K 40/11; A61K 40/31; A61K 40/42; A61K 47/68; A61P 35/00; C07K 14/705; C07K 16/28; C07K 16/40

(52) U.S. Cl. CPC A61K 35/17; A61K 40/11; A61K 40/31; A61K 40/4252; A61K 47/68031; A61K 47/6871; A61P 35/00; C07K 14/7051; C07K 16/2809; C07K 16/40; A61K 2239/13; C07K 2317/31; C07K 2317/34; C07K 2317/565; C07K 2317/567; C07K 2317/569; C07K 2317/77; C07K 2317/92; C07K 2317/94

(57) ABSTRACT

The present invention relates to alkaline phosphatase, placental type (ALPP) and/or alkaline phosphatase, germ cell type (ALPPL2) specific antigen binding molecules and associated fusion proteins and conjugates and methods of producing the same. In a further aspect, the present invention relates to conjugated single domain antibodies (VHH domains).

### FIELD OF INVENTION

The present invention relates to alkaline phosphatase, placental type (ALPP) and/or alkaline phosphatase, germ cell type (ALPPL2) specific antigen binding molecules and associated fusion proteins and conjugates and methods of producing the same. In a further aspect, the present invention relates to conjugated single domain antibodies (VHH domains).

### BACKGROUND

Alkaline phosphatases are a group of dimeric metalloenzymes found in a wide spread of organisms that catalyse the hydrolysis of phosphomonoesters (Le Du et al, J. Bio Chem. 2001, 276, 9158-9165). In humans there are four isozymes, placental (herein referred to as ALPP but also known as PLAP), germ cell type (herein referred to as ALPPL2, but also known as ALPG or GCAP), intestinal (ALPI) and tissue non-specific (herein referred to as ALPL, but also known as TNAP).

These four isozymes are all glycoproteins that are anchored to the cell surface via a C-terminal glycophosphatidylinositol post translational modification. ALPP, ALPPL2 and ALPI are all located on chromosome 2, whereas ALPL is located on chromosome 1. ALPP and ALPPL2 share high sequence homology at 98% and have a similar expression profile, being expressed in the placenta but largely absent in normal adult tissue. ALPI has relatively high homology to ALPP/ALPPL2 at around 87%. This isoform is widely expressed throughout the intestinal tract and has an important role in gut mucosal defence. The fourth isozyme, ALPL is widely expressed in many tissues including bone, liver and kidney. ALPL has a lower homology to ALPP/ALPPL2 at around 57%.

Upregulation of ALPP and ALPPL2 has been observed at the mRNA level and at the protein level in a broad range of cancers including ovarian cancer, endometrial cancer, cervical cancer, gastric cancer, oesophageal cancer, bladder cancer, lung cancer, pancreatic cancer and testicular cancer (Reiswich et al, J of Pathol. Clin. Res., 2021, 7, 577-589). High levels of ALPP/ALPPL2 have also been linked to poor prognosis in gastric and ovarian cancers (Orsaria et al, Cancer Biomarkers 2016, 17, 479-486; Liu et al, Human Pathol. 2019, 86, 49-56). Given its expression levels on cancer cells compared to its relative absence on normal tissue, ALPP/ALPPL2 is an attractive target for a cancer therapy.

Antibodies targeting ALPP or ALPPL2 have previously been described in the literature (Ravenni et al, MAbs, 2014, 6 (1), 86-94) and anti-ALPPL2 and anti-ALPP antibody drug conjugates (ADC) have been reported (Su et al, Cancer Res., 2020, 80, 4552-4564; WO2017095823; WO2022197890 (Seagen)). ALPP/ALPPL2 CAR-T therapies have also been described for use as an immunotherapy for treatment of colorectal and cervical cancer (Li et al, Front. Biosci. 2020, 25, 1765-1786; WO2019240934 (Promab); Yekehfallah et al, Biomolecules, 2022, 12, 1296).

Single domain antibodies (sdAbs) derived from camelid heavy chain only antibodies (HCAb) are small proteins consisting of a variable domain that can selectively bind an antigen with high specificity and affinity. These VHHs are playing an emergent role in numerous therapeutics and have several advantages over classical antibodies.

The small size of VHHs compared to a full length mAb (12-15 kDa compared to −150 kDa) allows for greater tissue and tumour penetration that can lead to greater efficacy. Another advantage of these small single domains is their relative simplicity. Unlike mAbs, VHH domains do not rely on a heavy chain-light chain pairing. The resulting modularity and ease of manufacturing makes VHH domains an attractive platform for engineering proteins and protein drug conjugates with improved therapeutic properties.

Conjugates of specific antigen binding molecules are described in PCT/EP2020/067210 filed on 19 Jun. 2020, hereby incorporated by reference in its entirety. PCT/EP2020/067210 describes anthracycline (PNU) derivatives suitable for use in drug conjugates. Specifically, derivatives of PNU159682 are provided, which lack the C14 carbon and attached hydroxyl functionality, and in which an ethylenediamino (EDA) group forms part of a linker region between the C13 carbonyl of PNU159682 and a maleimide group. Alternatively, the same molecules may be described with EDA-PNU as the “warhead” such that the EDA group is not considered part of the linker region. Where the linker comprises val-cit-PAB the maleimide group may be replaced with any reactive group suitable for a conjugation reaction. Such payloads are able to react with a free thiol group on another molecule. Where the free thiol is on a protein a protein-drug conjugate (PDC) may be formed.

The anthracycline derivative PNU-159682 has been described as a metabolite of nemorubicin (Quintieri et al. (2005) Clin. Cancer Res. 11, 1608-1617) and has been reported to exhibit extremely high potency for in vitro cell killing in the pico- to femtomolar range with one ovarian (A2780) and one breast cancer (MCF7) cell line (WO2012/073217 A1). Derivatives of PNU-159682 have also been described in WO2016/102679.

Conjugation of PNU-159682 derivatives to antibodies is described in WO2009/099741, WO2016/127081 and WO2016/102679, Yu et al, Clin. Cancer Res 2015, 21, 3298 and Stefan et al, Mol. Cancer. Ther., 2017, 16,879.

Auristatin E (AE) and monomethylauristatin E (MMAE) are synthetic analogues of the dolastatins, a special group of linear pseudopeptides originally isolated from marine sources, some of which have very potent cytotoxic activity against tumour cells. However, MMAE has the disadvantage of a comparatively high systemic toxicity. To improve the tumour selectivity MMAE is used in particular in conjunction with enzymatically cleavable valine citrulline linkers in the ADC setting for more targeted tumour therapy (see for example WO 2005/081711. After proteolytic cleavage, MMAE is preferably released intracellularly from corresponding ADCs. Monomethylauristatin F (MMAF) is an auristatin derivative having a C-terminal phenylalanine moiety. MMAF as well as various ester and amide derivatives thereof have been disclosed in WO 2005/081711. Further auristatin analogues with a C-terminal, amidically substituted phenylalanine unit are described in WO 01/18032. WO 02/088172 and WO 2007/008603 which claim MMAF analogues which relate to side-chain modifications of phenylalanine, and in WO 2007/008848 those in which the carboxyl group of the phenylalanine is modified. Auristatin conjugates linked via the C-terminus have been described in WO 2009/117531 and further conjugates are described in WO2013/087716.

Described herein are ALPP and/or ALPPL2-specific variant antigen binding molecules having advantageous properties and conjugates thereof to MMAE and derivatives of PNU-159682.

### SUMMARY OF INVENTION

The present invention generally relates to specific antigen binding molecules.

According to an aspect, the invention provides an alkaline phosphatase, placental type (ALPP) and/or alkaline phosphatase, germ cell type (ALPPL2) specific antigen binding molecule comprising an amino acid sequence represented by the formula (I):

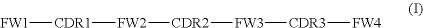

wherein
FW1 is a framework region;
CDR1 is a CDR sequence;
FW2 is a framework region;
CDR2 is a CDR sequence;
FW3 is a framework region;
CDR3 is a CDR sequence;
FW4 is a framework region.

According to a further aspect, the invention provides an alkaline phosphatase, placental type (ALPP) and/or alkaline phosphatase, germ cell type (ALPPL2) specific antigen binding molecule comprising an amino acid sequence represented by the formula (I):

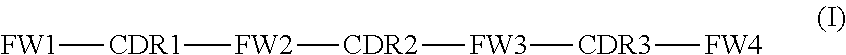

wherein

FW1 is a framework region;
CDR1 is a CDR sequence having an amino acid sequence selected from the group consisting of SEQ ID NOs: 11 to 15;
FW2 is a framework region;
CDR2 is a CDR sequence having an amino acid sequence selected from the group consisting of SEQ ID NOs: 6 to 10;
FW3 is a framework region;
CDR3 is a CDR sequence having an amino acid sequence selected from the group consisting of SEQ ID NOs: 1 to 5
FW4 is a framework region.

According to a further aspect, the invention provides a recombinant fusion protein comprising a specific antigen binding molecule as disclosed herein.

According to a further aspect, the invention provides a recombinant fusion protein comprising an antigen binding molecule comprising an amino acid sequence represented by the formula (I):

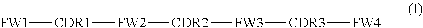

wherein
FW1 is a framework region;
CDR1 is a CDR sequence;
FW2 is a framework region;
CDR2 is a CDR sequence;
FW3 is a framework region;
CDR3 is a CDR sequence;
FW4 is a framework region.
or a functional variant thereof,
wherein the antigen binding molecule is fused to a fragment of an immunoglobulin Fc region, wherein the fragment of an immunoglobulin Fc region is engineered to dimerize with a second fragment of an immunoglobulin Fc region.

According to a further aspect, the invention provides a recombinant fusion protein dimer comprising
(a) a first recombinant fusion protein, wherein the first recombinant fusion protein is a recombinant fusion protein as disclosed herein, and
(b) a second recombinant fusion protein, wherein the second recombinant fusion protein comprises a second antigen binding molecule fused to a second fragment of an immunoglobulin Fc region engineered to dimerize with the first fragment of an immunoglobulin Fc region.

According to a further aspect, the invention provides an ALPP and/or ALPPL2-specific chimeric antigen receptor (CAR), comprising at least one ALPP and/or ALPPL2-specific antigen binding molecule as disclosed herein, fused or conjugated to at least one transmembrane region and at least one intracellular domain.

The present invention also provides a cell comprising a chimeric antigen receptor as disclosed herein, which cell is preferably an engineered T-cell.

In a further aspect of the invention, there is provided a nucleic acid sequence comprising a polynucleotide sequence that encodes a specific antigen binding molecule, recombinant fusion protein, recombinant fusion protein dimer or chimeric antigen receptor as disclosed herein.

There is also provided a vector comprising a nucleic acid sequence as disclosed herein and a host cell comprising such a nucleic acid.

A method for preparing a specific antigen binding molecule, recombinant fusion protein, recombinant fusion protein dimer or chimeric antigen receptor, as disclosed herein is provided, the method comprising cultivating or maintaining a host cell comprising the polynucleotide or vector described above under conditions such that said host cell produces the specific antigen binding molecule, recombinant fusion protein or chimeric antigen receptor, optionally further comprising isolating the specific antigen binding molecule, recombinant fusion protein, recombinant fusion protein dimer or chimeric antigen receptor.

In a further aspect of the invention, there is provided a pharmaceutical composition comprising the specific antigen binding molecule, fusion protein, recombinant fusion protein dimer or chimeric antigen receptor as disclosed herein. The pharmaceutical composition may contain a variety of pharmaceutically acceptable carriers. Pharmaceutical compositions of the invention may be for administration by any suitable method known in the art, including but not limited to intravenous, intramuscular, oral, intraperitoneal, or topical administration. In preferred embodiments, the pharmaceutical composition may be prepared in the form of a liquid, gel, powder, tablet, capsule, or foam.

The specific antigen binding molecule, recombinant fusion protein, recombinant fusion protein dimer or chimeric antigen receptor as disclosed herein may be for use in therapy. More specifically, the specific antigen binding molecule, recombinant fusion protein, recombinant fusion protein dimer or chimeric antigen receptor as disclosed herein may be for use in the treatment of cancer. Preferably, the cancer is an ALPP and/or ALPPL2-positive cancer type. More preferably, the cancer is selected from the group consisting of ovarian cancer, endometrial cancer, cervical cancer, gastric cancer, oesophageal cancer, bladder cancer, lung cancer, pancreatic cancer and testicular cancer.

Also provided herein is the use of a specific antigen binding molecule, recombinant fusion protein, recombinant fusion protein dimer or chimeric antigen receptor as disclosed herein in the manufacture of a medicament for the treatment of a disease in a patient in need thereof.

The specific antigen binding molecule, recombinant fusion protein, recombinant fusion protein dimer or chimeric antigen receptor as disclosed herein or pharmaceutical composition as disclosed herein may be administered in a single dose. As used herein “single dose” refers to a dosage regimen consisting of one dose. Alternatively, a multi-dose regimen may be used. Without being bound by theory, the advantages of the specific binding molecule, recombinant fusion protein, recombinant fusion protein dimer or chimeric antigen receptor or pharmaceutical composition as disclosed herein may be particularly apparent when administered in a single dose.

Furthermore, in accordance with the present invention there is provided a method of treatment of a disease in a patient in need of treatment comprising administration to said patient of a therapeutically effective dosage of a specific antigen binding molecule, recombinant fusion protein, recombinant fusion protein dimer or chimeric antigen receptor as disclosed herein or a pharmaceutical composition as disclosed herein.

Preferably, the cancer is an ALPP and/or ALPPL2-positive cancer type. More preferably, the cancer is selected from the group consisting of ovarian cancer, endometrial cancer, cervical cancer, gastric cancer, oesophageal cancer, bladder cancer, lung cancer, pancreatic cancer and testicular cancer.

Also provided herein is a method of assaying for the presence of a target analyte in a sample, comprising the addition of a detectably labelled specific antigen binding molecule as disclosed herein, or a recombinant fusion protein as disclosed herein, or a recombinant fusion protein dimer as disclosed herein, to the sample and detecting the binding of the molecule to the target analyte.

In addition, there is provided herein a method of imaging a site of disease in a subject, comprising administration of a detectably labelled specific antigen binding molecule as disclosed herein, or a detectably labelled recombinant fusion protein as disclosed herein, or a recombinant fusion protein dimer as disclosed herein.

There is also provided herein a method of diagnosis of a disease or medical condition in a subject comprising administration of a specific antigen binding molecule as disclosed herein, or a recombinant fusion protein as disclosed herein, or a recombinant fusion protein dimer as disclosed herein.

Also contemplated herein is an antibody, antibody fragment or antigen-binding molecule that competes for binding to ALPP and/or ALPPL2 with the ALPP and/or ALPPL2-specific antigen binding molecule as disclosed herein. The term “compete” when used in the context of antigen binding proteins (e.g., neutralizing antigen binding proteins or neutralizing antibodies) means competition between antigen binding proteins as determined by an assay in which the antigen binding protein (e.g., antibody or functional fragment thereof) under test prevents or inhibits specific binding of a the antigen binding molecule defined herein (e.g., specific antigen binding molecule of the first aspect) to a common antigen (e.g., ALPP and/or ALPPL2 in the case of the specific antigen binding molecules as disclosed herein).

Also described herein is a kit for diagnosing a subject suffering from cancer, or a pre-disposition thereto, or for providing a prognosis of the subject's condition, the kit comprising detection means for detecting the concentration of antigen present in a sample from a test subject, wherein the detection means comprises a ALPP and/or ALPPL2-specific antigen binding molecule as disclosed herein, a recombinant fusion protein as disclosed herein, or a recombinant fusion protein dimer as disclosed herein, a chimeric antigen receptor as disclosed herein or a nucleic acid sequence as disclosed herein, each being optionally derivatized, wherein presence of antigen in the sample suggests that the subject suffers from cancer. Preferably the antigen comprises ALPP and/or ALPPL2 protein, more preferably an extracellular domain thereof. More preferably, the kit is used to identify the presence or absence of ALPP and/or ALPPL2-positive cells in the sample, or determine the concentration thereof in the sample. The kit may also comprise a positive control and/or a negative control against which the assay is compared and/or a label which may be detected.

The present invention also provides a method for diagnosing a subject suffering from cancer, or a pre-disposition thereto, or for providing a prognosis of the subject's condition, the method comprising detecting the concentration of antigen present in a sample obtained from a subject, wherein the detection is achieved using a ALPP and/or ALPPL2-specific antigen binding molecule of the first or second aspect, a recombinant fusion protein of the third or fourth aspect, or a recombinant fusion protein dimer of the fifth aspect, a chimeric antigen receptor of the sixth aspect or a nucleic acid sequence of the seventh aspect, each being optionally derivatized, and wherein presence of antigen in the sample suggests that the subject suffers from cancer.

Also contemplated herein is a method of killing or inhibiting the growth of a cell expressing ALPP and/or ALPPL2 in vitro or in a patient, which method comprises administering to the cell a pharmaceutically effective amount or dose of (i) ALPP and/or ALPPL2-specific antigen binding molecule as disclosed herein, a recombinant fusion protein as disclosed herein, or a recombinant fusion protein dimer as disclosed herein, a nucleic acid sequence as disclosed herein, or the CAR or cell according as disclosed herein, or (ii) of a pharmaceutical composition as disclosed herein. Preferably, the cell expressing ALPP and/or ALPPL2 is a cancer cell. More preferably, the ALPP and/or ALPPL2 is human ALPP and/or ALPPL2.

According to a further aspect, the invention provides a specific antigen binding molecule comprising an amino acid sequence represented by the formula (II):

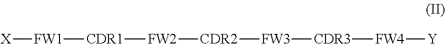

wherein
FW1-CDR1-FW2-CDR2-FW3-CDR3-FW4 is an ALPP and/or ALPPL2-specific antigen binding molecule as disclosed herein
X and Y are optional amino acid sequences
   wherein the specific antigen binding molecule is conjugated to a second moiety.

According to a further aspect, the invention provides a target-binding molecule-drug conjugate, comprising
(a) an ALPP and/or ALPPL2 specific antigen binding molecule, a recombinant fusion protein, or a recombinant fusion protein dimer as disclosed herein, and
(b) at least one cytotoxic or cytostatic agent.

According to a further aspect, (b) is an a MMAE derivative, wherein the target-binding molecule-drug conjugate has the structure of formula (VI):

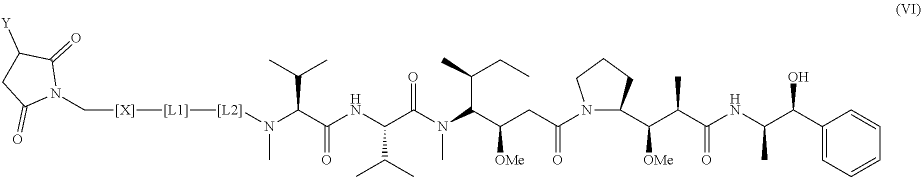

[X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, a dipeptide, a tripeptide, —(CH2)n-, —(CH2CH2O)n-, p-aminobenzyloxycarbonyl (PAB), Val-Cit-PAB, Val-Ala-PAB, Ala-Ala-Asn-PAB, Val-Ala, Asn-Ala, any amino acid, D-amino acids, Phe-Lys-PAB, and combinations thereof; and Y comprises an ALPP and/or ALPPL2 specific antigen binding molecule or recombinant fusion protein as disclosed herein.

According to a further aspect, the invention provides a target-binding molecule-drug conjugate, comprising
(a) an ALPP and/or ALPPL2 specific antigen binding molecule, a recombinant fusion protein, or a recombinant fusion protein dimer as disclosed herein, and
(b) an anthracycline (PNU) derivative, wherein the target-binding molecule-drug conjugate has the structure of formula (III):

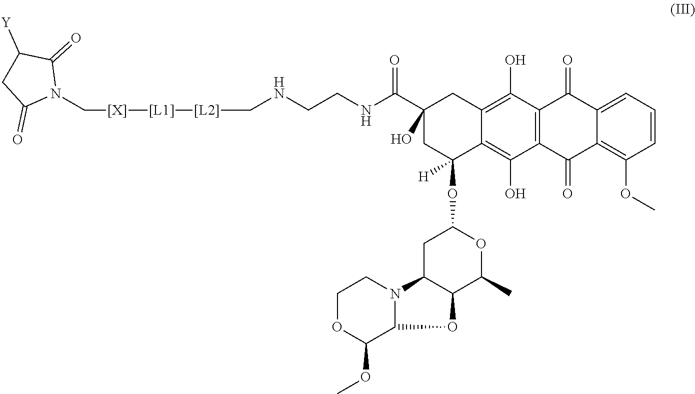

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof; and
Y comprises an ALPP and/or ALPPL2 specific antigen binding molecule, a recombinant fusion protein, or a recombinant fusion protein dimer as disclosed herein.

According to a further aspect, the invention provides a target-binding molecule-drug conjugate, comprising
(a) an ALPP and/or ALPPL2 specific antigen binding molecule, a recombinant fusion protein, or a recombinant fusion protein dimer as disclosed herein, and
(b) an anthracycline (PNU) derivative,
   wherein the target-binding molecule-drug conjugate has the structure of formula (IV):

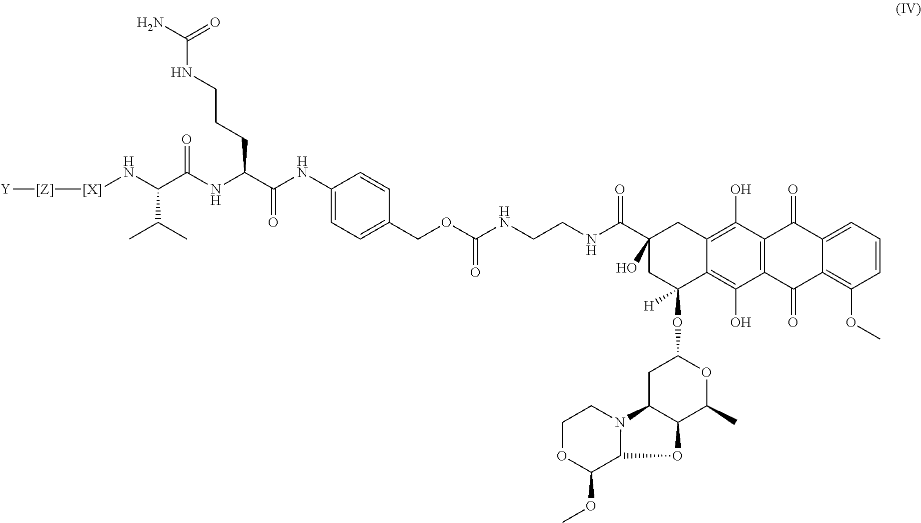

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[Z] is a linker derived from a reactive group used to conjugate the anthracycline (PNU) derivative and the target-binding molecule; and
Y comprises an ALPP and/or ALPPL2 specific antigen binding molecule, a recombinant fusion protein, or a recombinant fusion protein dimer as disclosed herein.

According to a further aspect, the invention provides an engineered ALPP and/or ALPPL2 monomer, wherein the engineered ALPP and/or ALPPL2 monomer is engineered to be capable of forming a covalent link with a further engineered ALPP and/or ALPPL2 monomer.

According to a further aspect, the invention provides an engineered ALPP and/or ALPPL2 homodimer, comprising a first and second engineered ALPP and/or ALPPL2 monomer as disclosed herein.

According to further aspect, the invention provides a method for generation of ALPP and/or ALPPL2 specific binding molecules comprising;
(a) immunising an animal with the engineered ALPP and/or ALPPL2 homodimer as disclosed herein,
(b) isolating Peripheral Blood Mononuclear Cells (PBMCs) from the animal,
(c) cloning into vectors RNA or cDNA sequences obtained from the PBMCs,
(d) transforming prokaryotic cells with the above vectors in conditions allowing the production of the specific binding molecules,
(e) selecting the specific binding molecules by subjecting them to antigen-affinity selection,
(f) recovering the specific binding molecules having the desired specificity.

According to a further aspect, the invention provides a bi-paratopic alkaline phosphatase, placental type (ALPP) and/or alkaline phosphatase, germ cell type (ALPPL2) specific antigen binding molecule.

According to a further aspect, the invention provides a bi-paratopic target-binding molecule-drug conjugate, comprising
(a) a bi-paratopic alkaline phosphatase, placental type (ALPP) and/or alkaline phosphatase, germ cell type (ALPPL2) specific antigen binding molecule, and
(b) at least one toxin, cytotoxic or cytostatic agent.

### DESCRIPTION OF FIGURES

FIG. 1: Cell surface binding of ALPP/ALPPL2 VHH domains (His₆ tag) to CHO cells stably transfected with ALPP, ALPPL2, ALPI or ALPL by flow cytometry.

FIG. 2: Human IgG sequences used in IgG Fc fusion proteins. Additionally, engineered hIgG1 Fc fusion proteins incorporate an engineered cysteine substitution in the hIgG1 Fc sequence, for example at position S239C or S442C or both positions (EU numbering) to enable site specific labelling.

FIG. 3: Cell surface binding of ALPP/ALPPL2 VHH-hFc fusion proteins to CHO cells stably transfected with ALPP, ALPPL2, ALPI or ALPL by flow cytometry.

FIG. 4: Structures of MC-vc-PAB-MMAE and of PNU-linker payloads MA-PEG-vc-PAB-EDA-PNU159682 and MA-PEG-va-EDA-PNU159682.

FIG. 5: In vitro potency of ALPP/ALPPL2 hFc PNU dug conjugates in killing CHO cells stably transfected with ALPP, ALPPL2, ALPI or ALPL.

FIG. 6: In vitro potency of ALPP/ALPPL2 hFc PNU and ALPP/ALPPL2 hFc MMAE drug conjugates in killing ALPP/ALPPL2 expressing cancer cell-lines.

FIG. 7: Cell surface binding of biparatopic ALPP/ALPPL2 VHH-hFc drug conjugates (vc PAB-MMAE) to CHO cells stably transfected with ALPP, ALPPL2, ALPI or ALPL by flow cytometry.

FIG. 8: In vitro potency of biparatopic ALPP/ALPPL2 hFc MMAE drug conjugates in killing CHO cells stably transfected with ALPP, ALPPL2, ALPI or ALPL.

FIG. 9: In vitro potency of biparatopic ALPP/ALPPL2 hFc MMAE drug conjugates and MMAE payload in killing ALPP/ALPPL2 expressing cancer cell-lines and for killing HeLa cancer cells, which express little to no ALPP/ALPPL2.

FIG. 10: In vitro potency of biparatopic ALPP/ALPPL2 hFc PNU drug conjugates in killing CHO cells stably transfected with ALPP, ALPPL2, ALPI or ALPL.

FIG. 11: In vitro potency of biparatopic ALPP/ALPPL2 hFc PNU drug conjugates in killing ALPP/ALPPL2 expressing cancer cell-lines.

FIG. 12: ALPP/ALPPL2 IHC staining of NCI-N87 (gastric cancer), HPAC (pancreatic adenocarcinoma), and Caov-3 (ovarian adenocarcinoma) mouse xenograft models.

FIG. 13: In vivo efficacy of biparatopic ALPP/ALPPL2 targeting VHH-hFc MMAE conjugates in the NCI-N87 gastric cancer cell-line derived xenograft model. Vehicle data plotted until the point when the first animal in the vehicle group reached humane tumour burden.

FIG. 14: In vivo efficacy of biparatopic ALPP/ALPPL2 targeting VHH-hFc MMAE conjugates in the HPAC pancreatic cancer cell-line derived xenograft model. Vehicle data plotted until the point when the first animal in the vehicle group reached humane tumour burden.

FIG. 15: Internalisation of monoparatopic ALPP/ALPPL2 targeting proteins.

FIG. 16: Internalisation of biparatopic ALPP/ALPPL2 targeting proteins & ALPP/ALPPL2 mAb hi2F3.

FIG. 17: in vitro potency of biparatopic vs monoparatopic ALPP/ALPPL2 targeting drug protein conjugates in killing ALPP/ALPPL2 expressing cancer cells (NSCLC cell-line NCI-H1651).

FIG. 18: in vitro potency of biparatopic ALPP/ALPPL2 targeting drug protein conjugates vs ALPP/ALPPL2 mAb h12F3 in killing CHO cells stably transfected with ALPP or ALPPL2.

FIG. 19: In vivo efficacy of biparatopic ALPP/ALPPL2 targeting VHH-hFc MMAE conjugates and the ALPP/ALPPL2 ADC h12F3-MMAE in the NCI-N87 gastric cancer cell-line derived xenograft model.

FIG. 20: In vivo efficacy of biparatopic ALPP/ALPPL2 targeting VHH-hFc-MMAE conjugates and the ALPP/ALPPL2 ADC h12F3-MMAE in the HPAC pancreatic cancer cell-line derived xenograft model.

FIG. 21: In vivo efficacy of biparatopic ALPP/ALPPL2 targeting VHH-hFc MMAE conjugates and the ALPP/ALPPL2 ADC h12F3-MMAE in the Caov-3 ovarian cancer cell-line derived xenograft model.

### DETAILED DESCRIPTION

The present invention generally relates to specific antigen binding molecules. Specifically, the invention provides single domain antibodies (VVHs) specific for ALPP and/or ALPPL2 and associated fusion proteins, chimeric antigen receptors, conjugates, and nucleic acids, as well as accompanying methods.

The ALPP and/or ALPPL2-specific VHH domains are described herein as ALPP and/or ALPPL2-specific antigen binding molecules.

Although antibodies to ALPP and/or ALPPL2 have been reported in the literature, the high sequence identity between ALPP, ALPPL2, ALPI and ALPL means generating high affinity ALPP-specific binding agents is not trivial. Additionally, the large size of antibodies compromises their ability to penetrate into solid tumours and renders regions of target proteins inaccessible due to steric factors, which can be particularly acute for cell-surface proteins where oligomerisation or receptor clustering is observed.

As a result there is a need in the art for improved anti-ALPP and/or ALPPL2 binding protein agents with different functional or physical characteristics or properties to antibodies and the development of therapeutics and diagnostic agents for malignancies associated with ALPP and/or ALPPL2 expression. The present invention provides such agents in the form of the ALPP and/or ALPPL2-specific antigen binding molecules described herein.

Without being bound by theory, the presently-described ALPP and/or ALPPL2-specific antigen binding molecules are thought to bind to both ALPP and ALPPL2, with the exception of D10 which preferentially binds ALPP. A number of variants, including A06, E06, E10 and F05 have been experimentally confirmed to bind to both ALPP and ALPPL2. Furthermore, the ALPP and/or ALPPL2 specific antigen binding molecules of the present invention may not bind to a number of related targets such as ALPI and ALPL. The variants disclosed herein bind to a number of different, non-competing epitopes. Therefore, a number of biparatopic ALPP and/or ALPPL2 specific antigen binding molecules have been developed.

Binding of ALPP and/or ALPPL2-specific antigen binding molecules of the invention to transfected CHO cell lines has been demonstrated. This confirms the potential for the use of such molecules in the treatment of cancers, specifically cancers which express ALPP and/or ALPPL2.

Various forms of the ALPP and/or ALPPL2-specific antigen binding molecules are described, including fusion proteins of several types. Fusion proteins including an immunoglobulin Fc region are described, as well as both homo and heterodimers. Fusion of proteins to an Fc domain can improve protein solubility and stability, markedly increase plasma half-life and improve overall therapeutic effectiveness. The present inventors have also created VHH molecules conjugated to a variety of moieties and payloads. The present invention therefore also provides chemically conjugated VHHs. More specifically, ALPP and/or ALPPL2-specific antigen binding molecules in several conjugated formats are provided.

According to an aspect, the invention provides an alkaline phosphatase, placental type (ALPP) and/or alkaline phosphatase, germ cell type (ALPPL2) specific antigen binding molecule comprising an amino acid sequence represented by the formula (I):

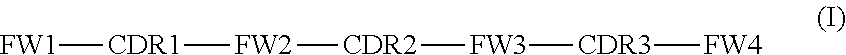

wherein
FW1 is a framework region;
CDR1 is a CDR sequence;
FW2 is a framework region;
CDR2 is a CDR sequence;
FW3 is a framework region;
CDR3 is a CDR sequence;
FW4 is a framework region.

According to a further aspect, the invention provides an alkaline phosphatase, placental type (ALPP) and/or alkaline phosphatase, germ cell type (ALPPL2) specific antigen binding molecule comprising an amino acid sequence represented by the formula (I):

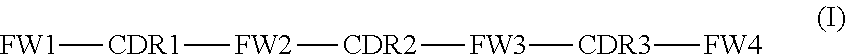

wherein
FW1 is a framework region;
CDR1 is a CDR sequence having an amino acid sequence selected from the group consisting of SEQ ID NOs: 11 to 15;
FW2 is a framework region;
CDR2 is a CDR sequence having an amino acid sequence selected from the group consisting of SEQ ID NOs: 6 to 10;
FW3 is a framework region;
CDR3 is a CDR sequence having an amino acid sequence selected from the group consisting of SEQ ID NOs: 1 to 5;
FW4 is a framework region.

In one embodiment of the ALPP and/or ALPPL2-specific antigen binding molecule:
CDR1 is a CDR sequence having an amino acid sequence selected from the group consisting of GRTFLSMG (SEQ ID NO: 11), GSIFSVNTMG (SEQ ID NO: 12), RRSFSEYAMG (SEQ ID NO: 13), GRALSDYNIG (SEQ ID NO: 14) and GRAFGDYNIG (SEQ ID NO: 15),
CDR2 is a CDR sequence having an amino acid sequence selected from the group consisting of AAISWTGGSTGYADS (SEQ ID NO: 6), AVATSGGTIHYADS (SEQ ID NO: 7), AAIGWSGVIEYADS (SEQ ID NO: 8), ARKTWDGSSTRYDDS (SEQ ID NO: 9) and ARRTWDGSSTRYDDS (SEQ ID NO: 10) and/or
CDR3 is a CDR sequence having an amino acid sequence selected from the group consisting of AARGDVYYGGDDATRYKY (SEQ ID NO: 1), NLVVPYGMIAGDHDY (SEQ ID NO: 2), AAGLGIRVVSQTPAY (SEQ ID NO: 3), AAGRYSRARGWEYDY (SEQ ID NO: 4) and AAGRYSRVRGWEYDY (SEQ ID NO: 5).

In one embodiment the ALPP and/or ALPPL2-specific antigen binding molecule comprises an amino acid sequence selected from the group consisting of:

(SEQ ID NO: 16)
QVQLVESGGGLVQAGASLTLSCAASGFTFNTRSIVWFRQAPGKEREAVSCISKVAGSTYYGDSVQGR
FSSSTDNAKNTVSLQMNSLKPEDTAVYTCAASFAPNCYGWSVAANMEYWGEGTMVTVSS
referred to herein as A05;
(SEQ ID NO: 17)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSS
referred to herein as A06;
(SEQ ID NO: 18)
QVQLVESGGGLVQAGESLRLACAASGTIFSGNAVGWYRQAPEKQREWVSSITDYGDTDYAEAVKGR
FTISRDNAKNTVYLQMNSLKPEDTAVYYCYVYQRGESEEYWGQGTKVTVSS
referred to herein as A08;
(SEQ ID NO: 19)
QVHLVECGGGSVQAGGFLRLSCAASGPSLSNYAMGRFRQAPGKEREFVAGISWGGGRTSYTDSVK
GRFTISRDNAKNTVYLQMNNLEPEDTGVYYCAGRYGRGRNVEYEYDYWGQGTKVTVSS
referred to herein as A12;
(SEQ ID NO: 20)
QVQLVESGGGLVQAGGSLRLSCEVTRSPMPIDTMAWYRQAPGRQRELVAIISRSGSTNYADSVKGR
FTISRNDTKSTMYKSTMYLKMNTLELEDTGVYYCNAVGLWNGAEYWGQGTQVTVSS
referred to herein as B01;
(SEQ ID NO: 21)
QVQLVESGGGSVQAGGSLRLSCAASGPSLSHYAMGWFRQAPGKEREFVAGISWGGGRTSYTDSVK
GRFTISRDNAKNTVYLQMNNLKPEDTGVYYCAGRYGRGRNVEYEYDYWGQGTQVTVSS
referred to herein as B09;
(SEQ ID NO: 22)
QVHLVESGGGSVQAGGSLRLSCAASGPSLSNYAMGWFRQAPGKEREFVAGISWGGGRTSYTDSVK
GRFTISRDNAKNTVYLQMNNLKPEDTGVYYCAGRYGRGRNVEYEYDYWGQGTQVTVSS
referred to herein as B11;
(SEQ ID NO: 23)
QVQLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSS
referred to herein as C05;
(SEQ ID NO: 24)
QVQLVESGGGLVQPGGSLRVSCAASGPSLSNYAMGWFRQAPGKEREFVAGISWGGGRTSYTDSVK
GRFTISRDNAKNTVYLQMNNLKPEDTGVYYCAGRYGRGRNVEYEYDYWGQGTQVTVSS
referred to herein as C06;
(SEQ ID NO: 25)
QVQLVESGGGLVQAGESLRLACAASGTIFSGNAVGWYRQAPEKQREWVSSITDYGDTDYAEAVKGR
FTISRDNAKNTVYLQMNSLKPEDTAVYYCYVYQRGESEEYWGQGTQVTVSS
referred to herein as C07;
(SEQ ID NO: 26)
QVQLVESGGGLVQPGGSLRLSCAASESTFSINVMGWYRQAPGKQRELVATITSGDITNYADSVKGRF
TISRDNAENTVYLRTNSLKPEDTAVYYCNARGEYHSGNEYDYWGQGTQVTVSS
referred to herein as D04;
(SEQ ID NO: 27)
QVHLVESGGGSVQAGGSLRLSCAASGPSLSNYAMGWFRQAPGKEREFVAGISWGGGRTSYTDSVK
GRFTISRDNAKNTVYLQMNNLKPEDTGVYYCAGRYGRGRNVEYEYDYWGQGTKVTVSS
referred to herein as D08;
(SEQ ID NO: 28)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSS
referred to herein as D10;
(SEQ ID NO: 29)
QVQLVESGGGSVQAGGSLRLSCAASGPSLSNYAMGWFRQAPGKEREFVAGISWGGGRTSYTDSVK
GRFTISRDNAKNTVYLQMNNLKPEDTGVYYCAGRYGRGRNVEYEYDYWGQGTMVTVSS
referred to herein as D11;
(SEQ ID NO: 30)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSS
referred to herein as E06;
(SEQ ID NO: 31)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSS
referred to herein as E10;
(SEQ ID NO: 32)
QVQLVESGGGSVQAGGSLRLSCAASGPSLSNFAMGWFRQPPGKEREFVAGISWGGGRTSYTDSVK
GRFTISRDNAKNTVYLQMNNLKPEDTGVYYCAGRYGRGRNVEYEYDYWGQGTQVTVSS
referred to herein as E11;
(SEQ ID NO: 33)
QVQLVESGGGSVQAGGSLRLSCAASGPSISNFAMGWFRQAPGKEREFVAGISWGGGRTNYLDSVK
GRFTISRDNAKNMVHLQMNSLKPEDTTVYYCAGRYGRGRDVEYEYDYWGQGTQVTVSS
referred to herein as E12;
(SEQ ID NO: 34)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSS
referred to herein as F05;
(SEQ ID NO: 35)
QVQLVESGGGSVQAGGSLRLSCAASGPSLSNYAMGWFRQAPGKEREFVAGISWGGGRTSYTDSVK
GRFTISRDNAKNTVYLQMNNLKPEDTGVYYCAGRYGRGRNVEYEYDYWGQGTQVTVSS
referred to herein as F08;
(SEQ ID NO: 36)
QVQLVESGGGSVQAGGSLRLSCAASGPSLSNYAMGWFRQAPGKEREFVAGISWGGGRTSCTDSVK
GRFTISRDNAKNTVYLQMNNLKPEDTGVYYCAGRYGRGRNVEYEYDYWGQGTQVTVSS
referred to herein as F09;
(SEQ ID NO: 37)
QVQLVESGGGLVEAGGSLRLSCAVSGSALSDYNIGWFRQAPGKEREFVARKTWDGSSTKYDDSVK
GRFTVSIDNAKNTAYLQMNSLKTEDTAVYYCAAGRYSRVRGWEYDYWGQRTQVTVSS
referred to herein as F12;
(SEQ ID NO: 38)
QVQLVESGGGTAQAGGSLRLACAASGRSVTMGWLGWVPRAERDDLEFITWFNWGTYYANSVNGRF
TISTDNADNTVYPQMNRLKPENPAVYYCASFTGYRPLINRLNDVSDCGHGALVAVSS
referred to herein as G03;
(SEQ ID NO: 39)
QVQLVESGGGSVQTGGSLRLSCAASGPSLSNYAMGWFRQAPGKEREFVAGISWGGGRTNYLDSVK
GRFTISRDNAKNTMYLQMNSLKPEDTAVYYCAGRYGRGRDVEYEYDYWGQGTQVTVSS
referred to herein as H09;
(SEQ ID NO: 40)
QVQLVESGGGSVQTGGSLRLSCAASGPSLSNYAMGWFRQAPGKEREFVAGISWGGGRTNYLDSVK
GRFTISRDNAKNTMYLQMNSLKPEDAAVYYCAGRYGRGRDVEYEYDYWGQGTQVTVSS
referred to herein as H10;

or a functional variant having CDR1, CDR2 and CDR3 sequences according to any thereof and having FW1, FW2, FW3 and FW4 sequences having a combined sequence identity of at least 45%, at least 55%, at least 75%, at least 85%, at least 90%, at least 95% or at least 99% to the combined FW1, FW2, FW3 and FW4 sequences of any thereof.

In a particularly preferred embodiment, the ALPP and/or ALPPL2-specific antigen binding molecule may comprise an amino acid sequence according to:

(SEQ ID NO: 17)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSS.

The ALPP and/or ALPPL2-specific antigen binding molecule may comprise an amino acid sequence according to SEQ ID NO: 17 (“A06”) or a functional variant thereof having CDR1, CDR2 and CDR3 sequences according to SEQ ID NO: 17 and having combined FW1, FW2, FW3 and FW4 sequences having a combined sequence identity of at least 45%, at least 55%, at least 75%, at least 85%, at least 90%, at least 95% or at least 99% to the combined FW1, FW2, FW3 and FW4 sequences of SEQ ID NO: 17

A06-hFc exhibits high affinity binding to ALPP and ALPPL2 and A06-hFc drug conjugates show potent killing of ALPP/APPL2 expressing cells.

In a particularly preferred embodiment, the ALPP and/or ALPPL2-specific antigen binding molecule may comprise an amino acid sequence according to:

(SEQ ID NO: 28)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSS.

The ALPP and/or ALPPL2-specific antigen binding molecule may comprise an amino acid sequence according to SEQ ID NO: 28 (“D10”) or a functional variant thereof having CDR1, CDR2 and CDR3 sequences according to SEQ ID NO: 28 and having combined FW1, FW2, FW3 and FW4 sequences having a combined sequence identity of at least 45%, at least 55%, at least 75%, at least 85%, at least 90%, at least 95% or at least 99% to the combined FW1, FW2, FW3 and FW4 sequences of SEQ ID NO: 28.

D10-hFc exhibits high affinity binding to ALPP and D10-hFc drug conjugates show potent killing of ALPP expressing cells.

In a particularly preferred embodiment, the ALPP and/or ALPPL2-specific antigen binding molecule may comprise an amino acid sequence according to:

(SEQ ID NO: 30)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSS.

The ALPP and/or ALPPL2-specific antigen binding molecule may comprise an amino acid sequence according to SEQ ID NO: 30 (“E06”) or a functional variant thereof having CDR1, CDR2 and CDR3 sequences according to SEQ ID NO: 30 and having combined FW1, FW2, FW3 and FW4 sequences having a combined sequence identity of at least 45%, at least 55%, at least 75%, at least 85%, at least 90%, at least 95% or at least 99% to the combined FW1, FW2, FW3 and FW4 sequences of SEQ ID NO: 30.

E06-hFc exhibits high affinity binding to ALPP and ALPPL2 and E06-hFc drug conjugates show potent killing of ALPP expressing cells.

In a particularly preferred embodiment, the ALPP and/or ALPPL2-specific antigen binding molecule may comprise an amino acid sequence according to:

(SEQ ID NO: 31)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSS.

The ALPP and/or ALPPL2-specific antigen binding molecule may comprise an amino acid sequence according to SEQ ID NO: 31 (“E10”) or a functional variant thereof having CDR1, CDR2 and CDR3 sequences according to SEQ ID NO: 31 and having combined FW1, FW2, FW3 and FW4 sequences having a combined sequence identity of at least 45%, at least 55%, at least 75%, at least 85%, at least 90%, at least 95% or at least 99% to the combined FW1, FW2, FW3 and FW4 sequences of SEQ ID NO: 31.

E10-hFc exhibits high affinity binding to ALPP and ALPPL2 and E10-hFc drug conjugates show potent killing of ALPP/ALPPL2 expressing cells.

In a particularly preferred embodiment, the ALPP and/or ALPPL2-specific antigen binding molecule may comprise an amino acid sequence according to:

(SEQ ID NO: 34)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSS.

The ALPP and/or ALPPL2-specific antigen binding molecule may comprise an amino acid sequence according to SEQ ID NO: 34 (“F05”) or a functional variant thereof having CDR1, CDR2 and CDR3 sequences according to SEQ ID NO: 34 and having combined FW1, FW2, FW3 and FW4 sequences having a combined sequence identity of at least 45%, at least 55%, at least 75%, at least 85%, at least 90%, at least 95% or at least 99% to the combined FW1, FW2, FW3 and FW4 sequences of SEQ ID NO: 34

F05-hFc exhibits high affinity binding to ALPP and ALPPL2 and F05-hFc drug conjugates show potent killing of ALPP/ALPPL2 expressing cells.

The ALPP and/or ALPPL2-specific antigen binding molecule may comprise the CDR sequences of a clone set out in Table 1 below. In preferred embodiments, the ALPP and/or ALPPL2 specific antigen binding molecule has the combined sequence of any of the clones set out in Table 1 below.

TABLE 1
ALPP and/or ALPPL2 specific antigen binding molecules

| Clone | | | | | | | |
|---|---|---|---|---|---|---|---|
| Name | FW1 | CDR1 | FW2 | CDR2 | FW3 | CDR3 | FW4 |
| A05 | QVQLVESGGGLVQ | GFTFNTRSIV | WFRQAPGKEREAV | SCISKVAGSTYYGDS | VQGRFSSSTDNAKN | AASFAPNCYGWSV | WGEGTMVTVSS |
| | AGASLTLSCAAS | (SEQ ID NO: 210) | (SEQ ID NO: 64) | (SEQ ID NO: 220) | TVSLQMNSLKPEDT | AANMEY | (SEQ ID NO: 81) |
| | (SEQ ID NO: 41) | | | | AVYTC | (SEQ ID NO: 229) | |
| | | | | | (SEQ ID NO: 66) | | |
| A06 | QVHLVESGGGLVQA | GRTFLSMG | WFRQAPGKEREFV | AAISWTGGSTGYAD | VKGRFTISRESDKET | AARGDVYYGGDDA | WGQGTQVTVSS |
| | GGSLRLSCAAS | (SEQ ID NO: 11) | (SEQ ID NO: 64) | S | MYLQMNSLKPEDT | TRYKY | (SEQ ID NO: 84) |
| | (SEQ ID NO: 53) | | | (SEQ ID NO: 6) | AVYYC | (SEQ ID NO: 1) | |
| | | | | | (SEQ ID NO: 76) | | |
| A08 | QVQLVESGGGLVQ | GTIFSGNAVG | WYRQAPEKQREWV | SSITDYGDTDYAEA | VKGRFTISRDNAKN | YVYQRGESEEY | WGQGTKVTVSS |
| | AGESLRLACAAS | (SEQ ID NO: 211) | (SEQ ID NO: 58) | (SEQ ID NO: 221) | TVYLQMNSLKPEDT | (SEQ ID NO: 230) | (SEQ ID NO: 85) |
| | (SEQ ID NO: 42) | | | | AVYYC | | |
| | | | | | (SEQ ID NO: 77) | | |
| A12 | QVHLVECGGGSVQ | GPSLSNYAMG | RFRQAPGKEREFV | AGISWGGGRTSYTD | VKGRFTISRDNAKN | AGRYGRGRNVEYEY | WGQGTKVTVSS |
| | AGGFLRLSCAAS | (SEQ ID NO: 212) | (SEQ ID NO: 59) | S | TVYLQMNNLEPEDT | DY | (SEQ ID NO: 85) |
| | (SEQ ID NO: 43) | | | (SEQ ID NO: 222) | GVYYC | (SEQ ID NO: 231) | |
| | | | | | (SEQ ID NO: 68) | | |
| B01 | QVQLVESGGGLVQ | RSPMPIDTMA | WYRQAPGRQRELV | AIISRSGSTNYADS | VKGRFTISRNDTKST | NAVGLWNGAEY | WGQGTQVTVSS |
| | AGGSLRLSCEVT | (SEQ ID NO: 213) | (SEQ ID NO: 60) | (SEQ ID NO: 223) | MYKSTMYLKMNTL | (SEQ ID NO: 232) | (SEQ ID NO: 84) |
| | (SEQ ID NO: 44) | | | | ELEDTGVYYC | | |
| | | | | | (SEQ ID NO: 69) | | |
| B09 | QVQLVESGGGSVQ | GPSLSHYAMG | WFRQAPGKEREFV | AGISWGGGRTSYTD | VKGRFTISRDNAKN | AGRYGRGRNVEYEY | WGQGTQVTVSS |
| | AGGSLRLSCAAS | (SEQ ID NO: 214) | (SEQ ID NO: 64) | S | TVYLQMNNLKPEDT | DY | (SEQ ID NO: 84) |
| | (SEQ ID NO: 45) | | | (SEQ ID NO: 222) | GVYYC | (SEQ ID NO: 231) | |
| | | | | | (SEQ ID NO: 70) | | |
| B11 | QVHLVESGGGSVQ | GPSLSNYAMG | WFRQAPGKEREFV | AGISWGGGRTSYTD | VKGRFTISRDNAKN | AGRYGRGRNVEYEY | WGQGTQVTVSS |
| | AGGSLRLSCAAS | (SEQ ID NO: 212) | (SEQ ID NO: 64) | S | TVYLQMNNLKPEDT | DY | (SEQ ID NO: 84) |
| | (SEQ ID NO: 46) | | | (SEQ ID NO: 222) | GVYYC | (SEQ ID NO: 231) | |
| | | | | | (SEQ ID NO: 70) | | |
| C05 | QVQLVESGGGLVQ | RRSFSEYAMG | WFRQAPGKEREFV | AAIGWSGVIEYADS | VKGRFTISRDNAKDT | AAGLGIRVVSQTPA | WGQGTQVTVSS |
| | AGGSLRLSCAAS | (SEQ ID NO: 13) | (SEQ ID NO: 64) | (SEQ ID NO: 8) | VYLQMNSLKPEDTA | Y | (SEQ ID NO: 84) |
| | (SEQ ID NO: 47) | | | | VYYC | (SEQ ID NO: 3) | |
| | | | | | (SEQ ID NO: 78) | | |
| C06 | QVQLVESGGGLVQP | GPSLSNYAMG | WFRQAPGKEREFV | AGISWGGGRTSYTD | VKGRFTISRDNAKN | AGRYGRGRNVEYEY | WGQGTQVTVSS |
| | GGSLRVSCAAS | (SEQ ID NO: 212) | (SEQ ID NO: 64) | S | TVYLQMNNLKPEDT | DY | (SEQ ID NO: 84) |
| | (SEQ ID NO: 48) | | | (SEQ ID NO: 222) | GVYYC | (SEQ ID NO: 231) | |
| | | | | | (SEQ ID NO: 70) | | |
| C07 | QVQLVESGGGLVQ | GTIFSGNAVG | WYRQAPEKQREWV | SSITDYGDTDYAEA | VKGRFTISRDNAKN | YVYQRGESEEY | WGQGTQVTVSS |
| | AGESLRLACAAS | (SEQ ID NO: 211) | (SEQ ID NO: 58) | (SEQ ID NO: 221) | TVYLQMNSLKPEDT | (SEQ ID NO: 230) | (SEQ ID NO: 84) |
| | (SEQ ID NO: 42) | | | | AVYYC | | |
| | | | | | (SEQ ID NO: 77) | | |
| D04 | QVQLVESGGGLVQP | ESTFSINVMG | WYRQAPGKQRELV | ATITSGDITNYADS | VKGRFTISRDNAENT | NARGEYHSGNEYDY | WGQGTQVTVSS |
| | GGSLRLSCAAS | (SEQ ID NO: 215) | (SEQ ID NO: 61) | (SEQ ID NO: 224) | VYLRTNSLKPEDTAV | (SEQ ID NO: 233) | (SEQ ID NO: 84) |
| | (SEQ ID NO: 49) | | | | YYC | | |
| | | | | | (SEQ ID NO: 71) | | |
| D08 | QVHLVESGGGSVQ | GPSLSNYAMG | WFRQAPGKEREFV | AGISWGGGRTSYTD | VKGRFTISRDNAKN | AGRYGRGRNVEYEY | WGQGTKVTVSS |
| | AGGSLRLSCAAS | (SEQ ID NO: 212) | (SEQ ID NO: 64) | S | TVYLQMNNLKPEDT | DY | (SEQ ID NO: 85) |
| | (SEQ ID NO: 46) | | | (SEQ ID NO: 222) | GVYYC | (SEQ ID NO: 231) | |
| | | | | | (SEQ ID NO: 70) | | |
| D10 | QVQLVESGGGLVQP | GSIFSVNTMG | WYRQAPGKQRDLV | AVATSGGTIHYADS | VKGRFTISRDNAKN | NLVVPYGMIAGDH | WGQGTKVTVSS |
| | GGSLRLSCAAS | (SEQ ID NO: 12) | (SEQ ID NO: 65) | (SEQ ID NO: 7) | TVYLQMNSLKPEDT | DY | (SEQ ID NO: 85) |
| | (SEQ ID NO: 49) | | | | AVYYC | (SEQ ID NO: 2) | |
| | | | | | (SEQ ID NO: 77) | | |
| D11 | QVQLVESGGGSVQ | GPSLSNYAMG | WFRQAPGKEREFV | AGISWGGGRTSYTD | VKGRFTISRDNAKN | AGRYGRGRNVEYEY | WGQGTMVTVSS |
| | AGGSLRLSCAAS | (SEQ ID NO: 212) | (SEQ ID NO: 64) | S | TVYLQMNNLKPEDT | DY | (SEQ ID NO: 86) |
| | (SEQ ID NO: 45) | | | (SEQ ID NO: 222) | GVYYC | (SEQ ID NO: 231) | |
| | | | | | (SEQ ID NO: 70) | | |
| E06 | QVHLVESGGGLVQA | RRSFSEYAMG | WFRQAPGKEREFV | AAIGWSGVIEYADS | VKGRFTISRDNAKDT | AAGLGIRVVSQTPA | WGQGTQVTVSS |
| | GGSLRLSCAAS | (SEQ ID NO: 13) | (SEQ ID NO: 64) | (SEQ ID NO: 8) | VYLQMNSLKPEDTA | Y | (SEQ ID NO: 84) |
| | (SEQ ID NO: 53) | | | | VYYC | (SEQ ID NO: 3) | |
| | | | | | (SEQ ID NO: 78) | | |
| E10 | QVQLVESGGGLVQ | GRALSDYNIG | WFRQAPGKEREFV | ARKTWDGSSTRYDD | VKGRFTVSIEDAKNT | AAGRYSRARGWEY | WGQGTQVTVSS |
| | AGGSLRLSCAVS | (SEQ ID NO: 14) | (SEQ ID NO: 64) | S | AYLQMSSLKTEDTA | DY | (SEQ ID NO: 84) |
| | (SEQ ID NO: 55) | | | (SEQ ID NO: 9) | VYYC | (SEQ ID NO: 4) | |
| | | | | | (SEQ ID NO: 79) | | |
| E11 | QVQLVESGGGSVQ | GPSLSNFAMG | WFRQPPGKEREFV | AGISWGGGRTSYTD | VKGRFTISRDNAKN | AGRYGRGRNVEYEY | WGQGTQVTVSS |
| | AGGSLRLSCAAS | (SEQ ID NO: 216) | (SEQ ID NO: 62) | S | TVYLQMNNLKPEDT | DY | (SEQ ID NO: 84) |
| | (SEQ ID NO: 45) | | | (SEQ ID NO: 222) | GVYYC | (SEQ ID NO: 231) | |
| | | | | | (SEQ ID NO: 70) | | |
| E12 | QVQLVESGGGSVQ | GPSISNFAMG | WFRQAPGKEREFV | AGISWGGGRTNYLD | VKGRFTISRDNAKN | AGRYGRGRDVEYEY | WGQGTQVTVSS |
| | AGGSLRLSCAAS | (SEQ ID NO: 217) | (SEQ ID NO: 64) | S | MVHLQMNSLKPED | DY | (SEQ ID NO: 84) |
| | (SEQ ID NO: 45) | | | (SEQ ID NO: 225) | TTVYYC | (SEQ ID NO: 234) | |
| | | | | | (SEQ ID NO: 72) | | |
| F05 | QVQLVESGGGLVQ | GRAFGDYNIG | WFRQAPGKEREFV | ARRTWDGSSTRYD | VKGRFTVSIDNAKN | AAGRYSRVRGWEY | WGQGTQVTVSS |
| | AGGSLRLSCAAS | (SEQ ID NO: 15) | (SEQ ID NO: 64) | DS | TAYLQMNSLKTEDT | DY | (SEQ ID NO: 84) |
| | (SEQ ID NO: 56) | | | (SEQ ID NO: 10) | AVYYC | (SEQ ID NO: 5) | |
| | | | | | (SEQ ID NO: 80) | | |
| F08 | QVQLVESGGGSVQ | GPSLSNYAMG | WFRQAPGKEREFV | AGISWGGGRTSYTD | VKGRFTISRDNAKN | AGRYGRGRNVEYEY | WGQGTQVTVSS |
| | AGGSLRLSCAAS | (SEQ ID NO: 212) | (SEQ ID NO: 64) | S | TVYLQMNNLKPEDT | DY | (SEQ ID NO: 84) |
| | (SEQ ID NO: 45) | | | (SEQ ID NO: 222) | GVYYC | (SEQ ID NO: 231) | |
| | | | | | (SEQ ID NO: 70) | | |
| F09 | QVQLVESGGGSVQ | GPSLSNYAMG | WFRQAPGKEREFV | AGISWGGGRTSCTD | VKGRFTISRDNAKN | AGRYGRGRNVEYEY | WGQGTQVTVSS |
| | AGGSLRLSCAAS | (SEQ ID NO: 212) | (SEQ ID NO: 64) | S | TVYLQMNNLKPEDT | DY | (SEQ ID NO: 84) |
| | (SEQ ID NO: 45) | | | (SEQ ID NO: 226) | GVYYC | (SEQ ID NO: 231) | |
| | | | | | (SEQ ID NO: 70) | | |
| F12 | QVQLVESGGGLVEA | GSALSDYNIG | WFRQAPGKEREFV | ARKTWDGSSTKYDD | VKGRFTVSIDNAKN | AAGRYSRVRGWEY | WGQRTQVTVSS |
| | GGSLRLSCAVS | (SEQ ID NO: 218) | (SEQ ID NO: 64) | S | TAYLQMNSLKTEDT | DY | (SEQ ID NO: 82) |
| | (SEQ ID NO: 50) | | | (SEQ ID NO: 227) | AVYYC | (SEQ ID NO: 5) | |
| | | | | | (SEQ ID NO: 80) | | |
| G03 | QVQLVESGGGTAQ | GRSVTMGWLG | WVPRAERDDLEFI | TWFNWGTYYANS | VNGRFTISTDNADN | ASFTGYRPLINRLND | CGHGALVAVSS |
| | AGGSLRLACAAS | (SEQ ID NO: 219) | (SEQ ID NO: 63) | (SEQ ID NO: 228) | TVYPQMNRLKPENP | VSD | (SEQ ID NO: 83) |
| | (SEQ ID NO: 51) | | | | AVYYC | (SEQ ID NO: 235) | |
| | | | | | (SEQ ID NO: 73) | | |
| H09 | QVQLVESGGGSVQT | GPSLSNYAMG | WFRQAPGKEREFV | AGISWGGGRTNYLD | VKGRFTISRDNAKN | AGRYGRGRDVEYEY | WGQGTQVTVSS |
| | GGSLRLSCAAS | | | S | TMYLQMNSLKPEDT | DY | (SEQ ID NO: 84) |
| | (SEQ ID NO: 52) | (SEQ ID NO: 212) | (SEQ ID NO: 64) | (SEQ ID NO: 225) | AVYYC | (SEQ ID NO: 234) | |
| | | | | | (SEQ ID NO: 74) | | |
| H10 | QVQLVESGGGSVQT | GPSLSNYAMG | WFRQAPGKEREFV | AGISWGGGRTNYLD | VKGRFTISRDNAKN | AGRYGRGRDVEYEY | WGQGTQVTVSS |
| | GGSLRLSCAAS | | | S | TMYLQMNSLKPED | DY | (SEQ ID NO: 84) |
| | | | | | AAVYYC | (SEQ ID NO: 234) | |
| | (SEQ ID NO: 52) | (SEQ ID NO: 212) | (SEQ ID NO: 64) | (SEQ ID NO: 225) | (SEQ ID NO: 75) | | |

All possible combinations and permutations of the framework regions, complementarity determining regions and hypervariable regions listed herein are explicitly contemplated herein.

Sequence identity referenced in relation to the molecules of the invention may be judged at the level of individual CDRs or FWs, combined CDRs or FWs, or it may be judged over the length of the entire molecule. The CDR and FW sequences described may also be longer or shorter, whether that be by addition or deletion of amino acids at the N- or C-terminal ends of the sequence or by insertion or deletion of amino acids with a sequence.

Framework region FW1 is preferably from 20 to 30 amino acids in length, more preferably from 22 to 28 amino acids in length, still more preferably from 24 to 26 amino acids in length. In certain preferred embodiments, FW1 is 24 amino acids in length. In certain preferred embodiments, FW1 is 25 amino acids in length.

In alternative definitions, CDR region CDR1 is preferably from 8 to 10 amino acids in length. In certain preferred embodiments, CDR1 is 10 amino acids in length. In other preferred embodiments, CDR1 is 8 amino acids in length.

Framework region FW2 is preferably from 10 to 15 amino acids in length, more preferably from 12 to 14 amino acids in length. In certain preferred embodiments, FW2 is 13 amino acids in length.

In alternative definitions, CDR region CDR2 is preferably from 10 to 20 amino acids in length, more preferably from 12 to 15 amino acids in length. In certain preferred embodiments, CDR2 is 12 amino acids in length. In other preferred embodiments, CDR2 is 14 amino acids in length. In other preferred embodiments, CDR2 is 15 amino acids in length.

Framework region FW3 is preferably from 30 to 40 amino acids in length, more preferably from 33 to 38 amino acids in length. In certain preferred embodiments, FW3 is 33 amino acids in length. In other preferred embodiments, FW3 is 38 amino acids in length.

In alternative definitions, CDR region CDR3 is preferably from 10 to 20 amino acids in length, more preferably from 11 to 19 amino acids in length. In certain preferred embodiments, CDR3 is 11 amino acids in length. In other preferred embodiments, CDR3 is 14 amino acids in length. In other preferred embodiments, CDR3 is 15 amino acids in length. In other preferred embodiments, CDR3 is 16 amino acids in length. In other preferred embodiments, CDR3 is 18 amino acids in length. In other preferred embodiments, CDR3 is 19 amino acids in length.

Framework region FW4 is preferably from 8 to 12 amino acids in length, more preferably from 10 to 11 amino acids in length. In certain preferred embodiments, FW4 is 11 amino acids in length.

In one embodiment of the ALPP and/or ALPPL2-specific antigen binding molecule:
FW1 is a framework region of 24 to 25 amino acids;
FW2 is a framework region of 13 amino acids;
FW3 is a framework region of 33 amino acids; and/or
FW4 is a framework region of 11 amino acids.

In one embodiment of the ALPP and/or ALPPL2-specific antigen binding molecule:
FW1 has an amino acid sequence selected from the group consisting of: QVHLVESGGGLVQAGGSLRLSCAAS (SEQ ID NO: 53), VQLVESGGGLVQPGGSLRLSCAAS (SEQ ID NO: 54), QVQLVESGGGLVQAGGSLRLSCAVS (SEQ ID NO: 55), and QVQLVESGGGLVQAGGSLRLSCAAS (SEQ ID NO: 56) or a functional variant of any thereof with a sequence identity of at least 45%, at least 55%, at least 75%, at least 85%, at least 90%, at least 95% or at least 99%;
FW2 has an amino acid sequence selected from the group consisting of: WFRQAPGKEREFV (SEQ ID NO: 64) and WYRQAPGKQRDLV (SEQ ID NO: 65), or a functional variant of any thereof with a sequence identity of at least 45%, at least 55%, at least 75%, at least 85%, at least 90%, at least 95% or at least 99%;
FW3 has an amino acid sequence selected from the group consisting of:
VKGRFTISRESDKETMYLQMNSLKPEDTAVYYC (SEQ ID NO: 76),
VKGRFTISRDNAKNTVYLQMNSLKPEDTAVYYC (SEQ ID NO: 77),
VKGRFTISRDNAKDTVYLQMNSLKPEDTAVYYC (SEQ ID NO: 78),
VKGRFTVSIEDAKNTAYLQMSSLKTEDTAVYYC (SEQ ID NO: 79) and
VKGRFTVSIDNAKNTAYLQMNSLKTEDTAVYYC (SEQ ID NO: 80), or a functional variant of any thereof with a sequence identity of at least 45%, at least 55%, at least 75%, at least 85%, at least 90%, at least 95% or at least 99%;
and/or
FW4 has an amino acid sequence selected from the group consisting of: WGQGTQVTVSS (SEQ ID NO: 84) and WGQGTKVTVSS (SEQ ID NO: 85) or a functional variant of any thereof with a sequence identity of at least 45%, at least 55%, at least 75%, at least 85%, at least 90%, at least 95% or at least 99%.

The ALPP and/or ALPPL2-specific antigen binding molecule of the present invention may be humanized. The ALPP and/or ALPPL2-specific antigen binding molecule of the present invention may be de-immunized.

It will be appreciated by the skilled person that the humanised ALPP and/or ALPPL2-specific antigen binding molecules described herein may be further humanised, for instance by substituting FW region amino acids with corresponding amino acids from human VH or VL sequences.

The ALPP and/or ALPPL2-specific antigen binding molecule of the present invention may also be conjugated to a detectable label, dye, toxin, drug, pro-drug, radionuclide or biologically active molecule.

Preferably, the ALPP and/or ALPPL2-specific antigen binding molecule binds to ALPP and ALPPL2. More preferably, the ALPP-specific antigen binding molecule does not bind to both ALPI and ALPL.

Preferably, the ALPP and/or ALPPL2-specific antigen binding molecule selectively interacts with ALPP protein with an affinity constant of approximately 0.001 to 50 nM, preferably 0.1 to 30 nM, even more preferably 0.1 to 10 nM. An affinity constant may be measured by Bio-layer interferometry (BLI). For monomers the interaction is 1:1. For the VHH-hFc format the inventors have used two approaches. One where the ALPP is immobilized and thus a bi-valent VHH-hFc binds with an apparent K_{D} as the avidity effect comes into play. The other approach is in a 1:1 format whereby the VHH-hFc is immobilized and ALPP is flowed across the surface thus giving the K_{D} for ‘true’ 1:1 binding. Typically, where used herein affinity constants refer to those measured by Bio-layer interferometry (BLI) using the 1:1 binding format. By this method, for example, E06 and E10 are within the 0.1-10 nM range.

Furthermore, the ALPP and/or ALPPL2-specific antigen binding molecule is preferably capable of mediating killing of ALPP and/or ALPPL2-expressing tumour cells or is capable of inhibiting cancer cell proliferation.

The ALPP and/or ALPPL2-specific antigen binding molecule may also be capable of being endocytosed upon binding to ALPP and/or ALPPL2. In other embodiments, the ALPP and/or ALPPL2-specific antigen binding molecule may not be endocytosed upon binding to ALPP and/or ALPPL2.

In a further aspect of the present invention, there is provided a recombinant fusion protein comprising a specific antigen binding molecule as disclosed herein. Preferably, in the recombinant fusion protein, the specific antigen binding molecule is fused to one or more biologically active proteins. The specific antigen binding molecule may be fused to one or more biologically active proteins via one or more linker domains. Preferred linkers include but are not limited to [G₄S]ₓ, where x is 1, 2, 3, 4, 5, or 6. Particular preferred linkers are G₄S (SEQ ID NO: 236), referred to herein as “short linker” and [G₄S]₃ (SEQ ID NO: 237) referred to herein as “long linker”. These linkers may be particularly useful when recombinant fusion proteins are expressed in different expression systems that differ in glycosylation patterns, such as CHO and insect, and those that do not glycosylate expressed proteins (e.g. E. coli). Any recombinant fusion protein sequence disclosed herein comprising a [G₄S]₃ linker may alternatively possess any other linker sequence disclosed herein.

It will also be appreciated that the fusion proteins of the invention can be constructed in any order, i.e., with the ALPP and/or ALPPL2-specific antigen binding molecule at the N-terminus, C-terminus, or at neither terminus (e.g. in the middle of a longer amino acid sequence).

Preferred biologically active proteins include, but are not limited to an immunoglobulin, an immunoglobulin Fc region, a fragment of an immunoglobulin Fc region, an Fc heavy chain, a CH2 region, a CH3 region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, or a scaffold protein (affibodies, centyrins, darpins etc.). A particularly preferred biologically active protein is an immunoglobulin Fc region. Other preferred fusion proteins include VHH-VHH and VHH-VHH-VHH.

In one embodiment, the at least one biologically active protein is an immunoglobulin Fc region.

Therefore, the recombinant fusion protein may comprise a sequence according to SEQ ID NO: 87 to SEQ ID NO: 91.

A06-hFc (long linker)
(SEQ ID NO: 87)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
D10-hFc (long linker)
(SEQ ID NO: 88)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPGK
E06-hFc (long linker)
(SEQ ID NO: 89)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
E10-hFc (long linker)
(SEQ ID NO: 90)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK
F05-hFc (long linker)
(SEQ ID NO: 91)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK

SEQ ID NOs 87 to 91 each comprise a (G₄S)₃ linker. Also, explicitly contemplated herein are the corresponding sequences wherein the (G₄S)₃ linker is replaced with a (G₄)₁ linker. For instance, the recombinant fusion protein may comprise one or more of the following SEQ ID NOs 92 to 96.

A06-hFc (short linker)
(SEQ ID NO: 92)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
D10-hFc (short linker)
(SEQ ID NO: 93)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVF
SCSVMHEALHNHYTQKSLSLSPGK
E06-hFc (short linker)
(SEQ ID NO: 94)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVF
SCSVMHEALHNHYTQKSLSLSPGK
E10-hFc (short linker)
(SEQ ID NO: 95)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQG
NVFSCSVMHEALHNHYTQKSLSLSPGK
F05-hFc (short linker)
(SEQ ID NO: 96)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQG
NVFSCSVMHEALHNHYTQKSLSLSPGK

In a further embodiment, at least one biologically active protein is an immunoglobulin Fc region further modified to comprise a Cys (C) mutation (i.e. a mutation of an Fc amino acid to a cysteine residue).

The C mutation may be at position S239 (EU numbering). Therefore, the recombinant fusion protein may comprise a sequence according to SEQ ID NO: 97, or SEQ ID NO: 101.

A06-hFc (long linker) S239C
(SEQ ID NO: 97)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPCVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK
D10-hFc (long linker) S239C
(SEQ ID NO: 98)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPCVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKLTV
DKSRWQQGNVFSCSVMHEALHNHYTQKSLSLSPGK
E06-hFc (long linker) S239C
(SEQ ID NO: 99)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPCVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSKLTVD
KSRWQQGNVFSCSVMHEALHNHYTQKSLSLSPGK
E10-hFc (long linker) S239C
(SEQ ID NO: 100)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK
F05-hFc (long linker) S239C
(SEQ ID NO: 101)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLSLSPGK

SEQ ID NOs 102 to 106 each comprise a (G₄S)₃ linker. Also, explicitly contemplated herein are the corresponding sequences wherein the (G₄S)₃ linker is replaced with a (G₄S)₁ linker. For instance, the recombinant fusion protein may comprise one or more of the following SEQ ID Nos 102 to 106.

A06-hFc (short linker) S239C
(SEQ ID NO: 102)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPCVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQ
GNVFSCSVMHEALHNHYTQKSLSLSPGK
D10-hFc (short linker) S239C
(SEQ ID NO: 103)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVF
SCSVMHEALHNHYTQKSLSLSPGK
E06-hFc (short linker) S239C
(SEQ ID NO: 104)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVF
SCSVMHEALHNHYTQKSLSLSPGK
E10-hFc (short linker) S239C
(SEQ ID NO: 105)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQG
NVFSCSVMHEALHNHYTQKSLSLSPGK
F05-hFc (short linker) S239C
(SEQ ID NO: 106)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQG
NVFSCSVMHEALHNHYTQKSLSLSPGK

The C mutation may be at position S442 (EU numbering). Therefore, the recombinant fusion protein may comprise a sequence according to SEQ ID NO: 107 to 111.

A06-hFc (long linker) S442C
(SEQ ID NO: 107)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLCLSPGK
D10-hFc (long linker) S442C
(SEQ ID NO: 108)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKLTV
DKSRWQQGNVFSCSVMHEALHNHYTQKSLCLSPGK
E06-hFc (long linker) S442C
(SEQ ID NO: 109)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSKLTVD
KSRWQQGNVFSCSVMHEALHNHYTQKSLCLSPGK
E10-hFc (long linker) S442C
(SEQ ID NO: 110)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLCLSPGK
F05-hFc (long linker) S442C
(SEQ ID NO: 111)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLCLSPGK

SEQ ID NOs 107 to 111 each comprise a (G₄S)₃ linker. Also, explicitly contemplated herein are the corresponding sequences wherein the (G₄S)₃ linker is replaced with a (G₄)₁ linker. For instance, the recombinant fusion protein may comprise one or more of the following SEQ ID Nos 112 to 116.

A06-hFc (short linker) S442C
(SEQ ID NO: 112)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQ
GNVFSCSVMHEALHNHYTQKSLCLSPGK
D10-hFc (short linker) S442C
(SEQ ID NO: 113)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVF
SCSVMHEALHNHYTQKSLCLSPGK
E06-hFc (short linker) S442C
(SEQ ID NO: 114)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVF
SCSVMHEALHNHYTQKSLCLSPGK
E10-hFc (short linker) S442C
(SEQ ID NO: 115)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQG
NVFSCSVMHEALHNHYTQKSLCLSPGK
F05-hFc (short linker) S442C
(SEQ ID NO: 116)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQG
NVFSCSVMHEALHNHYTQKSLCLSPGK

The C mutation may be at both position S239 and S442 (EU numbering). Therefore, the recombinant fusion protein may comprise a sequence according to SEQ ID NO: 117 to 121.

A06-hFc (long linker) S239C & S442C
(SEQ ID NO: 117)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPCVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLYS
KLTVDKSRWQQGNVFSCSVMHEALHNHYTQKSLCLSPGK
D10-hFc (long linker) S239C & S442
(SEQ ID NO: 118)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPCVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLYSKLTV
DKSRWQQGNVFSCSVMHEALHNHYTQKSLCLSPGK
E06-hFc (long linker) S239C & S442C
(SEQ ID NO: 119)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPCVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLYSKLTVD
KSRWQQGNVFSCSVMHEALHNHYTQKSLCLSPGK
E10-hFc (long linker) S239C & S442C
(SEQ ID NO: 120)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLCLSPGK
F05-hFc (long linker) S239C & S442C
(SEQ ID NO: 121)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLYSKL
TVDKSRWQQGNVFSCSVMHEALHNHYTQKSLCLSPGK

SEQ ID NOs 117 to 121 each comprise a (G₄S)₃ linker. Also, explicitly contemplated herein are the corresponding sequences wherein the (G₄S)₃ linker is replaced with a (G₄S)₁ linker. For instance, the recombinant fusion protein may comprise one or more of the following SEQ ID NOs 122 to 126.

A06-hFc (short linker) S239C & S442C
(SEQ ID NO: 122)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPCVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLYSKLTVDKSRWQQ
GNVFSCSVMHEALHNHYTQKSLCLSPGK
D10-hFc (short linker) S239C & S442C
(SEQ ID NO: 123)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVF
SCSVMHEALHNHYTQKSLCLSPGK
E06-hFc (short linker) S239C & S442C
(SEQ ID NO: 124)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLYSKLTVDKSRWQQGNVF
SCSVMHEALHNHYTQKSLCLSPGK
E10-hFc (short linker) S239C & S442C
(SEQ ID NO: 125)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQG
NVFSCSVMHEALHNHYTQKSLCLSPGK
F05-hFc (short linker) S239C & S442C
(SEQ ID NO: 126)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLYSKLTVDKSRWQQG
NVFSCSVMHEALHNHYTQKSLCLSPGK

In one embodiment, the at least one biologically active protein is a fragment of an immunoglobulin Fc region selected from the group consisting of an Fc heavy chain, a CH2 region and a CH3 region.

In one embodiment, the fragment of an immunoglobulin Fc region is an Fc heavy chain.

In one embodiment, the fragment of an immunoglobulin Fc region is engineered to dimerize with a second fragment of an immunoglobulin Fc region.

As used herein, an immunoglobulin Fc region that is “engineered to dimerise” may comprise at least one amino acid substitution. Typically, the at least one amino acid substitution promotes and/or makes more energetically favourable, an interaction and/or association with a second fragment of an immunoglobulin Fc region, which thus promotes dimerization and/or makes dimerization more energetically favourable. Such recombinant fusion proteins may have particular utility in the preparation of bi-specific and/or bi-paratopic binders.

Methods for generating Fc based bi-specific and/or bi-paratopic binders, through pairing of two distinct Fc heavy chains that are engineered to dimerize, are known in the art. These methods enable an Fc region to be assembled from two different heavy chains, each fused to a target binding domain or sequence with different binding characteristics. The target binding domains or sequences can be directed to different targets to generate multi-specific binders and/or to different regions or epitopes on the same target to generate bi-paratopic binding proteins. Multiple binding domains or sequences can be fused to the Fc sequences to create multi-specific or multi-paratopic binders or both multi-specific multi-paratopic binders within the same protein. Methods to generate these asymmetric bispecific and/or bi-paratopic binders through heterodimerisation of two different Fc heavy chains, or fragments thereof, include but are not limited to: Knobs-into-holes (Y-T), Knobs-into-holes (CW-CSAV), CH3 charge pair, Fab-arm exchange, SEED technology, BEAT technology, HA-TF, ZW1 approach, Biclonic approach, EW-RVT and Triomab See for example, Brinkman & Kontermann, (2017) mAbs, 9:2, 182-212; Klein et al (2012) mAbs 4:6, 653-663; Wang et al (2019) Antibodies, 8, 43; and Dietrich et al (2020) BBA-Proteins and Proteomics 1868 140250; each of which is incorporated herein by reference in its entirety.

In one embodiment, the fragment of an immunoglobulin Fc region is engineered to dimerize with the second fragment of an immunoglobulin Fc region by a method selected from the group consisting of knobs-into-holes (Y-T), knobs-into-holes (CW-CSAV), CH3 charge pairing, Fab-arm exchange, SEED technology, BEAT technology, HA-TF, ZW1 approach, Biclonic approach, EW-RVT and Triomab.

In one embodiment, one or more residues of the fragment of the immunoglobulin Fc region comprises one or more amino acid substitution suitable for heterodimerization with a second fragment of an immunoglobulin Fc region comprising one or more corresponding amino acid substitution.

In one embodiment, the one or more amino acid substitution is selected from the group consisting of T366Y, Y407T, S354C, T366W, Y349C, T366S, L368A and Y407V.

In one embodiment, the one or more amino acid substitution is selected from the group consisting of T366Y and Y407T.

Any part of the fusion protein of the invention may be engineered to enable conjugation. In a preferred example, where an immunoglobulin Fc region is used, it may be engineered to include a cysteine residue as a conjugation site. Preferred introduced cysteine residues include, but are not limited to, S252C and S473C (Kabat numbering), which correspond to S239C and S442C in EU numbering, respectively. In some embodiments, any of the fusion proteins disclosed herein may comprise the S239C point mutation. In some embodiments, any of the fusion proteins disclosed herein may comprise the S442C point mutation. In some embodiments, any of the fusion proteins disclosed herein may comprise both S239C and S442C point mutations. It is explicitly contemplated herein that sequence of any of the fusion proteins disclosed herein may be modified to include an S239C and/or S442C point mutation. In addition, cysteine residues maybe incorporated into linker regions, including but not limited to linkers between VHH and the hFc in VHH-hFc and hFc-VHH fusion proteins. Cysteine residues may also be added to the C-terminus of the Fc region either directly or as part of a C-terminal tag.

In accordance with an aspect, recombinant fusions comprising multiple VHH domains are provided.

Accordingly, the recombinant fusions of the invention may be dimers, trimers or higher order multimers of VHHs. In such recombinant fusions, the specificity of each VHH may be the same or different. Recombinant fusions of the invention include, but are not limited to, bi-specific or tri-specific molecules in which each VHH domain binds to a different antigen, or to different epitopes on a single antigen (bi-paratopic binders). The term “bi-paratopic” as used herein is intended to encompass molecules that bind to multiple epitopes on a given antigen. Molecules that bind three or more epitopes on a given antigen are also contemplated herein and where the term “bi-paratopic” is used, it should be understood that the potential for tri-paratopic or multi-paratopic molecules is also encompassed.

Also in accordance with an aspect, recombinant fusions are provided which include a ALPP and/or ALPPL2-specific antigen binding molecule as disclosed herein and a second ALPP and/or ALPPL2 binding molecule, optionally a second ALPP and/or ALPPL2 binding molecule as disclosed herein. Recombinant fusions are also provided which include a ALPP and/or ALPPL2-specific antigen binding molecule as disclosed herein and a peptide sequence, protein domain or protein that can extend the serum half-life of the ALPP and/ALPPL2 specific antigen binding molecule, such as HSA binding domains or fusions with Fc proteins.

Examples of bi-paratopic and multivalent fusion proteins include, but are not limited to:
A06-D10
A06-E06
A06-E10
A06-F05
D10-E06
D10-E10
D10-F05
E06-E10
E06-F05
E10-F05

### Wherein:

A06 is
(SEQ ID NO: 17)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSS
D10 is
(SEQ ID NO: 28)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSS
E06 is
(SEQ ID NO: 30)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSS
E10 is
(SEQ ID NO: 31)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSS
F05 is
(SEQ ID NO: 34)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSS

and
where (−) corresponds to a linker as defined herein. In one embodiment, the linker may be a [G₄S]x linker, such as G₄S (SEQ ID NO: 236) and [G₄S]₃ (SEQ ID NO: 237). Optionally, the linker may comprise a Cys residue. One example of a Cys containing linker is GCGGS[G₄S]₂ (SEQ ID NO: 274).

Recombinant bi-paratopic fusion protein dimers can also be made by fusing any recombinant fusion protein disclosed herein, in particular the ALPP and/or ALPPL2 binders disclosed herein, onto one arm of an Fc fusion and by fusing binders to a different ALPP and/or ALPPL2 epitope onto the other.

In certain embodiments, the specific binding molecules or recombinant fusions of the invention may be expressed with N- or C-terminal tags to assist with purification. Examples include but are not limited to His₆ and/or Myc. In addition, the N- or C-terminal tag may be further engineered to include additional cysteine residues to serve as conjugation points. It will therefore be appreciated that reference to specific binding molecules or recombinant fusions in all aspects of the invention is also intended to encompass such molecules with a variety of N- or C-terminal tags, which tags may also include additional cysteines for conjugation.

Additional recombinant fusions are listed below. It will be appreciated that not every combination of linker and VHH or fusion partner is listed below. However, all such combinations are expressly encompassed by the present invention.

### Biparatopic Dimers

A06-D10
A06-E06
A06-E10
A06-F05
D10-E06
D10-E10
D10-F05
E06-E10
E06-F05
E10-F05
D10-A06
E06-A06
E10-A06
F05-A06
E06-D10
E10-D10
F05-D10
E10-E06
F05-E06
F05-E10

Where the linkers between the VHH domains are preferentially, but not limited to (G₄S)₅ (SEQ ID NO: 242), (G₄S)₃ (SEQ ID NO: 237), (G₄S)₇ (SEQ ID NO: 243), and wherein different combinations of different linkers can be combined within the same construct. Linkers may contain a single cysteine residue or multiple cysteine residues to facilitate site-selective bioconjugation of payloads to the proteins, in this linker, using thiol mediated chemical coupling strategies. Cys-containing linkers include but are not limited to GCGGS[G₄S]₂ (SEQ ID NO: 274)

Whereby, additional C-terminal (or N-terminal) tag sequences may or may not be present.

C-terminal tags include, but are not limited to, tags that contain poly-Histidine sequences to facilitate purification (such as His₆), contain c-Myc sequences (such as EQKLISEEDL (SEQ ID NO: 245)) to enable detection and/or contain Cysteine residues to enable labelling and bioconjugation using thiol reactive payloads and probes and combinations thereof. Preferential C-terminal tags include but are not limited to:

(SEQ ID NO: 249)
AAAHHHHHHGAEFEQKLISEEDL
(SEQ ID NO: 250)
ACAHHHHHHGAEFEQKLISEEDL
(SEQ ID NO: 254)
AAAHHHHHH
(SEQ ID NO: 255)
ACAHHHHHH

ACA (SEQ ID NO: 259)

### Wherein:

A06 is
(SEQ ID NO: 17)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSS
D10 is
(SEQ ID NO: 28)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSS
E06 is
(SEQ ID NO: 30)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSS
E10 is
(SEQ ID NO: 31)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSS
F05 is
(SEQ ID NO: 34)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSS

As stated above, all combinations of VHH and linker are expressly encompassed herein. Humanised derivatives of the VHHs are also encompassed herein.

Also in accordance with an aspect, recombinant fusions are provided which include an ALPP and/or ALPPL2-specific antigen binding molecule as disclosed herein and a recombinant toxin. Examples of recombinant toxins include but are not limited to Pseudomonas exotoxin PE38 and diphtheria toxin.

Also in accordance with an aspect, recombinant fusions are provided which include a ALPP and/or ALPPL2-specific antigen binding molecule as disclosed herein and a recombinant CD3 binding protein. Examples of recombinant ALPP and/or ALPPL2 and CD3 binding agents include but are not limited to:
A06-CD3
D10-CD3
E06-CD3
E10-CD3
F05-CD3
CD3-A06
CD3-D10
CD3-E06
CD3-E10
CD3-F05

### Wherein:

A06 is
(SEQ ID NO: 17)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSS
D10 is
(SEQ ID NO: 28)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSS
E06 is
(SEQ ID NO: 30)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSS
E10 is
(SEQ ID NO: 31)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSS
F05 is
(SEQ ID NO: 34)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSS

Any CD3 binding sequence, and variants thereof, known in the art can be substituted in above. For example:

UCL OKT3 sequence (WO2019008379)
(SEQ ID NO: 261)
QVQLVQSGAEVKKPGSSVKVSCKASGYTFTRYTMHWVRQAPGQGLEWMGYINPSRGYTNYNQKFK
DRVTITADKSTSTAYMELSSLRSEDTAVYYCARYYDDHYCLDYWGQGTMVTVSSVEGGSGGSGGSG
GSGGVDDIQMTQSPSSLSASVGDRVTITCSASSSVSYMNWYQQKPGKAPKRLIYDTSKLASGVPSRF
SGSGSGTEFTLTISSLQPEDFATYYCQQWSSNPFTFGQGTKVEIK
Harpoon ID20 (WO2016187594)
(SEQ ID NO: 262)
DIKLQQSGAELARPGASVKMSCKTSGYTFTRYTMHWVKQRPGQGLEWIGYINPSRGYTNYNQKFKD
KATLTTDKSSSTAYMQLSSLTSEDSAVYYCARYYDDHYCLDYWGQGTTLTVSSVEGGSGGSGGSGG
SGGVDDIQLTQSPAIMSASPGEKVTMTCRASSSVSYMNWYQQKSGTSPKRWIYDTSKVASGVPYRF
SGSGSGTSYSLTISSMEAEDAATYYCQQWSSNPLTFGAGTKLELK

According to a further aspect, the invention provides a recombinant fusion protein comprising an antigen binding molecule comprising an amino acid sequence represented by the formula (I):

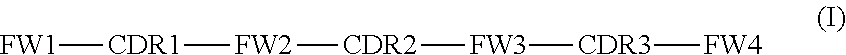

wherein
FW1 is a framework region;
CDR1 is a CDR sequence;
FW2 is a framework region;
CDR2 is a CDR sequence;
FW3 is a framework region;
CDR3 is a CDR sequence;
FW4 is a framework region.
   or a functional variant thereof,
wherein the antigen binding molecule is fused to a fragment of an immunoglobulin Fc region wherein the fragment of an immunoglobulin Fc region is engineered to dimerize with a second fragment of an immunoglobulin Fc region.

In one embodiment, the fragment of an immunoglobulin Fc region selected from the group consisting of an Fc heavy chain, a CH2 region and a CH3 region.

In one embodiment, the fragment of an immunoglobulin Fc region is an Fc heavy chain.

Fc regions may be engineered to reduce FcγR binding. Therefore, the Fc regions disclosed herein may be engineered to reduce FcγR binding.

As used herein, an immunoglobulin Fc region that is “engineered to dimerise” may comprise at least one amino acid substitution. Typically, the at least one amino acid substitution promotes and/or makes more energetically favourable, an interaction and/or association with a second fragment of an immunoglobulin Fc region, which thus promotes dimerization and/or makes dimerization more energetically favourable. Such recombinant fusion proteins may have particular utility in the preparation of bi-specific and/or bi-paratopic binders.

Methods for generating Fc based bi-specific and/or bi-paratopic binders, through pairing of two distinct Fc heavy chains that are engineered to dimerize, are known in the art. These methods enable an Fc region to be assembled from two different heavy chains, each fused to a target binding domain or sequence with different binding characteristics. The target binding domains or sequences can be directed to different targets to generate multi-specific binders and/or to different regions or epitopes on the same target to generate bi-paratopic binding proteins. Multiple binding domains or sequences can be fused to the Fc sequences to create multi-specific or multi-paratopic binders or both multi-specific multi-paratopic binders within the same protein. Methods to generate these asymmetric bispecific and/or bi-paratopic binders through heterodimerisation of two different Fc heavy chains, or fragments thereof, include but are not limited to: Knobs-into-holes (Y-T), Knobs-into-holes (CW-CSAV), CH3 charge pair, Fab-arm exchange, SEED technology, BEAT technology, HA-TF, ZW1 approach, Biclonic approach, EW-RVT and Triomab See for example, Brinkman & Kontermann, (2017) mAbs, 9:2, 182-212; Klein et al (2012) mAbs 4:6, 653-663; Wang et al (2019) Antibodies, 8, 43; and Dietrich et al (2020) BBA-Proteins and Proteomics 1868 140250; each of which is incorporated herein by reference in its entirety.

In one embodiment, the fragment of an immunoglobulin Fc region is engineered to dimerize with the second fragment of an immunoglobulin Fc region by a method selected from the group consisting of knobs-into-holes (Y-T), knobs-into-holes (CW-CSAV), CH3 charge pairing, Fab-arm exchange, SEED technology, BEAT technology, HA-TF, ZW1 approach, Biclonic approach, EW-RVT and Triomab.

Knobs-into-holes (Y-T) may comprise a T366Y substitution in a first CH3 domain and a Y407T substitution in a second CH3 domain.

Knobs-into-holes (CW-CSAV) may comprise one or more (preferably all) of the following substitutions in a first CH3 domain: S354C, T366W. Knobs-into-holes (CW-CSAV) may comprise one or more (preferably all) of the following substitutions in a second CH3 domain: Y349C, T366S, L368A, Y407V. Knobs-into-holes (CW-CSAV) may comprise a disulphide bond in CH3.

CH3 charge pairing, may comprise one or more (preferably all) of the following substitutions in a first CH3 domain: K392D, K409D. CH3 charge pairing may comprise one or more (preferably all) of the following substitutions in a second CH3 domain: E356K, D399K.

Fab-arm exchange, may comprise a K409R substitution in a first CH3 domain and a F405L substitution in a second CH3 domain. Fab arm exchange and DuoBody capture the same Fc changes. DuoBody technology, may therefore comprise a K409R substitution in a first CH3 domain and a F405L substitution in a second CH3 domain.

SEED technology may incorporate known substitutions and/or result in an IgG/A chimera. Complementarity in the CH3 interface allowing for a heterodimeric assembly of Fc chains was developed by designing strand-exchange engineered domain (SEED) heterodimers. These SEED CH3 domains are composed of alternating segments derived from human IgA and IgG CH3 sequences (AG SEED CH3 and GA SEED CH3) and were used to generate so-called SEEDbodies, Davis et al (2010) PEDS 23, 4, 195-202 hereby incorporated by reference in its entirety Because molecular models suggested that interaction with FcRn is impaired in the AG SEED CH3, residues at the CH2-CH3 junction were returned to IgG sequences. Pharmacokinetic studies confirmed that the half-life of SEEDbodies was comparable to other Fc fusion proteins and IgG1.

BEAT technology engineers the constant α and β domains of the human T cell receptor into the IgG1 CH3 dimer interface to drive heterodimerisation (Skegro et al (2017) JBC 292(23) 9745-9759). An additional D410Q mutation can further increase heterodimer formation in this system (Stutz & Blein 2020 JBC 295(28) 9392-9408).

HA-TF, may comprise one or more (preferably all) of the following substitutions in a first CH3 domain: S364H, F405A. HA-TF may comprise one or more (preferably all) of the following substitutions in a second CH3 domain: Y349T, T394F.

ZW1 approach, may comprise one or more (preferably all) of the following substitutions in a first CH3 domain: T350V, L351Y, F405A, Y407V. ZW1 approach, may comprise one or more (preferably all) of the following substitutions in a second CH3 domain: T350V, T366L, K392L, T394W.

Biclonic approach, may comprise one or more (preferably all) of the following substitutions in a first CH3 domain: 366K (+351K). Biclonic approach, may comprise one or more (preferably all) of the following substitutions in a second CH3 domain: 351 D or E or D at 349, 368, 349, or 349+355.

EW-RVT, may comprise one or more (preferably all) of the following substitutions in a first CH3 domain: K360E, K409W. EW-RVT, may comprise one or more (preferably all) of the following substitutions in a second CH3 domain: Q347R, D399V, F405T. EW-RVT may comprise a disulphide bond in CH3. A disulphide bridge may be supported by the further incorporation of Y349C to a first CH3 domain and S354C to a second CH3 domain.

Triomabs may be formed by fusing a mouse hybridoma with a rat hybridoma, resulting in production of a bispecific, assymmetric hybrid IgG molecule. Preferential pairing of light chains with its corresponding heavy chain may then occur.

In one embodiment, one or more residues of the fragment of the immunoglobulin Fc region comprises one or more amino acid substitution suitable for knobs-in-holes (KIH) dimerization with a second fragment of an immunoglobulin Fc region comprising one or more corresponding amino acid substitution.

In one embodiment, the one or more amino acid substitution is selected from the group consisting of T366Y, Y407T, S354C, T366W, Y349C, T366S, L368A and Y407V.

In one embodiment, the one or more amino acid substitution is selected from the group consisting of T366Y and Y407T.

In one embodiment, the antigen binding molecule is an ALPP and/or ALPPL2 specific antigen binding molecule.

The recombinant fusion protein may comprise a sequence according to SEQ ID NO: 127 to SEQ ID NO: 131.

A06-hFc (long linker) S239C + Y407T
(SEQ ID NO: 127)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPCVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLTS
KLTVDKSRWQQGNVFSCSVMHEALHNHYTQKSLSLSPGK
D10-hFc (long linker) S239C + Y407T
(SEQ ID NO: 128)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGGGGGS
GGGGSEPKSSDKTHTCPPCPAPELLGGPCVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLTSKLTV
DKSRWQQGNVFSCSVMHEALHNHYTQKSLSLSPGK
E06-hFc (long linker) S239C + Y407T
(SEQ ID NO: 129)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPCVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLTSKLTVD
KSRWQQGNVFSCSVMHEALHNHYTQKSLSLSPGK
E10-hFc (long linker) S239C + Y407T
(SEQ ID NO: 130)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLTSKL
TVDKSRWQQGNVFSCSVMHEALHNHYTQKSLSLSPGK
F05-hFc (long linker) S239C + Y407T
(SEQ ID NO: 131)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLTSKL
TVDKSRWQQGNVFSCSVMHEALHNHYTQKSLSLSPGK

SEQ ID NOs 127 to 131 each comprise a (G₄S)₃ linker. Also, explicitly contemplated herein are the corresponding sequences wherein the (G₄S)₃ linker is replaced with a (G₄S)₁ linker. For instance, the recombinant fusion protein may comprise one or more of the following SEQ ID NOs 132 to 136.

A06-hFc (short linker) S239C + Y407T
(SEQ ID NO: 132)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPCVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLTSKLTVDKSRWQQ
GNVFSCSVMHEALHNHYTQKSLSLSPGK
D10-hFc (short linker) S239C + Y407T
(SEQ ID NO: 133)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLTSKLTVDKSRWQQGNVF
SCSVMHEALHNHYTQKSLSLSPGK
E06-hFc (short linker) S239C + Y407T
(SEQ ID NO: 134)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLTSKLTVDKSRWQQGNVF
SCSVMHEALHNHYTQKSLSLSPGK
E10-hFc (short linker) S239C + Y407T
(SEQ ID NO: 135)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLTSKLTVDKSRWQQG
NVFSCSVMHEALHNHYTQKSLSLSPGK
F05-hFc (short linker) S239C + Y407T
(SEQ ID NO: 136)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLTSKLTVDKSRWQQG
NVFSCSVMHEALHNHYTQKSLSLSPGK

The recombinant fusion protein may comprise a sequence according to SEQ ID NO: 137 to 141.

A06-hFc (long linker) S239C + T366Y
(SEQ ID NO: 137)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPCVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLYCLVKGFYPSDIAVEWESNGQPENNYKTTPPVLDSDGSFFLYS
KLTVDKSRWQQGNVFSCSVMHEALHNHYTQKSLSLSPGK
D10-hFc (long linker) S239C + T366Y
(SEQ ID NO: 138)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPCVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLYCLVKGFYPSDIAVEWESNGQPENNYKTTPPVLDSDGSFFLYSKLTV
DKSRWQQGNVFSCSVMHEALHNHYTQKSLSLSPGK
E06-hFc (long linker) S239C + T366Y
(SEQ ID NO: 139)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPCVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLYCLVKGFYPSDIAVEWESNGQPENNYKTTPPVLDSDGSFFLYSKLTVD
KSRWQQGNVFSCSVMHEALHNHYTQKSLSLSPGK
E10-hFc (long linker) S239C + T366Y
(SEQ ID NO: 140)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLYCLVKGFYPSDIAVEWESNGQPENNYKTTPPVLDSDGSFFLYSKL
TVDKSRWQQGNVFSCSVMHEALHNHYTQKSLSLSPGK
F05-hFc (long linker) S239C + T366Y
(SEQ ID NO: 141)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLYCLVKGFYPSDIAVEWESNGQPENNYKTTPPVLDSDGSFFLYSKL
TVDKSRWQQGNVFSCSVMHEALHNHYTQKSLSLSPGK

SEQ ID NOs 137 to 141 each comprise a (G₄S)₃ linker. Also, explicitly contemplated herein are the corresponding sequences wherein the (G₄S)₃ linker is replaced with a (G₄S)l linker. For instance, the recombinant fusion protein may comprise one or more of the following SEQ ID NOs 142 to 146.

A06-hFc (short linker) S239C + T366Y
(SEQ ID NO: 142)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPCVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLYCLVKGFYPSDIAVEWESNGQPENNYKTTPPVLDSDGSFFLYSKLTVDKSRWQQ
GNVFSCSVMHEALHNHYTQKSLSLSPGK
D10-hFc (short linker) S239C + T366Y
(SEQ ID NO: 143)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLYCLVKGFYPSDIAVEWESNGQPENNYKTTPPVLDSDGSFFLYSKLTVDKSRWQQGNVF
SCSVMHEALHNHYTQKSLSLSPGK
E06-hFc (short linker) S239C + T366Y
(SEQ ID NO: 144)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLYCLVKGFYPSDIAVEWESNGQPENNYKTTPPVLDSDGSFFLYSKLTVDKSRWQQGNVF
SCSVMHEALHNHYTQKSLSLSPGK
E10-hFc (short linker) S239C + T366Y
(SEQ ID NO: 145)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLYCLVKGFYPSDIAVEWESNGQPENNYKTTPPVLDSDGSFFLYSKLTVDKSRWQQG
NVFSCSVMHEALHNHYTQKSLSLSPGK
F05-hFc (short linker) S239C + T366Y
(SEQ ID NO: 146)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLYCLVKGFYPSDIAVEWESNGQPENNYKTTPPVLDSDGSFFLYSKLTVDKSRWQQG
NVFSCSVMHEALHNHYTQKSLSLSPGK

The recombinant fusion protein may comprise a sequence according to SEQ ID NO: 147 to SEQ ID NO: 151.

A06-hFc (long linker) S442C + Y407T
(SEQ ID NO: 147)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLTS
KLTVDKSRWQQGNVFSCSVMHEALHNHYTQKSLCLSPGK
D10-hFc (long linker) S442C + Y407T
(SEQ ID NO: 148)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLTSKLTV
DKSRWQQGNVFSCSVMHEALHNHYTQKSLCLSPGK
E06-hFc (long linker) S442C + Y407T
(SEQ ID NO: 149)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLTSKLTVD
KSRWQQGNVFSCSVMHEALHNHYTQKSLCLSPGK
E10-hFc (long linker) S442C + Y407T
(SEQ ID NO: 150)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLTSKL
TVDKSRWQQGNVFSCSVMHEALHNHYTQKSLCLSPGK
F05-hFc (long linker) S442C + Y407T
(SEQ ID NO: 151)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLTSKL
TVDKSRWQQGNVFSCSVMHEALHNHYTQKSLCLSPGK

SEQ ID NOs 147 to 151 each comprise a (G₄S)₃ linker. Also, explicitly contemplated herein are the corresponding sequences wherein the (G₄S)₃ linker is replaced with a (G₄)₁ linker. For instance, the recombinant fusion protein may comprise one or more of the following SEQ ID NOs 152 to 156.

A06-hFc (short linker) S442C + Y407T
(SEQ ID NO: 152)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLTSKLTVDKSRWQQ
GNVFSCSVMHEALHNHYTQKSLCLSPGK
D10-hFc (short linker) S442C + Y407T
(SEQ ID NO: 153)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLTSKLTVDKSRWQQGNVF
SCSVMHEALHNHYTQKSLCLSPGK
E06-hFc (short linker) S442C + Y407T
(SEQ ID NO: 154)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLTSKLTVDKSRWQQGNVF
SCSVMHEALHNHYTQKSLCLSPGK
E10-hFc (short linker) S442C + Y407T
(SEQ ID NO: 155)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLTSKLTVDKSRWQQG
NVFSCSVMHEALHNHYTQKSLCLSPGK
F05-hFc (short linker) S442C + Y407T
(SEQ ID NO: 156)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLTSKLTVDKSRWQQG
NVFSCSVMHEALHNHYTQKSLCLSPGK

The recombinant fusion protein may comprise a sequence according to SEQ ID NO: 157 to 161.

A06-hFc (long linker) S442C + T366Y
(SEQ ID NO: 157)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLYCLVKGFYPSDIAVEWESNGQPENNYKTTPPVLDSDGSFFLYS
KLTVDKSRWQQGNVFSCSVMHEALHNHYTQKSLCLSPGK
D10-hFc (long linker) S442C + T366Y
(SEQ ID NO: 158)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLYCLVKGFYPSDIAVEWESNGQPENNYKTTPPVLDSDGSFFLYSKLTV
DKSRWQQGNVFSCSVMHEALHNHYTQKSLCLSPGK
E06-hFc (long linker) S442C + T366Y
(SEQ ID NO: 159)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLYCLVKGFYPSDIAVEWESNGQPENNYKTTPPVLDSDGSFFLYSKLTVD
KSRWQQGNVFSCSVMHEALHNHYTQKSLCLSPGK
E10-hFc (long linker) S442C + T366Y
(SEQ ID NO: 160)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLYCLVKGFYPSDIAVEWESNGQPENNYKTTPPVLDSDGSFFLYSKL
TVDKSRWQQGNVFSCSVMHEALHNHYTQKSLCLSPGK
F05-hFc (long linker) S442C + T366Y
(SEQ ID NO: 161)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLYCLVKGFYPSDIAVEWESNGQPENNYKTTPPVLDSDGSFFLYSKL
TVDKSRWQQGNVFSCSVMHEALHNHYTQKSLCLSPGK

SEQ ID NOs 157 to 161 each comprise a (G₄S)₃ linker. Also, explicitly contemplated herein are the corresponding sequences wherein the (G₄S)₃ linker is replaced with a (G₄S)₁ linker. For instance, the recombinant fusion protein may comprise one or more of the following SEQ ID NOs 162 to 166.

A06-hFc (short linker) S442C + T366Y
(SEQ ID NO: 162)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLYCLVKGFYPSDIAVEWESNGQPENNYKTTPPVLDSDGSFFLYSKLTVDKSRWQQ
GNVFSCSVMHEALHNHYTQKSLCLSPGK
D10-hFc (short linker) S442C + T366Y
(SEQ ID NO: 163)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLYCLVKGFYPSDIAVEWESNGQPENNYKTTPPVLDSDGSFFLYSKLTVDKSRWQQGNVF
SCSVMHEALHNHYTQKSLCLSPGK
E06-hFc (short linker) S442C + T366Y
(SEQ ID NO: 164)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLYCLVKGFYPSDIAVEWESNGQPENNYKTTPPVLDSDGSFFLYSKLTVDKSRWQQGNVF
SCSVMHEALHNHYTQKSLCLSPGK
E10-hFc (short linker) S442C + T366Y
(SEQ ID NO: 165)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLYCLVKGFYPSDIAVEWESNGQPENNYKTTPPVLDSDGSFFLYSKLTVDKSRWQQG
NVFSCSVMHEALHNHYTQKSLCLSPGK
F05-hFc (short linker) S442C + T366Y
(SEQ ID NO: 166)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLYCLVKGFYPSDIAVEWESNGQPENNYKTTPPVLDSDGSFFLYSKLTVDKSRWQQG
NVFSCSVMHEALHNHYTQKSLCLSPGK

The recombinant fusion protein may comprise a sequence according to SEQ ID NO: 167 to 171.

A06-hFc (long linker) S239C & S442C + Y407T
(SEQ ID NO: 167)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPCVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLTS
KLTVDKSRWQQGNVFSCSVMHEALHNHYTQKSLCLSPGK
D10-hFc (long linker) S239C & S442C + Y407T
(SEQ ID NO: 168)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPCVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLTSKLTV
DKSRWQQGNVFSCSVMHEALHNHYTQKSLCLSPGK
E06-hFc (long linker) S239C & S442C + Y407T
(SEQ ID NO: 169)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPCVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLTSKLTVD
KSRWQQGNVFSCSVMHEALHNHYTQKSLCLSPGK
E10-hFc (long linker) S239C & S442C + Y407T
(SEQ ID NO: 170)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLTSKL
TVDKSRWQQGNVFSCSVMHEALHNHYTQKSLCLSPGK
F05-hFc (long linker) S239C & S442C + Y407T
(SEQ ID NO: 171)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLTSKL
TVDKSRWQQGNVFSCSVMHEALHNHYTQKSLCLSPGK

SEQ ID NOs 167 to 171 each comprise a (G₄S)₃ linker. Also, explicitly contemplated herein are the corresponding sequences wherein the (G₄S)₃ linker is replaced with a (G₄S)l linker. For instance, the recombinant fusion protein may comprise one or more of the following SEQ ID NOs 172 to 176.

A06-hFc (short linker) S239C & S442C + Y407T
(SEQ ID NO: 172)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPCVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLTSKLTVDKSRWQQ
GNVFSCSVMHEALHNHYTQKSLCLSPGK
D10-hFc (short linker) S239C & S442C + Y407T
(SEQ ID NO: 173)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLTSKLTVDKSRWQQGNVF
SCSVMHEALHNHYTQKSLCLSPGK
E06-hFc (short linker) S239C & S442C + Y407T
(SEQ ID NO: 174)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLTSKLTVDKSRWQQGNVF
SCSVMHEALHNHYTQKSLCLSPGK
E10-hFc (short linker) S239C & S442C + Y407T
(SEQ ID NO: 175)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLTSKLTVDKSRWQQG
NVFSCSVMHEALHNHYTQKSLCLSPGK
F05-hFc (short linker) S239C & S442C + Y407T
(SEQ ID NO: 176)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLTSKLTVDKSRWQQG
NVFSCSVMHEALHNHYTQKSLCLSPGK

The recombinant fusion protein may comprise a sequence according to SEQ ID NO: 177 to 181.

A06-hFc (long linker) S239C & S442C + T366Y
(SEQ ID NO: 177)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPCVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLYCLVKGFYPSDIAVEWESNGQPENNYKTTPPVLDSDGSFFLYS
KLTVDKSRWQQGNVFSCSVMHEALHNHYTQKSLCLSPGK
D10-hFc (long linker) S239C & S442C + T366Y
(SEQ ID NO: 178)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPCVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLYCLVKGFYPSDIAVEWESNGQPENNYKTTPPVLDSDGSFFLYSKLTV
DKSRWQQGNVFSCSVMHEALHNHYTQKSLCLSPGK
E06-hFc (long linker) S239C & S442C + T366Y
(SEQ ID NO: 179)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPCVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLYCLVKGFYPSDIAVEWESNGQPENNYKTTPPVLDSDGSFFLYSKLTVD
KSRWQQGNVFSCSVMHEALHNHYTQKSLCLSPGK
E10-hFc (long linker) S239C & S442C + T366Y
(SEQ ID NO: 180)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLYCLVKGFYPSDIAVEWESNGQPENNYKTTPPVLDSDGSFFLYSKL
TVDKSRWQQGNVFSCSVMHEALHNHYTQKSLCLSPGK
F05-hFc (long linker) S239C & S442C + T366Y
(SEQ ID NO: 181)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPCVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLYCLVKGFYPSDIAVEWESNGQPENNYKTTPPVLDSDGSFFLYSKL
TVDKSRWQQGNVFSCSVMHEALHNHYTQKSLCLSPGK

SEQ ID NOs 177 to 181 each comprise a (G₄S)₃ linker. Also, explicitly contemplated herein are the corresponding sequences wherein the (G₄S)₃ linker is replaced with a (G₄S)₁ linker. For instance, the recombinant fusion protein may comprise one or more of the following SEQ ID NOs 182 to 186.

A06-hFc (short linker) S239C & S442C + T366Y
(SEQ ID NO: 182)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPCVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLYCLVKGFYPSDIAVEWESNGQPENNYKTTPPVLDSDGSFFLYSKLTVDKSRWQQ
GNVFSCSVMHEALHNHYTQKSLCLSPGK
D10-hFc (short linker) S239C & S442C + T366Y
(SEQ ID NO: 183)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLYCLVKGFYPSDIAVEWESNGQPENNYKTTPPVLDSDGSFFLYSKLTVDKSRWQQGNVF
SCSVMHEALHNHYTQKSLCLSPGK
E06-hFc (short linker) S239C & S442C + T366Y
(SEQ ID NO: 184)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPCVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLYCLVKGFYPSDIAVEWESNGQPENNYKTTPPVLDSDGSFFLYSKLTVDKSRWQQGNVF
SCSVMHEALHNHYTQKSLCLSPGK
E10-hFc (short linker) S239C & S442C + T366Y
(SEQ ID NO: 185)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLYCLVKGFYPSDIAVEWESNGQPENNYKTTPPVLDSDGSFFLYSKLTVDKSRWQQG
NVFSCSVMHEALHNHYTQKSLCLSPGK
F05-hFc (short linker) S239C & S442C + T366Y
(SEQ ID NO: 186)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPCVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLYCLVKGFYPSDIAVEWESNGQPENNYKTTPPVLDSDGSFFLYSKLTVDKSRWQQG
NVFSCSVMHEALHNHYTQKSLCLSPGK

The recombinant fusion protein may be a bi-paratopic dimer comprising any one or any two of SEQ ID NOs 87 to 186. The bi-paratopic dimer may comprise one of SEQ ID NOs 87 to 126. The bi-paratopic dimer may comprise one of SEQ ID NOs 127 to 136, 147 to 156, or 167 to 176 comprising the Y407T point mutation. The bi-paratopic dimer may comprise one of SEQ ID NOs 137 to 146, 157 to 166, or 177 to 186 comprising the T366Y point mutation.

In a preferred embodiment, the bi-paratopic dimer may comprise D10-hFc (long linker) S239C+T366Y (SEQ ID NO: 138) and E06-hFc (long linker) S239C Y407T (SEQ ID NO: 129).

In a preferred embodiment, the bi-paratopic dimer may comprise D10-hFc (long linker) S239C+T366Y (SEQ ID NO: 138) and E10-hFc (long linker) S239C Y407T (SEQ ID NO: 130).

In a preferred embodiment, the bi-paratopic dimer may comprise D10-hFc (long linker) S239C+T366Y (SEQ ID NO: 138) and F05-hFc (long linker) S239C Y407T (SEQ ID NO: 131).

In a preferred embodiment, the bi-paratopic dimer may comprise A06-hFc (long linker) S239C T366Y (SEQ ID NO: 137) and F05-hFc (long linker) S239C+Y407T (SEQ ID NO: 131).

In a preferred embodiment, the bi-paratopic dimer may comprise D10-hFc (short linker) S239C+T366Y (SEQ ID NO: 143) and E06-hFc (short linker) S239C+Y407T (SEQ ID NO: 154).

In a preferred embodiment, the bi-paratopic dimer may comprise D10-hFc (short linker) S239C+T366Y (SEQ ID NO: 143) and E10-hFc (short linker) S239C+Y407T (SEQ ID NO: 135).

In a preferred embodiment, the bi-paratopic dimer may comprise D10-hFc (short linker) S239C+T366Y (SEQ ID NO: 143) and F05-hFc (short linker) S239C+Y407T (SEQ ID NO: 136).

In a preferred embodiment, the bi-paratopic dimer may comprise A06-hFc (short linker) S239C+T366Y (SEQ ID NO: 137) and F05-hFc (short linker) S239C+Y407T (SEQ ID NO: 136).

In a preferred embodiment, the bi-paratopic dimer may comprise A06-hFc (short linker) S442C+T366Y (SEQ ID NO: 157) and F05-hFc (short linker) S442C+Y407T (SEQ ID NO: 156).

In a preferred embodiment, the bi-paratopic dimer may comprise A06-hFc (short linker) S239C & S442C+T366Y (SEQ ID NO: 177) and F05-hFc (short linker) S239C & S442C+Y407T (SEQ ID NO: 176).

In a preferred embodiment, the bi-paratopic dimer may comprise F05-hFc (short linker) S239C+T366Y (SEQ ID NO: 146) and A06-hFc (short linker) S239C+Y407T (SEQ ID NO: 132).

In a preferred embodiment, the bi-paratopic dimer may comprise F05-hFc (short linker) S442C+T366Y (SEQ ID NO: 166) and A06-hFc (short linker) S442C+Y407T (SEQ ID NO: 152).

In a preferred embodiment, the bi-paratopic dimer may comprise F05-hFc (short linker) S239C & S442C+T366Y (SEQ ID NO: 186) and A06-hFc (short linker) S239C & S442C+Y407T (SEQ ID NO: 172).

In a preferred embodiment, the bi-paratopic dimer may comprise D10-hFc (long linker) S239C & S442C+T366Y (SEQ ID NO: 178) and E06-hFc (long linker) S239C & 442C+Y407T (SEQ ID NO: 169).

In a preferred embodiment, the bi-paratopic dimer may comprise D10-hFc (long linker) S239C & S442C+T366Y (SEQ ID NO: 178) and E10-hFc (long linker) S239C & S442C+Y407T (SEQ ID NO: 170).

In a preferred embodiment, the bi-paratopic dimer may comprise D10-hFc (long linker) S239C & S442C+T366Y (SEQ ID NO: 178) and F05-hFc (long linker) S239C & S442C+Y407T (SEQ ID NO: 171).

In a preferred embodiment, the bi-paratopic dimer may comprise A06-hFc (long linker) S239C & S442C+T366Y (SEQ ID NO: 177) and F05-hFc (long linker) S239C & S442C+Y407T (SEQ ID NO: 171).

In a preferred embodiment, the bi-paratopic dimer may comprise D10-hFc (short linker) S239/442C T366Y (SEQ ID NO: 183) and E06-hFc (short linker) S239/442C Y407T (SEQ ID NO: 174).

In a preferred embodiment, the bi-paratopic dimer may comprise D10-hFc (short linker) S239C & S442C+T366Y (SEQ ID NO: 183) and E10-hFc (short linker) S239C & S442C+Y407T (SEQ ID NO: 175).

In a preferred embodiment, the bi-paratopic dimer may comprise D10-hFc (short linker) S239C & S442C+T366Y (SEQ ID NO: 183) and F05-hFc (short linker) S239C & S442C+Y407T (SEQ ID NO: 176).

Any of the recombinant fusion proteins disclosed herein may be associated with any of the linkers and payloads disclosed herein, Any of the bi-paratopic dimers disclosed herein may be associated with any of the linkers and payloads disclosed herein, Conjugation may be by any one or more S239C and/or S442C residue in the bi-paratopic dimer. Preferably, the bi-paratopic dimer may be associated with the linker and payload vc-MMAE. Preferably, the bi-paratopic dimer comprises those conjugates disclosed in Example 6 which have been shown to be highly efficacious in vivo.

SEQ ID Nos: 97-186 include an S239C, S442C or both an S239C and S442C mutation, for use in conjugation reactions. Where the recombinant fusion protein is not conjugated (for example to an anthracycline (PNU) derivative or an auristatin derivative (MMAE) the S239C and S442C mutation are not needed and position 239 and 442 may be an S rather than a C. Accordingly, in alternative embodiments the recombinant fusion protein or bi-paratopic dimer may comprise a sequence according to any one of SEQ ID Nos: 97-186 except that each sequence does not include an S239C and S442C mutation.

Therefore, the recombinant fusion protein may comprise a sequence according to SEQ ID NO: 188 to SEQ ID NO: 192.

A06-hFc (long linker) + Y407T
(SEQ ID NO: 188)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLTCLVKGFYPSDIAVEWESNGQPENNYKTTPPVLDSDGSFFLTS
KLTVDKSRWQQGNVFSCSVMHEALHNHYTQKSLSLSPGK
D10-hFc (long linker) + Y407T
(SEQ ID NO: 189)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLTCLVKGFYPSDIAVEWESNGQPENNYKTTPPVLDSDGSFFLTSKLTV
DKSRWQQGNVFSCSVMHEALHNHYTQKSLSLSPGK
E06-hFc (long linker) + Y407T
(SEQ ID NO: 190)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKTTPPVLDSDGSFFLTSKLTVD
KSRWQQGNVFSCSVMHEALHNHYTQKSLSLSPGK
E10-hFc (long linker) + Y407T
(SEQ ID NO: 191)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLTSKL
TVDKSRWQQGNVFSCSVMHEALHNHYTQKSLSLSPGK
F05-hFc (long linker) + Y407T
(SEQ ID NO: 192)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLTCLVKGFYPSDIAVEWESNGQPENNYKTTPPVLDSDGSFFLTSKL
TVDKSRWQQGNVFSCSVMHEALHNHYTQKSLSLSPGK

SEQ ID NOs 188 to 192 each comprise a (G₄S)₃ linker. Also, explicitly contemplated herein are the corresponding sequences wherein the (G₄S)₃ linker is replaced with a (G₄)₁ linker. For instance, the recombinant fusion protein may comprise one or more of the following SEQ ID NOs 193 to 197.

A06-hFc (short linker) + Y407T
(SEQ ID NO: 193)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLTCLVKGFYPSDIAVEWESNGQPENNYKTTPPVLDSDGSFFLTSKLTVDKSRWQQ
GNVFSCSVMHEALHNHYTQKSLSLSPGK
D10-hFc (short linker) + Y407T
(SEQ ID NO: 194)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLTSKLTVDKSRWQQGNVF
SCSVMHEALHNHYTQKSLSLSPGK
E06-hFc (short linker) + Y407T
(SEQ ID NO: 195)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSFFLTSKLTVDKSRWQQGNVF
SCSVMHEALHNHYTQKSLSLSPGK
E10-hFc (short linker) + Y407T
(SEQ ID NO: 196)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLTSKLTVDKSRWQQG
NVFSCSVMHEALHNHYTQKSLSLSPGK
F05-hFc (short linker) + Y407T
(SEQ ID NO: 197)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLTCLVKGFYPSDIAVEWESNGQPENNYKTTPPVLDSDGSFFLTSKLTVDKSRWQQG
NVFSCSVMHEALHNHYTQKSLSLSPGK

The recombinant fusion protein may comprise a sequence according to SEQ ID NO: 199 to SEQ ID NO: 203:

A06-hFc (long linker) + T366Y
(SEQ ID NO: 199)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSGG
GGSGGGGSEPKSSDKTHTCPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKVSNKALPAPIEKTISKAKG
QPREPQVYTLPPSRDELTKNQVSLYCLVKGFYPSDIAVEWESNGQPENNYKTTPPVLDSDGSFFLYS
KLTVDKSRWQQGNVFSCSVMHEALHNHYTQKSLSLSPGK
D10-hFc (long linker) + T366Y
(SEQ ID NO: 200)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSGGGGS
GGGGSEPKSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKAKGQPR
EPQVYTLPPSRDELTKNQVSLYCLVKGFYPSDIAVEWESNGQPENNYKTTPPVLDSDGSFFLYSKLTV
DKSRWQQGNVFSCSVMHEALHNHYTQKSLSLSPGK
E06-hFc (long linker) + T366Y
(SEQ ID NO: 201)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSGGGGSG
GGGSEPKSSDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPRE
PQVYTLPPSRDELTKNQVSLYCLVKGFYPSDIAVEWESNGQPENNYKTTPPVLDSDGSFFLYSKLTVD
KSRWQQGNVFSCSVMHEALHNHYTQKSLSLSPGK
E10-hFc (long linker) + T366Y
(SEQ ID NO: 202)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLYCLVKGFYPSDIAVEWESNGQPENNYKTTPPVLDSDGSFFLYSKL
TVDKSRWQQGNVFSCSVMHEALHNHYTQKSLSLSPGK
F05-hFc (long linker) + T366Y
(SEQ ID NO: 203)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSGGG
GSGGGGSEPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQ
PREPQVYTLPPSRDELTKNQVSLYCLVKGFYPSDIAVEWESNGQPENNYKTTPPVLDSDGSFFLYSKL
TVDKSRWQQGNVFSCSVMHEALHNHYTQKSLSLSPGK

SEQ ID NOs 199 to 203 each comprise a (G₄S)₃ linker. Also, explicitly contemplated herein are the corresponding sequences wherein the (G₄S)₃ linker is replaced with a (G₄)₁ linker. For instance, the recombinant fusion protein may comprise one or more of the following SEQ ID NOs 204 to 208.

A06-hFc (short linker) + T366Y
(SEQ ID NO: 204)
QVHLVESGGGLVQAGGSLRLSCAASGRTFLSMGWFRQAPGKEREFVAAISWTGGSTGYADSVKGR
FTISRESDKETMYLQMNSLKPEDTAVYYCAARGDVYYGGDDATRYKYWGQGTQVTVSSGGGGSEP
KSSDKTHTCPPCPAPELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEKTISKAKGQPREPQVYTLP
PSRDELTKNQVSLYCLVKGFYPSDIAVEWESNGQPENNYKTTPPVLDSDGSFFLYSKLTVDKSRWQQ
GNVFSCSVMHEALHNHYTQKSLSLSPGK
D10-hFc (short linker) + T366Y
(SEQ ID NO: 205)
QVQLVESGGGLVQPGGSLRLSCAASGSIFSVNTMGWYRQAPGKQRDLVAVATSGGTIHYADSVKGR
FTISRDNAKNTVYLQMNSLKPEDTAVYYCNLVVPYGMIAGDHDYWGQGTKVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLYCLVKGFYPSDIAVEWESNGQPENNYKTTPPVLDSDGSFFLYSKLTVDKSRWQQGNVF
SCSVMHEALHNHYTQKSLSLSPGK
E06-hFc (short linker) + T366Y
(SEQ ID NO: 206)
QVHLVESGGGLVQAGGSLRLSCAASRRSFSEYAMGWFRQAPGKEREFVAAIGWSGVIEYADSVKGR
FTISRDNAKDTVYLQMNSLKPEDTAVYYCAAGLGIRVVSQTPAYWGQGTQVTVSSGGGGSEPKSSD
KTHTCPPCPAPELLGGPSVFLFPPKPKDTLMISRTPEVTCVVVDVSHEDPEVKFNWYVDGVEVHNAK
TKPREEQYNSTYRVVSVLTVLHQDWLNGKEYKCKVSNKALPAPIEKTISKAKGQPREPQVYTLPPSRD
ELTKNQVSLYCLVKGFYPSDIAVEWESNGQPENNYKTTPPVLDSDGSFFLYSKLTVDKSRWQQGNVF
SCSVMHEALHNHYTQKSLSLSPGK
E10-hFc (short linker) + T366Y
(SEQ ID NO: 207)
QVQLVESGGGLVQAGGSLRLSCAVSGRALSDYNIGWFRQAPGKEREFVARKTWDGSSTRYDDSVK
GRFTVSIEDAKNTAYLQMSSLKTEDTAVYYCAAGRYSRARGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLYCLVKGFYPSDIAVEWESNGQPENNYKTTPPVLDSDGSFFLYSKLTVDKSRWQQG
NVFSCSVMHEALHNHYTQKSLSLSPGK
F05-hFc (short linker) + T366Y
(SEQ ID NO: 208)
QVQLVESGGGLVQAGGSLRLSCAASGRAFGDYNIGWFRQAPGKEREFVARRTWDGSSTRYDDSVK
GRFTVSIDNAKNTAYLQMNSLKTEDTAVYYCAAGRYSRVRGWEYDYWGQGTQVTVSSGGGGSEPK
SSDKTHTCPPCPAPELLGGPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEKTISKAKGQPREPQVYTLPP
SRDELTKNQVSLYCLVKGFYPSDIAVEWESNGQPENNYKTTPPVLDSDGSFFLYSKLTVDKSRWQQG
NVFSCSVMHEALHNHYTQKSLSLSPGK

According to a further aspect, the invention provides a recombinant fusion protein dimer comprising
(a) a first recombinant fusion protein, wherein the first recombinant fusion protein is a recombinant fusion protein as disclosed herein, and
(b) a second recombinant fusion protein, wherein the second recombinant fusion protein comprises a second antigen binding molecule fused to a second fragment of an immunoglobulin Fc region engineered to dimerize with the first fragment of an immunoglobulin Fc region.

In one embodiment, the second fragment of an immunoglobulin Fc region selected from the group 50 consisting of an Fc heavy chain, a CH2 region and a CH3 region.

In one embodiment, the second fragment of an immunoglobulin Fc region is an Fc heavy chain.

In one embodiment, the second fragment of an immunoglobulin Fc region is engineered to dimerize with the second fragment of an immunoglobulin Fc region by a method selected from the group consisting of knobs-into-holes (Y-T), knobs-into-holes (CW-CSAV), CH3 charge pairing, Fab-arm exchange, SEED technology, BEAT technology, HA-TF, ZW1 approach, Biclonic approach, EW-RVT and Triomab.

In one embodiment, one or more residues of the fragment of the immunoglobulin Fc region comprises one or more amino acid substitution suitable for knobs-in-holes (KIH) dimerization with a second fragment of an immunoglobulin Fc region comprising one or more corresponding amino acid mutation.

In one embodiment, the one or more amino acid substitution is selected from the group consisting of T366Y, Y407T, S354C, T366W, Y349C, T366S, L368A and Y407V.

In one embodiment, the one or more amino acid substitution is selected from the group consisting of T366Y and Y407T.

Any sequence of a recombinant fusion protein disclosed herein may comprise any one or more amino acid substitution selected from the group consisting of T366Y, Y407T, S354C, T366W, Y349C, T366S, L368A and Y407V. SEQ ID NO: 209 may therefore be modified by the incorporation of any one or more amino acid substitution selected from the group consisting of T366Y, Y407T, S354C, T366W, Y349C, T366S, L368A and Y407V and incorporated into a recombinant fusion protein as described herein in place of the human Fc region sequence.

(human Fc region)
SEQ ID NO: 209
EPKSSDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGK

In one embodiment, the second antigen binding molecule is an ALPP and/or ALPPL2 specific antigen binding molecule.

In one embodiment, the second specific antigen binding molecule is an immunoglobin, an immunoglobin Fab region, a Fab′, a Fv, a Fv-Fc, a single chain Fv (scFv), scFv-Fc, (scFv)₂, a diabody, a triabody, a tetrabody, a bispecific t-cell engager (BiTE), an intein, a VNAR domain, a single domain antibody (sdAb) or a VH domain.

Preferably, the recombinant fusion protein dimer is a bi-paratopic dimer comprising a first recombinant fusion protein and a second recombinant fusion protein that are specific to different regions or epitopes on the same target (e.g. ALPP and/or ALPPL2). Bi-paratopic dimers can comprise any ALPP and/or ALPPL2 specific antigen binding molecule or fusion protein, such as disclosed herein.

Epitope binning has been carried out for the ALPP and/or ALPPL2 specific antigen binding molecules disclosed herein. A05, B09, B111, E06, E10, F05, F08 and G03 are in epitope bin one. C07 and D04 are in epitope bin four. A06 is in epitope bin two. B01 is in epitope bin three. D10 is in epitope bin five. A ALPP and/or ALPPL2 specific antigen binding molecule or fusion protein thereof from one epitope bin can be combined with an ALPP and/or ALPPL2 specific antigen binding molecule or fusion protein thereof from another epitope bin to form a bi-paratopic dimer. Preferred pairings are given below:
D10-E06
D10-E10
D10-F05
A06-F05

In one embodiment:
(a) the first recombinant fusion protein comprises a sequence according to SEQ ID NO: 138 (D10-hFc (long linker) S239C+T366Y), and
(b) the second recombinant fusion protein comprises a sequence according to SEQ ID NO: 129 (E06-hFc (long linker) S239C+Y407T).

In one embodiment:
(a) the first recombinant fusion protein comprises a sequence according to SEQ ID NO: 138 (D10-hFc (long linker) S239C+T366Y), and
(b) the second recombinant fusion protein comprises a sequence according to SEQ ID NO: 130 (E10-hFc (long linker) S239C+Y407T).

In one embodiment:
(a) the first recombinant fusion protein comprises a sequence according to SEQ ID NO: 138 (D10-hFc (long linker) S239C+T366Y), and
(b) the second recombinant fusion protein comprises a sequence according to SEQ ID NO: 131 (F05-hFc (long linker) S239C+Y407T).

In one embodiment:
(a) the first recombinant fusion protein comprises a sequence according to SEQ ID NO: 137 (A06-hFc S239C T366Y), and
(b) the second recombinant fusion protein comprises a sequence according to SEQ ID NO: 131 (F05-hFc (long linker) S239C+Y407T).

In one embodiment:
(a) the first recombinant fusion protein comprises a sequence according to SEQ ID NO: 143 (D10-hFc (short linker) S239C+T366Y), and
(b) the second recombinant fusion protein comprises a sequence according to SEQ ID NO: 134 (E06-hFc (short linker) S239C+Y407T).

In one embodiment:
(a) the first recombinant fusion protein comprises a sequence according to SEQ ID NO: 143 (D10-hFc (short linker) S239C+T366Y), and
(b) the second recombinant fusion protein comprises a sequence according to SEQ ID NO: 135 (E10-hFc (short linker) S239C Y407T).

In one embodiment:
(a) the first recombinant fusion protein comprises a sequence according to SEQ ID NO: 143 (D10-hFc (short linker) S239C+T366Y), and
(b) the second recombinant fusion protein comprises a sequence according to SEQ ID NO: 136 (F05-hFc (short linker) S239C+Y407T).

In one embodiment:
(a) the first recombinant fusion protein comprises a sequence according to SEQ ID NO: 178 (D10-hFc S239C & S442C T366Y), and
(b) the second recombinant fusion protein comprises a sequence according to SEQ ID NO: 169 (E06-hFc S239C & S442C+Y407T).

In one embodiment:
(a) the first recombinant fusion protein comprises a sequence according to SEQ ID NO: 178 (D10-hFc S239C & S442C+T366Y), and
(b) the second recombinant fusion protein comprises a sequence according to SEQ ID NO: 170 (E10-hFc S239C & S442C+Y407T).

In one embodiment:
(a) the first recombinant fusion protein comprises a sequence according to SEQ ID NO: 178 (D10-hFc S239C & S442C+T366Y), and
(b) the second recombinant fusion protein comprises a sequence according to SEQ ID NO: 171 (F05-hFc S239C & S442C+Y407T).

In one embodiment:
(a) the first recombinant fusion protein comprises a sequence according to SEQ ID NO: 177 (A06-hFc S239C & S442C+T366Y), and
(b) the second recombinant fusion protein comprises a sequence according to SEQ ID NO: 171 (F05-hFc S239C & S442C+Y407T).

In one embodiment:
(a) the first recombinant fusion protein comprises a sequence according to SEQ ID NO: 183 (D10-hFc (short linker) S239C & S442C+T366Y), and
(b) the second recombinant fusion protein comprises a sequence according to SEQ ID NO: 174 (E06-hFc (short linker) S239C & S442C+Y407T).

In one embodiment:
(a) the first recombinant fusion protein comprises a sequence according to SEQ ID NO: 183 (D10-hFc (short linker) S239C & S442C+T366Y), and
(b) the second recombinant fusion protein comprises a sequence according to SEQ ID NO: 175 (E10-hFc (short linker) S239C & S442C+Y407T).

In one embodiment:
(a) the first recombinant fusion protein comprises a sequence according to SEQ ID NO: 183 (D10-hFc (short linker) S239C & S442C+T366Y), and
(b) the second recombinant fusion protein comprises a sequence according to SEQ ID NO: 176 (F05-hFc (short linker) S239C & S442C+Y407T).

According to a further aspect, the invention provides an ALPP and/or ALPPL2-specific chimeric antigen receptor (CAR), comprising at least one ALPP and/or ALPPL2-specific antigen binding molecule as disclosed herein, fused or conjugated to at least one transmembrane region and at least one intracellular domain.

The present invention also provides a cell comprising a chimeric antigen receptor as disclosed herein, which cell is preferably an engineered T-cell.

In a further aspect of the invention, there is provided a nucleic acid sequence comprising a polynucleotide sequence that encodes a specific antigen binding molecule, recombinant fusion protein, recombinant fusion protein dimer or chimeric antigen receptor as disclosed herein.

There is also provided a vector comprising a nucleic acid sequence as disclosed herein and a host cell comprising such a nucleic acid.

A method for preparing a specific antigen binding molecule, recombinant fusion protein, recombinant fusion protein dimer or chimeric antigen receptor, as disclosed herein, the method comprising cultivating or maintaining a host cell comprising the polynucleotide or vector described above under conditions such that said host cell produces the specific antigen binding molecule, recombinant fusion protein or chimeric antigen receptor, optionally further comprising isolating the specific antigen binding molecule, recombinant fusion protein or chimeric antigen receptor.

In a further aspect of the invention, there is provided a pharmaceutical composition comprising the specific antigen binding molecule, recombinant fusion protein, recombinant fusion protein dimer or chimeric antigen receptor as disclosed herein. The pharmaceutical composition may contain a variety of pharmaceutically acceptable carriers. Pharmaceutical compositions of the invention may be for administration by any suitable method known in the art, including but not limited to intravenous, intramuscular, oral, intraperitoneal, or topical administration. In preferred embodiments, the pharmaceutical composition may be prepared in the form of a liquid, gel, powder, tablet, capsule, or foam.

The specific antigen binding molecule, recombinant fusion protein or chimeric antigen receptor as disclosed herein may be for use in therapy. More specifically, the specific antigen binding molecule, recombinant fusion protein, recombinant fusion protein dimer or chimeric antigen receptor as disclosed herein may be for use in the treatment of cancer. Preferably, the cancer is a ALPP and/or ALPPL2-positive cancer type. More preferably, the cancer is selected from the group consisting of ovarian cancer, endometrial cancer, cervical cancer, gastric cancer, oesophageal cancer, bladder cancer, lung cancer, pancreatic cancer and testicular cancer.

Also provided herein is the use of a specific antigen binding molecule, recombinant fusion protein, recombinant fusion protein dimer or chimeric antigen receptor as disclosed herein in the manufacture of a medicament for the treatment of a disease in a patient in need thereof.

Furthermore, in accordance with the present invention there is provided a method of treatment of a disease in a patient in need of treatment comprising administration to said patient of a therapeutically effective dosage of a specific antigen binding molecule, recombinant fusion protein, recombinant fusion protein dimer or chimeric antigen receptor as disclosed herein or a pharmaceutical composition as disclosed herein.

Preferably, the cancer is a ALPP and/or ALPPL2-positive cancer type. More preferably, the cancer is selected from the group consisting of ovarian cancer, endometrial cancer, cervical cancer, gastric cancer, oesophageal cancer, bladder cancer, lung cancer, pancreatic cancer and testicular cancer.

Also provided herein is a method of assaying for the presence of a target analyte in a sample, comprising the addition of a detectably labelled specific antigen binding molecule as disclosed herein, or a recombinant fusion protein as disclosed herein, or a recombinant fusion protein dimer as disclosed herein, to the sample and detecting the binding of the molecule to the target analyte.

In addition, there is provided herein a method of imaging a site of disease in a subject, comprising administration of a detectably labelled specific antigen binding molecule as disclosed herein or a detectably labelled recombinant fusion protein as disclosed herein, or a recombinant fusion protein dimer as disclosed herein to a subject.

There is also provided herein a method of diagnosis of a disease or medical condition in a subject comprising administration of a specific antigen binding molecule as disclosed herein, or a recombinant fusion protein as disclosed herein, or a recombinant fusion protein dimer as disclosed herein.

Also contemplated herein is an antibody, antibody fragment or antigen-binding molecule that competes for binding to ALPP and/or ALPPL2 with the ALPP and/or ALPPL2-specific antigen binding molecule as disclosed herein. The term “compete” when used in the context of antigen binding proteins (e.g., neutralizing antigen binding proteins or neutralizing antibodies) means competition between antigen binding proteins as determined by an assay in which the antigen binding protein (e.g., antibody or functional fragment thereof) under test prevents or inhibits specific binding of a the antigen binding molecule defined herein (e.g., specific antigen binding molecule of the first aspect) to a common antigen (e.g., ALPP and/or ALPPL2 in the case of the specific antigen binding molecule as disclosed herein).

Also described herein is a kit for diagnosing a subject suffering from cancer, or a pre-disposition thereto, or for providing a prognosis of the subject's condition, the kit comprising detection means for detecting the concentration of antigen present in a sample from a test subject, wherein the detection means comprises an ALPP and/or ALPPL2-specific antigen binding molecule as disclosed herein, a recombinant fusion protein or recombinant fusion protein dimer as disclosed herein, a chimeric antigen receptor as disclosed herein or a nucleic acid sequence as disclosed herein, each being optionally derivatized, wherein presence of antigen in the sample suggests that the subject suffers from cancer.

Preferably the antigen comprises ALPP and/or ALPPL2 protein. More preferably, the kit is used to identify the presence or absence of ALPP and/or ALPPL2-positive cells in the sample, or determine the concentration thereof in the sample. The kit may also comprise a positive control and/or a negative control against which the assay is compared and/or a label which may be detected.

The present invention also provides a method for diagnosing a subject suffering from cancer, or a pre-disposition thereto, or for providing a prognosis of the subject's condition, the method comprising detecting the concentration of antigen present in a sample obtained from a subject, wherein the detection is achieved using a ALPP and/or ALPPL2-specific antigen binding molecule as disclosed herein, a recombinant fusion protein or recombinant fusion protein dimer as disclosed herein, a chimeric antigen receptor as disclosed herein or a nucleic acid sequence as disclosed herein, each being optionally derivatized, and wherein presence of antigen in the sample suggests that the subject suffers from cancer.

Also contemplated herein is a method of killing or inhibiting the growth of a cell expressing ALPP and/or ALPPL2 in vitro or in a patient, which method comprises administering to the cell a pharmaceutically effective amount or dose of (i) ALPP and/or ALPPL2-specific antigen binding molecule as disclosed herein, a recombinant fusion protein or recombinant fusion protein dimer as disclosed herein, a nucleic acid sequence as disclosed herein, or the CAR or cell as disclosed herein, or (ii) of a pharmaceutical composition as disclosed herein. Preferably, the cell expressing ALPP and/or ALPPL2 is a cancer cell. More preferably, the ALPP and/or ALPPL2 is human ALPP and/or ALPPL2.

According to a further aspect, the invention provides a specific antigen binding molecule comprising an amino acid sequence represented by the formula (II):

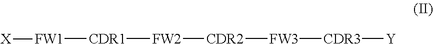

wherein
FW1-CDR1-FW2-CDR2-FW3-CDR3 is an ALPP and/or ALPPL2-specific antigen binding molecule as disclosed herein
X and Y are optional amino acid sequences
   wherein the specific antigen binding molecule is conjugated to a second moiety.

In certain preferred embodiments, the specific antigen binding molecule according to this aspect of the invention may additionally be conjugated to a third, fourth or fifth moiety. Conjugation of further moieties is also contemplated. In some cases, a third, fourth or fifth moiety may be conjugated to the second moiety. Accordingly, it will be understood that any of the moieties according to this aspect of the invention may have additional moieties conjugated thereto. Description of preferred features of the second moiety as set out below apply to the third, fourth, fifth or higher order moiety mutatis mutandis.

Preferably X or Y are individually either absent or selected from the group comprising an immunoglobulin, an immunoglobulin Fc region, a fragment of an immunoglobulin Fc region, an Fc heavy chain, a CH2 region, a CH3 region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, a scaffold protein (affibodies, centyrins, darpins etc.), or a toxin including but not limited to Pseudomonas exotoxin PE38, diphtheria toxin.

Preferably, the conjugation is via a cysteine residue in the amino acid sequence of the specific antigen binding molecule. The cysteine residue may be anywhere in the sequence, including in optional sequences X or Y (if present).

The conjugation may be via a thiol, aminoxy or hydrazinyl moiety incorporated at the N-terminus or C-terminus of the amino acid sequence of the specific antigen binding molecule.

Preferably, the second moiety is selected from the group comprising detectable label, dye, toxin, drug, pro-drug, radionuclide or biologically active molecule.

More preferably, the second moiety is at least one cytotoxic or cytostatic agent (also referred to herein as “toxins”).

More preferably, the second moiety is at least one toxin selected from the group comprising:
auristatins,
anthracyclines, preferably PNU-derived anthracyclines
maytansinoids,
amanitin derivatives, preferably α-amanitin derivatives
calicheamicins,
tubulysins
duocarmycins
radioisotopes—such as an alpha-emitting radionuclide, such as 227 Th and 225 Ac label
liposomes comprising a toxic payload,
protein toxins
taxanes
pyrrolbenzodiazepines and dimers thereof
indolinobenzodiazepine pseudodimers
spliceosome inhibitors
CDK11 inhibitors
nicotinamide phosphoribosyltransferase inhibitors (NAMPTi)
Pyridinobenzodiazepines and dimers thereof
Cyclopropapyrroloindole (CPI), cyclopropabenzindole (CBI) or cyclopropathienoindole (CTI) and optionally dimers thereof
Irinotecan or exatecan and their derivatives.

In other preferred embodiments in accordance with this aspect, the second moiety may be from the group comprising an immunoglobulin, an immunoglobulin Fc region, a fragment of an immunoglobulin Fc region, an Fc heavy chain, a CH2 region, a CH3 region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, a scaffold protein (affibodies, centyrins, darpins etc.), or a toxin including but not limited to Pseudomonas exotoxin PE38, diphtheria toxin.

In particularly preferred embodiments, the second moiety is a VHH domain, which may be the same or different to the specific antigen binding molecule according to this aspect. Accordingly, dimers, trimers or higher order multimers of VHH domains linked by chemical conjugation are explicitly contemplated herein. In such embodiments, each individual VHH domain may have the same antigen specificity as the other VHH domains, or they may be different.

In accordance with this aspect, the specific antigen binding molecule may comprise, for example, bi-paratopic specific antigen binding molecules as described herein fused to further biologically active molecules (including but not limited to Fc fusions) and then further conjugated to a second moiety, including but not limited to cytotoxic payloads

In accordance with this aspect, the specific antigen binding molecule may be an alkaline phosphatase, placental type (ALPP) specific antigen binding molecule and/or alkaline phosphatase, germ cell type (ALPPL2). This may be an ALPP and/or ALPPL2-specific antigen binding molecule disclosed herein.

The specific antigen binding molecule of this aspect may be for use in therapy. More specifically, the specific antigen binding molecule of this aspect may be for use in the treatment of cancer. Preferably, the cancer is an ALPP and/or ALPPL2-positive cancer type. More preferably, the cancer is selected from the group consisting of ovarian cancer, endometrial cancer, cervical cancer, gastric cancer, oesophageal cancer, bladder cancer, lung cancer, pancreatic cancer and testicular cancer.

Also provided herein is the use of a specific antigen binding molecule of this aspect in the manufacture of a medicament for the treatment of a disease in a patient in need thereof.

Pharmaceutical compositions comprising the specific antigen binding molecule of this aspect are also provided. The pharmaceutical composition may contain a variety of pharmaceutically acceptable carriers

Furthermore, in accordance with the present invention there is provided a method of treatment of a disease in a patient in need of treatment comprising administration to said patient of a therapeutically effective dosage of a specific antigen binding molecule of this aspect or a pharmaceutical composition comprising a specific antigen binding molecule of this aspect.

Preferably, the cancer is a ALPP and/or ALPPL2-positive cancer type. More preferably, the cancer is selected from the group consisting of ovarian cancer, endometrial cancer, cervical cancer, gastric cancer, oesophageal cancer, bladder cancer, lung cancer, pancreatic cancer and testicular cancer.

Also provided herein is a method of assaying for the presence of a target analyte in a sample, comprising the addition of a detectably labelled specific antigen binding molecule of this aspect to the sample and detecting the binding of the molecule to the target analyte.

In addition, there is provided herein a method of imaging a site of disease in a subject, comprising administration of a detectably labelled specific antigen binding molecule of this aspect to a subject.

There is also provided herein a method of diagnosis of a disease or medical condition in a subject comprising administration of a specific antigen binding molecule of this aspect.

According to a further aspect, the invention provides an engineered ALPP and/or ALPPL2 monomer, wherein the engineered ALPP and/or ALPPL2 monomer is engineered to be capable of forming a covalent link with a further engineered ALPP and/or ALPPL2 monomer.

In one embodiment, the covalent link is a disulphide bridge.

In one embodiment, the engineered ALPP and/or ALPPL2 monomer comprises an introduced cysteine residue.

In one embodiment, the engineered ALPP and/or ALPPL2 monomer comprises a G503C substitution in SEQ ID NO: 275, or an equivalent substitution in another ALPP and/or ALPPL2 monomer.

Human ALPP
(SEQ ID NO: 275)
MLGPCMLLLLLLLGLRLQLSLGIIPVEEENPDFWNREAAEALGAAKKLQPAQTAAKNLIIFLGDGMGVS
TVTAARILKGQKKDKLGPEIPLAMDRFPYVALSKTYNVDKHVPDSGATATAYLCGVKGNFQTIGLSAAA
RFNQCNTTRGNEVISVMNRAKKAGKSVGVVTTTRVQHASPAGTYAHTVNRNWYSDADVPASARQE
GCQDIATQLISNMDIDVILGGGRKYMFRMGTPDPEYPDDYSQGGTRLDGKNLVQEWLAKRQGARYV
WNRTELMQASLDPSVTHLMGLFEPGDMKYEIHRDSTLDPSLMEMTEAALRLLSRNPRGFFLFVEGGR
IDHGHHESRAYRALTETIMFDDAIERAGQLTSEEDTLSLVTADHSHVFSFGGYPLRGSSIFGLAPGKAR
DRKAYTVLLYGNGPGYVLKDGARPDVTESESGSPEYRQQSAVPLDEETHAGEDVAVFARGPQAHLV
HGVQEQTFIAHVMAFAACLEPYTACDLAPPAGTTDAAHPGRSVVPALLPLLAGTLLLLETATAP

In one embodiment, the engineered ALPP monomer comprises a sequence according to SEQ ID NO: 263.

According to a further aspect, the invention provides an engineered ALPP and/or ALPPL2 homodimer, comprising a first and second engineered ALPP and/or ALPPL2 monomer as disclosed herein.

Antibodies capable of binding to the target antigens as described herein may be isolated from a suitable antibody library via, for example, using the phage display, yeast display, ribosomal display, or mammalian display technology known in the art. The engineered ALPP and/or ALPPL2 monomer disclosed herein, or the engineered ALPP and/or ALPPL2 homodimer disclosed herein may be used the immunisation of an animal to generate ALPP and/or ALPPL2 specific binding molecules.

According to a further aspect, the invention provides a method for generation of ALPP and/or ALPPL2 specific binding molecules comprising;
(a) immunising an animal with the engineered ALPP and/or ALPPL2 homodimer of the thirteenth aspect,
(b) isolating Peripheral Blood Mononuclear Cells (PBMCs) from the animal,
(c) cloning into vectors RNA or cDNA sequences obtained from the PBMCs,
(d) transforming prokaryotic cells with the above vectors in conditions allowing the production of the specific binding molecules,
(e) selecting the specific binding molecules by subjecting them to antigen-affinity selection,
(f) recovering the specific binding molecules having the desired specificity.

In one embodiment, the animal is a llama.

In one embodiment, the antigen-affinity selection is performed by bio-panning.

Furthermore, any of the features described in respect of any of the above-mentioned aspects of the invention may be combined mutatis mutandis with the other aspects of the invention.

In addition to the sequences mentioned the following sequences are expressly disclosed. Certain of these sequences relate to examples of molecules of the invention described herein:

TABLE 2
additional sequences

| SEQ | | |
|---|---|---|
| ID NO: | Sequence name | Sequence |
| 209 | Human Fc region | EPKSSDKTHTCPPCPAPELLGGPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRDELTKNQVSLTCLVKGFYPSDIAV |
| | | EWESNGQPENNYKTTPPVLDSDGSFFLYSKLTVDKSRWQQGNVFSCSVMHEALHNHYTQKSLSLSPGK |
| 198 | hFc Y407T | EPKSSDKTHTCPPCPAPELLGGPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRDELTKNQVSLTCLVKGFYPSDIAV |
| | | EWESNGQPENNYKTTPPVLDSDGSFFLTSKLTVDKSRWQQGNVFSCSVMHEALHNHYTQKSLSLSPGK |
| 276 | hFc T366Y | EPKSSDKTHTCPPCPAPELLGGPSVFLFPPKPKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREPQVYTLPPSRDELTKNQVSLYCLVKGFYPSDIA |
| | | VEWESNGQPENNYKTTPPVLDSDGSFFLYSKLTVDKSRWQQGNVFSCSVMHEALHNHYTQKSLSLSPGK |
| 236 | G₄S Linker | GGGGS |
| 237 | [G₄S]₃ Linker | GGGGSGGGGSGGGGS |
| 242 | [G₄S]₅ Linker | GGGGSGGGGSGGGGSGGGGSGGGGS |
| 243 | [G₄S]₇ Linker | GGGGSGGGGSGGGGSGGGGSGGGGSGGGGSGGGGS |
| 245 | c-myc containing C- | EQKLISEEDL |
| | terminal tag | |
| 249 | C-terminal tag | AAAHHHHHHGAEFEQKLISEEDL |
| 250 | C-terminal tag | ACAHHHHHHGAEFEQKLISEEDL |
| 254 | C-terminal tag | AAAHHHHHH |
| 255 | C-terminal tag | ACAHHHHHH |
| 259 | C-terminal tag | ACA |
| 261 | UCL OKT3 | QVQLVQSGAEVKKPGSSVKVSCKASGYTFTRYTMHWVRQAPGQGLEWMGYINPSRGYTNYNQKFKDRVTITADKSTSTAY |
| | sequence | MELSSLRSEDTAVYYCARYYDDHYCLDYWGQGTMVTVSSVEGGSGGSGGSGGSGGVDDIQMTQSPSSLSASVGDRVTIT |
| | | CSASSSVSYMNWYQQKPGKAPKRLIYDTSKLASGVPSRFSGSGSGTEFTLTISSLOPEDFATYYCQQWSSNPFTFGQGTKV |
| | | EIK |
| 262 | Harpoon ID20 | DIKLQQSGAELARPGASVKMSCKTSGYTFTRYTMHWVKQRPGQGLEWIGYINPSRGYTNYNQKFKDKATLTTDKSSSTAY |
| | | MQLSSLTSEDSAVYYCARYYDDHYCLDYWGQGTTLTVSSVEGGSGGSGGSGGSGGVDDIQLTQSPAIMSASPGEKVTMT |
| | | CRASSSVSYMNWYQQKSGTSPKRWIYDTSKVASGVPYRFSGSGSGTSYSLTISSMEAEDAATYYCQQWSSNPLTFGAGTK |
| | | LELK |
| 263 | ALPP CysHis | IIPVEEENPDFWNREAAEALGAAKKLQPAQTAAKNLIIFLGDGMGVSTVTAARILKGQKKDKLGPEIPLAMDRFPYVALSKTYNVDKHVPDSG |
| | | ATATAYLCGVKGNFQTIGLSAAARFNQCNTTRGNEVISVMNRAKKAGKSVGVVTTTRVQHASPAGTYAHTVNRNWYSDADVPASARQEG |
| | | CQDIATQLISNMDIDVILGGGRKYMFRMGTPDPEYPDDYSQGGTRLDGKNLVQEWLAKRQGARYVWNRTELMQASLDPSVTHLMGLFEP |
| | | GDMKYEIHRDSTLDPSLMEMTEAALRLLSRNPRGFFLFVEGGRIDHGHHESRAYRALTETIMFDDAIERAGQLTSEEDTLSLVTADHSHVFSF |
| | | GGYPLRGSSIFGLAPGKARDRKAYTVLLYGNGPGYVLKDGARPDVTESESGSPEYRQQSAVPLDEETHAGEDVAVFARGPQAHLVHGVQEQ |
| | | TFIAHVMAFAACLEPYTACDLAPPACTTDGGGGSHHHHHH |
| 264 | ALPI His | VIPAEEENPAFWNRQAAEALDAAKKLQPIQKVAKNLILFLGDGLGVPTVTATRILKGQKNGKLGPETPLAMDRFPYLALSKTYNVDRQVPDS |
| | | AATATAYLCGVKANFQTIGLSAAARFNQCNTTRGNEVISVMNRAKQAGKSVGVVTTTRVQHASPAGTYAHTVNRNWYSDADMPASARQ |
| | | EGCQDIATQLISNMDIDVILGGGRKYMFPMGTPDPEYPADASQNGIRLDGKNLVQEWLAKHQGAWYVWNRTELMQASLDQSVTHLMGL |
| | | FEPGDTKYEIHRDPTLDPSLMEMTEAALRLLSRNPRGFYLFVEGGRIDHGHHEGVAYQALTEAVMFDDAIERAGQLTSEEDTLTLVTADHSH |
| | | VFSFGGYTLRGSSIFGLAPSKAQDSKAYTSILYGNGPGYVFNSGVRPDVNESESGSPDYQQQAAVPLSSETHGGEDVAVFARGPQAHLVHGV |
| | | QEQSFVAHVMAFAACLEPYTACDLAPPACTTDGGGGSHHHHHH |
| 265 | Human ALPP | IIPVEEENPDFWNREAAEALGAAKKLQPAQTAAKNLIIFLGDGMGVSTVTAARILKGQKKDKLGPEIPLAMDRFPYVALSKTYNVDKHVPDSG |
| | (leucine zipper) | ATATAYLCGVKGNFQTIGLSAAARFNQCNTTRGNEVISVMNRAKKAGKSVGVVTTTRVQHASPAGTYAHTVNRNWYSDADVPASARQEG |
| | | CQDIATQLISNMDIDVILGGGRKYMFRMGTPDPEYPDDYSQGGTRLDGKNLVQEWLAKRQGARYVWNRTELMQASLDPSVTHLMGLFEP |
| | | GDMKYEIHRDSTLDPSLMEMTEAALRLLSRNPRGFFLFVEGGRIDHGHHESRAYRALTETIMFDDAIERAGQLTSEEDTLSLVTADHSHVFSF |
| | | GGYPLRGSSIFGLAPGKARDRKAYTVLLYGNGPGYVLKDGARPDVTESESGSPEYRQQSAVPLDEETHAGEDVAVFARGPQAHLVHGVQEQ |
| | | TFIAHVMAFAACLEPYTACDLAPPAGTTDGGGGSCGGLTDTLQAETDQLEDEKSALQTEIANLLKEKEKLEFILAAHGGCGGHHHHHH |
| 266 | Human ALPPL2 | IIPVEEENPDFWNRQAAEALGAAKKLQPAQTAAKNLIIFLGDGMGVSTVTAARILKGQKKDKLGPETFLAMDRFPYVALSKTYSVDKHVPDS |
| | (leucine zipper) | GATATAYLCGVKGNFQTIGLSAAARFNQCNTTRGNEVISVMNRAKKAGKSVGVVTTTRVQHASPAGAYAHTVNRNWYSDADVPASARQE |
| | | GCQDIATQLISNMDIDVILGGGRKYMFPMGTPDPEYPDDYSQGGTRLDGKNLVQEWLAKHQGARYVWNRTELLQASLDPSVTHLMGLFE |
| | | PGDMKYEIHRDSTLDPSLMEMTEAALLLLSRNPRGFFLFVEGGRIDHGHHESRAYRALTETIMFDDAIERAGQLTSEEDTLSLVTADHSHVFSF |
| | | GGYPLRGSSIFGLAPGKARDRKAYTVLLYGNGPGYVLKDGARPDVTESESGSPEYRQQSAVPLDGETHAGEDVAVFARGPQAHLVHGVQEQ |
| | | TFIAHVMAFAACLEPYTACDLAPRAGTTDGGGGSCGGLTDTLQAETDQLEDEKSALQTEIANLLKEKEKLEFILAAHGGCGGHHHHHH |
| 267 | Human ALPL | LVPEKEKDPKYWRDQAQETLKYALELQKLNTNVAKNVIMFLGDGMGVSTVTAARILKGQLHHNPGEETRLEMDKFPFVALSKTYNTNAQV |
| | (leucine zipper) | PDSAGTATAYLCGVKANEGTVGVSAATERSRCNTTQGNEVTSILRWAKDAGKSVGIVTTTRVNHATPSAAYAHSADRDWYSDNEMPPEAL |
| | | SQGCKDIAYQLMHNIRDIDVIMGGGRKYMYPKNKTDVEYESDEKARGTRLDGLDLVDTWKSFKPRYKHSHFIWNRTELLTLDPHNVDYLLG |
| | | LFEPGDMQYELNRNNVTDPSLSEMVVVAIQILRKNPKGFFLLVEGGRIDHGHHEGKAKQALHEAVEMDRAIGQAGSLTSSEDTLTVVTADH |
| | | SHVFTFGGYTPRGNSIFGLAPMLSDTDKKPFTAILYGNGPGYKVVGGERENVSMVDYAHNNYQAQSAVPLRHETHGGEDVAVFSKGPMAH |
| | | LLHGVHEQNYVPHVMAYAACIGANLGHCAPASGGGGSCGGLTDTLQAETDQLEDEKSALQTEIANLLKEKEKLEFILAAHGGCGGHHHHHH |
| 268 | hFc S293C | EPKSSDKTHTCPPCPAPELLGGPCVFLFPPKPKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| 269 | hFc S442C | EPKSSDKTHTCPPCPAPELLGGPSVFLFPPKPKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLCLSPGK |
| 270 | hFc S293C+S442C | EPKSSDKTHTCPPCPAPELLGGPCVFLFPPKPKDTLMISRTPEVTCVVVDVSHEDPEVKFNWYVDGVEVHNAKTKPREEQYNSTYRVVSVLTV |
| | | LHQDWLNGKEYKCKVSNKALPAPIEKTISKAKGQPREPQVYTLPPSRDELTKNQVSLTCLVKGFYPSDIAVEWESNGQPENNYKTTPPVLDSD |
| | | GSFFLYSKLTVDKSRWQQGNVFSCSVMHEALHNHYTQKSLCLSPGK |
| 271 | Mouse ALPPL2 | VIPVEEENPAFWNRKAAEALDAAKKLKPIQTSAKNLVILMGDGMGVSTVTATRILKGQQQGHLGPETQLAMDRFPHMALSKTYNTDKQIP |
| | (leucine zipper) | DSAGTGTAFLCGVKTNMKVIGLSAAARFNQCNTTWGNEVVSVMHRAKKAGKSVGVVTTTSVQHASPAGTYAHTVNRGWYSDAQMPAS |
| | | ALQDGCKDISTQLISNMDIDVILGGGRKFMFPKGTPDQEYPTDTKQAGTRLDGRNLVQEWLAKHQGARYVWNRSELIQASLNRSVTHLMG |
| | | LFEPNDMKYEIHRDPAQDPSLAEMTEVAVRMLSRNPKGFYLFVEGGRIDHGHHETVAYRALTEAVMFDSAVDKADKLTSEQDTMILVTAD |
| | | HSHVFSFGGYTQRGASIFGLAPFKAEDGKSFTSILYGNGPGYKLHNGARADVTEEESSNPTYQQQAAVPLSSETHSGEDVAIFARGPQAHLVH |
| | | GVQEQNYIAHVMAFAACLEPYTDCGLASPAGQSSGGGGSCGGLTDTLQAETDQLEDEKSALQTEIANLLKEKEKLEFILAAHGGCGGHHHH |
| | | HH |
| 272 | Rhesus monkey | IIPVEEENPDFWNRQAAEALGAAKKLQPIQTAAKNLIIFLGDGMGVSTVTAARILKGQKEDKLGPETPLAMDHFPYVALSKTYSVDKHVPDSA |
| | ALPP | ATATAYLCGVKGNFQTIGLSAAARYNQCNTTRGNEVVSVMNRAKKAGKSVGVVTTTRVQHASPAGAYAHTVNRNWYSDANMPGSARRE |
| | (leucine zipper) | GCKDIATQLISNMDIDVILGGGRKYMFRMGAPDPEYPHDYSQDGTRMDGKNLVQEWLAKQQGARYVWNRTELMQASLDPSVTHLMGL |
| | | FEPGDMKYEIHRDPTLDPSLMEMTEAALRLLSRNPRGFFLFVEGGRIDHGHHESRAYRALTEAVMFDDAIERAGQLTSEEDTLTLVTADHSH |
| | | VFSFGAYPLRGSSIFGLAPGKAQDRKAYTALLYGNGPGYVLKDGARPDVTESESGSPEYRQQAAVPLDEETHAGEDVAVFARGPQAHLVHG |
| | | VQEQSFIAHVMAFAACLEPYTACDLAPPAGTTDGGGGSCGGLTDTLQAETDQLEDEKSALQTEIANLLKEKEKLEFILAAHGGCGGHHHHHH |
| 273 | Cynomolgus | IIPVEEENPDFWNRQAAEALGAAKKLQPIQTAAKNLIIFLGDGMGVSTVTAARILKGQKEEKLGPETPLAMDHFPYVALSKTYSVDKHVPDSA |
| | monkey ALPP | ATATAYLCGVKGNFQTIGLSAAARYNQCNTTRGNEVVSVMNRAKKAGKSVGVVTTTRVQHASPAGAYAHTVNRNWYSDANMPGSARRE |
| | (leucine zipper) | GCKDIATQLISNMDIDVILGGGRKYMFRMGAPDPEYPHDYSQDGTRMDGKNLVQEWLAKHQGARYVWNRTELMQASLDPSVTHLMGLF |
| | | EPGDMKYEIHRDPTLDPSLMEMTEAALRLLSRNPRGFFLFVEGGRIDHGHHESRAYRALTEAVMFDDAIERAGQLTSEEDTLTLVTADHSHV |
| | | FSFGAYPLRGSSIFGLAPGKAQDRKAYTALLYGNGPGYVLKDGARPDVTESESGSPEYRQQAAVPLDEETHAGEDVAVFARGPQAHLVHGV |
| | | QEQSFVAHVMAFAACLEPYTACDLAPPAGTTDGGGGSCGGLTDTLQAETDQLEDEKSALQTEIANLLKEKEKLEFILAAHGGCGGHHHHHH |

According to a further aspect, the invention provides a target-binding molecule-drug conjugate, comprising
(a) an ALPP and/or ALPPL2 specific antigen binding molecule, a recombinant fusion protein, or a recombinant fusion protein dimer as disclosed herein, and
(b) at least one toxin, cytotoxic or cytostatic agent.

The ALPP and/or specific antigen binding molecule may be conjugated to the toxin, cytotoxic or cytostatic agent via a linker.

The at least one toxin may be selected from the group comprising:
auristatins,
anthracyclines, preferably PNU-derived anthracyclines
maytansinoids,
amanitin derivatives, preferably α-amanitin derivatives
calicheamicins,
tubulysins
duocarmycins
radioisotopes—such as an alpha-emitting radionuclide, such as 227 Th and 225 Ac label
liposomes comprising a toxic payload,
protein toxins
taxanes
pyrrolbenzodiazepines and dimers thereof
indolinobenzodiazepine pseudodimers
spliceosome inhibitors
CDK11 inhibitors
nicotinamide phosphoribosyltransferase inhibitors (NAMPTi)
Pyridinobenzodiazepines and dimers thereof
Cyclopropapyrroloindole (CPI), cyclopropabenzindole (CBI) or cyclopropathienoindole (CTI) and optionally dimers thereof
Irinotecan or exatecan and their derivatives.

The toxin may be an auristatin. The auristatin may be Auristatin E (AE) or monomethylauristatin E (MMAE). The auristatin may be an MMAE derivative. Any of the spacer ([X]) and/or linker ([L1] and/or [L2]) groups described herein in connection with anthracycline toxins are also explicitly contemplated in connection with auristatins such as MMAE. In preferred embodiments, the target-binding molecule-drug conjugate may comprise Val-Cit-PAB-MMAE (vcPAB-MMAE).

In one aspect, (b) is an a MMAE derivative,

wherein the target-binding molecule-drug conjugate has the structure of formula (VI):

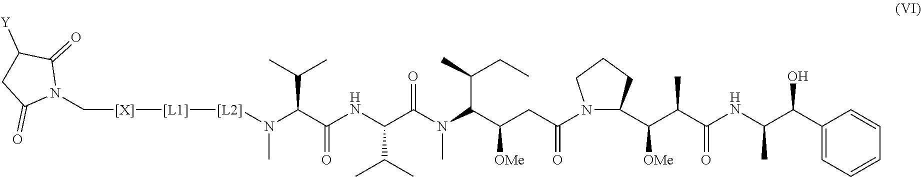

[X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, a dipeptide, a tripeptide, —(CH2)n-, —(CH2CH2O)n-, p-aminobenzyloxycarbonyl (PAB), Val-Cit-PAB, Val-Ala-PAB, Ala-Ala-Asn-PAB, Val-Ala, Asn-Ala, any amino acid, D-amino acids, Phe-Lys-PAB, and combinations thereof; and Y comprises an ALPP and/or ALPPL2 specific antigen binding molecule or recombinant fusion protein as disclosed herein.

Preferably, the target-binding molecule-drug conjugate has the structure of formula (VII):

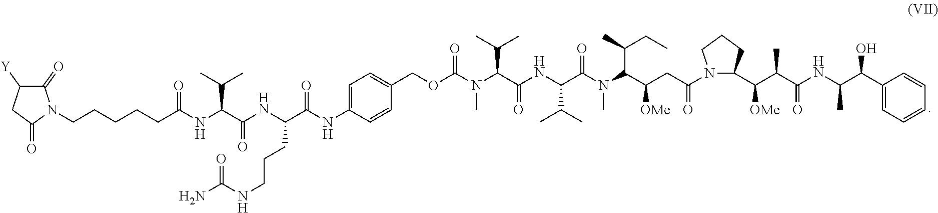

The toxin may be Exatecan or an Exatecan derivative.

The Exatecan derivative may have the structure of formula (X):

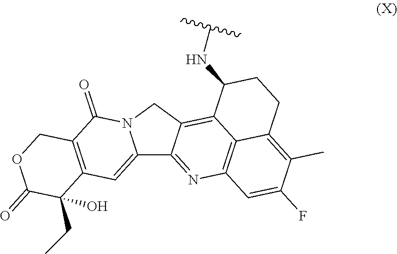 [SMILES: *N[C@H]1CCc2c(C)c(F)cc3nc4c(c1c23)Cn1c-4cc2c(c1=O)COC(=O)[C@]2(O)CC]

wherein the point of attachment to a linker is denoted by a wavy line.

A highly interesting class of DNA intercalating toxins for use as payloads for drug conjugates are anthracyclines, because of their proven clinical validation as chemotherapeutic drugs in cancer therapy. Stability of chemically-conjugated protein drug conjugates is an important consideration, since unintended release of a highly potent anthracycline toxin, like PNU-159682, in the circulation of a patient prior to targeting of the tumour cells would lead to off target effects and undesirable side effects. Some example molecules released from PNU conjugates include release of PNU159682 derivative from different Val-Cit-PAB containing drug linkers.

Potent toxins that can be linked to targeting proteins with high stability are therefore required in order to avoid, or at least reduce, unwanted side effects. Alternatively, linker payloads are designed such that extracellular cleavage releases derivatives of the payload with attenuated potency. However, sufficient potency needs to be retained in order to avoid any reduction in side effect being negated due to the need to administer higher doses to achieve efficacy.

Ease of conjugation is an important factor in producing easily manufacturable products. Payloads of the present disclosure may use a maleimide group, which can react to any available thiol group on a conjugation partner using straightforward and standard conditions. Furthermore, the use of maleimide/thiol chemistry for conjugation allows for site-specific conjugation to introduced thiol groups, for example on the side-chain of an engineered cysteine residue in a protein sequence. In some cases described herein, a cysteine may be introduced via the introduction of his-myc tag containing an engineered cysteine (example sequences include, but are not limited to, ACAHHHHHHGAEFEQKLISEEDL (SEQ ID NO: 250) at the C- or N-terminal of a protein.

Antibody/protein drug conjugates generated using non-selective labelling methods, such as through reaction with amino functionalities within proteins, deliver products containing multiple different species with differing drug to antibody ratios. This impacts the properties of the conjugate including potency and PK properties which impacts in vivo efficacy and toxicities. Therefore, thiol reactive payloads are of great importance, as these can be reacted in high yield, in a simple process, with naturally occurring cysteine residues in proteins or with a cysteine residue engineered into a specific site at any point within the sequence of proteins using molecular biology/recombinant protein expression or chemical synthesis or through chemical modification of expressed, synthetic or natural proteins. In some cases described herein, the cysteine is engineered into the Fc region of an Fc fusion protein.

The present disclosure provides anthracycline (PNU) derivatives suitable for use in drug conjugates. Specifically, derivatives of PNU159682 are provided, which lack the C14 carbon and attached hydroxyl functionality, and are functionalised with an ethylenediamino (EDA) group at the C13 carbonyl of PNU159682. This EDA-PNU159682 can in turn be functionalised, through the amino group of the EDA moiety, with a maleimide containing linker. A maleimide group is present in the anthracycline (PNU) derivatives of formula (V) and may also be present in the anthracycline (PNU) derivatives of formula (VI). Such payloads are able to react with a free thiol group on another molecule. Where the free thiol is on a protein, a protein-drug conjugate (PDC) may be formed.

Surprisingly, derivatives of PNU159682 functionalised with an ethylenediamino (EDA) group and linked to a thiol group via a maleimide group show higher stability compared to non-EDA payloads or liberated payload derivatives with slightly less potency. More stable payloads may be advantageous because of reduced off-target effects, which in turn may lead to reduced side effects and increased patient compliance.

PCT/EP2020/067210 describes anthracycline (PNU) derivatives of formula (V):

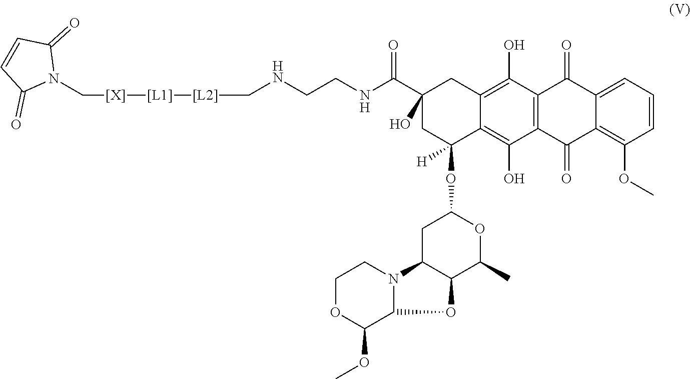

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof.

The anthracycline (PNU) derivative of formula (V) may comprise [L1], [L2] or [L1] and [L2].

Preferably, where [L1] and/or [L2] are peptides, said peptides do not contain glycine.

It will be clear to those of skill in the art that when optional spacers and/or optional linkers are absent a bond remains in their place.

Preferably, [X] is selected from the group comprising polyethylene glycol,

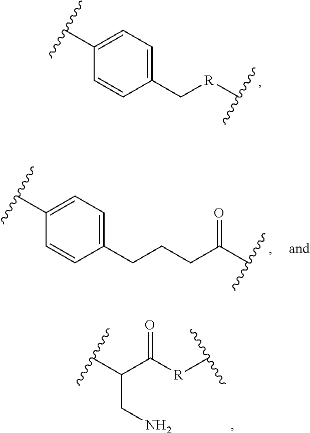

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

Preferably, [L2] is p-aminobenzyloxycarbonyl (PAB) or Alanine.

Preferably, the anthracycline (PNU) derivative comprises [L1] and/or [L2] and [X] is optional. Accordingly, [L1] and/or [L2] may be linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof. The anthracycline (PNU) derivative of formula (V) may comprise [L1], [L2] or [L1] and [L2]. The anthracycline (PNU) derivative of formula (V) may comprise [L1] and/or [L2].

PCT/EP2020/067210 describes anthracycline (PNU) derivatives of formula (V):

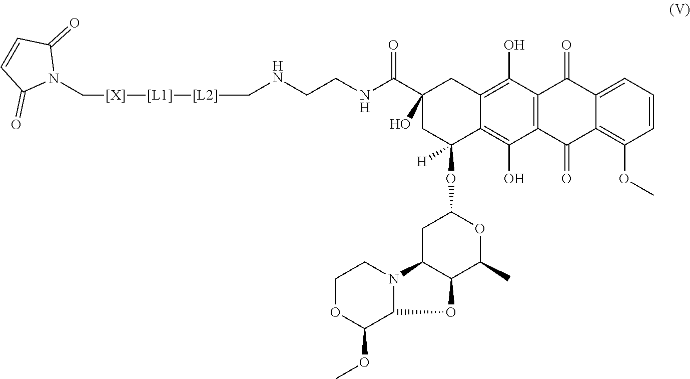

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and/or [L2] are linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof;
   wherein the anthracycline (PNU) derivative of formula (V) comprises [L1], [L2] or [L1] and [L2].

Preferably, [X] is selected from the group comprising polyethylene glycol,

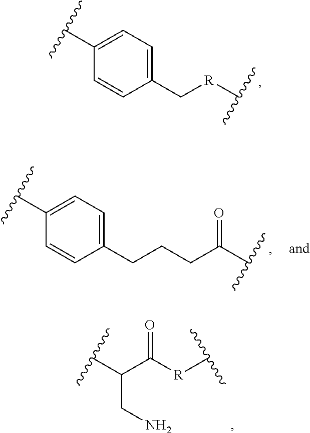

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

Preferably, [L2] is p-aminobenzyloxycarbonyl (PAB) or Alanine.

Preferably, the PNU derivative has a structure selected from:

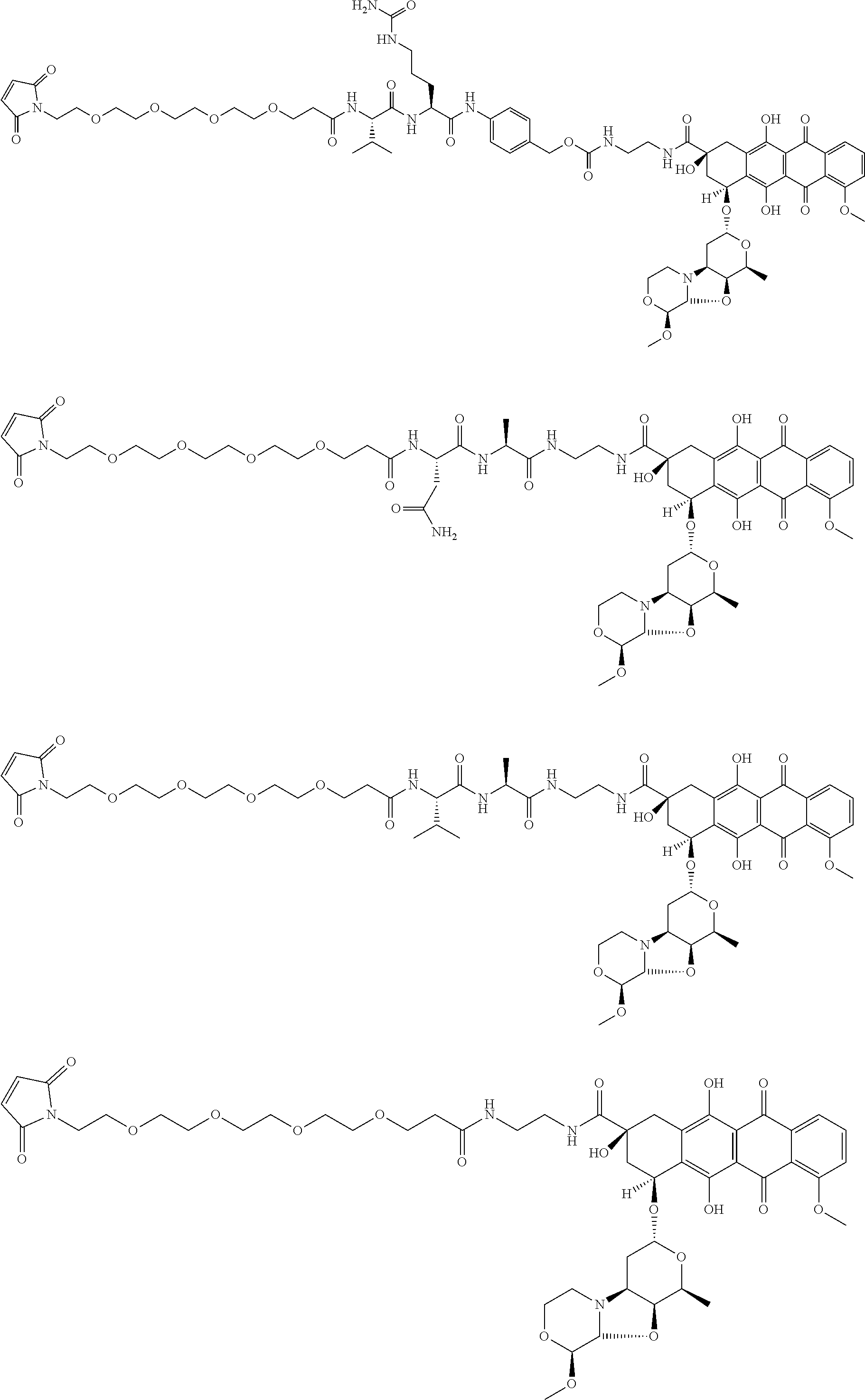

PCT/EP2020/067210 also describes anthracycline (PNU) derivatives of formula (VI):

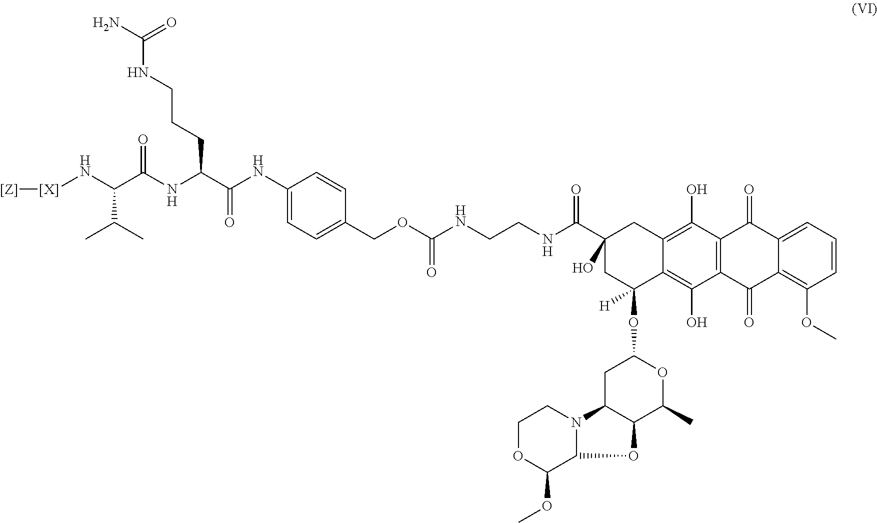

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
wherein [Z] is a reactive group. The reactive group may be any reactive group suitable for use in a conjugation reaction, particularly a conjugation reaction to a target binding molecule.
[Z] may therefore be a moiety comprising a functional group for use in bioconjugation reactions.

Functional groups for use in bioconjugation reactions include but are not limited to,
maleimides or alkyl halides for reaction with thiol groups or selenol groups on proteins through thioether and selonoether reactions;
sulphydryl groups for reaction with maleimide, alkyl halide or thiol functionalised molecules including the thiol groups of protein cysteine residues;
activated disulphides such as pyridyl dithiols (Npys thiols) or TNB thiols (5-thiol-2-nitrobenzoic acid) for reaction with thiol groups to form disulphide linkages through thiol disulphide exchange;
amino groups for attachment to carboxyl groups on proteins and biomolecules through amide bond forming reactions;
alkyne groups, particularly ring constrained alkynes such as dibenzocyclooctyne (DBCO) or bicyclo[6.1.0] nonyne (BCN) for the reaction with azido functionalised biomolecules through strain promoted alkyne-azide cycloaddition copper free chemistry. Azido functionalities can be introduced into proteins through, for example, the incorporation of the unnatural amino acid para-azidomethy-L-phenyalanine or into protein glycans using enzyme mediated glycoengineering to attach azido-containing sugar analogues;
azido groups for reaction with alkyne functionalised target-binding molecule through strain promoted alkyne-azide cycloaddition copper free chemistry;
aminoxy groups for reactions with aldehyde and ketone groups on biomolecules through oxime forming ligations. Ketones can be introduced into proteins through the use of amber stop codon technologies such as the incorporation of the non-natural amino acid, para-acetyl phenylalanine. Aldehydes can be found on biomolecules through the presence of reducing sugars and can be introduced into proteins through periodate oxidation of N-terminal serine residues or periodate oxidation of cis-glycol groups of carbohydrates. Aldehyde groups can also be incorporated into proteins through the conversion of protein cysteines, within specific sequences, to formyl glycine by formylglycine generating enzyme. In addition formylglycine containing proteins have been conjugation to payloads via the Hydrazino-Pictet-Spengler (HIPS) ligation;
aldehyde or ketone groups for the reaction with aminoxy or hydrazide or hydrazinyl functionalized biomolecules through oxime or hydrazine bond forming ligation reactions. Protein aminoxy and hydrazide functionalized proteins can be generated through cleavage of intein-fusion proteins.
[Z] may therefore be selected from the group consisting of a maleimide, an alkyl halide, a sulphydryl group, an activated disulphide (such as pyridyl dithiols (Npys thiols) or TNB thiols (5-thiol-2-nitrobenzoic acid)), an amino group, an alkyne group (such as ring constrained alkynes such as dibenzocyclooctyne (DBCO) or bicyclo[6.1.0] nonyne (BCN)), an azido group, an aminoxy group, an aldehyde group and a ketone group.
[Z] may also be a moiety for enzyme mediated bioconjugation reactions. Moieties for use in enzyme mediated conjugation reactions include but are not limited to polyGly [(Gly)ₙ] for use in sortase-enzyme mediated antibody conjugation or an appropriate primary amine for bacterial transglutaminase mediated conjugation to glutamine γ-carboxyamide groups contained with sequences such as Lys-Lys-Gln-Gly and Lys-Pro-Glu-Thr-Gly.
[Z] may therefore be selected from the group consisting of polyGly and a primary amine.

The PNU derivative according to formula (VI) may therefore correspond to a PNU derivative of formula (V) wherein L1 is Val-Cit-PAB, L2 is absent and wherein the maleimide group may be replaced with another Reactive Group as defined above.

Preferably, [X] is selected from the group comprising polyethylene glycol,

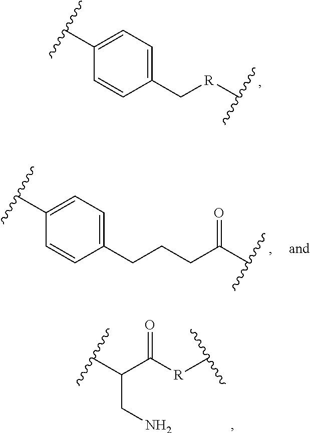

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

The PNU derivative according to formula (V) or formula (VI) may be conjugated to a ALPP and/or ALPPL2 specific antigen binding molecule according to the present invention or to a recombinant fusion protein or recombinant fusion protein dimer of the invention.

According to a further aspect, the invention provides a target-binding molecule-drug conjugate, comprising
(a) an ALPP and/or ALPPL2 specific antigen binding molecule as disclosed herein, or a recombinant fusion protein as disclosed herein or a recombinant fusion protein dimer as disclosed herein, and
(b) an anthracycline (PNU) derivative, wherein the target-binding molecule-drug conjugate has the structure of formula (III):

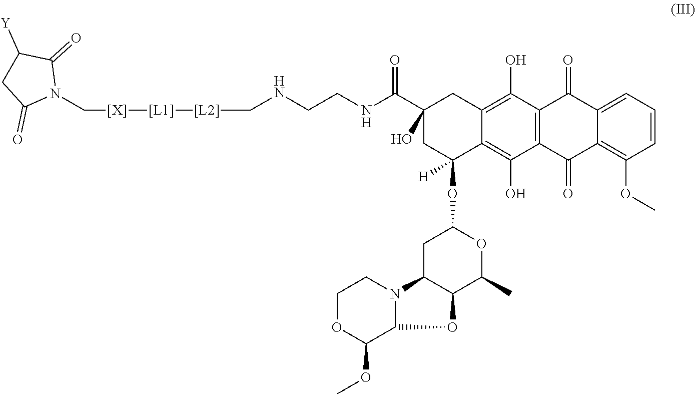

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH₂O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof; and
Y comprises an ALPP and/or ALPPL2 specific antigen binding molecule, a recombinant fusion protein, or a recombinant fusion protein dimer as disclosed herein.

The target-binding molecule-drug conjugate of formula (III) may comprise [L1], [L2] or [L1] and [L2].

Preferably, target-binding molecule-drug conjugate where [L1] and/or [L2] are peptides, said peptides do not contain glycine.

It will be clear to those of skill in the art that when optional spacers and/or optional linkers are absent a bond remains in their place.

Preferably, the target-binding molecule-drug conjugate has a structure selected from:

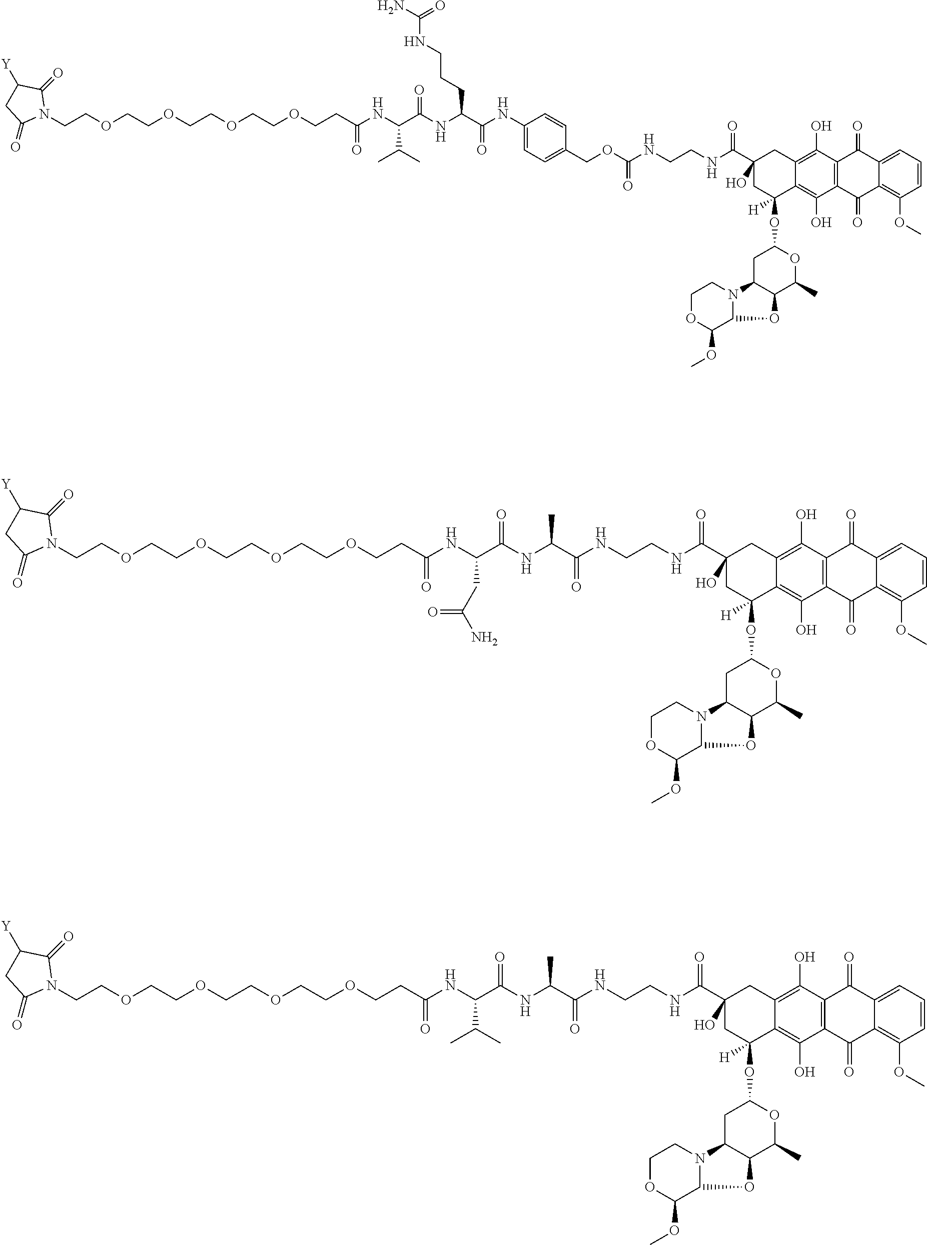

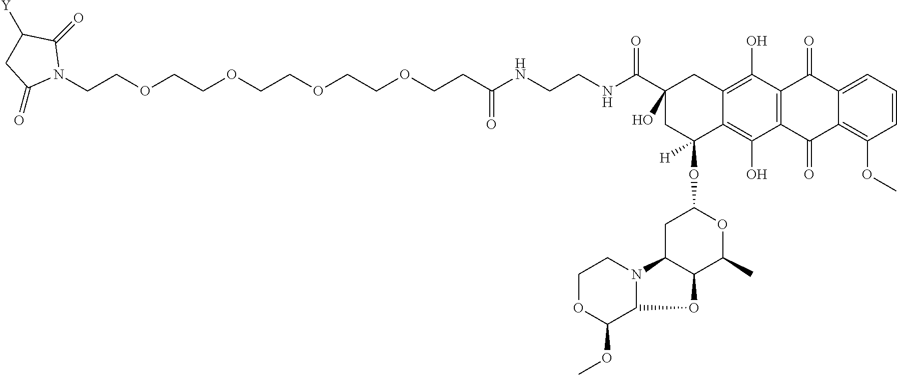

According to a further aspect, the invention provides a target-binding molecule-drug conjugate, comprising
(a) an ALPP and/or ALPPL2 specific antigen binding molecule as disclosed herein, or a recombinant fusion protein or recombinant fusion protein dimer as disclosed herein, and
(b) an anthracycline (PNU) derivative, wherein the target-binding molecule-drug conjugate has the structure of formula (IV):

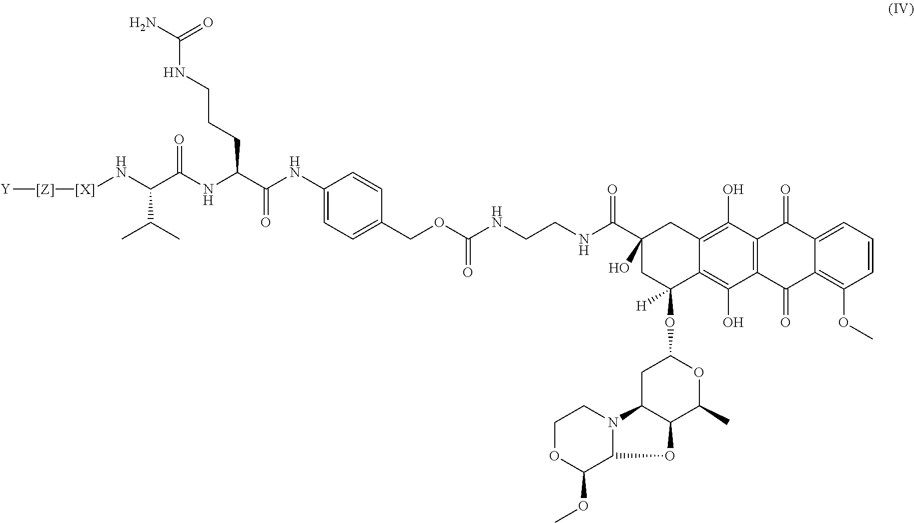

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[Z] is a linker derived from a reactive group used to conjugate the anthracycline (PNU) derivative and the target-binding molecule; and
Y comprises an ALPP specific antigen binding molecule according to the first, second or ninth aspect, or a recombinant fusion protein or recombinant fusion protein dimer according to the third, fourth or fifth aspect.

[Z] is a typically a moiety derived from a reactive group used to conjugate the anthracycline (PNU) derivative and the target-binding molecule. [Z] may be a moiety derived from a reactive group selected from the group consisting of a maleimide, an alkyl halide, a sulphydryl group, an activated disulphide, an amino group, an alkyne group, an azido group, an aminoxy group, an aldehyde group and a ketone group.

[Z] may therefore be selected from the group consisting of a disulphide bond, an amide bond, an oxime bond, a hydrazone bond, a thioether bond, a 1, 2, 3 triazole and polyGly.

Preferably, [X] is selected from the group comprising polyethylene glycol,

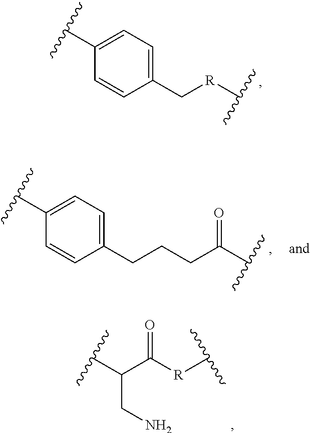

wherein [symbol] represents the point of attachment to the rest of the molecule and wherein [R] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof.

Most preferably, [X] is polyethylene glycol. The polyethylene glycol may be PEG4.

In one embodiment of the invention, the target-binding molecule-drug conjugate, Y comprises an ALPP and/or ALPPL2 specific antigen binding molecule as disclosed herein, conjugated to the PNU derivative via a human immunoglobulin Fc region or fragment thereof.

In one embodiment the fragment of the human immunoglobulin Fc region may be selected from the group consisting of an Fc heavy chain, a CH2 region and a CH3 region.

According to a further aspect, the invention provides a bi-paratopic alkaline phosphatase, placental type (ALPP) and/or alkaline phosphatase, germ cell type (ALPPL2) specific antigen binding molecule.

According to a further aspect, the invention provides a bi-paratopic target-binding molecule-drug conjugate, comprising
(a) a bi-paratopic alkaline phosphatase, placental type (ALPP) and/or alkaline phosphatase, germ cell type (ALPPL2) specific antigen binding molecule, and
(b) at least one toxin, cytotoxic or cytostatic agent.

In one embodiment of the bi-paratopic target-binding molecule-drug conjugate, the bi-paratopic ALPP and/or ALPPL2 specific antigen binding molecule is conjugated to the cytotoxic or cytostatic agent via a linker,

In one embodiment of the bi-paratopic target-binding molecule-drug conjugate, (b) is a toxin selected from the group consisting of:
auristatins,
anthracyclines, preferably PNU-derived anthracyclines
maytansinoids,
amanitin derivatives, preferably α-amanitin derivatives
calicheamicins,
tubulysins
duocarmycins
radioisotopes—such as an alpha-emitting radionuclide, such as 227 Th and 225 Ac label
liposomes comprising a toxic payload,
protein toxins
taxanes
pyrrolbenzodiazepines and dimers thereof
indolinobenzodiazepine pseudodimers
spliceosome inhibitors
CDK11 inhibitors
nicotinamide phosphoribosyltransferase inhibitors (NAMPTi)
Pyridinobenzodiazepines and dimers thereof
Cyclopropapyrroloindole (CPI), cyclopropabenzindole (CBI) or cyclopropathienoindole (CTI) and optionally dimers thereof
Irinotecan or exatecan and their derivatives.

In one embodiment of the bi-paratopic target-binding molecule-drug conjugate, the toxin is an auristatin.

In one embodiment of the bi-paratopic target-binding molecule-drug conjugate, the toxin is a monomethyl auristatin.

In one embodiment of the bi-paratopic target-binding molecule-drug conjugate, the toxin is monomethyl auristatin E.

In one embodiment of the bi-paratopic target-binding molecule-drug conjugate, (b) is an a MMAE derivative,

wherein the target-binding molecule-drug conjugate has the structure of formula (VI):

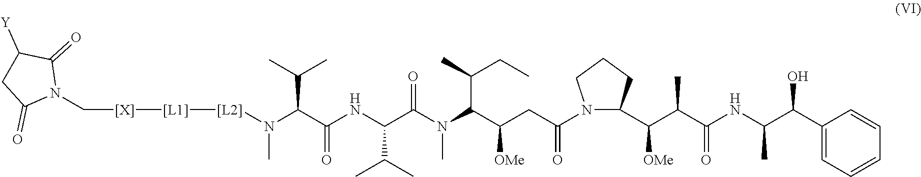

[X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, a dipeptide, a tripeptide, —(CH2)ₙ—, —(CH2CH2O)ₙ—, p-aminobenzyloxycarbonyl (PAB), Val-Cit-PAB, Val-Ala-PAB, Ala-Ala-Asn-PAB, Val-Ala, Asn-Ala, any amino acid, D-amino acids, Phe-Lys-PAB, and combinations thereof; and
Y comprises a bi-paratopic ALPP and/or ALPPL2 specific antigen binding molecule as disclosed herein.

In one embodiment of the bi-paratopic target-binding molecule-drug conjugate (b) is an a MMAE derivative,

wherein the target-binding molecule-drug conjugate has the structure of formula (VII):

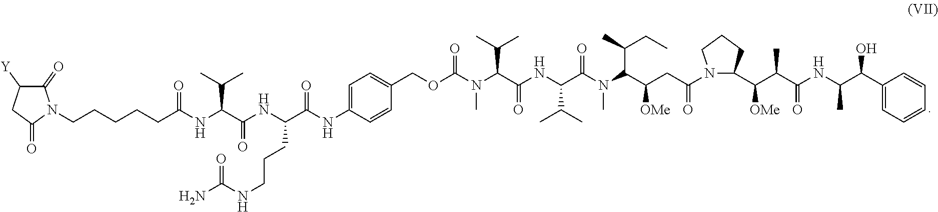

In one embodiment of the bi-paratopic target-binding molecule-drug conjugate, (b) is an anthracycline (PNU) derivative,

wherein the target-binding molecule-drug conjugate has the structure of formula (III):

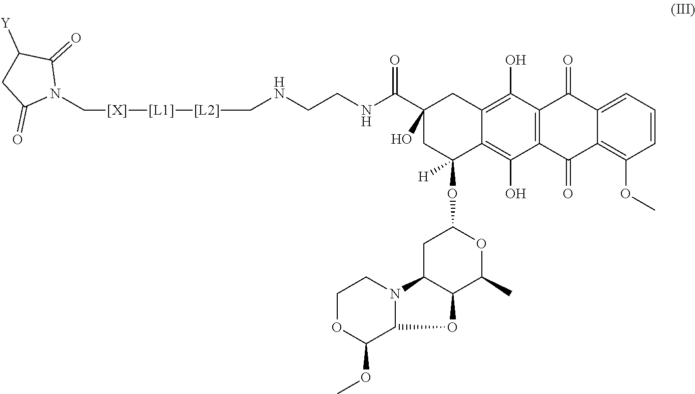

wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH2O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof; and
Y comprises a bi-paratopic ALPP and/or ALPPL2 specific antigen binding molecule as disclosed herein.

In one embodiment of the bi-paratopic target-binding molecule-drug conjugate, the target-binding molecule-drug conjugate has a structure selected from:

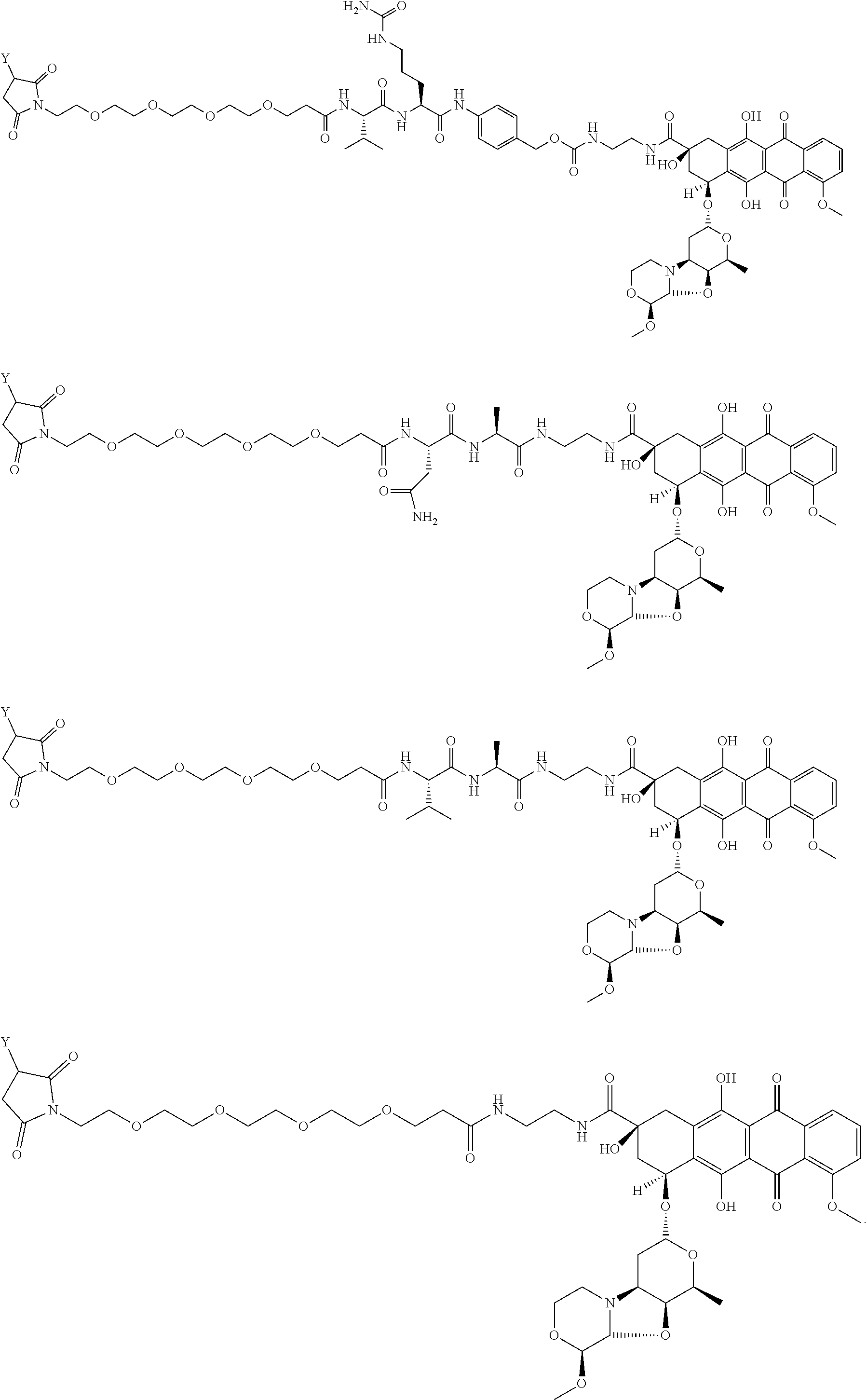

Also provided herein is the target-binding molecule-drug conjugate according to the above aspects, for use in therapy.

Also provided herein is the target-binding molecule-drug conjugate according to the above aspects, for use in the treatment of cancer.

Also provided herein is the use of a target-binding molecule-drug conjugate according to the above aspects in the manufacture of a medicament for the treatment of a disease in a patient in need thereof.

Also provided herein is a method of treatment of a disease in a patient in need of treatment comprising administration to said patient of a therapeutically effective dosage of a target-binding molecule-drug conjugate according to the above aspects. The disease may be cancer.

Preferably, the cancer is a ALPP and/or ALPPL2-positive cancer type. More preferably, the cancer is selected from the group consisting of ovarian cancer, endometrial cancer, cervical cancer, gastric cancer, oesophageal cancer, bladder cancer, lung cancer, pancreatic cancer and testicular cancer

Also provided herein is a pharmaceutical composition comprising a target-binding molecule-drug conjugate according to any of the above aspects, and at least one other pharmaceutically acceptable ingredient.

### Definitions

An antigen specific binding molecule of the invention comprises amino acid sequence derived from a synthetic library of VHH molecules, or from libraries derived from the immunization of a camelid. The terms VHH and single domain antibody (sdAb) may be used interchangeably also.

Amino acids are represented herein as either a single letter code or as the three letter code or both.

The term “affinity purification” means the purification of a molecule based on a specific attraction or binding of the molecule to a chemical or binding partner to form a combination or complex which allows the molecule to be separated from impurities while remaining bound or attracted to the partner moiety.

The term “Complementarity Determining Regions” or CDRs (i.e., CDR1, CDR2 and CDR3) refers to the amino acid residues of a VHH domain the presence of which are typically involved in antigen binding. Each VHH typically has three CDR regions identified as CDR1, CDR2 and CDR3.

“Framework regions” (FW) are those VHH residues other than the CDR residues. Each VHH typically has four framework regions identified as FW1, FW2, FW3 and FW4.

The boundaries between FW and CDR regions in VHHs are not intended to be fixed and accordingly some variation in the lengths and compositions of these regions is to be expected. This will be understood by those skilled in the art, particularly with reference to work that have been carried out in analyzing these regions. (Wesolowski et al., Med Microbiol Immunol (2009) 198, 157-174; Vattekatte et al., PeerJ (2020) e8408). The molecules of the present invention, although defined by reference to FW and CDR regions herein, are not limited to these strict definitions. Variation in line with the understanding in the art as the structure of the VHH domain is therefore expressly contemplated herein.

A “codon set” refers to a set of different nucleotide triplet sequences used to encode desired variant amino acids. A set of oligonucleotides can be synthesized, for example, by solid phase synthesis, including sequences that represent all possible combinations of nucleotide triplets provided by the codon set and that will encode the desired group of amino acids. A standard form of codon designation is that of the IUB code, which is known in the art and described herein.

A codon set is typically represented by 3 capital letters in italics, e.g. NNK, NNS, XYZ, DVK etc. A “non-random codon set” therefore refers to a codon set that encodes select amino acids that fulfill partially, preferably completely, the criteria for amino acid selection as described herein. Synthesis of oligonucleotides with selected nucleotide “degeneracy” at certain positions is well known in that art, for example the TRIM approach (Knappek et aL.; J. Mol. Biol. (1999), 296, 57-86); Garrard & Henner, Gene (1993), 128, 103). Such sets of oligonucleotides having certain codon sets can be synthesized using commercial nucleic acid synthesizers (available from, for example, Applied Biosystems, Foster City, CA), or can be obtained commercially (for example, from Life Technologies, Rockville, MD). A set of oligonucleotides synthesized having a particular codon set will typically include a plurality of oligonucleotides with different sequences, the differences established by the codon set within the overall sequence. Oligonucleotides used according to the present invention have sequences that allow for hybridization to a VHH nucleic acid template and also may where convenient include restriction enzyme sites.

“Cell”, “cell line”, and “cell culture” are used interchangeably (unless the context indicates otherwise) and such designations include all progeny of a cell or cell line. Thus, for example, terms like “transformants” and “transformed cells” include the primary subject cell and cultures derived therefrom without regard for the number of transfers. It is also understood that all progeny may not be precisely identical in DNA content, due to deliberate or inadvertent mutations. Mutant progeny that have the same function or biological activity as screened for in the originally transformed cell are included.

“Control sequences” when referring to expression means DNA sequences necessary for the expression of an operably linked coding sequence in a particular host organism. The control sequences that are suitable for prokaryotes, for example, include a promoter, optionally an operator sequence, a ribosome binding site, etc. Eukaryotic cells use control sequences such as promoters, polyadenylation signals, and enhancers.

The term “coat protein” means a protein, at least a portion of which is present on the surface of the virus particle. From a functional perspective, a coat protein is any protein which associates with a virus particle during the viral assembly process in a host cell, and remains associated with the assembled virus until it infects another host cell.

The “detection limit” for a chemical entity in a particular assay is the minimum concentration of that entity which can be detected above the background level for that assay. For example, in the phage ELISA, the “detection limit” for a particular phage displaying a particular antigen binding fragment is the phage concentration at which the particular phage produces an ELISA signal above that produced by a control phage not displaying the antigen binding fragment.

A “fusion protein” and a “fusion polypeptide” refer to a polypeptide having two portions covalently linked together, where each of the portions is a polypeptide having a different property. The property may be a biological property, such as activity in vitro or in vivo. The property may also be a simple chemical or physical property, such as binding to a target antigen, catalysis of a reaction, etc. The two portions may be linked directly by a single peptide bond or through a peptide linker containing one or more amino acid residues. Generally, the two portions and the linker will be in reading frame with each other. Preferably, the two portions of the polypeptide are obtained from heterologous or different polypeptides.

The term “fusion protein” in this text means, in general terms, one or more proteins joined together by chemical means, including hydrogen bonds or salt bridges, or by peptide bonds through protein synthesis or both. Typically fusion proteins will be prepared by DNA recombination techniques and may be referred to herein as recombinant fusion proteins.

“Heterologous DNA” is any DNA that is introduced into a host cell. The DNA may be derived from a variety of sources including genomic DNA, cDNA, synthetic DNA and fusions or combinations of these.

The DNA may include DNA from the same cell or cell type as the host or recipient cell or DNA from a different cell type, for example, from an allogenic or xenogenic source. The DNA may, optionally, include marker or selection genes, for example, antibiotic resistance genes, temperature resistance genes, etc.

A “highly diverse position” refers to a position of an amino acid located in the variable regions of the light and heavy chains that have a number of different amino acid represented at the position when the amino acid sequences of known and/or naturally occurring antibodies or antigen binding fragments are compared. The highly diverse positions are typically in the CDR or HV regions.

“Identity” describes the relationship between two or more polypeptide sequences or two or more polynucleotide sequences, as determined by comparing the sequences. Identity also means the degree of sequence relatedness (homology) between polypeptide or polynucleotide sequences, as the case may be, as determined by the match between strings of such sequences. While there exist a number of methods to measure identity between two polypeptide or two polynucleotide sequences, methods commonly employed to determine identity are codified in computer programs. Preferred computer programs to determine identity between two sequences include, but are not limited to, GCG program package (Devereux, et aL., Nucleic acids Research, 12, 387 (1984), BLASTP, BLASTN, and FASTA (Atschul et aL., J. Molec. Biol. (1990) 215, 403).

Preferably, the amino acid sequence of the protein has at least 45% identity, using the default parameters of the BLAST computer program (Atschul et aL., J. Mol. Biol. (1990) 215, 403-410) provided by HGMP (Human Genome Mapping Project), at the amino acid level, to the amino acid sequences disclosed herein.

More preferably, the protein sequence may have at least 45%, 46%, 47%, 48%, 49%, 50%, 55%, 60%, 65%, 66%, 67%, 68%, 69%, 70%, 75%, 80%, 85%, 90% and still more preferably 95% (still more preferably at least 96%, 97%, 98% or 99%) identity, at the nucleic acid or amino acid level, to the amino acid sequences as shown herein.

The protein may also comprise a sequence which has at least 45%, 46%, 47%, 48%, 49%, 50%, 52%, 55%, 60%, 65%, 66%, 67%, 68%, 69%, 70%, 75%, 80%, 85%, 90%, 95%, 96%, 97%, 98%, or 99% identity with a sequence disclosed herein, using the default parameters of the BLAST computer program provided by HGMP, thereto.

A “library” refers to a plurality of VHHs or VHH fragment sequences (for example, polypeptides of the invention), or the nucleic acids that encode these sequences, the sequences being different in the combination of variant amino acids that are introduced into these sequences according to the methods of the invention.

“Ligation” is the process of forming phosphodiester bonds between two nucleic acid fragments. For ligation of the two fragments, the ends of the fragments must be compatible with each other. In some cases, the ends will be directly compatible after endonuclease digestion. However, it may be necessary first to convert the staggered ends commonly produced after endonuclease digestion to blunt ends to make them compatible for ligation. For blunting the ends, the DNA is treated in a suitable buffer for at least 15 minutes at 15° C. with about 10 units of the Klenow fragment of DNA polymerase I or T4 DNA polymerase in the presence of the four deoxyribonucleotide triphosphates. The DNA is then purified by phenol-chloroform extraction and ethanol precipitation or by silica purification. The DNA fragments that are to be ligated together are put in solution in about equimolar amounts. The solution will also contain ATP, ligase buffer, and a ligase such as T4 DNA ligase at about 10 units per 0.5 μg of DNA. If the DNA is to be ligated into a vector, the vector is first linearized by digestion with the appropriate restriction endonuclease(s). The linearized fragment is then treated with bacterial alkaline phosphatase or calf intestinal phosphatase to prevent self-ligation during the ligation step.

A “mutation” is a deletion, insertion, or substitution of a nucleotide(s) relative to a reference nucleotide sequence, such as a wild type sequence.

“Natural” or “naturally occurring” VHHs, refers to VHHs identified from a non-synthetic source, for example, from a tissue source obtained ex vivo, or from the serum of an animal of the Camelid subclass. These VHHs can include VHHs generated in any type of immune response, either natural or otherwise induced. Natural VHHs include the amino acid sequences, and the nucleotide sequences that constitute or encode these antibodies. As used herein, natural VHHs are different than “synthetic VHHs”, synthetic VHHs referring to VHH sequences that have been changed from a source or template sequence, for example, by the replacement, deletion, or addition, of an amino acid, or more than one amino acid, at a certain position with a different amino acid, the different amino acid providing an antibody sequence different from the source antibody sequence.

The term “nucleic acid construct” generally refers to any length of nucleic acid which may be DNA, cDNA or RNA such as mRNA obtained by cloning or produced by chemical synthesis. The DNA may be single or double stranded. Single stranded DNA may be the coding sense strand, or it may be the non-coding or anti-sense strand. For therapeutic use, the nucleic acid construct is preferably in a form capable of being expressed in the subject to be treated.

“Operably linked” when referring to nucleic acids means that the nucleic acids are placed in a functional relationship with another nucleic acid sequence. For example, DNA for a presequence or secretory leader is operably linked to DNA for a polypeptide if it is expressed as a preprotein that participates in the secretion of the polypeptide; a promotor or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contingent and in reading frame. However, enhancers do not have to be contiguous. Linking is accomplished by ligation at convenient restriction sites. If such sites do not exist, the synthetic oligonucleotide adapters or linkers are used in accord with conventional practice.

The term “protein” means, in general terms, a plurality of amino acid residues joined together by peptide bonds. It is used interchangeably and means the same as peptide, oligopeptide, oligomer or polypeptide, and includes glycoproteins and derivatives thereof. The term “protein” is also intended to include fragments, analogues, variants and derivatives of a protein wherein the fragment, analogue, variant or derivative retains essentially the same biological activity or function as a reference protein. Examples of protein analogues and derivatives include peptide nucleic acids, and DARPins (Designed Ankyrin Repeat Proteins).

A fragment, analogue, variant or derivative of the protein may be at least 25 preferably 30 or 40, or up to 50 or 100, or 60 to 120 amino acids long, depending on the length of the original protein sequence from which it is derived. A length of 90 to 120, 100 to 110 amino acids may be convenient in some instances.

The fragment, derivative, variant or analogue of the protein may be (i) one in which one or more of the amino acid residues are substituted with a conserved or non-conserved amino acid residue (preferably, a conserved amino acid residue) and such substituted amino acid residue may or may not be one encoded by the genetic code, or (ii) one in which one or more of the amino acid residues includes a substituent group, or (iii) one in which the additional amino acids are fused to the mature polypeptide, such as a leader or auxiliary sequence which is employed for purification of the polypeptide. Such fragments, derivatives, variants and analogues are deemed to be within the scope of those skilled in the art from the teachings herein.

The term “VHH” as used herein means the variable domain of the heavy chain of a heavy chain antibody, which is naturally devoid of light chains. The amino acid sequence and structure of a VHH can be considered, without however being limited thereto, to be comprised of four framework regions or “FWs”, which are referred to in the art and hereinbelow as “framework region 1” or “FW1”; as “framework region 2” or “FW2”; as “framework region 3” or “FW3”; and as “framework region 4” or “FW4”, respectively, which framework regions are interrupted by three complementary determining regions or “CDR's”, which are referred to in the art as “complementarity determining region 1” or “CDR1”; as “complementarity determining region 2” or “CDR2”; and as “complementarity determining region 3” or “CDR3”, respectively. As also further described hereinbelow, the total number of amino acid residues in including a VHH can be in the region of 110-130, is preferably 112-115, and is most preferably 113. It should however be noted that parts, fragments or analogs of a VHH domain of an antibody are not particularly limited as to their length and/or size, as long as such parts, fragments or analogs retain (at least part of) the functional activity, such as the cell killing activity (as defined herein) and/or retain (at least part of) the binding specificity of the original VHH from which these parts, fragments or analogs are derived from. Parts, fragments or analogs retaining (at least part of) the functional activity, such as the cell killing activity (as defined herein) and/or retaining (at least part of) the binding specificity of the VHH from which these parts, fragments or analogs are derived from are also further referred to herein as “functional fragments” or “functional variants” of a VHH. A method for numbering the amino acid residues of VHH domains is the method described by Chothia et al. (Nature 342, 877-883 (1989)), the so-called “AbM definition” and the so-called “contact definition”. Herein, this is the numbering system adopted.

Alternatively, the amino acid residues of a variable domain of a heavy chain variable domain of an antibody (including a VHH) may be numbered according to the general numbering for heavy chain variable domains given by Kabat et al. (“Sequence of proteins of immunological interest”, US Public Health Services, NIH Bethesda, Md., Publication No. 91), as applied to VHH domains from Camelids in Riechmann and Muyldermans (J Immunol Methods. 1999 Dec. 10; 231(1-2):25-38).

“Oligonucleotides” are short-length, single- or double-stranded polydeoxynucleotides that are chemically synthesized by known methods (such as phosphotriester, phosphite, or phosphoramidite chemistry, using solid-phase techniques). Further methods include the polymerase chain reaction (PCR) used if the entire nucleic acid sequence of the gene is known, or the sequence of the nucleic acid complementary to the coding strand is available. Alternatively, if the target amino acid sequence is known, one may infer potential nucleic acid sequences using known and preferred coding residues for each amino acid residue. The oligonucleotides can be purified on polyacrylamide gels or molecular sizing columns or by precipitation. DNA is “purified” when the DNA is separated from non-nucleic acid impurities (which may be polar, non-polar, ionic, etc.).

A “source” or “template” VHH, as used herein, refers to a VHH or VHH antigen binding fragment whose antigen binding sequence serves as the template sequence upon which diversification according to the criteria described herein is performed. An antigen binding sequence generally includes within a VHH preferably at least one CDR, preferably including framework regions.

A “transcription regulatory element” will contain one or more of the following components: an enhancer element, a promoter, an operator sequence, a repressor gene, and a transcription termination sequence.

“Transformation” means a process whereby a cell takes up DNA and becomes a “transformant”. The DNA uptake may be permanent or transient. A “transformant” is a cell which has taken up and maintained DNA as evidenced by the expression of a phenotype associated with the DNA (e.g., antibiotic resistance conferred by a protein encoded by the DNA).

A “variant” or “mutant” of a starting or reference polypeptide (for example, a source VHH or a CDR thereof), such as a fusion protein (polypeptide) or a heterologous polypeptide (heterologous to a phage), is a polypeptide that (1) has an amino acid sequence different from that of the starting or reference polypeptide and (2) was derived from the starting or reference polypeptide through either natural or artificial mutagenesis. Such variants include, for example, deletions from, and/or insertions into and/or substitutions of, residues within the amino acid sequence of the polypeptide of interest. For example, a fusion polypeptide of the invention generated using an oligonucleotide comprising a nonrandom codon set that encodes a sequence with a variant amino acid (with respect to the amino acid found at the corresponding position in a source VHH or antigen binding fragment) would be a variant polypeptide with respect to a source VHH or antigen binding fragment. Thus, a variant CDR refers to a CDR comprising a variant sequence with respect to a starting or reference polypeptide sequence (such as that of a source VHH or antigen binding fragment). A variant amino acid, in this context, refers to an amino acid different from the amino acid at the corresponding position in a starting or reference polypeptide sequence (such as that of a source VHH or antigen binding fragment). Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

A “wild-type” or “reference” sequence or the sequence of a “wild-type” or “reference” protein/polypeptide, such as a coat protein, or a CDR of a source VHH, may be the reference sequence from which variant polypeptides are derived through the introduction of mutations. In general, the “wild-type” sequence for a given protein is the sequence that is most common in nature. Similarly, a “wild-type” gene sequence is the sequence for that gene which is most commonly found in nature. Mutations may be introduced into a “wild-type” gene (and thus the protein it encodes) either through natural processes or through man induced means. The products of such processes are “variant” or “mutant” forms of the original “wild-type” protein or gene.

A “humanised” antigen specific antigen binding molecule may be modified at one or more amino acid sequence position to reduce the potential for immunogenicity in vivo, while retaining functional binding activity for the specific epitopes on the specific antigen.

Humanization of antibody variable domains is a technique well-known in the art to modify an antibody which has been raised, in a species other than humans, against a therapeutically useful target so that the humanized form may avoid unwanted immunological reaction when administered to a human subject. The methods involved in humanization are summarized in Almagro J. C and William Strohl W. Antibody Engineering: Humanization, Affinity Maturation, and Selection Techniques in Therapeutic Monoclonal Antibodies: From Bench to Clinic. Edited by An J. 2009 John Wiley & Sons, Inc and in Strohl W. R. and Strohl L. M., Therapeutic Antibody Engineering, Woodhead Publishing 2012.

Humanization of single domain antibodies is a technique well-known in the art so that the humanized form may avoid unwanted immunological reaction when administered to a human subject. The methods involved in humanization are summarized in Vincke et al., Prot Structure and Folding (2009) 284 (5) 3273-3284; Rossotti et al., FEBS J (2022) 289 (14) 3917-4328; Sulea T, Methods Mol Biol (2022) 2446.

The term “chimeric antigen receptors (CARs),” as used herein, may refer to artificial T-cell receptors, chimeric T-cell receptors, or chimeric immunoreceptors, for example, and encompass engineered receptors that graft an artificial specificity onto a particular immune effector cell. CARs may be employed to impart the specificity of an antigen-specific binding protein, such as a monoclonal antibody or VHH domains, onto a T cell, thereby allowing a large number of specific T cells to be generated, for example, for use in adoptive cell therapy. CARs may direct the specificity of the cell to a tumour associated antigen, for example. CARs may comprise an intracellular activation domain, a transmembrane domain, and an extracellular domain comprising a tumour associated antigen binding region. In particular aspects, CARs comprise fusions of single-chain variable fragments (scFv) derived from monoclonal antibodies fused to CD3-zeta transmembrane and endodomains. In other particular aspects, CARs comprise fusions of the VHH domains described herein with CD3-zeta transmembrane and endodomains. The specificity of other CAR designs may be derived from ligands of receptors (e.g., peptides) or from pattern-recognition receptors, such as Dectins. In particular embodiments, one can target malignant B cells by redirecting the specificity of T cells by using a CAR specific for the B-lineage molecule, CD 19. In certain cases, the spacing of the antigen-recognition domain can be modified to reduce activation-induced cell death. In certain cases, CARs comprise domains for additional co-stimulatory signalling, such as CD3-zeta, FcR, CD27, CD28, CD 137, DAP 10, and/or OX40. In some cases, molecules can be co-expressed with the CAR, including co-stimulatory molecules, reporter genes for imaging (e.g., for positron emission tomography), gene products that conditionally ablate the T cells upon addition of a pro-drug, homing receptors, chemokines, chemokine receptors, cytokines, and cytokine receptors.

The term “conjugation” as used herein may refer to any method of chemically linking two or more chemical moieties. Typically, conjugation will be via covalent bond. In the context of the present invention, at least one of the chemical moieties will be a polypeptide and in some cases the conjugation will involve two or more polypeptides, one or more of which may be generated by recombinant DNA technology. A number of systems for conjugating polypeptides are known in the art. For example, conjugation can be achieved through a lysine residue present in the polypeptide molecule using N-hydroxy-succinimide or through a cysteine residue present in the polypeptide molecule using maleimidobenzoyl sulfosuccinimide ester. In some embodiments, conjugation occurs through a short-acting, degradable linkage including, but not limited to, physiologically cleavable linkages including ester, carbonate ester, carbamate, sulfate, phosphate, acyloxyalkyl ether, acetal, and ketal, hydrazone, oxime and disulphide linkages. In some embodiments linkers that are cleavable by intracellular or extracellular enzymes, such as cathepsin family members or glucuronidase family members such as beta-glucuronidases, cleavable under reducing conditions or acidic pH are incorporated to enable releases of conjugated moieties from the polypeptide or protein to which it is conjugated.

A particularly preferred method of conjugation is through cysteine residues engineered at specific positions within an Fc sequence. Alternatively, to naturally occurring cysteine residues within an Fc sequence, including but not limited to cysteine residues involved in interchain disulphide bonds.

A particularly preferred method of conjugation is the use of intein-based technology (US2006247417) Briefly, the protein of interest is expressed as an N terminal fusion of an engineered intein domain (Muir 2006 Nature 442, 517-518). Subsequent N to S acyl shift at the protein-intein union results in a thioester linked intermediate that can be chemically cleaved with bis-aminoxy agents or amino-thiols to give the desired protein C-terminal aminoxy or thiol derivative, respectively. These C-terminal aminoxy and thiol derivatives can be reacted with aldehyde/ketone and maleimide functionalised moieties, respectively, in a chemoselective fashion to give the site-specific C-terminally modified protein.

In another preferred method of conjugation, the VHHs are directly expressed with an additional cysteine at or near the C-terminal region of the VHH or incorporated within a short C-terminal tag sequence enabling conjugation with thiol reactive payloads such as maleimide functionalised moieties.

Conjugation as referred to herein is also intended to encompass the use of a linker moiety, which may impart a number of useful properties. Linker moieties include, but are not limited to, peptide sequences such as poly-glycine, gly-ser, val-cit or val-ala. In certain cases, the linker moiety may be selected such that it is cleavable under certain conditions, for example via the use of enzymes, nucleophilic/basic reagents, reducing agents, photo-irradiation, electrophilic/acidic reagents, organometallic and metal reagents, or oxidizing reagents, or the linker may be specifically selected to resist cleavage under such conditions.

In certain cases, the linker may contain a PAB (used interchangeably with PABC) moiety as shown below

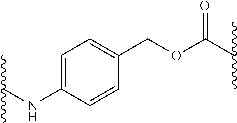 [SMILES: *Nc1ccc(COC(*)=O)cc1]

In certain cases, the linker may contain a val-cit-PAB moiety as shown below

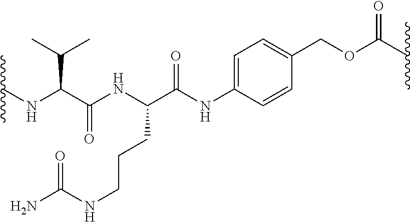 [SMILES: *N[C@H](C(=O)N[C@@H](CCCNC(N)=O)C(=O)Nc1ccc(COC(*)=O)cc1)C(C)C]

Polypeptides may be conjugated to a variety of functional moieties in order to achieve a number of goals. Examples of functional moieties include, but are not limited to, polymers such as polyethylene glycol in order to reduce immunogenicity and antigenicity or to improve solubility. Further non-limiting examples include the conjugation of a polypeptide to a therapeutic agent or a cytotoxic agent.

The term “detectable label” is used herein to specify that an entity can be visualized or otherwise detected by spectroscopic, photochemical, biochemical, immunochemical, electrical, optical, chemical or other means. The detectable label may be selected such that it generates a signal which can be measured and whose intensity is proportional to the amount of bound entity. A wide variety of systems for labelling and/or detecting proteins and peptides are known in the art. A label may be directly detectable (i.e., it does not require any further reaction or manipulation to be detectable, e.g., a fluorophore is directly detectable) or it may be indirectly detectable (i.e., it is made detectable through reaction or binding with another entity that is detectable, e.g., a hapten is detectable by immunostaining after reaction with an appropriate antibody comprising a reporter such as a fluorophore). Suitable detectable agents include, but are not limited to, radionuclides, fluorophores, chemiluminescent agents, microparticles, enzymes, colorimetric labels, magnetic labels, haptens, molecular beacons, and aptamer beacons.

Methods of killing or inhibiting the growth of a cells expressing ALPP and/or ALPPL2 in vitro or in a patient are contemplated herein, in general, the term “killing” as used herein in the context of cells means causing a cell death. This may be achieved by a number of mechanisms, such as necrosis or other cell injury, or the induction of apoptosis. The phrases “inhibiting the growth” or “inhibiting proliferation” when used herein are intended to encompass the prevention of cell development, more specifically the prevention of cell division.

As used herein, an alkyl group is a straight chain or branched, substituted or unsubstituted group (preferably unsubstituted) containing from 1 to 40 carbon atoms. An alkyl group may optionally be substituted at any position. The term “alkenyl,” as used herein, denotes a group derived from the removal of a single hydrogen atom from a straight- or branched-chain aliphatic moiety having at least one carbon-carbon double bond. The term “alkynyl,” as used herein, refers to a group derived from the removal of a single hydrogen atom from a straight- or branched-chain aliphatic moiety having at least one carbon-carbon triple bond.

The term ‘alkyl’, ‘aryl’, ‘heteroaryl’ etc also include multivalent species, for example alkylene, arylene, ‘heteroarylene’ etc. Examples of alkylene groups include ethylene (—CH₂—CH₂—), and propylene (—CH₂—CH₂—CH₂—). An exemplary arylene group is phenylene (—C₆H₄—), and an exemplary heteroarylene group is pyridinylene (—C₅H₃N—).

Aromatic rings are cyclic aromatic groups that may have 0, 1, 2 or more, preferably 0, 1 or 2 ring heteroatoms. Aromatic rings may be optionally substituted and/or may be fused to one or more aromatic or non-aromatic rings (preferably aromatic), which may contain 0, 1, 2, or more ring heteroatoms, to form a polycyclic ring system.

Aromatic rings include both aryl and heteroaryl groups. Aryl and heteroaryl groups may be mononuclear, i.e. having only one aromatic ring (like for example phenyl or phenylene), or polynuclear, i.e. having two or more aromatic rings which may be fused (like for example napthyl or naphthylene), individually covalently linked (like for example biphenyl), and/or a combination of both fused and individually linked aromatic rings. Preferably the aryl or heteroaryl group is an aromatic group which is substantially conjugated over substantially the whole group. Aryl groups may contain from 5 to 40 ring carbon atoms, from 5 to 25 carbon atoms, from 5 to 20 carbon atoms, or from 5 to 12 carbon atoms. Heteroaryl groups may be from 5 to 40 membered, from 5 to 25 membered, from 5 to 20 membered or from 5 to 12 membered rings, containing 1 or more ring heteroatoms selected from N, O, S and P. An aryl or heteroaryl may be fused to one or more aromatic or non-aromatic rings (preferably an aromatic ring) to form a polycyclic ring system.

Aryl and heteroaryl preferably denote a mono-, bi- or tricyclic aromatic or heteroaromatic group with up to 25 ring atoms that may also comprise condensed rings and is optionally substituted. Preferred aryl groups include, without limitation, benzene, biphenylene, triphenylene, [1,1′:3′,1″]terphenyl-2′-ylene, naphthalene, anthracene, binaphthylene, phenanthrene, pyrene, dihydropyrene, chrysene, perylene, tetracene, pentacene, benzopyrene, fluorene, indene, indenofluorene, spirobifluorene, etc.

Preferred heteroaryl groups include, without limitation, 5-membered rings like pyrrole, pyrazole, silole, imidazole, 1,2,3-triazole, 1,2,4-triazole, tetrazole, furan, thiophene, selenophene, oxazole, isoxazole, 1,2-thiazole, 1,3-thiazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 6-membered rings like pyridine, pyridazine, pyrimidine, pyrazine, 1,3,5-triazine, 1,2,4-triazine, 1,2,3-triazine, 1,2,4,5-tetrazine, 1,2,3,4-tetrazine, 1,2,3,5-tetrazine, and fused systems like carbazole, indole, isoindole, indolizine, indazole, benzimidazole, benzotriazole, purine, naphthimidazole, phenanthrimidazole, pyridimidazole, pyrazin-imidazole, quinoxalinimidazole, benzoxazole, naphthoxazole, anthroxazole, phenanthroxazole, isoxazole, benzothiazole, benzofuran, isobenzofuran, dibenzofuran, quinoline, isoquinoline, pteridine, benzo-5,6-quinoline, benzo-6,7-quinoline, benzo-7,8-quinoline, benzoisoquinoline, acridine, phenothiazine, phenoxazine, benzopyridazine, benzopyrimidine, quinoxaline, phenazine, naphthyridine, azacarbazole, benzocarboline, phenanthridine, phenanthroline, thieno[2,3b]thiophene, thieno[3,2b]thiophene, dithienothiophene, dithienopyridine, isobenzothiophene, dibenzothiophene, benzothiadiazothiophene, 2,5-dihydropyrrolo[3,4-c]pyrrol-1,4-dione (diketopyrrolopyrrole, DPP), 2-oxo-1H-indol-3-ylidene, [3,3′-bipyrrolo[2,3-b]pyridinylidene]-2,2′(1H,1′H)-dione (pyridine isoindigo) and (3E)-3-(2-oxo-1H-indol-3-ylidene)-1H-indol-2-one (isoindigo), or combinations thereof. The heteroaryl groups may be substituted with alkyl, alkoxy, thioalkyl, fluoro, fluoroalkyl or further aryl or heteroaryl substituents. Preferably a heteroaryl group is thiophene.

Particularly preferred heteroatoms are selected from O, S, N, P and Si. Typically, hydrogen will complete the valency of a heteroatom included in the molecules of the invention, e.g. for N there may be —NH— or —NH₂ where one or two other groups are involved.

As used herein, the term “optionally substituted” means that one or more of the hydrogen atoms in the optionally substituted moiety is replaced by a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable compounds. The term “stable”, as used herein, refers to compounds that are chemically feasible and can exist for long enough at room temperature (i.e. 16-25° C.) to allow for their detection, isolation and/or use in chemical synthesis.

Any of the above groups (for example, those referred to herein as “optionally substituted”, including alkyl, aryl and heteroaryl groups) may optionally comprise one or more substituents, preferably selected from silyl, sulfo, sulfonyl, formyl, amino, imino, nitrilo, mercapto, cyano, nitro, halogen, —NCO, —NCS, —OCN, —SCN, —C(═O)NR⁰R⁰⁰, —C(═O)X⁰, —C(═O)R⁰, —NR⁰R⁰⁰, C₁₋₁₂alkyl, C₁₋₁₂alkenyl, C₁₋₁₂alkynyl, C₆₋₁₂aryl, C₃₋₁₂cycloalkyl, heterocycloalkyl having 4 to 12 ring atoms, heteroaryl having 5 to 12 ring atoms, C₁₋₁₂ alkoxy, hydroxy, C₁₋₁₂ alkylcarbonyl, C₁₋₁₂ alkoxy-carbonyl, C₁₋₁₂ alkylcarbonyloxy or C₁₋₁₂ alkoxycarbonyloxy wherein one or more H atoms are optionally replaced by F or Cl and/or combinations thereof; wherein X⁰ is halogen and R⁰ and R⁰⁰ are, independently, H or optionally substituted C₁₋₁₂alkyl.

The optional substituents may comprise all chemically possible combinations in the same group and/or a plurality of the aforementioned groups (for example amino and sulfonyl if directly attached to each other represent a sulfamoyl radical). In one embodiment, the substituent is not acyl. As used herein acyl refers to an acyl group which is a moiety derived by the removal of one or more hydroxyl groups from an oxoacid, such as a carboxylic acid. It contains a double-bonded oxygen atom and an alkyl group.

In some embodiments the groups may be unsubstituted. For example, the anthracycline (PNU) derivative may be of formula (V):

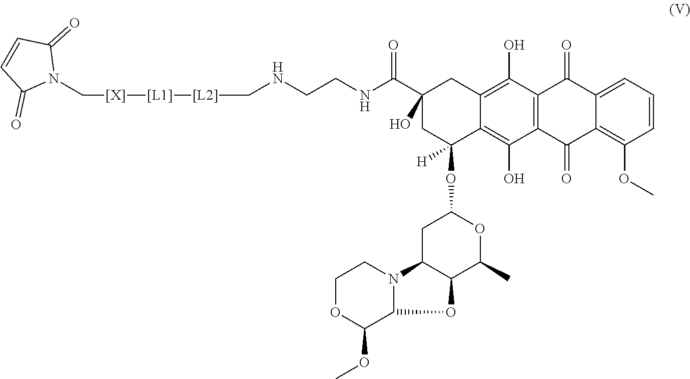

wherein [X] is an optional spacer selected from the group comprising unsubstituted alkyl groups, unsubstituted heteroalkyl groups, unsubstituted aryl groups, unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH2O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof.

In embodiments wherein the groups are unsubstituted, [X] is preferably selected from the group comprising polyethylene glycol and

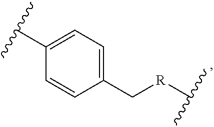

wherein [symbol] represents the point of attachment to the rest of the molecule and wherein [R] is an optional spacer selected from the group comprising unsubstituted alkyl groups, unsubstituted heteroalkyl groups, unsubstituted aryl groups, unsubstituted heteroaryl groups, one or more heteroatoms, polyethylene glycol, or a combination thereof.

In general, the term PAB is intended to mean p-aminobenzyloxycarbonyl. Occasionally in the literature, the term PAB may be used to indicated p-aminobenzyl. In the present specification, PAB is intended to indicate p-aminobenzyloxycarbonyl. The terms “vc-PAB-MMAE”, “vcPAB-MMAE”, “vcMMAE” and “MMAE” are used herein interchangeably to describe conjugates resulting from conjugation with MC-vc-PAB-MMAE

The term “target-binding molecule” refers to any molecule that binds to a given target. In this context, “target” and “antigen” may be used interchangeably. Examples of target-binding molecules include natural or recombinant proteins including immunoglobulins or antibodies, immunoglobulin Fc regions, immunoglobulin Fab regions, Fab, Fab′, Fv, Fv-Fc, single chain Fv (scFv), scFv-Fc, (scFv)₂, diabodies, triabodies, tetrabodies, bispecific t-cell engagers, inteins, intein fusions, VNAR domains, single domain antibodies (sdAb), VH domains, scaffold proteins (affibodies, centyrins, darpins etc.) and nucleic acids including aptamers or small molecules or natural products that have been developed to bind to the target or naturally bind to the target.

Chemical modification of proteins and biomolecules to introduce thiols is well established. Methods include reaction of amine groups with 2-iminothiolane (Traut's reagent), modification of amine groups with NHS-ester containing heterobifunctional agents such as N-succinimidyl S-acetylthiolate (SATA) or N-succinimidyl-4-(2-pyridyldithio)butanoate (SPDB), followed by treatment with hydroxylamine and reducing agents respectively and cleavage of engineered intein-fusion proteins with cysteamine to generate C-terminal thiol proteins and peptides.

The phrase “selected from the group comprising” may be substituted with the phrase “selected from the group consisting of” and vice versa, wherever they occur herein.

The PNU derivatives described herein may be prepared accordingly to standard synthesis methods. Mass spectrometry may be used to verify that the correct molecules have been produced (Table 4).

TABLE 3
Characterisation of PNU derivatives by mass spectrometry

| | | Exp. | Obs. |
|---|---|---|---|
| | | mass, | mass, |
| Name | Structure | Da | Da |
| MA-PEG4-vc-PAB- EDA-PNU159682 | 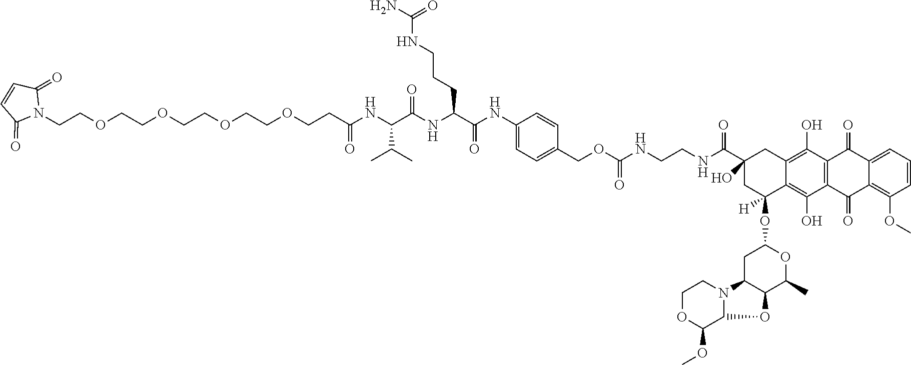 | 1402.5 | 1402.1 |
| MA-PEG4-va-EDA-PNU159682 | 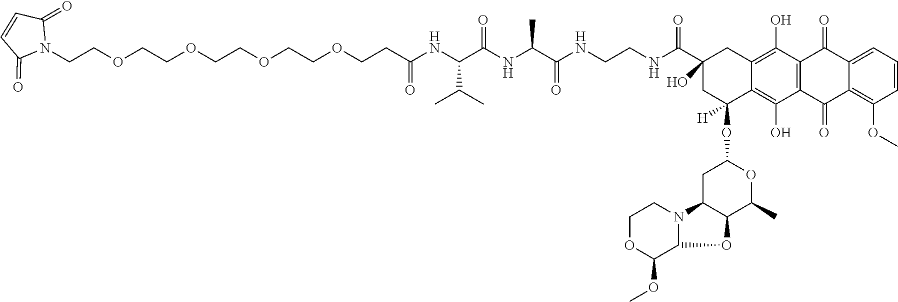 | 1167.2 | 1167.6 |
| MA-PEG4-na-EDA-PNU159682 | 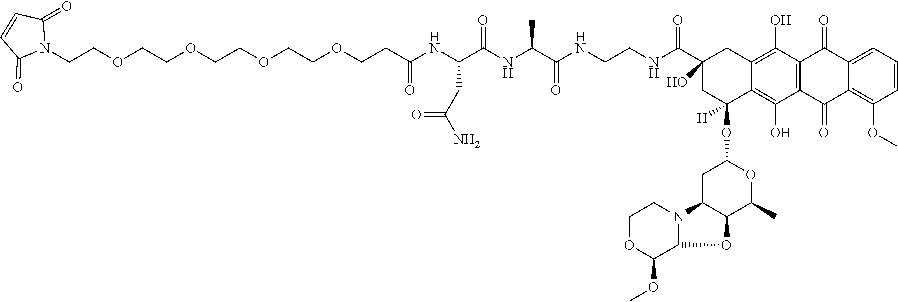 | 1182.2 | 1182.4 |
| MA-PEG4-EDA-PNU159682 | 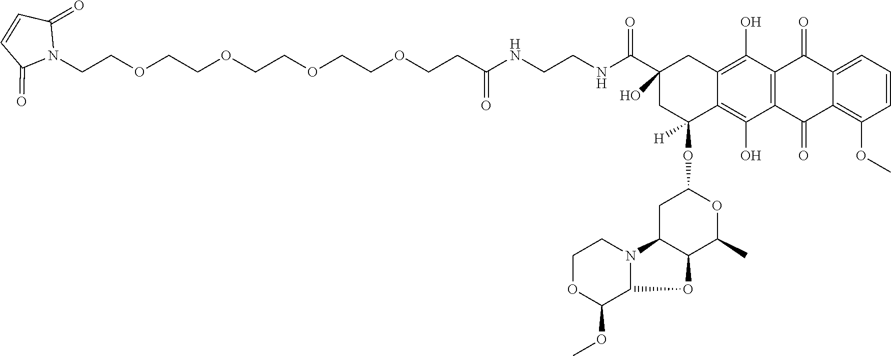 | 997.01 | 996.8 |
| MA-PEG4-vc-PAB- DMAE-PNU159682 | 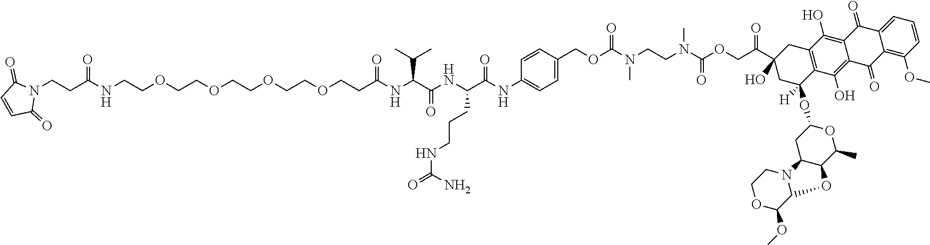 | 1559.6 | 1558.7 |

The present invention will be further understood by reference to the following examples.

### EXAMPLES

### Example 1—Generation of the Anti-ALPP and ALPPL2 Binders

### VHH Production

VHH's targeting ALPP and ALPPL2 were generated through immunisation of Llamas with an engineered ALPP homodimer (ALPP CysHis, SEQ ID NO: 263). The engineered ALPP homodimer was used for immunisation to ensure the dimeric form of the protein was maintained in solution. Briefly, two llamas were immunised 5 times with ALPP CysHis and antibody titres checked regularly for 98 days before bleeds were taken and PBMC's isolated. PBMC's from both animals were combined and a single domain antibody library was created from the RNA/cDNA. Three rounds of bio-panning were conducted against ALPP CysHis with negative selection against ALPI His homodimer (SEQ ID NO: 264 at each stage).

After panning, 94 individual clones were screened for ALPP specific binding expressed as monoclonal phage. These were analysed by ELISA for specific binding to ALPP and any off-target binding to ALPI. Clones with high specificity for ALPP and little ALPI binding were selected for off-phage testing. E. coli periplasmic expression of 25 hits identified from the phage library screen were conducted and C-terminally His-tagged proteins extracted by osmotic shock and purified by IMAC chromatography using cOmplete™ His-Tag Purification Resin (Roche). The eluted samples were dialyzed into PBS pH 7.4. The concentration of each sample was calculated from OD280 readings using the predicted extinction coefficient based on the nanobody's sequence. All proteins were then characterised by SDS-PAGE under reducing and non-reducing conditions, size exclusion chromatography (SEC) and electrospray mass spectrometry to determine expression levels, purity, monomericity and to confirm protein identity. Binding to ALPP and ALPI was assessed by ELISA, thermal stability by thermal shift assay and ALPP and ALPI binding kinetics (including K_{D}) by BLI. A subset of monomers was also tested against ALPPL2 by BLI to determine cross reactivity between the homologues.

TABLE 4
ALPP targeting VHH sequences

| Name | Sequence |
|---|---|
| A05 | QVQLVESGGGLVQAGASLTLSCAASGFTFNTRSIVWFRQAPGKEREAVSCISKVAGSTYYGDSV |
| | QGRFSSSTDNAKNTVSLQMNSLKPEDTAVYTCAASFAPNCYGWSVAANMEYWGEGTMVTVSS |
| A06 | QVHLVESGGGLVQAGGSLRLSCAASGRTFLSMGWFRQAPGKEREFVAAISWTGGSTGYADSVK |
| | GRFTISRESDKETMYLQMNSLKPEDTAVYYCAARGDVYYGGDDATRYKYWGQGTQVTVSS |
| A08 | QVQLVESGGGLVQAGESLRLACAASGTIFSGNAVGWYRQAPEKQREWVSSITDYGDTDYAEAV |
| | KGRFTISRDNAKNTVYLQMNSLKPEDTAVYYCYVYQRGESEEYWGQGTKVTVSS |
| A12 | QVHLVECGGGSVQAGGFLRLSCAASGPSLSNYAMGRFRQAPGKEREFVAGISWGGGRTSYTD |
| | SVKGRFTISRDNAKNTVYLQMNNLEPEDTGVYYCAGRYGRGRNVEYEYDYWGQGTKVTVSS |
| B01 | QVQLVESGGGLVQAGGSLRLSCEVTRSPMPIDTMAWYRQAPGRQRELVAIISRSGSTNYADSVK |
| | GRFTISRNDTKSTMYKSTMYLKMNTLELEDTGVYYCNAVGLWNGAEYWGQGTQVTVSS |
| B09 | QVQLVESGGGSVQAGGSLRLSCAASGPSLSHYAMGWFRQAPGKEREFVAGISWGGGRTSYTD |
| | SVKGRFTISRDNAKNTVYLQMNNLKPEDTGVYYCAGRYGRGRNVEYEYDYWGQGTQVTVSS |
| B11 | QVHLVESGGGSVQAGGSLRLSCAASGPSLSNYAMGWFRQAPGKEREFVAGISWGGGRTSYTD |
| | SVKGRFTISRDNAKNTVYLQMNNLKPEDTGVYYCAGRYGRGRNVEYEYDYWGQGTQVTVSS |
| C05 | QVQLVESGGGLVQAGGSLRLSCAASRRSFSEYAMGWFRQAPGKEREFVAAIGWSGVIEYADSV |
| | KGRFTISRDNAKDTVYLQMNSLKPEDTAVYYCAAGLGIRVVSQTPAYWGQGTQVTVSS |
| C06 | QVQLVESGGGLVQPGGSLRVSCAASGPSLSNYAMGWFRQAPGKEREFVAGISWGGGRTSYTD |
| | SVKGRFTISRDNAKNTVYLQMNNLKPEDTGVYYCAGRYGRGRNVEYEYDYWGQGTQVTVSS |
| C07 | QVQLVESGGGLVQAGESLRLACAASGTIFSGNAVGWYRQAPEKQREWVSSITDYGDTDYAEAV |
| | KGRFTISRDNAKNTVYLQMNSLKPEDTAVYYCYVYQRGESEEYWGQGTQVTVSS |
| D04 | QVQLVESGGGLVQPGGSLRLSCAASESTFSINVMGWYRQAPGKQRELVATITSGDITNYADSVK |
| | GRFTISRDNAENTVYLRTNSLKPEDTAVYYCNARGEYHSGNEYDYWGQGTQVTVSS |
| D08 | QVHLVESGGGSVQAGGSLRLSCAASGPSLSNYAMGWFRQAPGKEREFVAGISWGGGRTSYTD |
| | SVKGRFTISRDNAKNTVYLQMNNLKPEDTGVYYCAGRYGRGRNVEYEYDYWGQGTKVTVSS |
| D10 | QVQLVESGGGLVQPGGSLRLSCAASGSIFSVNTMGWYRQAPGKQRDLVAVATSGGTIHYADSV |
| | KGRFTISRDNAKNTVYLQMNSLKPEDTAVYYCNLVVPYGMIAGDHDYWGQGTKVTVSS |
| D11 | QVQLVESGGGSVQAGGSLRLSCAASGPSLSNYAMGWFRQAPGKEREFVAGISWGGGRTSYTD |
| | SVKGRFTISRDNAKNTVYLQMNNLKPEDTGVYYCAGRYGRGRNVEYEYDYWGQGTMVTVSS |
| E06 | QVHLVESGGGLVQAGGSLRLSCAASRRSFSEYAMGWFRQAPGKEREFVAAIGWSGVIEYADSV |
| | KGRFTISRDNAKDTVYLQMNSLKPEDTAVYYCAAGLGIRVVSQTPAYWGQGTQVTVSS |
| E10 | QVQLVESGGGLVQAGGSLRLSCAVSGRALSDYNIGWFRQAPGKEREFVARKTWDGSSTRYDDS |
| | VKGRFTVSIEDAKNTAYLQMSSLKTEDTAVYYCAAGRYSRARGWEYDYWGQGTQVTVSS |
| E11 | QVQLVESGGGSVQAGGSLRLSCAASGPSLSNFAMGWFRQPPGKEREFVAGISWGGGRTSYTD |
| | SVKGRFTISRDNAKNTVYLQMNNLKPEDTGVYYCAGRYGRGRNVEYEYDYWGQGTQVTVSS |
| E12 | QVQLVESGGGSVQAGGSLRLSCAASGPSISNFAMGWFRQAPGKEREFVAGISWGGGRTNYLDS |
| | VKGRFTISRDNAKNMVHLQMNSLKPEDTTVYYCAGRYGRGRDVEYEYDYWGQGTQVTVSS |
| F05 | QVQLVESGGGLVQAGGSLRLSCAASGRAFGDYNIGWFRQAPGKEREFVARRTWDGSSTRYDD |
| | SVKGRFTVSIDNAKNTAYLQMNSLKTEDTAVYYCAAGRYSRVRGWEYDYWGQGTQVTVSS |
| F08 | QVQLVESGGGSVQAGGSLRLSCAASGPSLSNYAMGWFRQAPGKEREFVAGISWGGGRTSYTD |
| | SVKGRFTISRDNAKNTVYLQMNNLKPEDTGVYYCAGRYGRGRNVEYEYDYWGQGTQVTVSS |
| F09 | QVQLVESGGGSVQAGGSLRLSCAASGPSLSNYAMGWFRQAPGKEREFVAGISWGGGRTSCTD |
| | SVKGRFTISRDNAKNTVYLQMNNLKPEDTGVYYCAGRYGRGRNVEYEYDYWGQGTQVTVSS |
| F12 | QVQLVESGGGLVEAGGSLRLSCAVSGSALSDYNIGWFRQAPGKEREFVARKTWDGSSTKYDDS |
| | VKGRFTVSIDNAKNTAYLQMNSLKTEDTAVYYCAAGRYSRVRGWEYDYWGQRTQVTVSS |
| G03 | QVQLVESGGGTAQAGGSLRLACAASGRSVTMGWLGWVPRAERDDLEFITWFNWGTYYANSVN |
| | GRFTISTDNADNTVYPQMNRLKPENPAVYYCASFTGYRPLINRLNDVSDCGHGALVAVSS |
| H09 | QVQLVESGGGSVQTGGSLRLSCAASGPSLSNYAMGWFRQAPGKEREFVAGISWGGGRTNYLD |
| | SVKGRFTISRDNAKNTMYLQMNSLKPEDTAVYYCAGRYGRGRDVEYEYDYWGQGTQVTVSS |
| H10 | QVQLVESGGGSVQTGGSLRLSCAASGPSLSNYAMGWFRQAPGKEREFVAGISWGGGRTNYLD |
| | SVKGRFTISRDNAKNTMYLQMNSLKPEDAAVYYCAGRYGRGRDVEYEYDYWGQGTQVTVSS |

### Binding to ALPP and ALPI by ELISA

ELISA was used to test the binding and specificity of the VHHs. ALPP CysHis (SEQ ID NO: 263), ALPI His homodimer (SEQ ID NO: 264) or BSA (Thermo Fisher) were coated onto plates and 500 ng of each purified VHH added. Anti-Llama—HRP antibody was used for detection.

### Size Exclusion Chromatography

The monomericity and biophysical properties of the VHH variants were assessed by size-exclusion chromatography (SEC) using an analytical SEC column (Superdex 75 10/300 GL). Chromatography was carried out in PBS pH 7.4.

### Binding to Human ALPP/ALPPL2, ALPI and ALPL by BLI

Binding kinetics were determined using the Biolayer Interferometry (BLI) Octet K2 system (Sartorius). Human ALPP, ALPPL2, ALPL (each fused with a cysteine stabilised leucine zipper) and ALPI, (SEQ ID NO: 265, SEQ ID NO: 266 and SEQ ID NO: 267 and SEQ ID NO: 264 respectively) were immobilised in sodium acetate pH5 buffer to AR2G sensors using amine coupling. All assays were conducted in PBS with a plate temperature of 25° C. Ka (M⁻¹s⁻¹), K_{d} (s⁻¹) and K_{D} (nM) values were determined using Octet Data Analysis High Throughput software (ForteBio) for Biolayer Interferometry.

### Thermal Stability Experiments

Thermal stability assays used Applied Biosystems StepOne Real Time PCR system with the Protein Thermal Shift™ dye kit (Thermo Fisher). The assay mix was set up so that the protein was at a final concentration of 20 M in 20 μL. 5 μL of Thermal Shift™ buffer was added alongside 2.5 μL 8× Thermal Shift™ Dye. Assays were run using the StepOne software and data analysed using Protein Thermal Shift™ software. All data are from first derivative analysis.

TABLE 5
Characterisation of VHH domains targeting ALPP/ALPPL2

| | SEC | | hALPP | | | hALPPL2 | | | | |
|---|---|---|---|---|---|---|---|---|---|---|
| | RT | Tm | K_{D} | Kₐ | K_{d} | K_{D} | Kₐ | K_{d} | ELISA | |
| Name | (ml) | ° C. | (nM) | (M⁻¹s⁻¹) | (s⁻¹) | (nM) | (M⁻¹s⁻¹) | (s⁻¹) | hALPP | hALPI |
| A05 | 14.2 | 69.5 | <0.01 | 260000 | <1.0E−07 | | | | * | No Binding |
| A06 | 15.3 | 73.2 | 1.32 | 117000 | 0.000154 | 5.04 | 133000 | 0.000672 | ** | No Binding |
| A08 | 13.1 | 71.4 | 0.03 | 429000 | 0.000012 | | | | * | No Binding |
| A12 | 14.7 | 76.3 | <0.01 | 360000 | <1.0E−07 | | | | ** | No Binding |
| B01 | 14.8 | 82.7 | 1.18 | 162000 | 0.000191 | 0.29 | 482000 | 0.00014 | * | No Binding |
| B09 | 14.8 | 77.1 | <0.01 | 237000 | <1.0E−07 | | | | ** | No Binding |
| B11 | 14.8 | 77.4 | 0.008 | 213000 | 0.000017 | | | | ** | No Binding |
| C05 | 14.2 | 80.6 | <0.01 | 394000 | <1.0E−07 | | | | ** | No Binding |
| C06 | 14.7 | 71.6 | <0.01 | 368000 | <1.0E−07 | | | | ** | No Binding |
| C07 | 13.9 | 70.7 | 0.22 | 129000 | 0.000028 | 0.61 | 471000 | 0.000288 | * | No Binding |
| D04 | 13.8 | 59.8 | <0.01 | 85100 | <1.0E−07 | 2.92 | 754000 | 0.00022 | ** | No Binding |
| D08 | 14.7 | 64.9 | <0.01 | 175000 | <1.0E−07 | | | | *** | No Binding |
| D10 | 14.5 | 69.9 | 0.2 | 67300 | 0.000013 | 1.22 | 2300000 | 0.0028 | *** | No Binding |
| D11 | 14.7 | 78.1 | <0.01 | 97700 | <1.0E−07 | | | | ** | No Binding |
| E06 | 14.2 | 81.1 | <0.01 | 106000 | <1.0E−07 | 0.0834 | 1050000 | 0.0000875 | ** | No Binding |
| E10 | 14.0 | 71.8 | <0.01 | 54200 | <1.0E−07 | 0.0862 | 1640000 | 0.000141 | * | No Binding |
| E11 | 14.7 | 78.0 | 0.001 | 149000 | 0.0000018 | | | | ** | No Binding |
| E12 | 14.6 | 55.5 | 0.04 | 39200 | 0.000028 | | | | * | No Binding |
| F05 | 14.2 | 70.6 | <0.01 | 13900 | <1.0E−07 | 0.0954 | 3740000 | 0.000356 | ** | No Binding |
| F08 | 14.7 | 77.6 | <0.01 | 252000 | <1.0E−07 | <0.01 | 684000 | <1.0E−07 | ** | No Binding |
| F09 | 14.5 | 72.1 | <0.01 | 40600 | <1.0E−07 | | | | ** | No Binding |
| F12 | 13.9 | 62.9 | <0.01 | 38600 | <1.0E−07 | | | | * | No Binding |
| G03 | 14.6 | 74.0 | <0.01 | 47400 | <1.0E−07 | | | | ** | No Binding |
| H09 | 14.6 | 68.8 | <0.01 | 58600 | <1.0E−07 | | | | ** | No Binding |
| H10 | 14.6 | 64.9 | 0.19 | 45900 | 0.0000085 | | | | ** | No Binding |

As shown in Table 5, high affinity ALPP and ALPPL2 specific binders with good thermal stability have been generated. No binding to ALPI or ALPL could be detected by BLI.

### Epitope Binning Experiments

To determine epitope cross reactivity BLI competition assays were conducted. AR2G sensors were used and ALPP immobilised as above. ALPP sensors were then dipped into a well containing a saturating concentration of the initial binder for ten minutes to get an association curve. The sensor was then dipped into a well containing the same concentration of initial binder plus 20 nM of a secondary binding.

An increase in the association curve was indicative that the second VHH bound to a distinct epitope from the initial binder. Monomers with identical CDR3s were grouped as likely to be in the same epitope bin and only one from each group used in the epitope binning experiment.

The epitope binning experiments described above (Table 6) indicates that the 25 different binding sequences could be grouped into 5 distinct epitope bins. A05, B09, B11, E06, E10, F05, F08 and G03 appear to cross compete and so are in epitope bin one. C07 and D04 make up epitope bin four while A06, B01 and D10 do not cross react with any other VHH and so make up epitope bins two, three and five respectively.

### Binding of VHH Proteins to Cell-Surface ALPP, ALPPL2, ALPI and ALPL by Flow Cytometry

C-terminal His tagged VHH proteins were assessed for ALPP, ALPPL2, ALPI and ALPL cell-surface binding by flow cytometry. Binders encompassing different epitope bins were screened on CHO cells stably expressing ALPP, ALPPL2, ALPI or ALPL. High levels of cell-surface proteins expression were confirmed for each of the cell-lines by flow cytometry.

Cell surface binding of test agents to hALPP isoforms was characterized in four CHO-ALPP isoform stably transfected cell lines (CHO-ALPP, CHO-ALPPL2, CHO-ALPI and CHO-ALPL) and the resulting K_{Dapp} values determined. Adherent cells were detached from tissue culture flasks by incubating with 0.1% EDTA/PBS solution at 37° C. for ~10 minutes or until cells detached easily. Cells were re-suspended in ice-cold PBS/2% FCS in 15 ml tubes and centrifuged at 1700 rpm for 5 mins at 4° C. Supernatant was removed and the cell pellet re-suspended in PBS/2% FCS. A cell count was performed using a Chemometec Nucleocounter NC-202 and 2.5×10\hat{ }5 cells were aliquoted per test sample into a 96 well plate. Cells were incubated with 100 μl of test agents at a range of concentrations, plus controls for 1 hr on ice. The sample plate was centrifuged at 1700 rpm for 5 mins at 4° C. The supernatant was removed, and a wash performed by re-suspending the cell pellets in 0.25 mL of ice-cold PBS/2% FCS using a multichannel pipette. Samples were again centrifuged at 1700 rpm for 5 min at 4° C. Supernatant was removed and two further washes performed as described. After the final wash and centrifugation step, excess liquid was removed by blotting the plate on tissue paper. Binding of VHH (His6 tagged) agents was determined by adding 100 μl of anti-x6His tag Ab (Abcam) per cell pellet sample as appropriate and incubating on ice for 30 mins. Wash steps were performed as described previously. Fitc-anti-mouse antibody (ThermoFisher) was used to detect binding of the VHH (His6 tagged) agents by incubating with the appropriate samples for 30 min on ice in the dark. Wash steps were performed as described previously. All cell pellets were finally re-suspended in 0.28m of ice-cold PBS/2% FCS and left on ice in the dark prior to analysis on a Merck-Millipore Guava EasyCyte HT or Thermo Fisher Attune N×T flow cytometer.

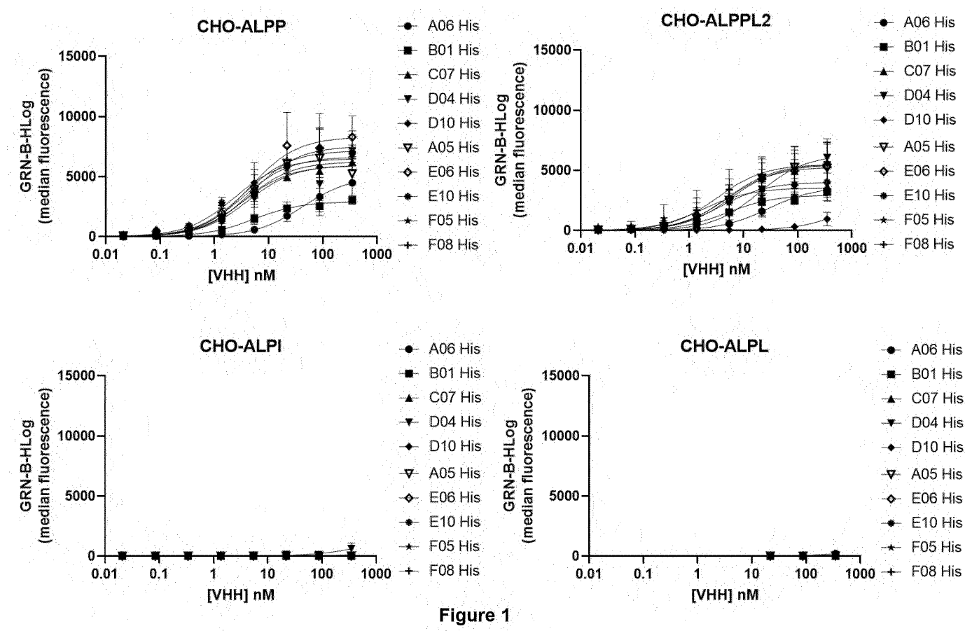

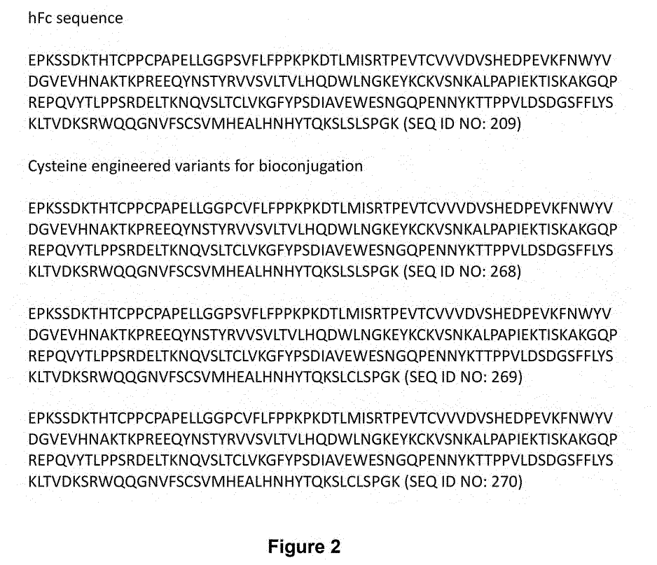

Binding of the different domains to the different cell-surface ALPP forms is summarized in Table 7 and in FIG. 1.

TABLE 7
Binding of different VHH proteins to ALPP, ALPPL2,
ALPI and ALPL expressed on the surface of CHO cells

| | | K_{D}app (nM) | | | |
|---|---|---|---|---|---|
| ID | | CHO- | CHO- | | |
| # | Molecule | ALPP | ALPPL2 | CHO-ALPI | CHO-ALPL |
| 249 | A05 His | 3.8 | 6.0 | No binding | No binding |
| 264 | E06 His | 4.6 | 4.9 | No binding | No binding |
| 265 | E10 His | 3.5 | 3.8 | No binding | No binding |
| 268 | F05 His | 2.3 | 1.8 | No binding | No binding |
| 269 | F08 His | 5.0 | 3.3 | Minimal | No binding |
| | | | | binding* | |
| 250 | A06 His | 42.4 | 28.3 | No binding | No binding |
| 253 | B01 His | 5.3 | 5.0 | No binding | No binding |
| 258 | C07 His | 3.5 | 5.1 | No binding | No binding |
| 259 | D04 His | 4.2 | 19.5 | Minimal | No binding |
| | | | | binding* | |
| 262 | D10 His | 3.4 | Binding | No binding | No binding |
| | | | K_{D}app ND | | |

ND = not-determinable (i.e. MFI binding to CHO-ALPI cells at highest conc of 350 nM ≤9.5% of MFI binding to CHO-ALPP cells)

Minimal binding observed at highest concentration (350 nM) of F08-His and D04-His. F08-His at 350 nM MFI=99 for binding to CHO-ALPI compared to a MFI of 7372 for binding to CHO ALPP (ALPI MFI is 1.3% of the ALPP MFI).

For D04-His at 350 nM, MFI=629 for binding to CHO-ALPI compared to a MFI of 6652 for binding to CHO ALPP (ALPI MFI is 9.5% of the ALPP MFI).

As shown in Table 7 and FIG. 1 VHH protein binders that are highly specific for ALPP and ALPPL2 have been generated, which show no binding to ALPI or ALPL even at very high concentrations.

D10 shows preferential binding to ALPP over ALPPL2. D04 displays weak binding to ALPI. F08 displays minimal binding with the MFI at saturation (350 nM) is 75-fold lower for ALPI binding versus ALPP.

### Example 2—VHH Reformatting as Fc Fusion Proteins

### Generation of VHH Fc Fusion Proteins

Fusion of proteins to an Fc domain can improve protein solubility and stability, markedly increase plasma half-life and improve overall therapeutic effectiveness. A human IgG1 Fc sequence and further examples are shown below.

Human IgG1 Fc (hFc)
(SEQ ID NO: 209)
EPKSSDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGK
hFc S293C
(SEQ ID NO: 268)
EPKSSDKTHTCPPCPAPELLGGPCVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSR
WQQGNVFSCSVMHEALHNHYTQKSLSLSPGK
hFc S442C
(SEQ ID NO: 269)
EPKSSDKTHTCPPCPAPELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSR
WQQGNVFSCSVMHEALHNHYTQKSLCLSPGK
hFc S293C + S442C
(SEQ ID NO: 270)
EPKSSDKTHTCPPCPAPELLGGPCVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPIEKTISKAKGQPREPQVY
TLPPSRDELTKNQVSLTCLVKGFYPSDIAVEWESNGQPENNYKTTPPVLDSDGSFFLYSKLTVDKSR
WQQGNVFSCSVMHEALHNHYTQKSLCLSPGK

VHH variants were genetically fused via either a long [G₄S]₃ linker or a shorter [G₄S]₁ linker to engineered hIgG1 Fc domains that contained various cysteine substitutions in the hIgG1 Fc sequence (see sequence listing), including S239C, S442C and S239C+S442C (EU numbering). The VHH-hFc fusion proteins were transiently expressed as secreted protein in CHO K1 cells and purified from the media using MabSelect™ SuRe™ (Evitria, Switzerland). Purified proteins were exchanged into PBS pH 7.4 or PBS+100 mM Arg pH 7.4 and analysed by SEC (AdvanceBio, Agilent, running buffer DPBS pH 7.4 or Superdex200 Increase 10/300, Cytivia, running buffer PBS pH 7), SDS PAGE and mass spectrometry under reducing and native conditions to confirm sequence and protein integrity. For MS analysis proteins were deglycosylated with PNGaseF, reduced with 50 mM DTT (reduced mass) or analysed without reduction (intact mass) and run on Sciex X500B QTOF via MassPREP desalting column (Waters). Characterisation data for VHH-hFc proteins is shown in Table 8.

TABLE 8
Characterisation of ALPP/ALPPL2 targeting VHH-hFc proteins

| | | | Expression, | | Reduced Mass, Da | | Intact Mass, Da | |
|---|---|---|---|---|---|---|---|---|
| Protein | Linker | # | mg/L | Monomericity | Expected | Observed | Expected | Observed |
| F08-hFc (S239C) | [G₄S]₃ | 338 | 410 | 97.9 | 40236.5 | 40240.7 | — | — |
| F08-hFc (S442C) | [G₄S]₃ | 339 | 570 | 95.3 | 40236.5 | 40240.9 | — | — |
| A06-hFc (S239C) | [G₄S]₃ | 340 | 730 | 97.3 | 40210.6 | 40214.6 | — | — |
| B01-hFc (S239C) | [G₄S]₃ | 341 | 600 | 97.6 | 40295.9 | 42361.3 | — | — |
| | | | | | | (aberrant | | |
| | | | | | | glycosylation) | | |
| C07-hFc (S239C) | [G₄S]₃ | 342 | 450 | 97.5 | 39804.9 | 39809.4 | — | — |
| D04-hFc (S239C) | [G₄S]₃ | 371 | 145 | 95.35 | 39964.1 | 39968.6 | — | — |
| D10-hFc (S239C) | [G₄S]₃ | 372 | 125 | 96.7 | 39789.3 | 39794.0 | — | — |
| D10-hFc (S239C) | [G₄S]₁ | 373 | 145 | 96.4 | 39158.7 | 39163.4 | — | — |
| A05-hFc (S239C) | [G₄S]₃ | 433 | 340 | 97.6 | 40166.3 | 40158.8 | — | — |
| E06-hFc (S239C) | [G₄S]₃ | 434 | 475 | 96.3 | 39934.1 | 39930.9 | — | — |
| E10-hFc (S239C) | [G₄S]₃ | 435 | 525 | 97.4 | 40347.3 | 40341.7 | — | — |
| F05-hFc (S239C) | [G₄S]₃ | 436 | 726 | 98.4 | 40391.3 | 40385.6 | — | — |
| E06 linkerCys-hFc (S442C) | GCGGS[G₄S]₂ | 512 | 840 | 78.9 | 39970.5 | 39976.8 | — | — |
| D10 linkerCys-hFc (S442C) | GCGGS[G₄S]₂ | 515 | 560 | 69.8 | 39834.4 | 39840.6 | — | — |
| E06 linkerCys-hFc | GCGGS[G₄S]₂ | 528 | 800 | 94.3 | 39955.5 | 39960.2 | — | — |
| E06-hFc (S239C + S442C) | [G₄S]₃ | 558 | 870 | 90.33 | 39950.6 | 39948.0 | — | — |
| F05-hFc (S442C) | [G₄S]₃ | 862 | 211 | 88.1 | — | — | 80761.3 | 81012.3 (2* Cys |
| | | | | | | | | adduct) |

Purified target proteins were generated in good yield and showed high percentage monomericity as determined by SEC.

### Binding of VHH-hFc Proteins to ALPP Homologues by BLI

Binding kinetics were determined using the Biolayer Interferometry (BLI) Octet K2 or R4 system (Sartorius). Human ALPP, ALPPL2 and ALPL all fused with a disulphide stabilised leucine zipper (SEQ ID NO: 265, SEQ ID NO: 266, and SEQ ID NO: 267) and ALPI His (SEQ ID NO: 264) were used in BLI experiments. For K_{D} apparent measurements, the ALPP proteins were immobilised in sodium acetate pH5 buffer to AR2G sensors using amine coupling. To measure 1:1 interactions, AHC biosensors were used to immobilise Fc-fused proteins. All assays were conducted in HBS-EP with a plate temperature of 25° C. Ka (M⁻¹s⁻¹), Kd (s⁻¹) and K_{D} (nM) values were determined using Octet Data Analysis High Throughput software (Sartorius) for Biolayer Interferometry.

TABLE 9
Binding of VHH-hFc fusion proteins to ALPP, ALPPL2, ALPI & ALPL by
BLI (K_{D} apparent). All proteins below contain the [G₄S]₃ linker.

| | | hALPP | | | hALPPL2 | | | | |
|---|---|---|---|---|---|---|---|---|---|
| | | K_{D} | Kₐ | K_{d} | K_{D} | Kₐ | K_{d} | | |
| Name | # | (nM) | (M⁻¹s⁻¹) | (s⁻¹) | (nM) | (M⁻¹s⁻¹) | (s⁻¹) | hALPI | hALPL |
| A05-hFc (S239C) | 443 | <0.01 | 304000 | <1.0E−07 | <0.01 | 526000 | <1.0E−07 | No binding | No binding |
| A06-hFc (S239C) | 340 | <0.01 | 203000 | 0.00000018 | 0.584 | 368000 | 0.000215 | No binding | No binding |
| B01-hFc (S239C) | 341 | <0.01 | 140000 | <1.0E−07 | 0.201 | 308000 | 0.00000618 | Low level binding | No binding |
| C07-hFc (S239C) | 342 | <0.01 | 151000 | <1.0E−07 | 0.449 | 253000 | 0.000114 | No binding | No binding |
| F08-hFc (S239C) | 338 | <0.01 | 192000 | <1.0E−07 | — | — | — | Low level binding | No binding |
| D04-hFc (S239C) | 371 | 0.0214 | 311000 | 0.00000665 | 2.30 | 104000 | 0.000239 | No binding | No binding |
| D10-hFc (S239C) | 372 | 0.0677 | 160000 | 0.0000108 | 9.94 | 187000 | 0.00186 | No binding | No binding |
| E06-hFc (S239C) | 434 | 0.038 | 321000 | 0.0000122 | <0.01 | 688000 | <1.0E−07 | No binding | No binding |
| E10-hFc (S239C) | 435 | 0.012 | 382000 | 0.00000469 | <0.01 | 288000 | <1.0E−07 | No binding | No binding |
| F05-hFc (S239C) | 436 | 0.063 | 330000 | 0.0000206 | 0.03 | 350000 | 0.0000106 | No binding | No binding |

BLI data (Table 9) confirms the high affinity binding of VHH-hFc fusion proteins to ALPP and ALPPL2. B01 and F08 exhibited very low-level binding to ALPI, with K_{D} values of 148 and 10.8 nM respectively but both proteins gave very low response values in comparison to those observed against ALPP/ALPPL2. All other fusions exhibited no binding to ALPI or ALPL.

D04-hFc and D10-hFc show a slight drop-off in binding affinity for ALPPL2 compared to ALPP with lower response values also observed.

### ALPP Cross-Species Reactivity of VHH-hFc Proteins

To assess cross-species reactivity of the ALPP/ALPPL2 targeting VHH-hFc fusions, BLI was conducted using mouse ALPPL2 (NCBI #NP_031459), rhesus monkey ALPP (NCBI #XP_028687061) and cynomolgus monkey ALPP (NCBI #XP_045223825). Each of these were expressed as cysteine-stabilised leucine zipper fusions (SEQ ID NO: 271, SEQ ID NO: 272 and SEQ ID NO: 273). BLI was conducted as described above. Data is shown in Table 10.

TABLE 10
Cross-species reactivity of VHH-hFc proteins by BLI (K_{D} apparent)

| | | mALPPL2 | | | rmALPP | | | CynoALPP | | |
|---|---|---|---|---|---|---|---|---|---|---|
| | | K_{D} | Kₐ | K_{d} | K_{D} | Kₐ | K_{d} | K_{D} | Kₐ | K_{d} |
| Name | # | (nM) | (M⁻¹s⁻¹) | (s⁻¹) | (nM) | (M⁻¹s⁻¹) | (s⁻¹) | (nM) | (M⁻¹s⁻¹) | (s⁻¹) |
| A05-hFc | 443 | 0.225 | 462000 | 0.00104 | <0.01 | 562000 | <1.0E−07 | | | |
| A06-hFc | 340 | 0.047 | 392000 | 0.0000182 | <0.01 | 228000 | <1.0E−07 | <0.01 | 664200 | <1.0E−07 |
| B01-hFc | 341 | No Binding | | | 0.025 | 194000 | 0.00000481 | | | |
| C07-hFc | 342 | No Binding | | | 0.721 | 667000 | 0.000481 | | | |
| F08-hFc | 338 | No Binding | | | <0.01 | 297000 | <1.0E−07 | | | |
| D04-hFc | 371 | No Binding | | | <0.01 | 672000 | <1.0E−07 | | | |
| D10-hFc | 372 | No Binding | | | <0.01 | 126000 | <1.0E−07 | <0.01 | 1090000 | <1.0E−07 |
| E06-hFc | 434 | No Binding | | | <0.01 | 752000 | <1.0E−07 | 0.23 | 973900 | 0.00025 |
| E10-hFc | 435 | 2.59 | 1620000 | 0.0042 | 0.015 | 544000 | <1.0E−07 | <0.01 | 622400 | <1.0E−07 |
| F05-hFc | 436 | 0.72 | 3910000 | 0.00228 | 0.276 | 1030000 | 0.0000869 | <0.01 | 105800 | <1.0E−07 |

A05, A06, E10 and F05-hFc exhibit binding to mouse ALPPL2, though the response values were much lower than those observed for binding to human ALPP and ALPPL2. All the hFc fusions bind with high affinity to rhesus macaque and cynomolgus ALPP proteins.

### Binding of VHH-hFc Proteins to Cell-Surface ALPP, ALPPL2, ALPI and ALPL by Flow Cytometry

VHH-hFc proteins were assessed for human ALPP, ALPPL2, ALPI and ALPL cell-surface binding by flow cytometry. Binders were screened on CHO cells stably expressing human ALPP, ALPPL2, ALPI or ALPL.

Cell surface binding of test agents to hALPP isoforms was characterized in four CHO-ALPP isoform transfectant cell lines (CHO-ALPP, CHO-ALPPL2, CHO-ALPI and CHO-ALPL) and the resulting K_{Dapp} values determined. Adherent cells were detached from tissue culture flasks by incubating with 0.1% EDTA/PBS solution at 37° C. for ~10 minutes or until cells detached easily. Cells were re-suspended in ice-cold PBS/2% FCS in 15 ml tubes and centrifuged at 1700 rpm for 5 mins at 4° C. Supernatant was removed and the cell pellet re-suspended in PBS/2% FCS. A cell count was performed using a Chemometec Nucleocounter NC-202 and 2.5×10\hat{ }5 cells were aliquoted per test sample into a 96 well plate. Cells were incubated with 100 μl of test agents at a range of concentrations, plus controls for 1 hr on ice. The sample plate was centrifuged at 1700 rpm for 5 mins at 4° C. The supernatant was removed, and a wash performed by re-suspending the cell pellets in 0.25 mL of ice-cold PBS/2% FCS using a multichannel pipette. Samples were again centrifuged at 1700 rpm for 5 min at 4° C. Supernatant was removed and two further washes performed as described. After the final wash and centrifugation step, excess liquid was removed by blotting the plate on tissue paper. Binding of VHH-hFc agents was determined by adding 100 μl of Fitc-anti-human Ab (ThermoFisher) and incubating for 30 mins on ice in the dark. Wash steps were performed as described previously. All cell pellets were finally re-suspended in 0.28 ml of ice-cold PBS/2% FCS and left on ice in the dark prior to analysis on a Merck-Millipore Guava EasyCyte HT or Thermo Fisher Attune N×T flow cytometer.

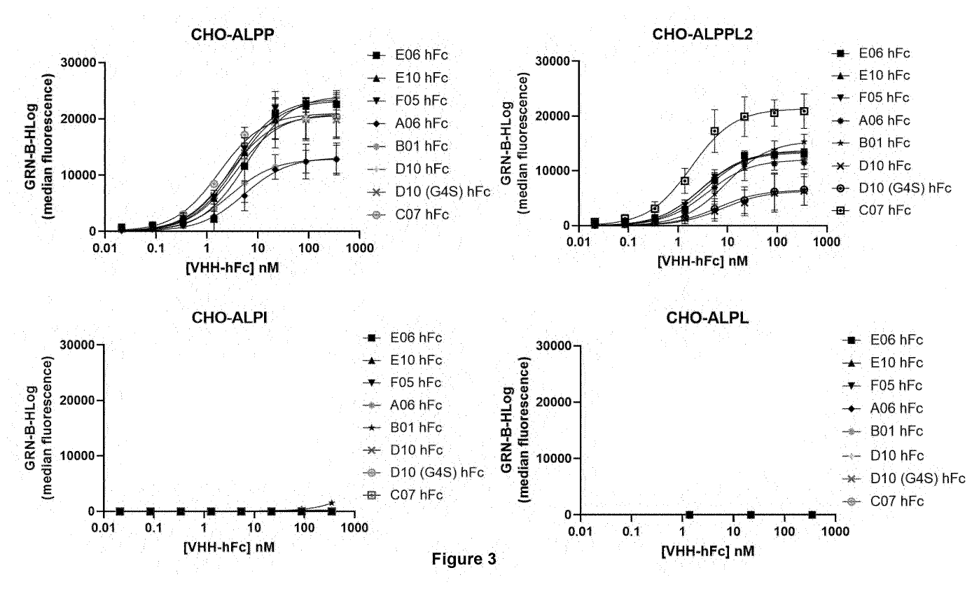

Binding of the different domains to the different cell-surface ALPP forms is summarised in Table 11 and in FIG. 3.

TABLE 11
Binding of different VHH-hFc proteins to ALPP, ALPPL2,
ALPI and ALPL expressed on the surface of CHO cells

| | | K_{D}app (nM) | | | |
|---|---|---|---|---|---|
| ID | | CHO- | CHO- | | |
| # | Molecule | ALPP | ALPPL2 | CHO-ALPI | CHO-ALPL |
| 434 | E06 hFc | 6.0 | 3.7 | No binding | No binding |
| | (S239C) | | | | |
| 435 | E10 hFc | 3.7 | 3.0 | No binding | No binding |
| | (S239C) | | | | |
| 436 | F05 hFc | 3.3 | 2.9 | No binding | No binding |
| | (S239C) | | | | |
| 340 | A06 hFc | 5.4 | 3.9 | No binding | No binding |
| | (S239C) | | | | |
| 341 | B01 hFc | 2.9 | 9.3 | ND | No binding |
| | (S239C) | | | | |
| 372 | D10 hFc | 3.6 | 8.0 | No binding | No binding |
| | (S239C) | | | | |
| 373 | D10 (G₄S)₁ | 2.7 | 6.2 | No binding | No binding |
| | hFc (S239C) | | | | |
| 342 | C07 hFc | 1.7 | 1.8 | No binding | No binding |
| | (S239C) | | | | |

ND = not-determinable [weak binding only observed at highest protein concentration used (350 nM)]

FIG. 3 and Table 11 show that the VHH-hFc fusions specifically bind ALPP/ALPPL2 on the surface of cells with no binding to ALPI or ALPL. B01-hFc showed very weak binding to CHO-ALPI cells at the highest concentration of protein used (350 nM).

### Example 3 Biparatopic ALPP/ALPPL2 Binding Proteins

### Biparatopic VHH-Fc Fusion Proteins

ALPP/ALPPL2 binding VHH proteins were genetically fused via long [G₄S]₃ or short [G₄S]₁ linkers to human IgG1 hFc engineered for heterodimerisation (Ridgway 1996 Protein Engineering 9(7):617-21). The Knob variant has a tyrosine substitution at position 366 (T366Y) and the Hole variant has a Threonine substitution at position 407 (Y407T) (EU numbering). This approach was used to generate bi-paratopic ALPP/ALPPL2 binders where one arm comprises an ALPP/ALPPL2 VHH protein and the other arm comprises a second different ALPP/ALPPL2 binding VHH domain.

In addition, cysteine substitutions were incorporated in the hIgG1 Fc sequence of both Knob and Hole variants to facilitate bioconjugation with different payloads.

The human IgG1 sequence contained 1 or 2 site specific Cys substitutions (S239C±S442C) for site specific conjugation and either the T366Y or Y407T (EU numbering) knobs-into-holes (KIH) mutation to facilitate biparatopic chain pairing through heterodimerisation of the Fc chains.

The biparatopic VHH-hFc fusion proteins were transiently co-expressed as secreted protein in CHO K1 cells and purified from the media using MabSelect™ SuRe™ (Evitria, Switzerland). Purified proteins were exchanged into PBS pH 7.4 and analysed by SEC (AdvanceBio, Agilent, running buffer DPBS), SDS PAGE and mass spectrometry to confirm sequence and protein integrity and the correct bi-specific pairing. For MS analysis proteins were deglycosylated with PNGaseF, reduced with 50 mM DTT (if required) and run on Sciex X500B QTOF via MassPREP desalting column (Waters). Characterisation data for biparatopic VHH-hFc proteins is shown in Table 12.

TABLE 12
Characterisation of Biparatopic ALPP/ALPPL2 targeting VHH-hFc proteins

| | | | Expres- | % | | | | | |
|---|---|---|---|---|---|---|---|---|---|
| | | | sion, | Monomer- | Reduced Mass, Da | | Intact Mass, Da | | |
| Protein | Linker | # | mg/L | icity | Expected | Observed | Expected | Observed | |
| D10 A06-hFc (S239C) | [G₄S]₃ | 605 | 88 | 95.4 | — | — | 79999.9 | 80249.4 | (2*Cys adduct) |
| A06 E10-hFc (S239C) | [G₄S]₃ | 606 | 96 | 96.7 | — | — | 80547.2 | 80796.2 | (2*Cys adduct) |
| D10 E10-hFc (S239C) | [G₄S]₃ | 607 | 82 | 93.0 | — | — | 80125.9 | 80375.4 | (2*Cys adduct) |
| D10 F05-hFc (S239C) | [G₄S]₃ | 608 | 102 | 95.4 | — | — | 80169.9 | 80417.5 | (2*Cys adduct) |
| A06 E06-hFc (S239C) | [G₄S]₃ | 609 | 202 | 99.1 | — | — | 80136.0 | 80385.5 | (2*Cys adduct) |
| D10 E06-hFc (S239C) | [G₄S]₃ | 610 | 338 | 94.7 | — | — | 79714.7 | 79963.5 | (2*Cys adduct) |
| A06 F05-hFc (S239C) | [G₄S]₃ | 611 | 235 | 95.5 | — | — | 80591.2 | 80843.6 | (2*Cys adduct) |
| D10 A06-hFc | [G₄S]₃ | 625 | 58 | 93.9 | — | — | 80033.0 | 80523.1 | (4*Cys adduct) |
| (S239C + S442C) | | | | | | | | | |
| D10 E06-hFc | [G₄S]₃ | 626 | 214 | 86.7 | — | — | 79744.85 | 80239.5 | (4*Cys adduct) |
| (S239C + S442C) | | | | | | | | | |
| D10 E10-hFc | [G₄S]₃ | 627 | 282 | 85.4 | — | — | 80156.0 | 80653.2 | (4*Cys adduct) |
| (S239C + S442C) | | | | | | | | | |
| D10 F05-hFc | [G₄S]₃ | 628 | 337 | 87 | — | — | 80200.0 | 80697.0 | (4*Cys adduct) |
| (S239C + S442C) | | | | | | | | | |
| A06 E06-hFc | [G₄S]₃ | 629 | 242 | 88.0 | — | — | 80166.1 | 80661.4 | (4*Cys adduct) |
| (S239C + S442C) | | | | | | | | | |
| A06 E10-hFc | [G₄S]₃ | 631 | 50 | 94.1 | — | — | 80580.3 | 81070.9 | (4*Cys adduct) |
| (S239C + S442C) | | | | | | | | | |
| A06 F05-hFc | [G₄S]₃ | 632 | 238 | 89.7 | — | — | 80621.3 | 81118.9 | (4*Cys adduct) |
| (S239C + S442C) | | | | | | | | | |
| D10 A06-hFc | [G₄S]₁ | 666 | 100 | 89.2 | — | — | 78768.9 | 79441.8 | (3*Cys, 1*GSH) |
| (S239C + S442C) | | | | | | | | | |
| D10 E06-hFc | [G₄S]₁ | 667 | 337 | 87.4 | — | — | 78483.7 | 78980.6 | (4*Cys adduct) |
| (S239C + S442C) | | | | | | | | | |

| D10 E10-hFc | [G₄S]₁ | 668 | 143 | 87.7 | — | | — | | 78894.9 | 79390.0 |
|---|---|---|---|---|---|---|---|---|---|---|
| (S239C + S442C) | | | | | | | | | | |
| D10 F05-hFc | [G₄S]₁ | 669 | 310 | 88.3 | — | | — | | 78938.9 | 79433.7 |
| (S239C + S442C) | | | | | | | | | | |
| A06 E06-hFc | [G₄S]₁ | 671 | 120 | 89.3 | 39657.1 | (A06), | 39661.9 | (A06), | — | — |
| (S239C + S442C) | | | | | 39247.9 | (E06) | 39253.1 | (E06) | | |
| A06 E10-hFc | [G₄S]₁ | 672 | 93 | 89.2 | 39657.1 | (A06), | 39662.7 (overlapping | | — | — |
| (S239C + S442C) | | | | | 39659.0 | (E10) | peak for A06 and E10) | | | |
| A06 F05-hFc | [G₄S]₁ | 673 | 128 | 91.1 | 39657.1 | (A06), | 39661.7 | (A06), | — | — |
| (S239C + S442C) | | | | | 39703.0 | (F05) | 39707.4 | (F05) | | |

| | | | | | | | | | |
|---|---|---|---|---|---|---|---|---|---|
| D10 A06-hFc | D10 - [G₄S]₁ | 702 | 144 | 90.2 | — | — | 79399.4 | 79885.2 | (4*Cys adduct) |
| (S239C + S442C) | A06 - [G₄S]₃ | | | | | | | | |
| D10 E06-hFc | D10 - [G₄S]₁ | 703 | 112 | 89.8 | — | — | 79114.3 | 79598.1 | (4*Cys adduct) |
| (S239C + S442C) | E06 - [G₄S]₃ | | | | | | | | |
| D10 E10-hFc | D10 - [G₄S]₁ | 704 | 100 | 89.6 | — | — | 79525.5 | 80009.9 | (4*Cys adduct) |
| (S239C + S442C) | E10 - [G₄S]₃ | | | | | | | | |
| A06 E06-hFc | A06 - [G₄S]₁ | 706 | 72 | 89.2 | — | — | 79535.6 | 80018.8 | (4*Cys adduct) |
| (S239C + S442C) | E06 - [G₄S]₃ | | | | | | | | |
| A06 E10-hFc | A06 - [G₄S]₁ | 707 | 140 | 90.9 | — | — | 79946.74 | 80426.1 | (4* Cys adduct) |
| (S239C + S442C) | E10 - [G₄S]₃ | | | | | | | | |
| A06 F05-hFc | A06 - [G₄S]₁ | 708 | 162 | 92.1 | — | — | 79990.8 | 80480.7 | (4* Cys adduct) |
| (S239C + S442C) | F05 - [G₄S]₃ | | | | | | | | |
| D10 F05-hFc | D10 - [G₄S]₁ | 720 | 112 | 90.1 | — | — | 79574.5 | 80061.7 | (4* Cys adduct) |
| (S239C + S442C) | F05 - [G₄S]₃ | | | | | | | | |
| D10 E06-hFc (S239C) | [G₄S]₁ | 793 | 186 | 95.3 | — | — | 78453.6 | 78705.5 | (2* Cys adduct) |
| D10 E10-hFc (S239C) | [G₄S]₁ | 794 | 132 | 97.5 | — | — | 78864.8 | 79098.8 | (2* Cys adduct) |
| D10 F05-hFc (S239C) | [G₄S]₁ | 795 | 326 | 96.7 | — | — | 78908.8 | 79158.0 | (2* Cys adduct) |
| D10 F05-hFc (S442C) | [G₄S]₃ | 863 | 150 | 87.6 | — | — | 80169.9 | 80421.9 | (2* Cys adduct) |

Purified target biparatopic proteins were generated in good yield and showed high percentage monomericity as determined by SEC, i.e. the desired heterodimer product is obtained with low amounts of high molecular weight aggregates.

### Binding of Biparatopic VHH-hFc Proteins to ALPP Homologues by BLI

Binding kinetics were determined using the Biolayer Interferometry (BLI) Octet K2 or R4 system (Sartorius). Human ALPP, ALPPL2 and ALPL all fused with a disulphide stabilised leucine zipper (SEQ ID NO: 265, SEQ ID NO: 266, and SEQ ID NO: 267) and ALPI His (SEQ ID NO:264) were used in BLI experiments. For K_{D} apparent measurements, the ALPP proteins were immobilised in sodium acetate pH5 buffer to AR2G sensors using amine coupling. To measure 1:1 interactions, AHC biosensors were used to immobilise Fc-fused proteins. All assays were conducted in HBS-EP with a plate temperature of 25° C. Ka (M⁻¹s⁻¹), Kd (s⁻¹) and K_{D} (nM) values were determined using Octet Data Analysis High Throughput software (Sartorius) for Biolayer Interferometry. Binding kinetics are shown in Tables 13 and 14.

TABLE 13
Binding of Biparatopic VHH-hFc fusion proteins with [G₄S]₃ linkers to ALPP, ALPPL2, ALPI and ALPL by BLI

| | | hALPP (K_{D} app) | | | hALPP (K_{D} 1:1) | | | hALPPL2 (K_{D} app) | | |
|---|---|---|---|---|---|---|---|---|---|---|
| | | K_{D} | Kₐ | K_{d} | K_{D} | Kₐ | K_{d} | K_{D} | Kₐ | K_{d} |
| Name | # | (nM) | (M⁻¹s⁻¹) | (s⁻¹) | (nM) | (M⁻¹s⁻¹) | (s⁻¹) | (nM) | (M⁻¹s⁻¹) | (s⁻¹) |
| D10 A06-hFc | 605 | <0.01 | 283500 | <1.0E−07 | <0.01 | 443400 | <1.0E−07 | 1.218 | 540200 | 0.000658 |
| D10 E06-hFc | 610 | <0.01 | 355700 | <1.0E−07 | <0.01 | 494200 | <1.0E−07 | 0.192 | 331000 | 0.0000636 |
| D10 E10-hFc | 607 | <0.01 | 402300 | <1.0E−07 | <0.01 | 445100 | <1.0E−07 | 0.286 | 267700 | 0.0000764 |
| D10 F05-hFc | 608 | 0.014 | 341700 | 0.000004816 | <0.01 | 389400 | <1.0E−07 | 0.236 | 608800 | 0.0001436 |
| A06 E06-hFc | 609 | <0.01 | 443000 | <1.0E−07 | <0.01 | 449900 | <1.0E−07 | 0.0086 | 433800 | 0.0000374 |
| A06 E10-hFc | 606 | <0.01 | 473800 | <1.0E−07 | <0.01 | 451800 | <1.0E−07 | 0.138 | 355700 | 0.0000491 |
| A06 F05-hFc | 611 | 0.0204 | 359400 | 0.00000733 | <0.01 | 398500 | <1.0E−07 | 0.272 | 530900 | 0.000144 |

| | hALPPL2 (K_{D} 1:1) | | | | |
|---|---|---|---|---|---|
| | K_{D} | Kₐ | K_{d} | | |
| Name | (nM) | (M⁻¹s⁻¹) | (s⁻¹) | hALPI | hALPL |
| D10 A06-hFc | <0.01 | 504800 | <1.0E−07 | No binding | No binding |
| D10 E06-hFc | <0.01 | 711500 | <1.0E−07 | No binding | No binding |
| D10 E10-hFc | <0.01 | 678100 | <1.0E−07 | No binding | No binding |
| D10 F05-hFc | <0.01 | 442200 | <1.0E−07 | No binding | No binding |
| A06 E06-hFc | <0.01 | 689000 | <1.0E−07 | No binding | No binding |
| A06 E10-hFc | <0.01 | 624400 | <1.0E−07 | No binding | No binding |
| A06 F05-hFc | <0.01 | 600300 | <1.0E−07 | No binding | No binding |

TABLE 14
Binding of Biparatopic VHH-hFc fusion proteins with
short or mixed linker lengths to ALPP by BLI

| | | | hALPP (K_{D} app) | | |
|---|---|---|---|---|---|
| Name | Linker | # | K_{D} (nM) | Kₐ (M⁻¹s⁻¹) | K_{d} (s⁻¹) |
| D10 A06-hFc | [G₄S]₁ | 666 | <0.01 | 332300 | 0.000000828 |
| D10 E06-hFc | [G₄S]₁ | 667 | 0.048 | 270800 | 0.00001298 |
| D10 E10-hFc | [G₄S]₁ | 668 | <0.01 | 374200 | 0.000000786 |
| D10 F05-hFc | [G₄S]₁ | 669 | <0.01 | 430200 | <1.0E−07 |
| A06 E06-hFc | [G₄S]₁ | 671 | 0.0202 | 602400 | 0.0000122 |
| A06 E10-hFc | [G₄S]₁ | 672 | 0.047 | 515500 | 0.000021 |
| A06 F05-hFc | [G₄S]₁ | 673 | 0.0635 | 422300 | 0.0000268 |
| D10 A06-hFc | Mixed | 702 | <0.01 | 343400 | <1.0E−07 |
| | ([G₄S]₁/ | | | | |
| | [G₄S]₃ | | | | |
| D10 E06-hFc | Mixed | 703 | 0.11 | 464800 | 0.00005088 |
| | ([G₄S]₁/ | | | | |
| | [G₄S]₃ | | | | |
| D10 E10-hFc | Mixed | 704 | <0.01 | 384800 | <1.0E−07 |
| | ([G₄S]₁/ | | | | |
| | [G₄S]₃ | | | | |
| D10 F05-hFc | Mixed | 720 | — | — | — |
| | ([G₄S]₁/ | | | | |
| | [G₄S]₃ | | | | |
| A06 E06-hFc | Mixed | 706 | <0.01 | 429300 | <1.0E−07 |
| | ([G₄S]₁/ | | | | |
| | [G₄S]₃ | | | | |
| A06 E10-hFc | Mixed | 707 | <0.01 | 421300 | <1.0E−07 |
| | ([G₄S]₁/ | | | | |
| | [G₄S]₃ | | | | |
| A06 F05-hFc | Mixed | 708 | <0.01 | 425800 | <1.0E−07 |
| | ([G₄S]₁/ | | | | |
| | [G₄S]₃ | | | | |

BLI data confirms the high affinity binding of Biparatopic VHH-hFc fusion proteins to ALPP and ALPPL2. No binding to ALPI or ALPL was observed.

### Example 4—ALPP/ALPPL2 Protein Drug Conjugates

### VHH-hFc Drug Conjugates

An approach for generating ADCs is to engineer cysteine substitutions or additions at positions on the light and heavy chains of antibodies and these cysteines provide reactive thiol groups for site specific labelling (Junutula 2008 Nature Biotechnology 26, 925-932, Jeffrey 2013, Sutherland 201 6).

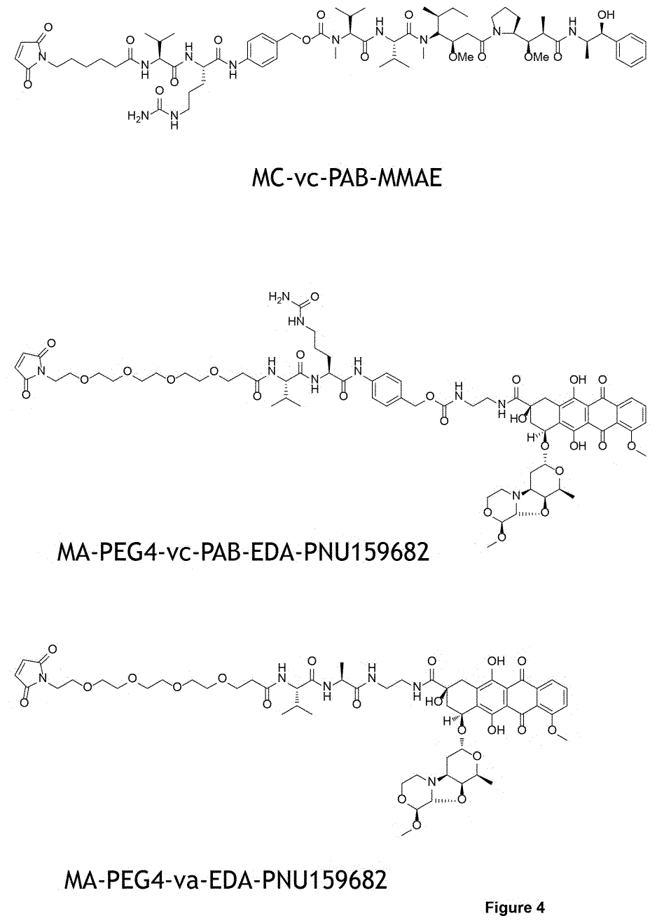

The anti ALPP/ALPPL2 VHH-hFc fusions were generated with additional cysteine residues engineered into the Fc region as described previously, which enabled site specific labelling with maleimide derivatives of payloads and cytotoxic drugs (MC-vc-PAB-MMAE, MA-PEG4-vc-PAB-EDA-PNU1 59682 and MA-PEG4-va-PAB-EDA-PNU159682) (FIG. 4).

### Generation of VHH-hFc—MMAE Conjugates

Using a partial reduction, refolding and labelling method adapted from the literature [Junutula et al, 2008 Nat Biotech, Jeffrey et al, 2013 Bioconj Chem], these proteins were site specific labelled with the maleimide MMAE derivatives. Briefly, 1-6 mg/ml VHH hFc solutions were prepared in PBS pH 7.4 with 1 mM EDTA, 20-40 molar equivalents TCEP was added and incubated at 4° C. for 16 hours. 30-50 molar equivalents DHAA is then added, pH adjusted to 6.5 and incubated at room temperature for 1 hour. Refolded VHH Fc was extensively dialysed or buffer exchanged into PBS and quantified by UV before reacting with 4-8 molar equivalents maleimide MMAE solution, room temperature for 2 hours. Conjugates were purified by SEC and analysed by reducing and non-reducing SDS-PAGE and LC-MS. Table 15 summaries the conjugates prepared.

TABLE 15
Summary of characteristics of VHH-hFc conjugated to MMAE

| | | | | | Reduced, deglycosylated | | Intact, deglycosylated | | |
|---|---|---|---|---|---|---|---|---|---|
| | | | | DAR | mass, Da | | mass, Da | | |
| Protein | Linker | Payload | # | (by MS) | Expected | Observed | Expected | Observed | Yield, % |
| A06-hFc (S239C) | [G₄S]₃ | MC-vc-PAB- | 376 | 2.0 | 41526.58 | 41531.6 | — | — | 63 |
| | | MMAE | | | | | | | |
| C07-hFc (S239C) | [G₄S]₃ | MC-vc-PAB- | 377 | 2.0 | 41147.3 | 41126 | — | — | 63 |
| | | MMAE | | | | | | | |
| F08-hFc (S239C) | [G₄S]₃ | MC-vc-PAB- | 378 | 2.0 | 41578.8 | 41557.4 | — | — | 63 |
| | | MMAE | | | | | | | |
| D04-hFc (S239C) | [G₄S]₃ | MC-vc-PAB- | 381 | 2.0 | 41280.1 | 41285.6 | — | — | 73.5 |
| | | MMAE | | | | | | | |
| D10-hFc (S239C) | [G₄S]₃ | MC-vc-PAB- | 382 | 2.0 | 41105.3 | 41112.4 | — | — | 53.2 |
| | | MMAE | | | | | | | |
| D10-hFc (S239C) | [G₄S]₁ | MC-vc-PAB- | 383 | 2.0 | 40474.7 | 40481.9 | — | — | 55.1 |
| | | MMAE | | | | | | | |
| A05-hFc (S239C) | [G₄S]₃ | MC-vc-PAB- | 442 | 1.92 | 41601.6 | 41603.8 | — | — | 85 |
| | | MMAE | | | | | | | |
| E06-hFc (S239C) | [G₄S]₃ | MC-vc-PAB- | 443 | 1.87 | 41373.5 | 41375.4 | — | — | 67.2 |
| | | MMAE | | | | | | | |
| E10-hFc (S239C) | [G₄S]₃ | MC-vc-PAB- | 444 | 1.94 | 41784.7 | 41785.2 | — | — | 75.6 |
| | | MMAE | | | | | | | |
| F05-hFc (S239C) | [G₄S]₃ | MC-vc-PAB- | 445 | 1.87 | 41828.6 | 41829.1 | — | — | 62.8 |
| | | MMAE | | | | | | | |
| E06 linkerCys-hFc | GCGGS | MC-vc-PAB- | 542 | 4.1 | — | — | 85205 | 85226.4 | 50.7 |
| (S442C) | [G₄S]₂ | MMAE | | | | | | | |
| D10 linkerCys-hFc | GCGGS | MC-vc-PAB- | 543 | 4.1 | — | — | 84932.8 | 84952.3 | 56.8 |
| (S442C) | [G₄S]₂ | MMAE | | | | | | | |

SDS-PAGE and mass spectrometry analysis of the final conjugates determined that the labelling had proceeded in a quantitative fashion to give highly pure homogenous protein drug conjugates with the desired drug to antibody ratios (DAR of 2 or 4).

### Generation of VHH-hFc—PNU Conjugates

A similar partial reduction, refolding and labelling method was used for PNU conjugation as above with some modifications. Briefly, 1-6 mg/ml VHH hFc solutions were prepared in PBS+100 mM L-Arg pH 7.4 with 1 mM EDTA. 20 molar equivalents TCEP added and incubated at 4° C. for 16 hours. 30 molar equivalents DHAA is then added, pH adjusted to 6.5 and incubated at room temperature for 3 hours. Refolded VHH Fc was buffer exchanged into PBS using NAP25 columns and quantified by UV. Propylene glycol was then added to a 20% final concentration before addition of 4 molar equivalents maleimide PNU solution. This was incubated at room temperature for 2 hours. Conjugates were purified using activated carbon and NAP-25 desalting columns before being analysed by reducing and non-reducing SDS-PAGE, analytical HIC and LC-MS. Table 16 summarizes the conjugates prepared.

TABLE 16
Summary of characteristics of VHH-hFc conjugated to PNU

| | | | | | Intact, deglycosylated | | |
|---|---|---|---|---|---|---|---|
| | | | | | mass, Da | | |
| Protein | Linker | Payload | # | DAR | Expected | Observed | Yield, % |
| A06-hFc (S239C) | [G₄S]₃ | MA-PEG4-vc-PAB- | 776 | 1.93 | 83226.2 | 83230 | 53 |
| | | EDA-PNU159682 | | | | | |
| D10-hFc (S239C) | [G₄S]₃ | MA-PEG4-vc-PAB- | 777 | 2 | 82383.6 | 82388 | 54 |
| | | EDA-PNU159682 | | | | | |
| E06-hFc (S239C) | [G₄S]₃ | MA-PEG4-vc-PAB- | 778 | 1.97 | 82655.9 | 82660 | 49 |
| | | EDA-PNU159682 | | | | | |
| E10-hFc (S239C) | [G₄S]₃ | MA-PEG4-vc-PAB- | 779 | 1.98 | 83484.2 | 83482 | 57 |
| | | EDA-PNU159682 | | | | | |
| F05-hFc (S239C) | [G₄S]₃ | MA-PEG4-vc-PAB- | 780 | 1.94 | 83566.3 | 83570 | 56 |
| | | EDA-PNU159682 | | | | | |

SDS-PAGE, SEC and mass spectrometry analysis of the final conjugates determined that the labelling had proceeded in a quantitative fashion to give highly pure homogenous protein drug conjugates with the desired drug to antibody ratio (DAR of 2) and with low levels of high molecular weight aggregates (<4%).

### Characterisation of Cancer Cell-Lines for ALPP/ALPPL2 Expression

The following cell lines were characterised for cell surface ALPP isoform expression analysis and most were also used in cell viability assays.

CHO-ALPP isoform stable transfectants were generated using CHO-K1 cells (CCL-61) provided by the American Type Culture Collection (ATCC). Grown in ATCC-formulated F-12K medium+10% FCS.

MKN1 cells (RCB1003, Riken BRC). Ref: Cancer Sci 2006; 97:387-394, were provided by the RIKEN BRC through the National BioResource Project of the MEXT, Japan Grown in RPMI1640+10% FCS.

NCI-H1651 cells, CRL-5884 provided by the American Type Culture Collection (ATCC). Grown in ATCC DMEM:F12 medium+10% FCS plus supplements required to make ACL-4 media as recommended by ATCC.

NCI-N87 cells, CRL-5822 provided by the American Type Culture Collection (ATCC). Grown in RPM11640+10% FCS.

HPAC cells, CRL-2119 provided by the American Type Culture Collection (ATCC). Grown in 1:1 DMEM:Ham's F12+5% FCS plus supplements as recommended by ATCC.

Caov3 cells, HTB-75 provided by the American Type Culture Collection (ATCC). Grown in DMEM+10% FCS.

RMUG-S cells, IFO50320 provided by the Japanese Collection of Research Bioresouces (JCRB) via Tebu-bio. Ref: M. Sakayori et. aL. Hum Cell 1990 March;3(1):52-6. Grown in Ham's F12 media+10% FCS.

HeLa cells, CCL-2 provided by the American Type Culture Collection (ATCC). Grown in EMEM+10% FCS

MDA-MB-468 cells, HTB-132 provided by the American Type Culture Collections (ATCC). Grown in DMEM+10% FCS

ALPP/ALPPL2 protein expression levels on the surface of different cancer cell-lines were determined by flow cytometry. ALPP/ALPPL2 was assessed using an anti-PLAP mAb (PE-8B6 from Santa Cruz).

Adherent cells were detached from tissue culture flasks by incubating with 0.1% EDTA/PBS solution at 37° C. for ~10 minutes or until cells detached easily. Cells were re-suspended in ice-cold PBS/2% FCS in 15 ml tubes and centrifuged at 1700 rpm for 5 mins at 4° C. Supernatant was removed and the cell pellet re-suspended in PBS/2% FCS. A cell count was performed using a Chemometec Nucleocounter NC-202 and 2.5×10≡cells were aliquoted per test sample into a 96 well plate. Cells were incubated with 100 μl of PE-anti-PLAP mAb or controls for 1 hr on ice. The sample plate was centrifuged at 1700 rpm for 5 mins at 4° C. The supernatant was removed, and a wash performed by re-suspending the cell pellets in 0.25 mL of ice-cold PBS/2% FCS using a multichannel pipette. Samples were again centrifuged at 1700 rpm for 5 min at 4° C. Supernatant was removed and two further washes performed as described. After the final wash and centrifugation step, excess liquid was removed by blotting the plate on tissue paper. The cell pellets were re-suspended in 0.28 ml of ice-cold PBS/2% FCS and left on ice in the dark prior to analysis on a Merck-Millipore Guava EasyCyte HT or Thermo Fisher Attune N×T flow cytometer.

Protein expression levels and mRNA expression values are shown in the Table 17 below.

ALPP/ALPPL2 mRNA expression levels were obtained from the Cancer Cell-Line Encyclopaedia (CCLE).

TABLE 17
ALPP/ALPPL2 expression on cancer cell-lines

| | | Cell | ALPP | ALPPL2 |
|---|---|---|---|---|
| | | surface | mRNA | mRNA |
| | | MFI | (log 2 | (log 2 |
| | | (PE-8B6 | TPM | TPM |
| Cell line | Indication | signal) | CCLE) | CCLE) |
| MKN-1 | Gastric cancer | ++++ | 8.7 | 6 |
| NCI-H1651 | Lung | +++ | 8.9 | 10.7 |
| | adenocarcinoma | | | |
| | (NSCLC) | | | |
| NCI-N87 | Gastric cancer | +++ | 6.92 | 6 |
| HPAC | Pancreatic | ++ | 7.93 | 6.43 |
| | adenocarcinoma | | | |
| Caov-3 | Ovarian | ++ | 7.18 | 2.59 |
| | adenocarcinoma | | | |
| RMUG-S | Ovarian mucinous | ++++ | 7.67 | 3.1 |
| | cystadenocarcinoma | | | |
| HeLa | Cervical | − | 0.8 | −1.9 |
| | adenocarcinoma | | | |

++++ >100000 MFI
+++ 50000-100000 MFI
++ 10000-50000 MFI
+ 100-10000 MFI
− ≤100 MFI

In vitro cell viability assays for cancer cells treated with anti ALPP/ALPPL2 drug conjugates Cells were seeded in the appropriate complete growth media, as described above, into white, clear bottom 96 well plates (Costar) and incubated at 37° C., 5% C02 for 24 hours. On the following day, dilution series were set up for each test agent at ×10 working stocks. The dose response X10 stock was e.g.: 10000, 5000, 1000, 500, 100, 50, 10, 5, 1, 0.5 nM etc. 10 μL of the X10 stock solutions were added to the cell plates (90 μl per well) using a multichannel pipette. This resulted in a 1:10 dilution into the well and dose responses ranging from 1000 nM (column 1) to 0.05 nM (column 10) or continued to 0.5 fM, if required, for the most sensitive cells lines. 10 μl of vehicle control (PBS) was added to the control wells (columns 11 and 12). Plates were incubated at 37° C., 5% CO₂ for 96 hours. Promega Cell Titre Glo reagent was used as per the manufacturer's instructions to assess cell viability. Briefly, assay plates were removed from incubator and allowed to equilibrate to room temperature before adding 100 μl of room temperature Cell Titre Glo reagent to each 100 μl assay well. Plates were placed on a plate shaker for 2 minutes at 600 rpm. Plates were allowed to sit for a further 10 minutes at room temperature prior to measuring luminescence read-out using a Clariostar plate-reader (BMG). Data was analysed by calculating the average for untreated (vehicle only) control wells and determining the % of control for each treated well. % of control data was then plotted against Log [Treatment] concentration and the IC50 value derived using non-linear regression fitting in GraphPad Prism software.

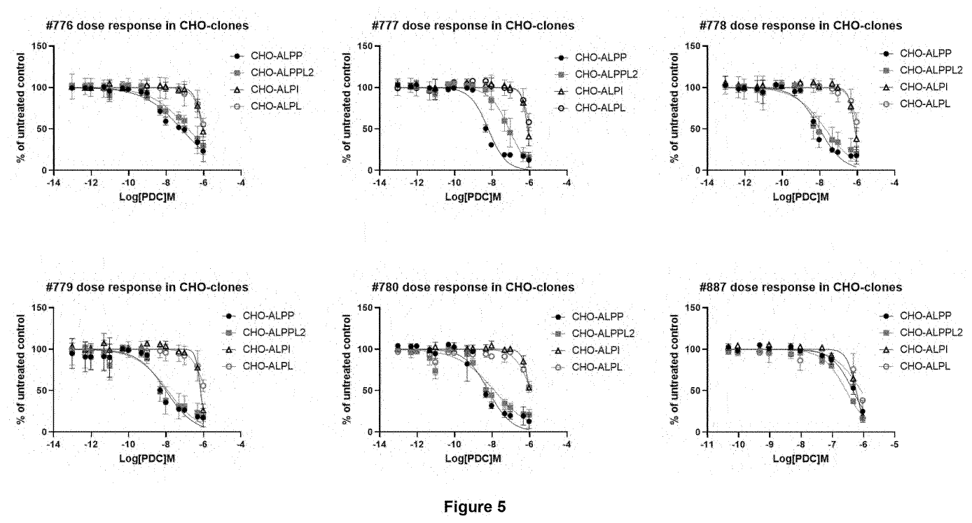

Table 18 shows IC₅₀ values, for cell-killing of CHO-ALPP isoform transfectant cell lines stably expressing either ALPP, ALPPL2, ALPI or ALPL by monoparatopic VHH-hFc drug conjugates [-vc-PAB-MMAE (DAR4) or -PEG4-vc-PAB-EDA-PNU159682 (DAR2)]. αGFP-hFc (S239C+S442C)-vc-PAB-MMAE (DAR4) and αGFP-hFc (S239C)-vc-PAB-EDA-PNU159682 (DAR2) agents were included as non-binding negative controls. αGFP-hFc is a control non-binding protein, which consists of a GFP targeting VHH domain fused to human IgG1 Fc in an analogous fashion to the ALPP/ALPPL2 targeting proteins. FIG. 5 shows representative dose response curves, for cell-killing of the CHO-ALPP, CHO-ALPPL2, CHO-ALPI and CHO-ALPL cells by the VHH-hFc-PNU drug conjugates.

TABLE 18
Calculated IC₅₀ values (nM) for the cell-killing of CHO-ALPP,
CHO-ALPPL2, CHO-ALPI, CHO-ALPL by VHH-hFc drug conjugates.

| | | | IC₅₀ @ 96 h (nM) | | | |
|---|---|---|---|---|---|---|
| ID # | Molecule | DAR | CHO-ALPP | CHO-ALPPL2 | CHO-ALPI | CHO-ALPL |
| 543 | D10 linker Cys-hFc-vcPAB-MMAE | 4.1 | 36.9 | 52.2 | >664 | >664 |
| | (S442C) | | | | | |
| 542 | E06 linker Cys-hFc-vcPAB-MMAE | 4.1 | 56.7 | 43.7 | >592 | >592 |
| | (S442C) | | | | | |
| 721 | αGFP-hFc-vcPAB-MMAE | 4 | >1000 | >1000 | >1000 | >1000 |
| 776 | A06-hFc-vcPAB-EDA-PNU | 1.93 | 64.1 | 147.8 | 883.2 | >1000 |
| 777 | D10-hFc-vcPAB-EDA-PNU | 2 | 6.2 | 79.9 | 797.6 | >1000 |
| 778 | E06-hFc-vcPAB-EDA-PNU | 1.97 | 9.8 | 19.8 | 751.7 | >1000 |
| 779 | E10-hFc-vcPAB-EDA-PNU | 1.98 | 9.8 | 12.3 | 699.8 | >1000 |
| 887 | αGFP-hFc-vcPAB-EDA-PNU | 2 | 460.9 | 279.7 | 579.1 | 834.7 |
| | MMAE | N/A | 19.1, 19.5 | 24.4, 16.6 | 11.4, 15.9 | 29.4, 25.8 |
| | EDA-PNU | N/A | 5.3 | 5.6 | 4.3 | 5.5 |

As shown in Table 18 and FIG. 5 VHH-hFc-MMAE and PNU conjugates have been generated, which show potent and selective killing of ALPP and ALPPL2 expressing CHO cells with much lower potencies for killing of ALPI and ALPL expressing CHO cells. This high selectivity for killing of ALPP and ALPPL2 expressing cells is observed for VHH-Fc proteins conjugated with both MMAE and PNU payloads. D10-hFc drug conjugates show increased potency for ALPP expressing CHO cells versus ALPPL2-expressing CHO-cells.

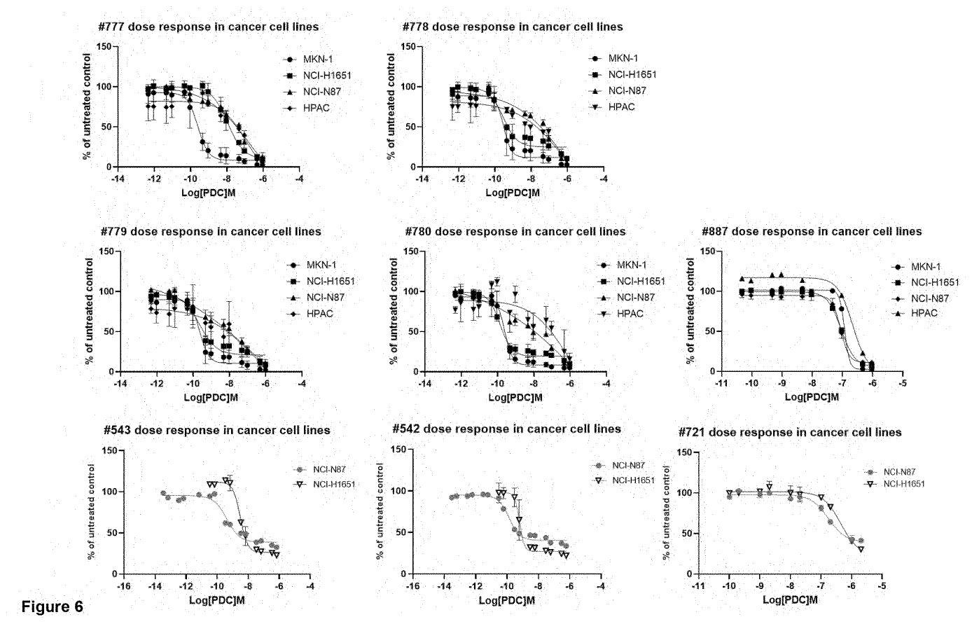

Table 19 and FIG. 6 show dose response curves, with corresponding IC₅₀ values, for cell-killing of ALPP/ALPPL2 positive cancer cells by the monoparatopic VHH-hFc drug conjugates [-vc-PAB-MMAE (DAR4) or -PEG4-vc-PAB-EDA-PNU159682 (DAR2)]. αGFP-hFc (S239C+S442C)-vc-PAB-MMAE (DAR4) and αGFP-hFc (S239C)-vc-PAB-EDA-PNU159682 (DAR2) agents were included as non-binding negative controls.

TABLE 19
Calculated IC₅₀ values (nM) for the cell-killing of different
cancer cell-lines by VHH-hFc drug conjugates.

| ID | | | IC₅₀ @ 96 h (nM) and (% viability remaining) | | | |
|---|---|---|---|---|---|---|
| # | Molecule | DAR | MKN1 | NCI-H1651 | NCI-N87 | HPAC |
| 543 | D10 linker Cys-hFc- | 4.1 | ND | 5.2 (26.4) | 1.6 (38.7) | ND |
| | vcPAB-MMAE | | | | | |
| | (S442C) | | | | | |
| 542 | E06 linker Cys-hFc- | 4.1 | ND | 0.81 (27.1) | 0.46 (40.5) | ND |
| | vcPAB-MMAE | | | | | |
| | (S442C) | | | | | |
| 721 | αGFP-hFc-vcPAB- | 4 | | 682.3 (23.1) | 465.0 (39.5) | |
| | MMAE | | | | | |
| 777 | D10hFc-vcPAB- | 2 | 0.25 (8.4%) | 13.8 (10%) | 35.1 (0%) | 35.6 (0%) |
| | EDA-PNU | | | | | |
| 778 | E06hFc-vcPAB- | 1.97 | 0.29 (11.6%) | 0.54 (24.5%) | 33.5 (0%) | 20.0 (0%) |
| | EDA-PNU | | | | | |
| 779 | E10hFc-vcPAB- | 1.98 | 0.23 (10.5%) | 0.38 (21.1%) | 9.3 (0%) | 8.0 (0%) |
| | EDA-PNU | | | | | |
| 780 | F05hFc-vcPAB- | 1.94 | 0.24 (8.5%) | 0.2 (19.5%) | 10.5 (0%) | 94.6 (0%) |
| | EDA-PNU | | | | | |
| 887 | αGFP-hFc-vcPAB- | 2.1 | 120.0 (2.7%) | 96.0 (5.6%) | 91.0 (11.1%) | 254.0 (0%) |
| | EDA-PNU | | | | | |

As shown in Table 19 and FIG. 6, ALPP/ALPPL2 VHH-hFc-MMAE and PNU conjugates have been generated, which show potent killing of ALPP/ALPPL2 expressing cancer cells.

Large windows for cell-killing are observed between the ALPP/ALPPL2 targeting VHH-hFc-MMAE and PNU conjugates compared to the respective non-binding αGFP-hFc conjugate controls.

Therefore, highly selective and potent cell-killing for ALPP and ALPPL2 expressing cancer cells is observed for the VHH-Fc proteins conjugated with both MMAE and PNU payloads.

### Example 5—Biparatopic ALPP/ALPPL2 Drug Conjugates

### Biparatopic VHH-hFc Drug Conjugates

Biparatopic anti ALPP/ALPPL2 VHH-hFc fusions were generated with additional cysteine residues engineered into the Fc region as described previously, which enabled site specific labelling with maleimide derivatives of payloads and cytotoxic drugs (MC-vc-PAB-MMAE and MA-PEG4-vc-PAB-EDA PNU159682).

Biparatopic anti ALPP/ALPPL2 VHH-hFc proteins were conjugated with either MMAE or PNU derivatives and analysed as described in Example 4. Table 20 and Table 21 summarises the conjugates prepared.

TABLE 20
Summary of characteristics of Bi-paratopic VHH-hFc conjugated to MMAE

| | | | | | Intact, | | |
|---|---|---|---|---|---|---|---|
| | | | | | deglycosylated | | |
| | | | | | mass, Da | | |
| Protein | Linker | Payload | # | DAR | Expected | Observed | Yield, % |
| D10 A06-hFc (S239C | [G₄S]₃ | MC-vc-PAB- | 613 | 1.9 | 82629.9 | 82641.9 | 40.5 |
| T366Y/Y407T) | | MMAE | | | | | |
| A06 E10-hFc (S239C | [G₄S]₃ | MC-vc-PAB- | 614 | 1.8 | 83177.2 | 83189.4 | 46.5 |
| T366Y/Y407T) | | MMAE | | | | | |
| D10 E10-hFc (S239C | [G₄S]₃ | MC-vc-PAB- | 615 | 1.9 | 82755.9 | 82766.7 | 44.5 |
| T366Y/Y407T) | | MMAE | | | | | |
| D10 F05-hFc (S239C | [G₄S]₃ | MC-vc-PAB- | 616 | 2 | 82801.9 | 82813 | 73 |
| T366Y/Y407T) | | MMAE | | | | | |
| A06 F05-hFc (S239C | [G₄S]₃ | MC-vc-PAB- | 618 | 1.92 | 83221.4 | 83234.5 | 41.8 |
| T366Y/Y407T) | | MMAE | | | | | |
| A06 E06-hFc (S239C | [G₄S]₃ | MC-vc-PAB- | 619 | 1.92 | 82766.01 | 82777.7 | 49.7 |
| T366Y/Y407T) | | MMAE | | | | | |
| D10 A06-hFc (S239/442C | [G₄S]₃ | MC-vc-PAB- | 633 | 4 | 85289.9 | 85304.3 | 47 |
| T366Y/Y407T) | | MMAE | | | | | |
| D10 E06-hFc (S239/442C | [G₄S]₃ | MC-vc-PAB- | 634 | 4 | 85010.9 | 85032.4 | 77.4 |
| T366Y/Y407T) | | MMAE | | | | | |
| D10 E10-hFc (S239/442C | [G₄S]₃ | MC-vc-PAB- | 636 | 4 | 85420 | 85437.8 | 71.7 |
| T366Y/Y407T) | | MMAE | | | | | |
| D10 F05-hFc (S239/442C | [G₄S]₃ | MC-vc-PAB- | 637 | 4 | 85464 | 85479.7 | 71.3 |
| T366Y/Y407T) | | MMAE | | | | | |
| A06 E06-hFc (S239/442C | [G₄S]₃ | MC-vc-PAB- | 639 | 4 | 85426.1 | 85438.6 | 55 |
| T366Y/Y407T) | | MMAE | | | | | |
| A06 E10-hFc (S239/442C | [G₄S]₃ | MC-vc-PAB- | 664 | 4 | 85837.3 | 85849.1 | 58 |
| T366Y/Y407T) | | MMAE | | | | | |
| A06 F05-hFc (S239/442C | [G₄S]₃ | MC-vc-PAB- | 665 | 4 | 85885.3 | 85903.4 | 73.3 |
| T366Y/Y407T) | | MMAE | | | | | |
| D10 A06-hFc (S239/442C | [G₄S]₁ | MC-vc-PAB- | 676 | 4 | 81455.1 | 84045.8 | 60 |
| T366Y/Y407T) | | MMAE | | | | | |
| D10 E06-hFc (S239/442C | [G₄S]₁ | MC-vc-PAB- | 677 | 4 | 83747.7 | 83764 | 70.3 |
| T366Y/Y407T) | | MMAE | | | | | |
| D10 E10-hFc (S239/442C | [G₄S]₁ | MC-vc-PAB- | 678 | 4 | 84158.9 | 84172.4 | 65.3 |
| T366Y/Y407T) | | MMAE | | | | | |
| D10 F05-hFc (S239/442C | [G₄S]₁ | MC-vc-PAB- | 679 | 4 | 84202.9 | 84215.9 | 73.7 |
| T366Y/Y407T) | | MMAE | | | | | |
| A06 E06-hFc (S239/442C | [G₄S]₁ | MC-vc-PAB- | 710 | 4 | 84168.9 | 84179.5 | 51.5 |
| T366Y/Y407T) | | MMAE | | | | | |
| A06 E10-hFc (S239/442C | [G₄S]₁ | MC-vc-PAB- | 711 | 4 | 84580.2 | 84591.6 | 53 |
| T366Y/Y407T) | | MMAE | | | | | |
| A06 F05-hFc (S239/442C | [G₄S]₁ | MC-vc-PAB- | 712 | 4 | 84624.2 | 84635.5 | 51 |
| T366Y/Y407T) | | MMAE | | | | | |
| D10 E06-hFc (S239/442C | D10 - [G₄S]₁ | MC-vc-PAB- | 717 | 4 | 84378.3 | 84391.5 | 50 |
| T366Y/Y407T) | E06 - [G₄S]₃ | MMAE | | | | | |
| D10 A06-hFc (S239/442C | D10 - [G₄S]₁ | MC-vc-PAB- | 718 | 4 | 84662.4 | 84678.8 | 51 |
| T366Y/Y407T) | A06 - [G₄S]₃ | MMAE | | | | | |
| D10 E10-hFc (S239/442C | D10 - [G₄S]₁ | MC-vc-PAB- | 722 | 4 | 84789.5 | 84806 | 51.5 |
| T366Y/Y407T) | E10 - [G₄S]₃ | MMAE | | | | | |
| A06 E06-hFc (S239/442C | A06 - [G₄S]₁ | MC-vc-PAB- | 728 | 4 | 84799.6 | 84813.1 | 56.8 |
| T366Y/Y407T) | E06 - [G₄S]₃ | MMAE | | | | | |
| A06 F05-hFc (S239/442C | A06 - [G₄S]₁ | MC-vc-PAB- | 729 | 4 | 85254.7 | 85267.8 | 58.2 |
| T366Y/Y407T) | F05 - [G₄S]₃ | MMAE | | | | | |
| A06 E10-hFc (S239/442C | A06 - [G₄S]₁ | MC-vc-PAB- | 730 | 4 | 85210.7 | 85222.9 | 57 |
| T366Y/Y407T) | E10 - [G₄S]₃ | MMAE | | | | | |
| D10 F05-hFc (S239/442C | D10 - [G₄S]₁ | MC-vc-PAB- | 731 | 4 | 84833.5 | 84847.5 | 65 |
| T366Y/Y407T) | F05 - [G₄S]₃ | MMAE | | | | | |

SEC and SDS-PAGE and mass spectrometry analysis of the final conjugates determined that the labelling had proceeded in a quantitative fashion to give highly pure homogenous protein drug conjugates with the desired drug to antibody ratio (either DAR2 or DAR4) and with low levels of high molecular weight aggregates observed.

TABLE 21
Summary of characteristics of Bi-paratopic VHH-hFc conjugated to PNU

| | | | | | Intact, | | |
|---|---|---|---|---|---|---|---|
| | | | | | deglycosylated | | |
| | | | | | mass, Da | | |
| Protein | Linker | Payload | # | DAR | Expected | Observed | Yield, % |
| D10 E10-hFc (S239C | [G₄S]₃ | MA-PEG4-vc-PAB- | 880 | 2.02 | 82930.9 | 82935.9 | 64 |
| T366Y/Y407T) | | EDA-PNU159682 | | | | | |
| D10 E06-hFc (S239C | [G₄S]₃ | MA-PEG4-vc-PAB- | 881 | 2.02 | 82519.7 | 82525.1 | 65 |
| T366Y/Y407T) | | EDA-PNU159682 | | | | | |
| D10 F05-hFc (S239C | [G₄S]₃ | MA-PEG4-vc-PAB- | 882 | 1.94 | 82974.9 | 82980 | 64 |
| T366Y/Y407T) | | EDA-PNU159682 | | | | | |
| A06 F05-hFc (S239C | [G₄S]₃ | MA-PEG4-vc-PAB- | 883 | 2.03 | 83396.2 | 83400.1 | 64 |
| T366Y/Y407T) | | EDA-PNU159682 | | | | | |
| D10 E06-hFc (S239C | [G₄S]₁ | MA-PEG4-vc-PAB- | 884 | 2.03 | 81258.6 | 81267.5 | 59 |
| T366Y/Y407T) | | EDA-PNU159682 | | | | | |
| D10 E10-hFc (S239C | [G₄S]₁ | MA-PEG4-vc-PAB- | 885 | 2.02 | 81669.8 | 81674.1 | 62 |
| T366Y/Y407T) | | EDA-PNU159682 | | | | | |
| D10 F05-hFc (S239C | [G₄S]₁ | MA-PEG4-vc-PAB- | 886 | 2.01 | 81713.8 | 81718.2 | 68 |
| T366Y/Y407T) | | EDA-PNU159682 | | | | | |

SDS-PAGE, SEC and mass spectrometry analysis of the final conjugates determined that the labelling had proceeded in a quantitative fashion in good overall yield to give highly pure homogenous protein drug conjugates with the desired drug to antibody ratio (DAR of 2) and with low levels of high molecular weight aggregates (<4%).

### Binding of Biparatopic VHH-hFc Drug Conjugates to Cell-Surface ALLP, ALPPL2, ALPI and ALPL by Flow Cytometry

Biparatopic VHH-hFc drug conjugates were assessed for human ALPP, ALPPL2, ALPI and ALPL cell-surface binding by flow cytometry. Binders were screened on CHO cells stably expressing human ALPP, ALPPL2, ALPI or ALPL.

Cell surface binding of test agents to hALPP isoforms was characterized in four CHO-ALPP isoform transfectant cell lines (CHO-ALPP, CHO-ALPPL2, CHO-ALPI and CHO-ALPL) and the resulting K_{Dapp} values determined. Adherent cells were detached from tissue culture flasks by incubating with 0.1% EDTA/PBS solution at 37° C. for −10 minutes or until cells detached easily. Cells were re-suspended in ice-cold PBS/2% FCS in 15 ml tubes and centrifuged at 1700 rpm for 5 mins at 4° C. Supernatant was removed and the cell pellet re-suspended in PBS/2% FCS. A cell count was performed using a Chemometec Nucleocounter NC-202 and 2.5×10\hat{ }5 cells were aliquoted per test sample into a 96 well plate. Cells were incubated with 100 μl of test agents at a range of concentrations, plus controls for 1 hr on ice. The sample plate was centrifuged at 1700 rpm for 5 mins at 4° C. The supernatant was removed, and a wash performed by re-suspending the cell pellets in 0.25 mL of ice-cold PBS/2% FCS using a multichannel pipette. Samples were again centrifuged at 1700 rpm for 5 min at 4° C. Supernatant was removed and two further washes performed as described. After the final wash and centrifugation step, excess liquid was removed by blotting the plate on tissue paper. Binding of VHH-hFc agents was determined by adding 100 μl of Fitc-anti-human Ab (ThermoFisher) and incubating for 30 mins on ice in the dark. Wash steps were performed as described previously. All cell pellets were finally re-suspended in 0.28 ml of ice-cold PBS/2% FCS and left on ice in the dark prior to analysis on a Merck-Millipore Guava EasyCyte HT or Thermo Fisher Attune N×T flow cytometer.

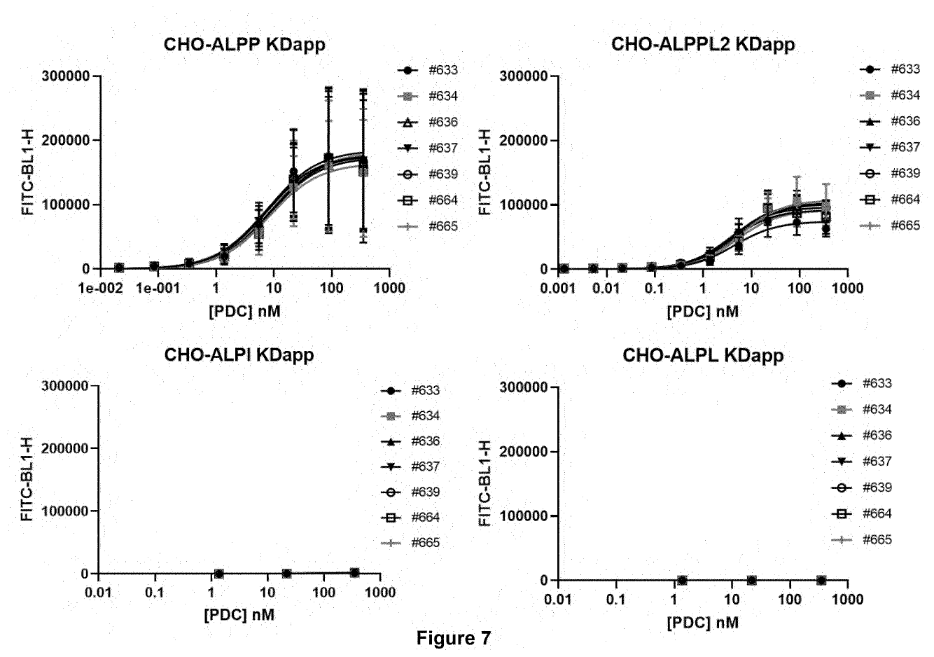

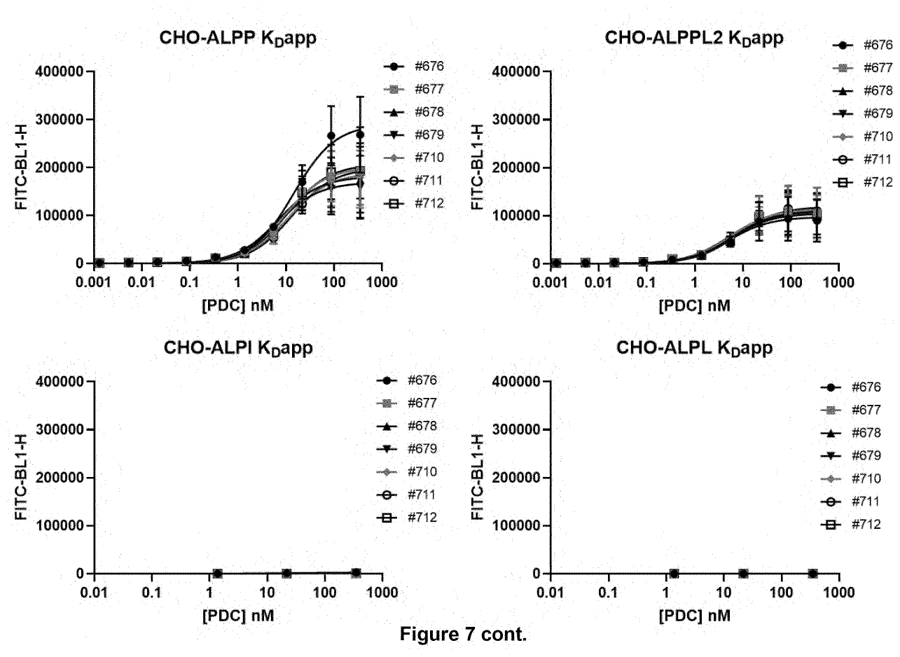

Binding of the different domains to the different cell-surface ALPP forms is summarized in Table 22 and in FIG. 7.

TABLE 22
Binding of different Biparatopic VHH-hFc drug conjugates to ALPP,
ALPPL2, ALPI and ALPL expressed on the surface of CHO cells

| | | | K_{D}app in CHO- | K_{D}app in CHO- | K_{D}app in CHO- | K_{D}app in CHO- |
|---|---|---|---|---|---|---|
| ID # | Linker | Molecule | ALPP cells | ALPPL2 cells | ALPI cells | ALPL cells |
| 633 | [G₄S]₃ | D10-A06 hFc-vcPAB-MMAE | 7.6 nm | 4.4 nm | ND | No binding |
| 634 | [G₄S]₃ | D10-E06 hFc-vcPAB-MMAE | 8.3 nm | 5.9 nm | No binding | No binding |
| 636 | [G₄S]₃ | D10-E10 hFc-vcPAB-MMAE | 6.8 nm | 4.7 nm | No binding | No binding |
| 637 | [G₄S]₃ | D10-F05 hFc-vcPAB-MMAE | 7.4 nm | 3.5 nm | No binding | No binding |
| 639 | [G₄S]₃ | A06-E06 hFc-vcPAB-MMAE | 8.5 nm | 4.2 nm | ND | No binding |
| 664 | [G₄S]₃ | A06-E10 hFc-vcPAB-MMAE | 8.5 nm | 3.7 nm | ND | No binding |
| 665 | [G₄S]₃ | A06-F05 hFc-vcPAB-MMAE | 9.8 nm | 4.9 nm | ND | No binding |
| 676 | [G₄S]₁ | D10-A06-hFc-vcPAB-MMAE | 14.4 nm | 5.0 nm | ND | No binding |
| 677 | [G₄S]₁ | D10-E06-hFc-vcPAB-MMAE | 9.4 nm | 5.5 nm | No binding | No binding |
| 678 | [G₄S]₁ | D10-E10-hFc-vcPAB-MMAE | 6.9 nm | 5.0 nm | No binding | No binding |
| 679 | [G₄S]₁ | D10-F05-hFc-vcPAB-MMAE | 6.6 nm | 5.2 nm | No binding | No binding |
| 710 | [G₄S]₁ | A06-E06-hFc-vcPAB-MMAE | 11.0 nm | 5.6 nm | ND | No binding |
| 711 | [G₄S]₁ | A06-E10-hFc-vcPAB-MMAE | 13.4 nm | 7.6 nm | ND | No binding |
| 712 | [G₄S]₁ | A06-F05-hFc-vcPAB-MMAE | 10.64 nm | 5.7 nm | ND | No binding |

ND = not-determinable (MFI binding to CHO-ALPI cells at highest concentration of 350 nM ≤1% of MFI binding to CHO-ALPP cells).

FIG. 7 and Table 22 show that the biparatopic VHH-hFc drug conjugates specifically bind ALPP/ALPPL2 on the surface of cells.

In vitro cell viability assays for cancer cells treated with anti ALPP/ALPPL2 Biparatopic drug conjugates Cell Titre G assays were performed as described in Examples 4 and 5. Cells were incubated with Biparatopic VHH-hFc-drug conjugates at 3700, 5% 002 for 96 hours and the % of cell viability determined as a function of dose response. The % of control data was plotted against Log [Treatment] concentration and the 1050 value derived using non-linear regression fitting in GraphPad Prism software.

### MMAE Conjugates

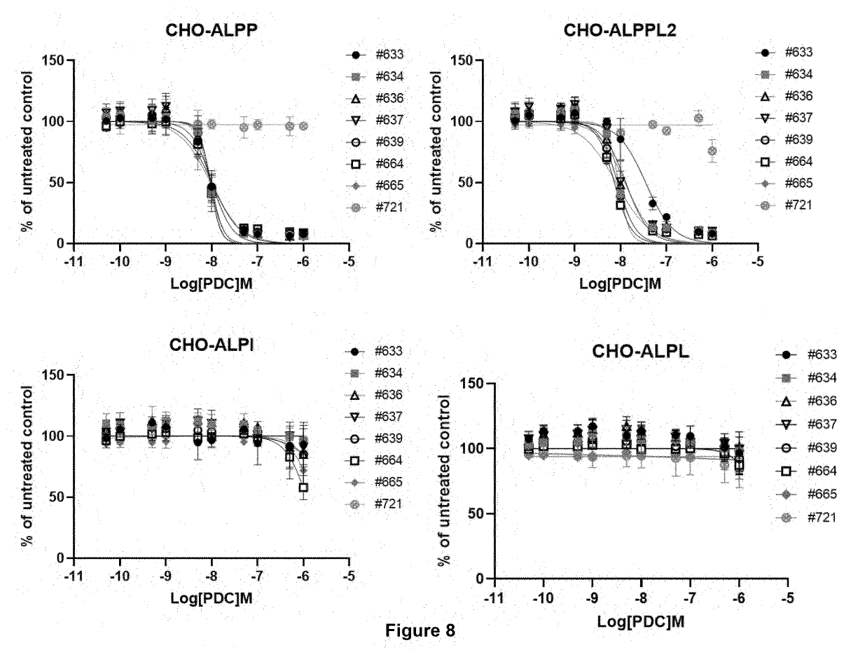

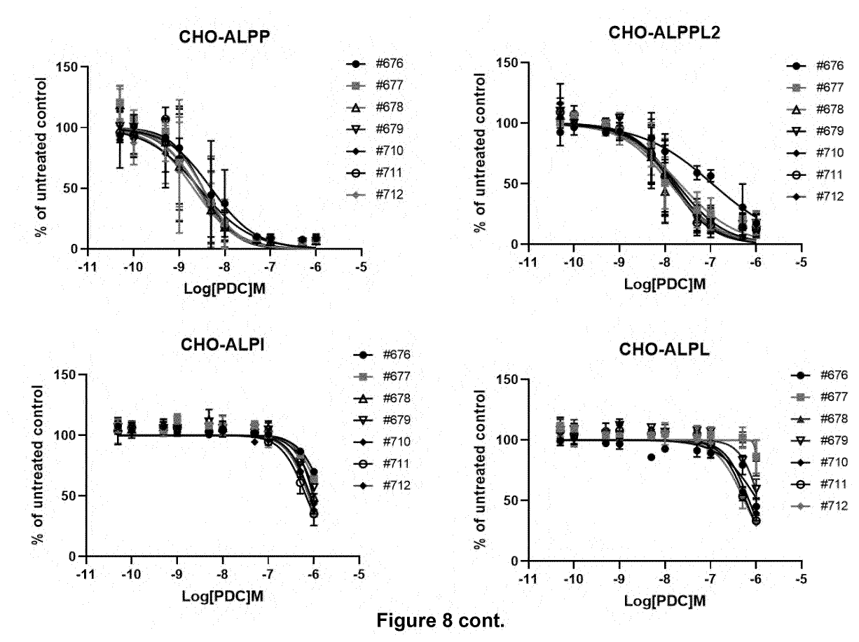

Table 23 shows IC₅₀ values, for cell-killing of CHO-ALPP isoform transfectant cell lines stably expressing either ALPP, ALPPL2, ALPI or ALPL by biparatopic VHH-hFc-vc-PAB-MMAE (DAR4) drug conjugates. The corresponding αGFP-hc (S239C+S442C)-vc-PAB-MMAE (DAR4) agent was included as a non-binding negative control. FIG. 8 shows representative dose response curves, for cell-killing of the CHO-ALPP, CHO-ALPPL2, CHO-ALPI and CHO-ALPL cells by the biparatopic VHH-hFc MMAE drug conjugates

TABLE 23
Calculated IC₅₀ values (nM) for the cell-killing of CHO-ALPP, CHO-ALPPL2, CHO-
ALPI, CHO-ALPL by biparatopic VHH-hFc-vc-PAB-MMAE (DAR4) drug conjugates.

| | | | | IC₅₀ at 96 h (nM) | | | |
|---|---|---|---|---|---|---|---|
| | | | | CHO- | CHO- | CHO- | CHO- |
| ID # | Molecule | Linker | DAR | ALPP | ALPPL2 | ALPI | ALPL |
| 633 | D10-A06-hFc-vc-MMAE | [G₄S]₃ | 4 | 10.1 | 35.2 | >1000 | >1000 |
| 634 | D10-E06 hFc-vc-MMAE | [G₄S]₃ | 4 | 9.2 | 9.1 | >1000 | >1000 |
| 636 | D10-E10 hFc-vc-MMAE | [G₄S]₃ | 4 | 9.2 | 12.5 | >1000 | >1000 |
| 637 | D10-F05 hFc-vc-MMAE | [G₄S]₃ | 4 | 9.2 | 13.0 | >1000 | >1000 |
| 639 | A06-E06hFc-vc-MMAE | [G₄S]₃ | 4 | 9.6 | 8.6 | >1000 | >1000 |
| 664 | A06-E10hFc-vc-MMAE | [G₄S]₃ | 4 | 10.1 | 7.3 | >1000 | >1000 |
| 665 | A06-F05 hFc-vc-MMAE | [G₄S]₃ | 4 | 9.5 | 7.9 | >1000 | >1000 |
| 676 | D10-A06 hFc-vc-MMAE | [G₄S]₁ | 4 | 5.0 | 108.8 | >1000 | >1000 |
| 677 | D10-E06 hFc-vc-MMAE | [G₄S]₁ | 4 | 3.3 | 21.1 | >1000 | >1000 |
| 678 | D10-E10 hFc-vc-MMAE | [G₄S]₁ | 4 | 2.5 | 12.8 | 972* | >1000 |
| 679 | D10-F05 hFc-vc-MMAE | [G₄S]₁ | 4 | 2.6 | 16.1 | >1000 | >1000 |
| 710 | A06-E06 hFc-vc-MMAE | [G₄S]₁ | 4 | 2.7 | 18.1 | 846* | 699* |
| 711 | A06-E10 hFc-vc-MMAE | [G₄S]₁ | 4 | 3.3 | 14.1 | 673* | 597* |
| 712 | A06-F05 hFc-vc-MMAE | [G₄S]₁ | 4 | 2.1 | 14.8 | 776* | 551* |
| 721 | αGFP-hFc-vc-MMAE | [G₄S]₃ | 4 | >1000 | >1000 | >1000 | >1000 |
| | MMAE | | N/A | 19.1 | 24.4 | 11.4 | 29.4 |
| | | | | 19.5 | 15.6 | 15.9 | 25.8 |

*Estimated IC₅₀ from data extrapolation

As shown in Table 23 and FIG. 8 biparatopic VHH-hFc-MMAE conjugates have been generated, which show potent and selective killing of ALPP and ALPPL2 expressing CHO cells, with very large drop-off in potencies for killing of ALPI and ALPL expressing CHO cells.

Table 24 shows IC₅₀ values, for cell-killing of ALPP/ALPL2 positive cancer cells (MKN1, NCI-H1651 and NCI-N87) and ALPP/ALPPL2 negative HeLa cancer cell-lines by biparatopic VHH-hFc-vc-PAB-MMAE (DAR4) drug conjugates. The corresponding αGFP-hFc (S239C+S442C)-vc PAB MMAE (DAR4) agents was included as a non-binding negative control.

TABLE 24
Calculated IC₅₀ values (nM) for the cell-killing of ALPP/ALPPL2 positive cancer
cells (MKN1, NCI-H1651 and NCI-N87) and for the ALPP/ALPPL2 negative HeLa
cancer cell-line by biparatopic VHH-hFc-vc PAB MMAE (DAR4) drug conjugates.

| | | | | IC₅₀ at 96 h (nM) and (% viability remaining) | | | |
|---|---|---|---|---|---|---|---|
| ID # | Molecule | Linker | DAR | MKN1 | NCI-H1651 | NCI-N87 | HeLa |
| 633 | D10-A06-hFc-vc- | [G₄S]₃ | 4 | 1.4 (27.9) | 5.4 (25.4) | 4.9 (27.3) | >1000 (52.5) |
| | MMAE | | | | | | |
| 634 | D10-E06-hFc-vc- | [G₄S]₃ | 4 | 2.6 (31.1) | 1.1 (28.5) | 2.4 (27.6) | >1000 (53.6) |
| | MMAE | | | | | | |
| 636 | D10-E10 hFc-vc- | [G₄S]₃ | 4 | 1.9 (30.0) | 0.9 (28.9) | 2.1 (26.2) | >1000 (69.9) |
| | MMAE | | | | | | |
| 637 | D10-F05 hFc-vc- | [G₄S]₃ | 4 | 1.3 (28.1) | 1.2 (28.0) | 5.1 (28.9) | >1000 (62.6) |
| | MMAE | | | | | | |
| 639 | A06-E06hFc-vc- | [G₄S]₃ | 4 | 2.3 (30.6) | 0.9 (28.0) | 12.9 (27.8) | >1000 (52.9) |
| | MMAE | | | | | | |
| 664 | A06-E10hFc-vc- | [G₄S]₃ | 4 | 0.9 (29.7) | 0.6 (27.2) | 4.7 (28.7) | 767.4 (36.5) |
| | MMAE | | | | | | |
| 665 | A06-F05 hFc-vc- | [G₄S]₃ | 4 | 0.7 (31.6) | 0.4 (25.0) | 5.8 (27) | 628.0 (30) |
| | MMAE | | | | | | |
| 677 | D10-E06 hFc-vc- | [G₄S]₁ | 4 | 31.8 (5.3) | 1.3 (28.9) | 0.6 (25.5) | >1000 (62) |
| | MMAE | | | | | | |
| 678 | D10-E10 hFc-vc- | [G₄S]₁ | 4 | 8.1 (24.5) | 0.6 (31.0) | 0.5 (23.6) | >1000 (58) |
| | MMAE | | | | | | |
| 679 | D10-F05 hFc-vc- | [G₄S]₁ | 4 | 9.7 (25.2) | 0.8 (31.2) | 0.6 (21.8) | >1000 (51) |
| | MMAE | | | | | | |
| 712 | A06-F05 hFc-vc- | [G₄S]₁ | 4 | 45.9 (27.1) | 0.9 (31.3) | 0.7 (20.7) | 881 (44.5) |
| | MMAE | | | | | | |
| 721 | αGFP-hFc-vc- | [G₄S]₃ | 4 | >1000 (42.5) | 682.3 (30.4) | 464.5 (39.5) | >1000 (47.4) |
| | MMAE | | | | | | |
| | MMAE | | N/A | 1.4 (23.1) | 0.8 (28.1) | 0.3 (27.9) | 1.8 (5.1) |

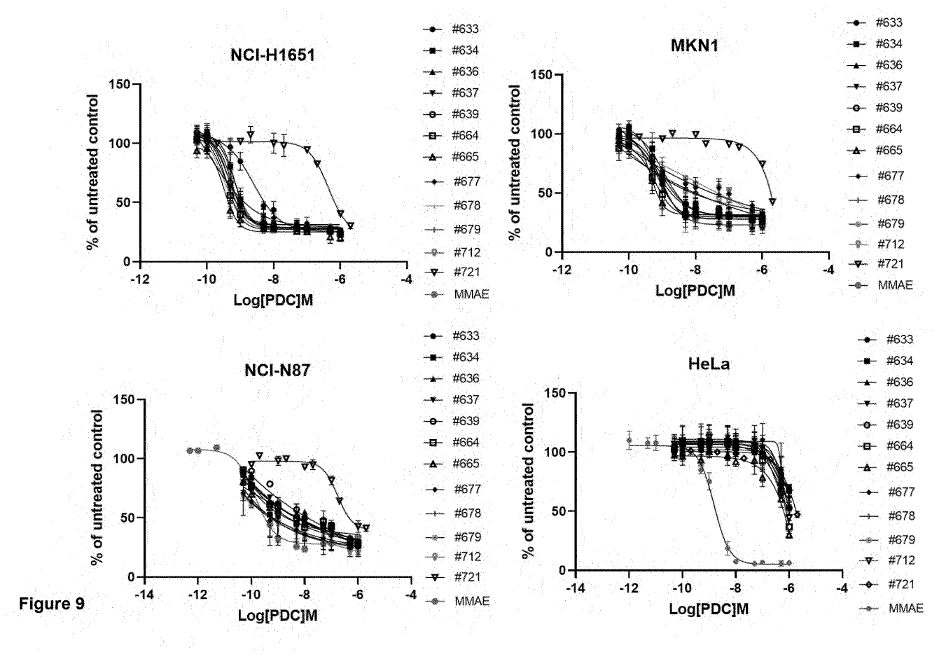

As shown in Table 24 and FIG. 9 biparatopic VHH-hFc-MMAE conjugates have been generated, which show potent and selective killing of ALPP/ALPPL2 expressing cancer cells but with little potency against HeLa cancer cells, which express little to no ALPP/ALPPL2.

A large window to the control non-binding protein drug conjugate, αGFP-hFc (S239C+S442C)-vc PAB MMAE (DAR4) was observed.

Dose responses, with IC₅₀ values, are also shown for treatment of the cancer cell-lines with the free MMAE payload. At maximal concentrations of MMAE the remaining % cell viability matches the remaining % cell viability observed for the VHH-Fc-MMAE drug conjugate binders in ALPP/ALPPL2 positive cancer cell lines.

### PNU Conjugates

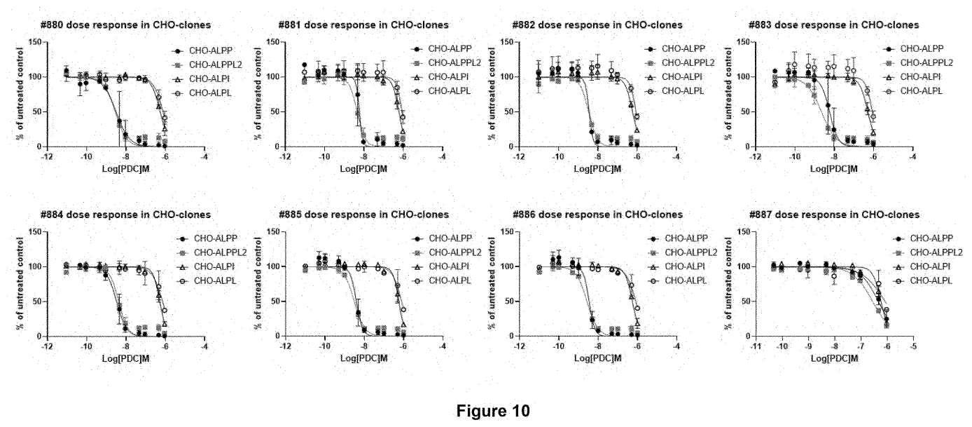

Table 25 shows IC₅₀ values, for cell-killing of CHO-ALPP isoform transfectant cell lines stably expressing either ALPP, ALPPL2, ALPI or ALPL by biparatopic VHH-hFc-PEG4-vc-PAB-EDA-PNU159682 (DAR2) drug conjugates. The corresponding αGFP-hFc (S239C)-PEG4-vc-PAB-EDA-PNU159682 (DAR2) agent was included as a non-binding negative control. FIG. 10 shows representative dose response curves, for cell-killing of the CHO-ALPP, CHO-ALPPL2, CHO-ALPI and CHO-ALPL cells by the biparatopic VHH-hFc—PNU drug conjugates.

TABLE 25
Calculated IC₅₀ values (nM) for the cell-killing of CHO-ALPP, CHO-ALPPL2, CHO-ALPI,
CHO-ALPL by biparatopic VHH-hFc-PEG-vc-PAB-EDA-PNU (DAR2) drug conjugates.

| | | | | IC₅₀ at 96 h (nM) | | | |
|---|---|---|---|---|---|---|---|
| ID # | Molecule | Linker | DAR | CHO-ALPP | CHO-ALPPL2 | CHO-ALPI | CHO-ALPL |
| 880 | D10-E10-hFc-vcPAB-EDA- | [G₄S]₃ | 2.02 | 3.2 | 2.9 | 548.8 | 773.5 |
| | PNU | | | | | | |
| 881 | D10-E06-hFc-vcPAB-EDA- | [G₄S]₃ | 2.02 | 5.9 | 4.6 | 600.1 | 866.8 |
| | PNU | | | | | | |
| 882 | D10-F05-hFc-vcPAB-EDA- | [G₄S]₃ | 1.94 | 3.6 | 3.5 | 586.4 | 879.6 |
| | PNU | | | | | | |
| 883 | A06-F05-hFc-vcPAB-EDA- | [G₄S]₃ | 2.03 | 5.7 | 2.2 | 512.8 | 857.9 |
| | PNU | | | | | | |
| 884 | D10-E06-hFc-vcPAB-EDA- | [G₄S]₁ | 2.03 | 3.4 | 4.3 | 524.9 | 724.0 |
| | PNU | | | | | | |
| 885 | D10-E10-hFc-vcPAB-EDA- | [G₄S]₁ | 2.02 | 4.0 | 3.2 | 579.3 | 800.5 |
| | PNU | | | | | | |
| 886 | D10-F05-hFc-vcPAB-EDA- | [G₄S]₁ | 2.01 | 3.5 | 2.7 | 552.2 | 830.8 |
| | PNU | | | | | | |
| 887 | αGFP-hFc-vcPAB-EDA- | [G₄S]₃ | 2.1 | 460.9 | 279.7 | 579.1 | 834.7 |
| | PNU | | | | | | |
| | EDA-PNU | | | 5.3 | 5.6 | 4.3 | 5.5 |

As shown in Table 25 and FIG. 10 biparatopic VHH-hFc-PNU conjugates have been generated, which show potent and selective killing of ALPP and ALPPL2 expressing CHO cells, with much lower potencies for killing of ALPI and ALPL expressing CHO cells.

A large window in 1050 values was observed for the ALPP/ALPPL2 targeting VHH-hFc-PNU conjugates versus the control non-binding αGFP-hFc—PNU control for killing of the CHO-ALPP and CHO-ALPPL2. This cell-kill window between agents and control is lost for the CHO-ALPI and CHO-ALPL indicative of the highly selective nature of the ALPP/ALPPL2 mediated cell-killing.

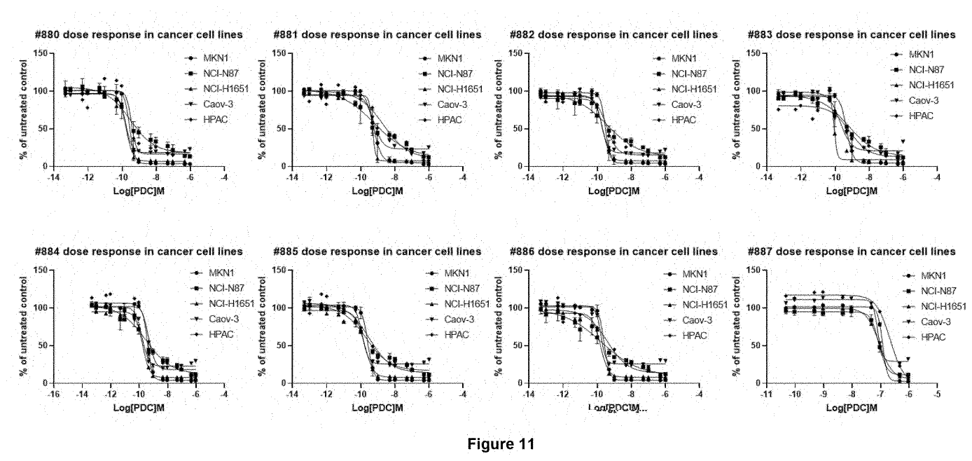

Table 26 and FIG. 11 show IC₅₀ values and dose-response curves, for cell-killing of ALPP/ALPPL2 positive cancer cells (MKN1, NCI-N87, NNC—H1651, Caov-3 and HPAC) by biparatopic VHH-hFc-PEG4-vc-PAB-EDA-PNU (DAR2) drug conjugates. The corresponding αGFP-hFc (S239C)—PEG4-vc-PAB-EDA-PNU (DAR4) agents was included as a non-binding negative control.

TABLE 26
Calculated IC₅₀ values (nM) for the cell-killing of ALPP/ALPPL2 positive
cancer cells MKN1, NCI-N87, NCI-H1651, Caov-3 and HPAC by biparatopic
VHH-hFc-PEG4-vc-PAB-EDA-PNU (DAR2) drug conjugates.

| | | | | IC₅₀ at 96 h (nM) and (% viability remaining) | | | | |
|---|---|---|---|---|---|---|---|---|
| | | | | | | NCI- | | |
| ID # | Molecule name | Linker | DAR | MKN1 | NCI-N87 | H1651 | Caov-3 | HPAC |
| 880 | D10-E10hFc- | [G₄S]₃ | 2.02 | 0.29 | 1.5 | 0.18 | 0.26 | 0.48 |
| | vcPAB-EDA-PNU | | | (3%) | (13%) | (7%) | (23%) | (4%) |
| 881 | D10-E06hFc- | [G₄S]₃ | 2.02 | 0.6 | 1.98 | 0.38 | 1.97 | 4.22 |
| | vcPAB-EDA-PNU | | | (3%) | (12%) | (7%) | (18%) | (8%) |
| 882 | D10-F05hFc- | [G₄S]₃ | 1.94 | 0.3 | 0.88 | 0.21 | 0.28 | 0.63 |
| | vcPAB-EDA-PNU | | | (3%) | (12%) | (7%) | (20%) | (4%) |
| 883 | A06-F05hFc- | [G₄S]₃ | 2.03 | 0.45 | 1.1 | 0.12 | 0.55 | 0.34 |
| | vcPAB-EDA-PNU | | | (4%) | (12%) | (7%) | (17%) | (6%) |
| 884 | D10-E06hFc- | [G₄S]₁ | 2.03 | 0.41 | 0.86 | 0.19 | 0.41 | 1.3 |
| | vcPAB-EDA-PNU | | | (3%) | (12%) | (8%) | (26%) | (9%) |
| 885 | D10-E10hFc- | [G₄S]₁ | 2.02 | 0.29 | 0.88 | 0.18 | 0.58 | 1.0 |
| | vcPAB-EDA-PNU | | | (4%) | (12%) | (8%) | (27%) | (9%) |
| 886 | D10-F05hFc- | [G₄S]₁ | 2.01 | 0.3 | 0.39 | 0.16 | 0.69 | 0.84 |
| | vcPAB-EDA-PNU | | | (4%) | (12%) | (9%) | (30%) | (7%) |
| 887 | αGFP-hFc- | [G₄S]₃ | 2.1 | 102 | 108 | 102 | 233 | 294 |
| | vcPAB-EDA-PNU | | | (2%) | (11%) | (7%) | (27%) | (7%) |

As shown in Table 26 and FIG. 11 biparatopic VHH-hFc-PNU conjugates have been generated, which show potent and selective killing of a range of different ALPP/ALPPL2 expressing cancer cell-lines.

A large window between the ALPP/ALPPL2 targeting PDCs and the non-binding αGFP-hFc (S239C)-PEG4-vc-PAB-EDA-PNU (DAR2) control was observed across all of the cancer cell-lines.

It was observed that for the same cancer cell-lines, the % cell viability remaining at maximal concentrations for the VHH-Fc-PNU drug conjugates is lower than the remaining % cell viability for the corresponding VHH-hFc-MMAE drug conjugates (see data above).

### Example 6—In Vivo Efficacy of ALPP/ALPPL2 Protein-Drug Conjugates

All procedures and protocols involving animals were conducted after review and approval by the Institutional Animal Care and Use Committee (IACUC) of the facility, with the care and use of animals conducted in accordance with the regulations of the Association for Assessment and Accreditation of Laboratory Animal Care (AAALAC).

### NCI-N87 Human Gastric Carcinoma CDX and HPAC Human Pancreatic Ductal Adenocarcinoma CDX—Biparatopic VHH-hFc-vcMMAE Drug Conjugates

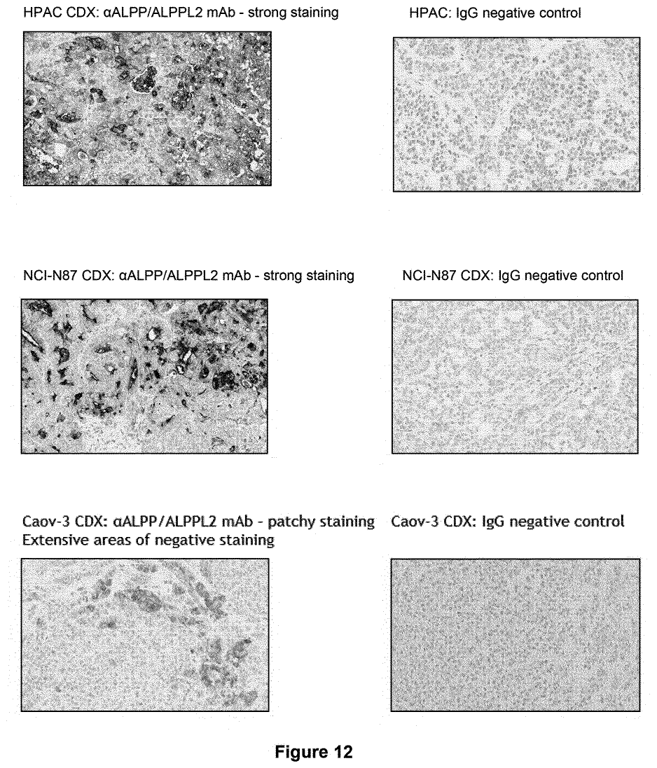

Efficacy studies in the ALPP/ALPPL2+ NCI-N87 gastric carcinoma xenograft model and the HPAC human pancreatic ductal adenocarcinoma xenograft model were performed at Crown Bioscience (Beijing). These CDX models were shown to express ALPP/ALPPL2 by IHC (FIG. 12).

ALPP/ALPPL2 expression was confirmed in both NCI-N87 and HPAC cell-line-derived xenograft FFPE samples using anti-ALPP/ALPPL2 antibody (MS Validated Antibodies) and Rabbit Isotype negative control by immunohistochemical (IHC) staining.

For the NCI-N87 studies, female BALB/c nude mice were inoculated with NCI-N87 human gastric carcinoma cells (ATCC, Crown ID CL-00455). Each mouse was inoculated subcutaneously in the right flank with NCI-N87 tumor cells (1×10⁷) in 0.1 mL of a 1:1 PBS/Matrigel suspension.

For the HPAC studies, female NOD/SCID mice were inoculated with HPAC human pancreatic ductal adenocarcinoma cells (ATCC, Crown ID CL-00891). Each mouse was inoculated subcutaneously in the right front flank region with HPAC tumor cells (1×10⁷) in 0.1 mL of a 1:1 PBS/Matrigel suspension.

Mice were monitored until the tumour volume reached the recruitment criteria of around 200 mm³ in a sufficient number of animals. Mice were randomised to treatment groups (n=5) such that the mean tumour volume for each group was ~200 mm³ and there was no statistical difference between tumour volumes across the groups. The day of randomisation was designated as Day 0 and mice were treated with vehicle or with the protein-drug conjugates (see Table 27 below) on Day 1. Protein drug conjugates were dosed intravenously once every four days (Q4D×4) at either 1 mg/kg or 3 mg/kg. All mice were pre-primed with mouse IgG 20 h before first PDC dose.

TABLE 27
Protein drug conjugates

| | ID | | | | Nominal |
|---|---|---|---|---|---|
| PDC | # | Batch | Molecule | Linker | DAR |
| PDC1 | 634 | 2 | D10-E06 hFc-vc-MMAE | [G₄S]₃ | 4 |
| PDC2 | 636 | 2 | D10-E10 hFc-vc-MMAE | [G₄S]₃ | 4 |
| PDC3 | 637 | 4 | D10-F05 hFc-vc-MMAE | [G₄S]₃ | 4 |
| PDC4 | 665 | 2 | A06-F05 hFc-vc-MMAE | [G₄S]₃ | 4 |
| PDC5 | 677 | 2 | D10-E06 hFc-vc-MMAE | [G₄S]₁ | 4 |
| PDC6 | 678 | 2 | D10-E10 hFc-vc-MMAE | [G₄S]₁ | 4 |
| PDC7 | 679 | 2 | D10-F05 hFc-vc-MMAE | [G₄S]₁ | 4 |
| PDC8 | 616 | 2 | D10-F05 hFc-vc-MMAE | [G₄S]₃ | 2 |

Tumour volume was evaluated by measuring perpendicular tumour diameters, with a calliper, three times a week during the experimental period. Absolute tumour volume (ATV) was calculated using the formula TV (mm³)=[length (mm)×width (mm)×width (mm)]×0.5, where the length (L) is the longest tumour dimension and the width (W) is the longest tumour dimension perpendicular to L. All animals were weighed at the same time as tumour size measurement and also on dosing days. Mice were observed and documented daily for changes in physical appearance, behaviour, adverse clinical signs and general welfare in line with local welfare and best veterinary practice guidelines.

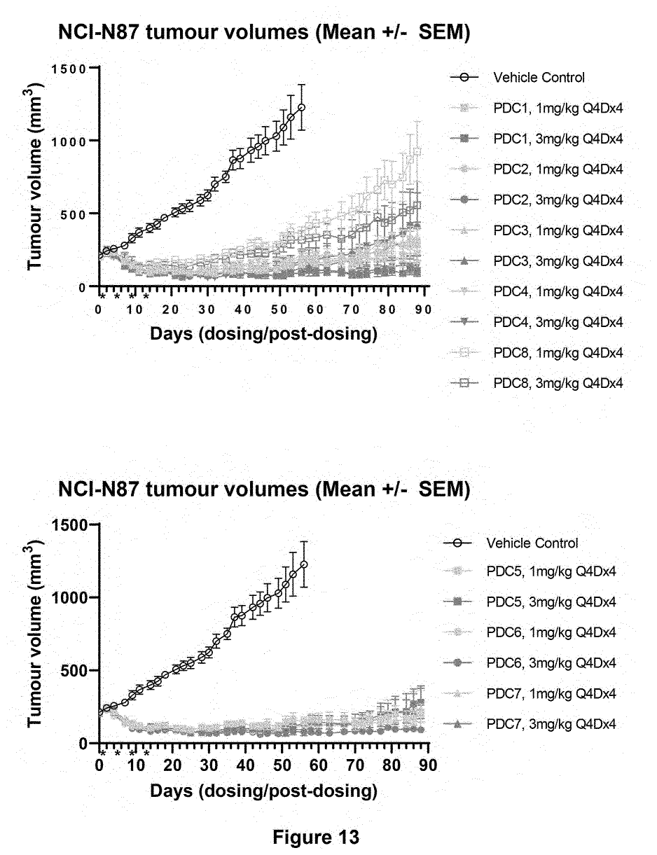

FIG. 13 shows the effect of the protein-drug conjugates on tumour growth versus vehicle control for the NCI-N87 CDX study. All protein drug conjugates were well tolerated and showed highly statistically significant in vivo efficacy at both 1 mg/kg and 3 mg/kg dose levels compared to the vehicle treated group. PDC8, which has a DAR of 2, showed marginally lower tumour growth inhibition compared to the corresponding DAR4 version at equivalent doses. Vehicle data is plotted to the last day when tumour volumes for all animals within the group were within ethical tumour size limits.

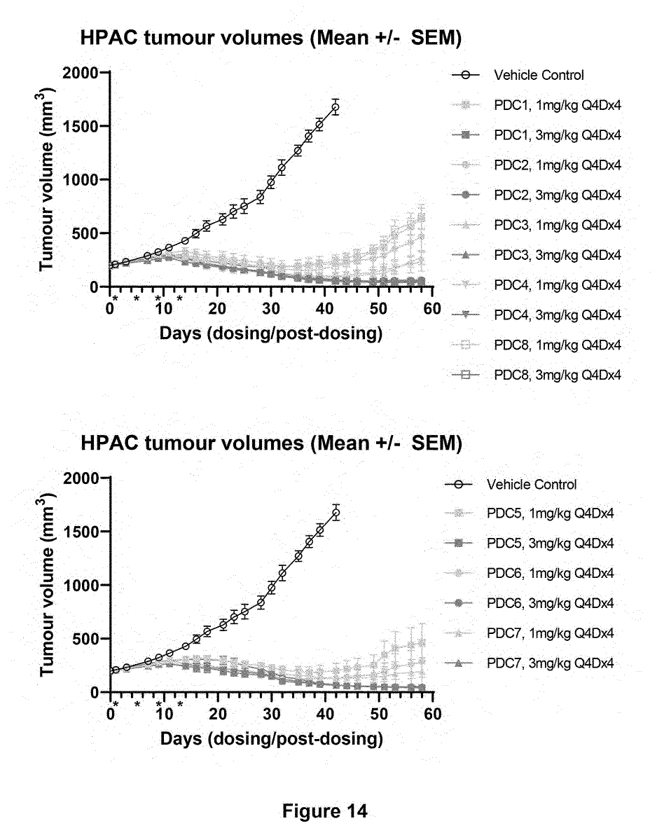

FIG. 14 shows the effect of the protein-drug conjugates on tumour growth versus vehicle control for the HPAC CDX study. All protein drug conjugates were well tolerated and showed highly statistically significant in vivo efficacy at both 1 mg/kg and 3 mg/kg dose levels compared to the vehicle treated group. PDC8, which has a DAR of 2, showed marginally lower tumour growth inhibition compared to the corresponding DAR4 version at equivalent doses. Vehicle data is plotted to the last day when tumour volumes for all animals within the group were within ethical tumour size limits.

Conjugates according to the invention were produced as either DAR2 or DAR4 molecules. The DAR, or drug-to-antibody ratio, represents the average number of drug molecules conjugated to the antigen binding domains of the invention. The exact DAR values for each batch of PDC molecules are shown in Table 28 below.

TABLE 28
Protein drug conjugate DAR values

| | | | | | | Intact, | | |
|---|---|---|---|---|---|---|---|---|
| | | | | | | deglycosylated | | |
| | | | | | | mass, Da | | |
| Protein | Linker | Payload | # | Batch# | DAR | Expected | Observed | Yield, % |
| D10 E06-hFc | [G₄S]₃ | MC-vc-PAB- | 634 | 2 | 3.6 | 85010.9 | 85024.2 | 77.4 |
| (S239/442C | | MMAE | | | | | | |
| T366Y/Y407T) | | | | | | | | |
| D10 F05-hFc | [G₄S]₃ | MC-vc-PAB- | 637 | 4 | 3.5 | 85464 | 85479.8 | 71.3 |
| (S239/442C | | MMAE | | | | | | |
| T366Y/Y407T) | | | | | | | | |
| A06 F05-hFc | [G₄S]₃ | MC-vc-PAB- | 665 | 2 | 3.3 | 85885.3 | 85899.9 | 73.3 |
| (S239/442C | | MMAE | | | | | | |
| T366Y/Y407T) | | | | | | | | |
| D10 F05-hFc | [G₄S]₁ | MC-vc-PAB- | 679 | 2 | 3.7 | 84202.9 | 84219.0 | 73.7 |
| (S239/442C | | MMAE | | | | | | |
| T366Y/Y407T) | | | | | | | | |
| D10 F05-hFc | [G₄S]₃ | MC-vc-PAB- | 616 | 2 | 1.7 | 82801.9 | 82815.1 | 73 |
| (S239C | | MMAE | | | | | | |
| T366Y/Y407T) | | | | | | | | |
| D10 E06-hFc | [G4S]3 | MC-vc-PAB- | 634 | 3 | 4.0 | 85010.9 | 85027.2 | 55.4 |
| (S239/442C | | MMAE | | | | | | |
| T366Y/Y407T) | | | | | | | | |
| D10 F05-hFc | [G4S]3 | MC-vc-PAB- | 637 | 5 | 3.9 | 85464 | 85475.0 | 66.2 |
| (S239/442C | | MMAE | | | | | | |
| T366Y/Y407T) | | | | | | | | |
| A06 F05-hFc | [G4S]3 | MC-vc-PAB- | 665 | 3 | 4.0 | 85885.3 | 85907.9 | 70.8 |
| (S239/442C | | MMAE | | | | | | |
| T366Y/Y407T) | | | | | | | | |
| D10 F05-hFc | [G4S]1 | MC-vc-PAB- | 679 | 3 | 4.0 | 84202.9 | 84211.2 | 65 |
| (S239/442C | | MMAE | | | | | | |
| T366Y/Y407T) | | | | | | | | |
| D10 F05-hFc | [G4S]3 | MC-vc-PAB- | 616 | 3 | 2.0 | 82801.9 | 82816.9 | 68.6 |
| (S239C | | MMAE | | | | | | |
| T366Y/Y407T) | | | | | | | | |
| h12F3 | NA | MC-vc-PAB- | 1033 | 1 | 4.0 | NA | NA | 76 |
| | | MMAE | | | | | | |

DAR calculations shown to one decimal place.

### Example 7—ALPP/ALPPL2 Bispecific T-Cell Engaging Proteins

ALPP/ALPPL2×CD3 bispecific sequences combining N-terminal ALPP/ALPPL2 VHHs with a 0-terminal anti-CD3 scFv (clone OKT3) via 2 different length G4S linkers are expressed in CHO cells (Evitria) and purified by IMAC (HisTrap Excel, GE Healthcare) followed by SEC (Superdex 200 26/60, GE Healthcare). Similarly, biparatopic ALPP/ALPPL2×CD3 bispecific sequences combining N-terminal biparatopic ALPP/ALPPL2 VHHs with the C-terminal anti-CD3 scFv are also expressed in CHO (Evitria).

CD3 BiTE-like approach; examples of CD3 binding sequences for use as an ALPP/ALPPL2 VHH bispecific Anti CD3 scFv clone OKT3 (WO 2014028776 Zyngenia) and orientation and humanised derivatives thereof

VH-[G₄S]₃-VL
(SEQ ID NO: 149)
DIKLQQSGAELARPGASVKMSCKTSGYTFTRYTMHWVKQRPGQGLEWIGYINPSRGYTNYNQKFKD
KATLTTDKSSSTAYMQLSSLTSEDSAVYYCARYYDDHYCLDYWGQGTTLTVSSGGGGSGGGGSGG
GGSDIQLTQSPAIMSASPGEKVTMTCRASSSVSYMNWYQQKSGTSPKRWIYDTSKVASGVPYRFSG
SGSGTSYSLTISSMEAEDAATYYCQQWSSNPLTFGAGTKLELKS

Humanised anti CD3 scFv UCHT1 (Arnett et al PNAS 2004 101(46) 16268-16273) and derivatives thereof

VL-[G₄S]₃-VH
(SEQ ID NO: 150)
MDIQMTQTTSSLSASLGDRVTISCRASQDIRNYLNWYQQKPDGTVKLLIYYTSRLHSGVPSKFSGSGS
GTDYSLTISNLEQEDIATYFCQQGNTLPWTFAGGTKLEIKGGGGSGGGGSGGGGSEVQLQQSGPEL
VKPGASMKISCKASGYSFTGYTMNWVKQSHGKNLEWMGLINPYKGVSTYNQKFKDKATLTVDKSSS
TAYMELLSLTSEDSAVYYCARSGYYGDSDWYFDVWGQGTTLTVFS

### Example 8—ALPP and ALPPL2 CAR-T Approaches

Chimeric antigen receptors (CARs) based on the ALPP and ALPPL2-specific antigen binding molecules described in the present application are generated. Furthermore, engineered T cells expressing such a CAR may also be generated, which may then be used in, for example, adoptive cell therapy.

In brief, a nucleic acid construct encoding a ALPP or ALPPL2-specific CAR are produced. The ALPP/ALPPL2-specific CAR may include an intracellular activation domain, a transmembrane domain, and an extracellular domain comprising the ALPP/ALPPL2-specific antigen binding molecule described herein. The nucleic acid construct is incorporated into a viral vector, such as a retroviral vector (e.g., a lentiviral vector).

T cells may be isolated from a patient in need of treatment, which are then modified to express the nucleic acid construct encoding the CAR, for example by retroviral transfection or gene-editing using approaches such as CRISPR-CAS-9.

The engineered T cells are then be re-infused into the patient in order to treat the condition, such as treatment of cancer.

### Example 9—Internalisation of ALPP/ALPPL2 Binding Proteins

Internalisation of ALPP/ALPPL2-targeting VHH-hFc binders, ALPP/ALPPL2-targeting monoclonal antibody (h12F3) and an αGFP non-binding control VHH-hFc fusion was assessed in Hep2 cells using an IncuCyte S3 live cell analysis instrument (Sartorius). Cells were seeded at a density of 3000 cells/well into a black clear-bottom 96-well plate (Corning, #3340) and left to adhere at 37° C. and 5% C02 for 24 hrs. The test agents were mixed with FabFluor-pH Red Antibody Labeling Reagent (Sartorius, #4722) at a molar ratio of 1:2 in media, ×2 final assay concentration, and incubated for 15 minutes at 37° C. to allow conjugation. 50 μl of the resulting mixtures was added to appropriate wells containing cells (50 μl) to result in a final concentration of 25 nM of each test agent. Images were captured every hour for 30 hours and three regions of interest were imaged from each well. Cell-by-cell analysis was performed using Incucyte integrated module. Data is presented as average red mean intensity (Red Calibrated Unit; RCU) over time. Assay was performed in triplicates.

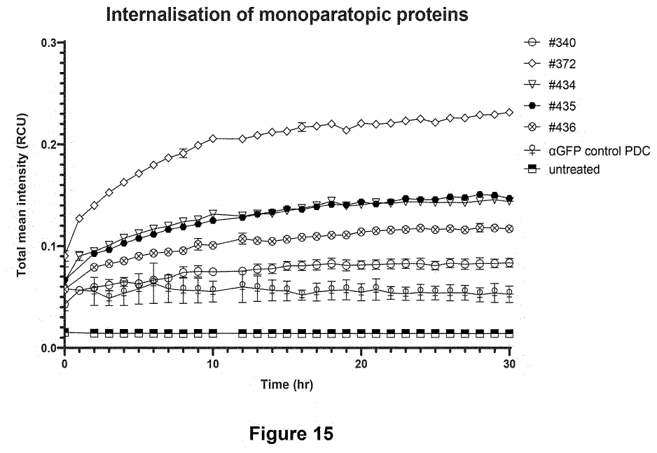

As shown in FIG. 15, ALPP/ALPPL2-targeting VHH-hFc fusions that bind in a monoparatopic fashion were internalised into Hep2 cells, whilst a non-binding control αGFP VHH-hFc protein showed no internalisation.

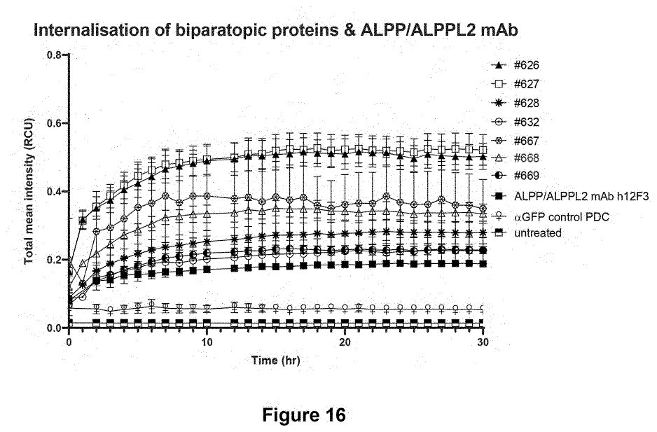

Internalisation of biparatopic ALPP/ALPPL2-targeting VHH-hFc fusion proteins were similarly assessed. As shown in FIG. 16 the biparatopic VHH-hFc fusion proteins showed enhanced internalisation into Hep2 cells. These biparatopic proteins showed increased internalisation over the ALPP/ALPPL2 targeting monoclonal antibody h12F3 HGLF. This humanised monoclonal antibody h12F3 HGLF (described in WO2022197890 as SEQ ID NO: 40 and SEQ ID NO: 50) was transiently expressed as secreted protein in CHO K1 cells and purified from the media using MabSelect™ SuRe™ (Evitria, Switzerland). Purified protein was exchanged into PBS pH 7.4 and analysed by SEC (AdvanceBio, Agilent, running buffer DPBS pH 7.4 or Superdex200 Increase 10/300, Cytivia, running buffer PBS pH 7), SDS PAGE and mass spectrometry under reducing and native conditions to confirm sequence and protein integrity.

### Example 10—Potency of Biparatopic and Monoparatopic ALPP/ALPPL2 Targeting PDCs and ADCs (DAR2 MMAE Formats)

Generation of DAR2 MMAE Biparatopic VHH-hFc (F405L/K409R) Drug Conjugates and h12F3 HGLF Drug Conjugates

A series of biparatopic VHH-hFc proteins were generated using FAB-arm exchange methods. For this, homomeric ALPP/ALPPL2 VHH-hFc proteins with a [G₄S]₃ linker were expressed with either a F405L or a K409R substitution, each also contained a S239C substitution for downstream conjugation.

The VHH-hFc fusion proteins were transiently expressed as secreted protein in CHO Ki cells and purified from the media using MabSelect™ SuRe™ (Evitria, Switzerland). Purified proteins were exchanged into PBS pH 7.4 and analysed by SEC (AdvanceBio, Agilent, running buffer DPBS), SDS PAGE and mass spectrometry to confirm sequence and protein integrity.

To form the biparatopics, an equal ratio of two homomeric proteins, one with the F405L and the other with K409R, were mixed and 200 mM cysteamine added before being incubated at 30 00 for 5 hours, followed by 16 hrs at 4° C. Resultant heterodimeric biparatopic VHH-hFc proteins were then purified by SEC and mass spectrometry used to confirm generation of the desired biparatopic VHH-hFc proteins.

Biparatopic anti ALPP/ALPPL2 VHH-hFc proteins generated through FAB-arm exchange were conjugated with MMAE (MC-vc-PAB-MMAE) using methods and analysis as described in Example 4 to give the corresponding DAR2 vc-PAB-MMAE PDCs.

The ALPP/ALPPL2 targeting monoclonal antibody h12F3 HGLF, as described in Example 9, was similarly expressed with the S239 substitution. This protein was then conjugated with MMAE (MC-vc-PAB-MMAE) using methods and analysis as described in Example 4, to give the corresponding DAR2 vc-PAB-MMAE ADO. Table 29 summarises the DAR2 MMAE conjugates prepared.

TABLE 29
Summary of characteristics of biparatopic VHH-hFc (F405L/K409R)
and h12F3 HGLF monoclonal antibody conjugated to MMAE.

| | | | | Intact, | | |
|---|---|---|---|---|---|---|
| | | | | deglycosylated | | |
| | | | | mass, Da | | |
| Protein | Payload | ID | DAR | Expected | Observed | Yield, % |
| A06 E06-hFc (S239C | MC-vc-PAB- | #524 | 2 | 82767.6 | 82771.3 | 61.5 |
| F405L/K409R) | MMAE | | | | | |
| A06 E10-hFc (S239C | MC-vc-PAB- | #525 | 2 | 83182.0 | 83181.4 | 65 |
| F405L/K409R) | MMAE | | | | | |
| A06 F05-hFc (S239C | MC-vc-PAB- | #526 | 2 | 83226.0 | 83226.8 | 72.5 |
| F405L/K409R) | MMAE | | | | | |
| D10 A06-hFc (S239C | MC-vc-PAB- | #527 | 2.8 | 82635.5 | 82634.4 | 59.1 |
| F405L/K409R) | MMAE | | | | | |
| D10 E06-hFc (S239C | MC-vc-PAB- | #531 | 2 | 82351.3 | 82351.6 | 85.5 |
| F405L/K409R) | MMAE | | | | | |
| D10 E10-hFc (S239C | MC-vc-PAB- | #532 | 2 | 82761.8 | 82762.3 | 82.1 |
| F405L/K409R) | MMAE | | | | | |
| D10 F05-hFc (S239C | MC-vc-PAB- | #533 | 2 | 82808.7 | 82805.4 | 76.6 |
| F405L/K409R) | MMAE | | | | | |
| h12F3 HGLF | MC-vc-PAB- | #873 | 2 | 148915.0 | 148936.0 | 82.4 |
| (S239C) | MMAE | | | | | |

FF In Vitro Cell Viability Assays for Cancer Cells Treated with Anti ALPP/ALPPL2 DAR2 MMAE Biparatopic VHH-hFc (F405L/K409R) Drug Conjugates and h12F3 HGLF Drug Conjugates

Cell Titre Glo assays were performed as described in Example 5. Cells were incubated with VHH-hFc-drug conjugates and h12F3 drug conjugates at 37° C., 5% CO₂ for 96 hours and the % of cell viability determined as a function of dose response. The % of control data was plotted against Log [Treatment] concentration and the IC₅₀ value derived using non-linear regression fitting in GraphPad Prism software.

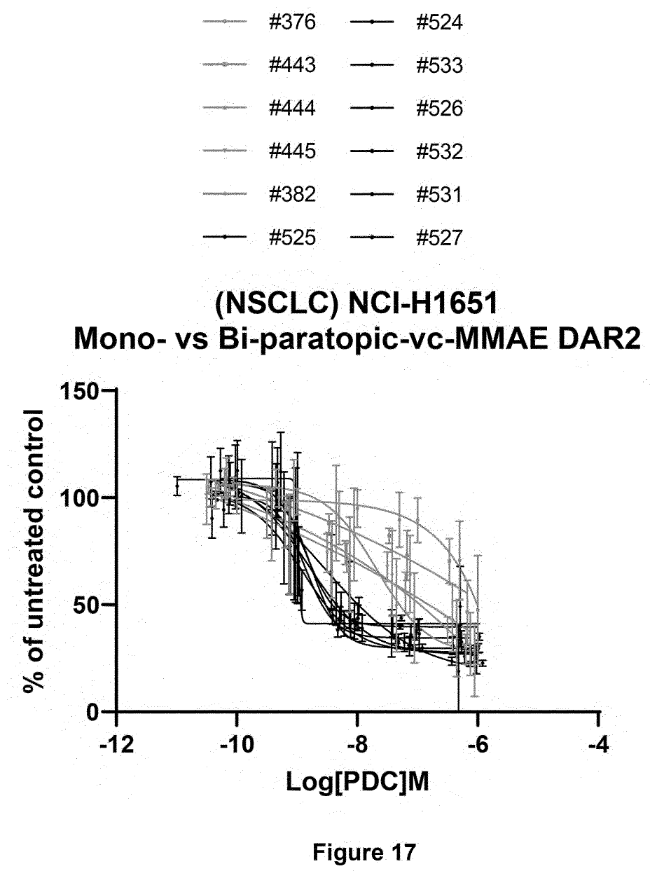

Table 30 and FIG. 17 shows IC₅₀ values and corresponding dose response plots, for cell-killing of ALPP/ALPL2 positive cancer cells (NCI-H1651) by mono- and biparatopic VHH-hFc-vc-PAB-MMAE (DAR2) drug conjugates.

TABLE 30
Calculated IC₅₀ values (nM) for the cell-killing of ALPP/ALPPL2
positive cancer cells (NCI-H1651) by mono- and biparatopic
VHH-hFc- vc PAB MMAE (DAR2) drug conjugates.

| | | | NCI-H1651 |
|---|---|---|---|
| ID | Name | DAR | IC₅₀ @ 96 h (nM) |
| #376 | A06-hFc vc MMAE | 2.0 | >1000 |
| #443 | E06-hFc vc MMAE | 1.87 | >1000 |
| #444 | E10-hFc vc MMAE | 1.94 | 154 |
| #445 | F05-hFc vc MMAE | 1.87 | 110 |
| #382 | D10-hFc vc MMAE | 2.0 | 50.2 |
| #525 | A06-E10-hFc-vcPAB-MMAE | 2.0 | 3.0 |
| #524 | A06-E06-hFc-vcPAB-MMAE | 2.0 | 2.6 |
| #533 | D10-F05-hFc-vcPAB-MMAE | 2.0 | 3.0 |
| #526 | A06-F05-hFc-vcPAB-MMAE | 2.0 | 4.6 |
| #532 | D10-E10-hFc-vcPAB-MMAE | 2.0 | 3.9 |
| #531 | D10-E06-hFc-vcPAB-MMAE | 2.0 | 1.2 |
| #527 | D10-A06-hFc-vcPAB-MMAE | 2.8 | 10.4 |

As shown in Table 30 and FIG. 17, biparatopic VHH-hFc-MMAE (DAR2) conjugates are more potent at killing of ALPP/ALPPL2 expressing cancer cells than the corresponding monoparatopic VHH-hFc-MMAE (DAR2) conjugates.

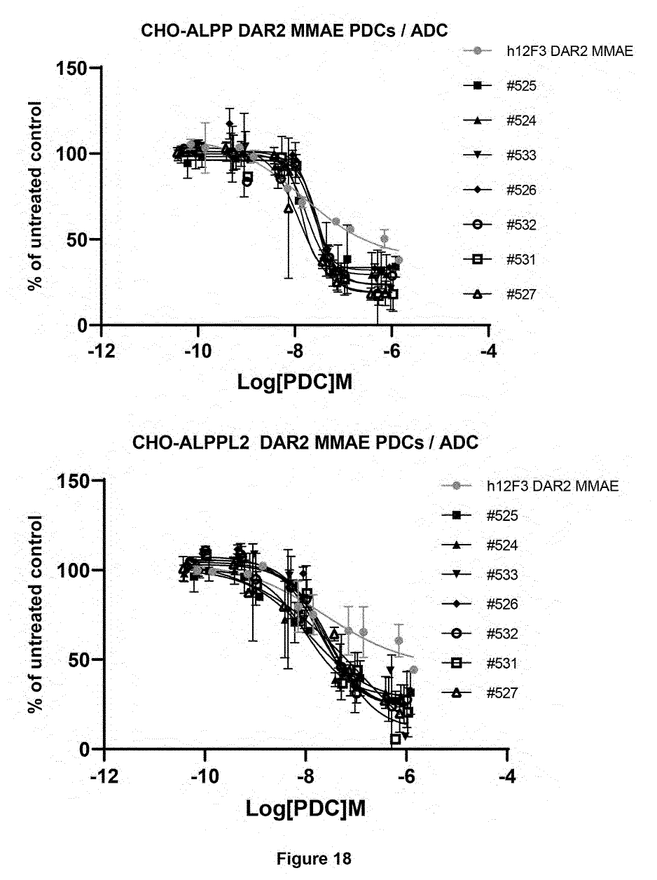

Table 31 and FIG. 18 shows IC₅₀ values and dose response plots, for cell-killing of CHO-ALPP isoform transfectant cell lines stably expressing either ALPP or ALPPL2, by biparatopic VHH-hFc-vc-PAB-MMAE (DAR2) drug conjugates and the monoclonal antibody h12F3 HGLF-vc-PAB-MMAE (DAR2) drug conjugate.

TABLE 31
Calculated IC₅₀ values (nM) for the cell-killing
of CHO-ALPP and CHO-ALPPL2 cells by biparatopic
VHH-hFc- vc PAB MMAE (DAR2) drug conjugates and
h12F3 HGLF -vc-PAB-MMAE (DAR2) drug conjugate.

| | | | IC₅₀ @ 96 h (nM) | |
|---|---|---|---|---|
| ID | | | CHO- | CHO- |
| # | Molecule | DAR | ALPP | ALPPL2 |
| #525 | A06-E10-hFc-vcPAB-MMAE | 2.0 | 18.8 | 34.9 |
| #524 | A06-E06-hFc-vcPAB- MMAE | 2.0 | 27.1 | 23.8 |
| #533 | D10-F05-hFc-vcPAB- MMAE | 2.0 | 37.2 | 38.6 |
| #526 | A06-F05-hFc-vcPAB- MMAE | 2.0 | 36.4 | 45.5 |
| #532 | D10-E10-hFc-vcPAB- MMAE | 2.0 | 38.9 | 39.3 |
| #531 | D10-E06-hFc-vcPAB- MMAE | 2.0 | 35.0 | 46.9 |
| #527 | D10-A06-hFc-vcPAB- MMAE | 2.8 | 18.0 | 625.5 |
| #873 | h12F3-vcPAB-MMAE | 2.0 | 263 | >1000 |

As shown in Table 31 and FIG. 18, biparatopic VHH-hFc-MMAE (DAR2) conjugates are more potent at killing of ALPP and ALPPL2 expressing CHO cells than the monoparatopic h12F3 HGLF-vc-PAB-MMAE (DAR2) antibody drug conjugate.

### Example 11 In Vitro Cell Viability Assays for Cancer Cells Treated with Anti ALPP/ALPPL2 DAR4 MMAE Biparatopic VHH-hFc Drug Conjugates and h12F3 HGLF MMAE (DAR4) Antibody Drug Conjugate

Biparatopic VHH-hFc-MMAE (DAR4) drug conjugates are as described in Examples 5 and 6. The ALPP/ALPPL2 targeting monoclonal antibody h12F3 HGLF, as described in Example 9, was stochastically labelled with MMAE (MC-vc-PAB-MMAE) using endogenous cysteine residues within the antibody sequence to give the corresponding vc-PAB-MMAE antibody drug conjugate with average DAR of 4. Briefly, h12F3 HGLF antibody solution was pH adjusted using 2% v/v 0.5 M Tris, 0.0025 M EDTA, pH8.5. 2.2 molar equivalents of TCEP were then added and left to reduce for 1 hour at 20° C. Next, seven molar equivalents of MC-vc-PAB-MMAE were added to the reduced protein with 10% v/v DMA. The reaction was left to proceed for 1 hr at 20° C. before being quenched with the addition of the same molar equivalents of N-acetyl cysteine as the payload. Free payload was removed by desalting into PBS pH 7.4 using a Desalt Hiprep 26/10 followed by addition of 1 mg carbon per mg of ADC. Carbon was removed by centrifugation after 2 hrs.

Cell Titre Glo assays were performed as described in Example 5. Cells were incubated with biparatopic VHH-hFc-drug conjugates and h12F3 drug conjugate at 37° C., 5% CO₂ for 96 hours and the % of cell viability determined as a function of dose response. The % of control data was plotted against Log [Treatment] concentration and the IC₅₀ value derived using non-linear regression fitting in GraphPad Prism software.

Table 32 shows IC₅₀ values for cell-killing of ALPP/ALPPL2 positive cancer cells (MKN-1, NCI-H1651, NCI-N87, MDA-MB-468) by biparatopic VHH-hFc-vc-PAB-MMAE (DAR4) drug conjugates and h12F3-vc-PAB-MMAE (DAR4) ADC.

TABLE 32
Calculated IC₅₀ values (nM) for the cell-killing of ALPP/ALPPL2
cancer cells (MKN1, NCI-H1651, NCI-N87 and MDA-MB-468) by mono-
and biparatopic VHH-hFc-vc PAB MMAE (DAR4) drug conjugates and
mono-paratopic ALPP/ALPPL2 targeting ADC h12F3-vc PAB MMAE (DAR4).

| | | | | | IC₅₀ at 96 h (nM) and (% viability remaining) | | | |
|---|---|---|---|---|---|---|---|---|
| | | | | | | | | MDA-MB- |
| ID # | Batch | Molecule | Linker | DAR | MKN-1 | NCI-H1651 | NCI-N87 | 468 |
| 634 | 3 | D10-E06-hFc- | [G₄S]₃ | 4.0 | 0.1 (12) | 0.53 (21) | 3.6 (34) | 1.55 (16) |
| | | vc-MMAE | | | | | | |
| 637 | 5 | D10-F05-hFc- | [G₄S]₃ | 3.9 | 0.12 (13) | 0.74 (22) | 1.8 (37) | 0.96 (17) |
| | | vc-MMAE | | | | | | |
| 665 | 3 | A06-F05-hFc- | [G₄S]₃ | 4.0 | 0.26 (16) | 0.47 (22) | 7.9 (36) | 11.96 (17) |
| | | vc-MMAE | | | | | | |
| 679 | 3 | D10-F05-hFc- | [G₄S]₁ | 4.0 | 0.14 (16) | 0.85 (22) | 2 (35) | 2.36 (17) |
| | | vc-MMAE | | | | | | |
| 616 | 3 | D10-F05-hFc- | [G₄S]₃ | 4.0 | 4.5 (26) | 35.7 (31) | >1000 | 237.4 (32) |
| | | vc-MMAE | | | | | | |
| 1033 | 1 | h12F3-vc- | | 4.0 | 2.9 (17) | 0.62 (28) | 9.31 (30) | 12.37 (16) |
| | | MMAE | | | | | | |
| 721 | | αGFP-hFc-vc- | [G₄S]₃ | 4.0 | 759 (33) | 809 (71) | 401 | 292.2 (19) |
| | | MMAE | | | | | | |

As shown in Table 32 biparatopic VHH-hFc-vc-PAB-MMAE (DAR4) conjugates are consistently more potent at killing of ALPP and ALPPL2 expressing cancer cells than the antibody drug conjugate hi 2F3 HGLF-vc-PAB-MMAE (DAR4).

αGFP-hFc-vc-MMAE is a control non-binding VHH-hFc-vc-PAB-MMAE (DAR4) conjugate, generated in the same way as the biparatopic ALPP/ALPPL2 targeting VHH-Fc-MMAE conjugates (DAR4) but where the VHH domains recognise the GFP protein.

### Example 12 In Vivo Efficacy of ALPP/ALPPL2 Biparatopic VHH-hFc MMAE Drug Conjugates and h12F3 HGLF MMAE (DAR4) Antibody Drug Conjugate

### NCI-N87 Human Gastric Carcinoma CDX—Biparatopic VHH-hFc-vcMMAE Drug Conjugates

Efficacy studies in the ALPP/ALPPL2+ NCI-N87 gastric carcinoma xenograft model were performed at Crown Bioscience (Taicang) as described in Example 6, but using single i.v. doses of the agents (the agents used in this study are described in Table 33).

Female BALB/c nude mice were inoculated with NCI-N87 human gastric carcinoma cells (ATCC, Crown ID CL-00455). Each mouse was inoculated subcutaneously in the right flank with NCI-N87 tumor cells (1×10⁷) in 0.1 mL of a 1:1 PBS/Matrigel suspension.

Mice were monitored until the tumour volume reached the recruitment criteria of around 200 mm³ in a sufficient number of animals. Mice were randomised to treatment groups (n=5) such that the mean tumour volume for each group was ~200 mm³ and there was no statistical difference between tumour volumes across the groups. The day of randomisation was designated as Day 0 and mice were treated with vehicle or with the biparatopic protein-drug conjugates or with ADC h12F3-MMAE (DAR4) (see Table 33 below) on Day 1.

Biparatopic VHH-hFc-MMAE (DAR4) protein drug conjugates were intravenously administered as a Single Dose at either 1.25 mg/kg or 2.5 mg/kg.

Biparatopic VHH-hFc-MMAE (DAR2) protein drug conjugate was intravenously administered as a Single Dose at either 1.25 mg/kg or 2.5 mg/kg.

h12F3-MMAE (DAR4) antibody drug conjugate was intravenously administered as a Single Dose at either 1.25 mg/kg or 2.5 mg/kg.

All mice were pre-primed with mouse IgG 20 h before the first PDC dose.

TABLE 33
Protein drug conjugates

| | | | | | Nominal | Actual |
|---|---|---|---|---|---|---|
| PDC | ID # | Batch | Molecule | Linker | DAR | DAR |
| PDC1 | 634 | 2 | D10-E06 hFc- | [G₄S]₃ | 4 | 3.6 |
| | | | vc-PAB-MMAE | | | |
| PDC2 | 637 | 4 | D10-F05 hFc- | [G₄S]₃ | 4 | 3.5 |
| | | | vc-PAB-MMAE | | | |
| PDC3 | 665 | 2 | A06-F05 hFc- | [G₄S]₃ | 4 | 3.3 |
| | | | vc-PAB-MMAE | | | |
| PDC4 | 679 | 2 | D10-F05 hFc- | [G₄S]₁ | 4 | 3.7 |
| | | | vc-MMAE | | | |
| PDC5 | 616 | 2 | D10-F05 hFc- | [G₄S]₃ | 2 | 1.7 |
| | | | vc-PAB-MMAE | | | |
| PDC6 | 1033 | 1 | h12F3-vc- | | 4 | 4.0 |
| | | | PAB-MMAE | | | |

Tumour volume was evaluated by measuring perpendicular tumour diameters, with a calliper, three times a week during the experimental period. Absolute tumour volume (ATV) was calculated using the formula TV (mm³)=[length (mm)×width (mm)×width (mm)]×0.5, where the length (L) is the longest tumour dimension and the width (W) is the longest tumour dimension perpendicular to L. All animals were weighed at the same time as tumour size measurement and also on dosing days. Mice were observed and documented daily for changes in physical appearance, behaviour, adverse clinical signs and general welfare in line with best veterinary practice and welfare guidelines.

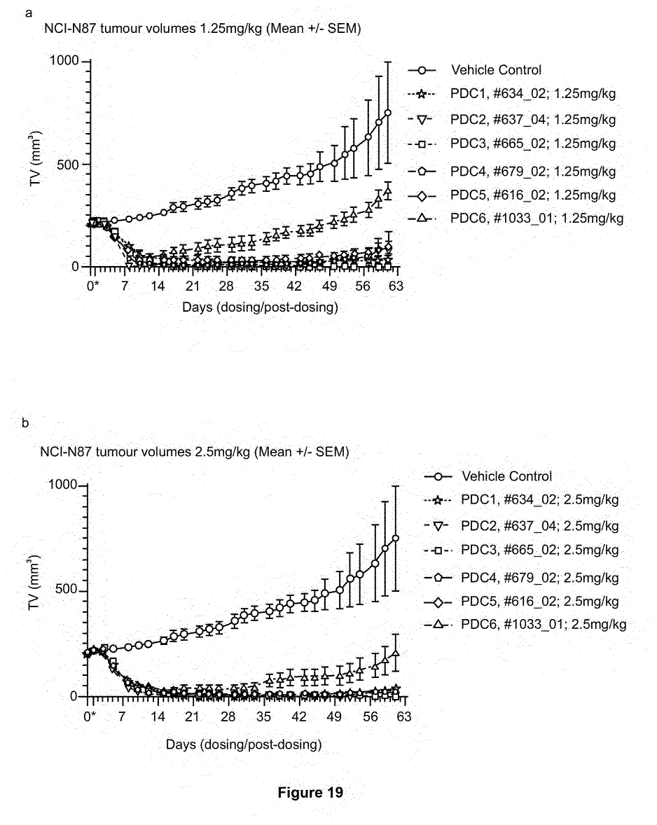

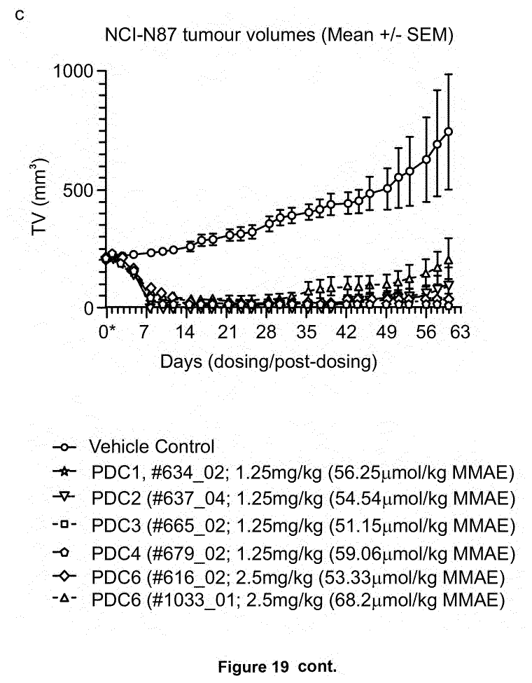

FIG. 19a & b show the effect of the ALPP/ALPPL2-targeting ADC and biparatopic protein-drug conjugates on tumour growth versus vehicle control for the NCI-N87 CDX study. Highly statistically significant in vivo efficacy was observed at the 2.5 mg/kg dose level compared to the vehicle treated group for all biparatopic protein-drug conjugates (PDCs1-5), but not for the ADC h12F3-MMAE (PDC6). Statistically significant anti-tumour efficacy was also observed at 1.25 mg/kg dose level for all of the DAR4 biparatopic protein-drug conjugates (PDCs1-4) but not for the ADC h12F3-MMAE (PDC6). Complete regressions were observed in all of the biparatopic PDC treated groups (PDCs1-5) at 1.25 mg/kg dose level, but no complete regressions (0/5) were observed for the ADC h12F3-MMAE (PDC6). Complete regressions were defined as mice with TV=0 mm³ at End of Study (Day 61).

FIG. 19c shows the same anti-tumour efficacy data plotted on a mol of MMAE payload/kg basis.

As shown in FIG. 19c, biparatopic VHH-hFc-MMAE (DAR4 and DAR2) conjugates of the invention are more efficacious than the ADC h12F3-MMAE (DAR4) based on an MMAE payload equivalents basis (i.e. based on the total number of mols of MMAE payload administered to the mice). Biparatopic VHH-hFc-MMAE (DAR4 and DAR2) conjugates induce stronger anti-tumour responses than the ADC h12F3-MMAE (DAR4) using less MMAE payload.

### HPAC Human Pancreatic Ductal Adenocarcinoma CDX—Biparatopic VHH-hFc-vcMMAE Drug Conjugates

Efficacy studies in the ALPP/ALPPL2+ HPAC human pancreatic ductal adenocarcinoma xenograft model were performed at Crown Bioscience (Taicang) as described in Example 6, but using single i.v. doses of the agents (agents used in this study are described in the Table 34).

Female NOD/SCID mice were inoculated with HPAC human pancreatic ductal adenocarcinoma cells (ATCC, Crown ID CL-00891). Each mouse was inoculated subcutaneously in the right front flank region with HPAC tumor cells (1×10⁷) in 0.1 mL of a 1:1 PBS/Matrigel suspension.

Mice were monitored until the tumour volume reached the recruitment criteria of around 200 mm³ in a sufficient number of animals. Mice were randomised to treatment groups (n=5) such that the mean tumour volume for each group was ~200 mm³ and there was no statistical difference between tumour volumes across the groups. The day of randomisation was designated as Day 0 and mice were treated with vehicle or with the biparatopic protein-drug conjugates or with the ADC h12F3-vc-PAB-MMAE (DAR4) (see Table 34) on Day 1.

Biparatopic VHH-hFc-MMAE (DAR4) protein-drug conjugates were intravenously administered as a Single Dose at either 5 mg/kg or 10 mg/kg.

Biparatopic VHH-hFc-MMAE (DAR2) protein-drug conjugate was intravenously administered as a Single Dose at either 5 mg/kg or 10 mg/kg.

h12F3-MMAE (DAR4) antibody-drug conjugate was intravenously administered as a Single Dose at either 5 mg/kg or 10 mg/kg.

All mice were pre-primed with mouse IgG 20 h before the first PDC dose.

TABLE 34
Protein drug conjugates

| | | | | | Nominal | Actual |
|---|---|---|---|---|---|---|
| PDC | ID # | Batch | Molecule | Linker | DAR | DAR |
| PDC1 | 634 | 2 | D10-E06 hFc-vc-PAB-MMAE | [G₄S]₃ | 4 | 3.6 |
| PDC2 | 637 | 4 | D10-F05 hFc-vc-PAB-MMAE | [G₄S]₃ | 4 | 3.5 |
| PDC3 | 665 | 2 | A06-F05 hFc-vc-PAB-MMAE | [G₄S]₃ | 4 | 3.3 |
| PDC4 | 679 | 2 | D10-F05 hFc-vc-PAB-MMAE | [G₄S]₁ | 4 | 3.7 |
| PDC5 | 616 | 2 | D10-F05 hFc-vc-PAB-MMAE | [G₄S]₃ | 2 | 1.7 |
| PDC6 | 1033 | 1 | h12F3-vc-PAB-MMAE | | 4 | 4.0 |

Tumour volume was evaluated by measuring perpendicular tumour diameters, with a calliper, three times a week during the experimental period. Absolute tumour volume (ATV) was calculated using the formula TV (mm³)=[length (mm)×width (mm)×width (mm)]×0.5, where the length (L) is the longest tumour dimension and the width (W) is the longest tumour dimension perpendicular to L. All animals were weighed at the same time as tumour size measurement and also on dosing days. Mice were observed and documented daily for changes in physical appearance, behaviour, adverse clinical signs and general welfare in line with best veterinary practice and welfare guidelines.

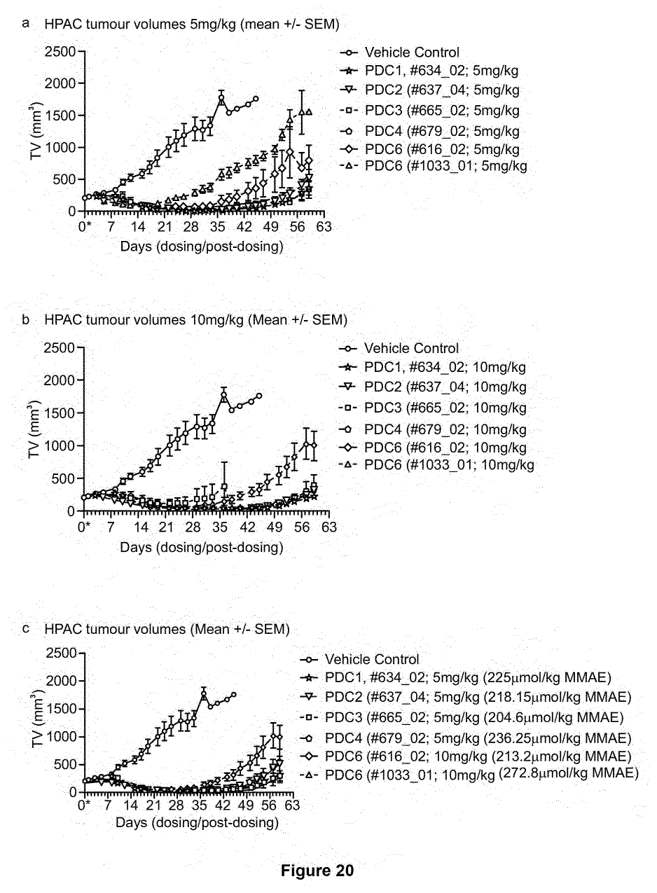

FIG. 20a & b show the effect of the VHH-hFc-MMAE (DAR4 and DAR2) conjugates and the ADC h12F3-MMAE (DAR4) agent on tumour growth versus vehicle control for the HPAC CDX study. All molecules were well tolerated and showed highly statistically significant in vivo efficacy at both 5 mg/kg and 10 mg/kg dose levels compared to the vehicle treated group. Complete regressions were observed in the groups treated with biparatopic PDCs #1-5 (Complete regressions were defined as mice with TV=0 mm³ for 3 consecutive measurements). No complete regressions were observed, at any point, for the ADC h12F3-vc-PAB-MMAE (PDC6) treated groups.

FIG. 20c shows the same anti-tumour efficacy data plotted on a mol of MMAE payload/kg basis.

As shown in FIG. 20c, biparatopic VHH-hFc-MMAE (DAR4 and DAR2) conjugates of the invention are more efficacious than ADC h12F3-MMAE (DAR4) based on an MMAE payload equivalents basis (i.e. based on total number of mols of MMAE payload administered to the mice). Biparatopic VHH-hFc-MMAE (DAR4 and DAR2) conjugates of the invention induce stronger anti-tumour responses with less MMAE payload than ADC h12F3-MMAE (DAR4).

### Caov-3 High Grade Ovarian Serous Adenocarcinoma CDX—Biparatopic VHH-hFc-vcMMAE Drug Conjugates

Efficacy studies in the ALPP/ALPPL2+ Caov-3 ovarian cancer xenograft model were performed at Crown Bioscience (Beijing). This CDX model was shown to express ALPP/ALPPL2 by IHC but has weaker staining than the NCI-N87 and HPAC CDX models (FIG. 12).

ALPP/ALPPL2 expression was confirmed in Caov-3 cell-line-derived xenograft FFPE samples using anti-ALPP/ALPPL2 antibody (MS Validated Antibodies) and Rabbit Isotype negative control by immunohistochemical (IHC) staining.

For the Caov-3 studies, female NCG mice were inoculated with Caov-3 ovarian adenocarcinoma cells (Peking Union Medical College, Crown ID CL-00780). Each mouse was inoculated at the mammary fat pad with Caov-3 tumor cells (1×10⁷) in 0.2 mL of a 1:1 PBS/Matrigel suspension.

Mice (n=3/group) were monitored until the tumour volume reached the recruitment criteria of around 200 mm³ in a sufficient number of animals. Mice were randomised to treatment groups such that the mean tumour volume for each group was −200 mm³ and there was no statistical difference between tumour volumes across the groups. The day of randomisation was designated as Day 0 and mice were treated with vehicle or with the biparatopic protein-drug conjugates or with ADC h12F3-MMAE (DAR4) (see Table 35 below) on Day 1. Protein-drug conjugates and h12F3 ADC were dosed intravenously once every four days (Q4D×4) at either 1 mg/kg or 3 mg/kg. All mice were pre-primed with mouse IgG 20h before first PDC dose.

TABLE 35
Protein drug conjugates

| | | | | | Nominal | Actual |
|---|---|---|---|---|---|---|
| PDC | ID # | Batch | Molecule | Linker | DAR | DAR |
| PDC1 | 634 | 3 | D10-E06 hFc-vc-PAB-MMAE | [G₄S]₃ | 4 | 4.0 |
| PDC2 | 637 | 5 | D10-F05 hFc-vc-PAB-MMAE | [G₄S]₃ | 4 | 3.9 |
| PDC3 | 665 | 3 | A06-F05 hFc-vc-PAB-MMAE | [G₄S]₃ | 4 | 4.0 |
| PDC4 | 679 | 3 | D10-F05 hFc-vc-PAB-MMAE | [G₄S]₁ | 4 | 4.0 |
| PDC5 | 616 | 3 | D10-F05 hFc-vc-PAB-MMAE | [G₄S]₃ | 2 | 2.0 |
| PDC6 | 1033 | 1 | h12F3-vc-PAB-MMAE | | 4 | 4.0 |

Tumour volume was evaluated by measuring perpendicular tumour diameters, with a calliper, twice a week during the experimental period. Absolute tumour volume (ATV) was calculated using the formula TV (mm³)=[length (mm)×width (mm)×width (mm)]×0.5, where the length (L) is the longest tumour dimension, and the width (W) is the longest tumour dimension perpendicular to L. All animals were weighed at the same time as tumour size measurement and also on dosing days. Mice were observed and documented daily for changes in physical appearance, behaviour, adverse clinical signs and general welfare in line with best veterinary practice and welfare guidelines.

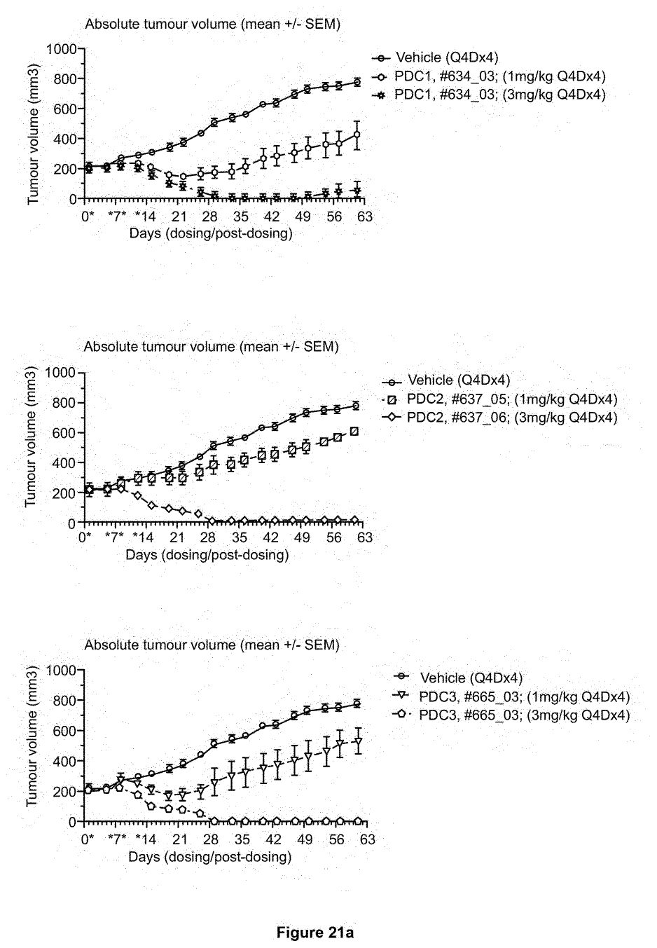

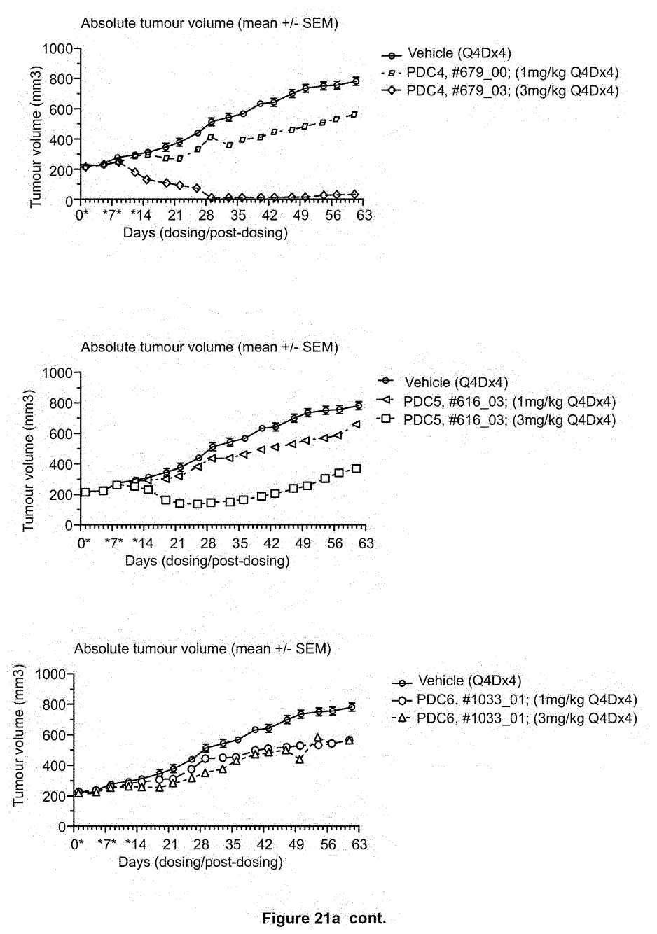

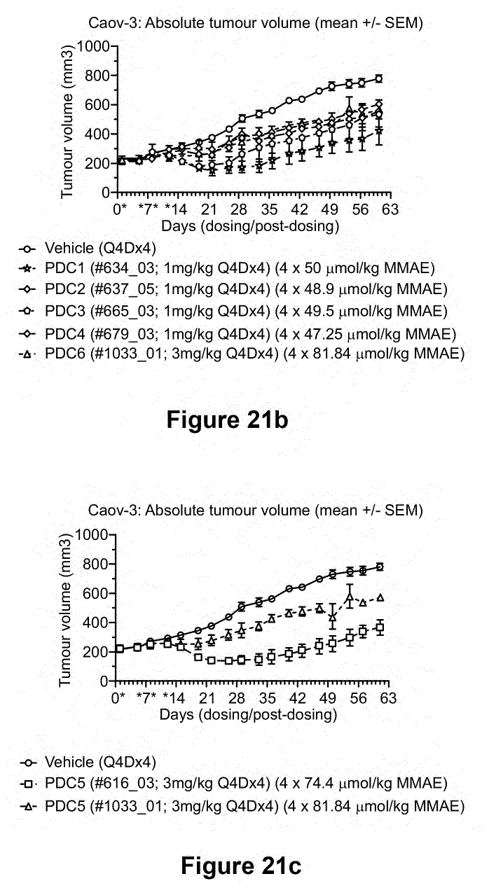

FIG. 21a shows the effect of the biparatopic VHH-hFc-MMAE (DAR4 and DAR2) conjugates and ADC hF12-MMAE (DAR4) on tumour growth versus vehicle control for the Caov-3 ovarian CDX model. Treatment with all biparatopic VHH-hFc-MMAE agents (PDCs 1-5) produced statistically significant anti-tumour efficacy. ADC h12F3-MMAE (PDC6) did not produce statistically significant anti-tumour efficacy at either dose tested. One animal from the ADC h12F3-MMAE (PDC6) 3 mg/kg Q4D×4 group died on Day 49 and a further animal was humanely euthanized on Day 55. Mice with complete regressions (CRs) were observed in the 3 mg/kg Q4D×4 groups for PDC1-4 at the end of the study period (Day 61), but not for ADC h12F3-MMAE (PDC6).

FIG. 21b shows the same anti-tumour efficacy data for biparatopic PDCs 1-4 at 1 mg/kg and the ADC h12F3-MMAE (PDC6) at 3 mg/kg re-plotted on a mol of MMAE payload/kg basis.

As shown in FIG. 21b Biparatopic VHH-hFc-MMAE PDCs delivered similar or better anti-tumour efficacy using much less MMAE payload than the ADC h12F3-MMAE (PDC6). This is also demonstrated in FIG. 21c where the DAR2 Biparatopic VHH-hFc-MMAE (PDC5) induced a stronger anti-tumour response with less MMAE payload, than the DAR4 ADC h12F3-MMAE conjugate.

1. An alkaline phosphatase, placental type (ALPP) and/or alkaline phosphatase, germ cell type (ALPPL2) specific antigen binding molecule comprising an amino acid sequence represented by the formula (I): 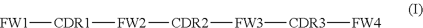
wherein
FW1 is a framework region;
CDR1 is a CDR sequence;
FW2 is a framework region;
CDR2 is a CDR sequence;
FW3 is a framework region;
CDR3 is a CDR sequence;
FW4 is a framework region.

2. The ALPP and/or ALPPL2 specific antigen binding molecule of claim 1, wherein
CDR1 is a CDR sequence having an amino acid sequence selected from the group consisting of SEQ ID NOs: 11 to 15;
FW2 is a framework region;
CDR2 is a CDR sequence having an amino acid sequence selected from the group consisting of SEQ ID NOs: 6 to 10;
FW3 is a framework region;
CDR3 is a CDR sequence having an amino acid sequence selected from the group consisting of SEQ ID NOs: 1 to 5;
FW4 is a framework region.

3. The ALPP and/or ALPPL2 specific antigen binding molecule of any one of claims 1 to 2, wherein
CDR1 is a CDR sequence having an amino acid sequence of SEQ ID NO: 11, CDR2 is a CDR sequence having an amino acid sequence of SEQ ID NO: 6, and CDR3 is a CDR sequence having an amino acid sequence of SEQ ID NO: 1; or
CDR1 is a CDR sequence having an amino acid sequence of SEQ ID NO: 12, CDR2 is a CDR sequence having an amino acid sequence of SEQ ID NO: 7, and CDR3 is a CDR sequence having an amino acid sequence of SEQ ID NO: 2; or
CDR1 is a CDR sequence having an amino acid sequence of SEQ ID NO: 13, CDR2 is a CDR sequence having an amino acid sequence of SEQ ID NO: 8, and CDR3 is a CDR sequence having an amino acid sequence of SEQ ID NO: 3;
CDR1 is a CDR sequence having an amino acid sequence of SEQ ID NO: 14, CDR2 is a CDR sequence having an amino acid sequence of SEQ ID NO: 9, and CDR3 is a CDR sequence having an amino acid sequence of SEQ ID NO: 4; and/or
CDR1 is a CDR sequence having an amino acid sequence of SEQ ID NO: 15, CDR2 is a CDR sequence having an amino acid sequence of SEQ ID NO: 10, and CDR3 is a CDR sequence having an amino acid sequence of SEQ ID NO: 5.

4. The ALPP and/or ALPPL2 specific antigen binding molecule of any preceding claim, having an amino acid sequence of any one of SEQ ID NOs: 17, 28, 30, 31 or 34.

5. A recombinant fusion protein comprising the ALPP and/or ALPPL2 specific antigen binding molecule of any preceding claim.

6. The recombinant fusion protein of claim 5, wherein the ALPP and/or ALPPL2 specific antigen binding molecule is fused to one or more biologically active proteins via one or more linker domains.

7. The recombinant fusion protein of claim 6, wherein the linker is selected from the group consisting of G₄S (SEQ ID NO: 236) and [G₄S]₃ (SEQ ID NO: 237).

8. The recombinant fusion protein of any one of claims 5 to 7, wherein the at least one biologically active molecule is an immunoglobulin, an immunoglobulin Fc region, a fragment of an immunoglobulin Fc region, an Fc heavy chain, a CH2 region, a CH3 region, an immunoglobulin Fab region, a Fab′, a Fv, a Fv-Fc, a single chain Fv (scFv), scFv-Fc, (scFv)₂, a diabody, a triabody, a tetrabody, a bispecific t-cell engager, an intein, a VNAR domain, a single domain antibody (sdAb), a VH domain, or a scaffold protein (affibodies, centyrins, darpins etc.).

9. The recombinant fusion protein of any one of claims 5 to 8, wherein the at least one biologically active protein is an immunoglobulin Fc region or fragment thereof.

10. The recombinant fusion protein of claim 5 to 9, having an amino acid sequence according to SEQ ID NO: 87 to 96.

11. The recombinant fusion protein of any one of claims 5 to 10, wherein the immunoglobulin Fc region comprises a mutation to Cys (C), optionally wherein the C mutation is at position S239 and/or at position S442.

12. The recombinant fusion protein of any one of claims 5 to 11, having an amino acid sequence of SEQ ID NO: 97 to 121.

13. The recombinant fusion protein of any one of claims 5 to 12, wherein the fragment of an immunoglobulin Fc region is engineered to dimerize with a second fragment of an immunoglobulin Fc region.

14. The recombinant fusion protein of any one of claims 5 to 13, wherein one or more residues of the first fragment of an immunoglobulin Fc region comprise one or more amino acid substitutions suitable for knobs-in-holes (KIH) dimerization with the second fragment of an immunoglobulin Fc region, and wherein one or more residues of the second fragment of an immunoglobulin Fc region comprise one or more amino acid substitutions suitable for knobs-in-holes (KIH) dimerization with the first fragment of an immunoglobulin Fc region.

15. The recombinant fusion protein of any one of claims 5 to 14, wherein the fragment of an immunoglobulin Fc region comprises a T366Y substitution or a Y407T substitution.

16. The recombinant fusion protein of any one of claims 5 to 15, having an amino acid sequence of SEQ ID NO: 188 to 208

17. The recombinant fusion protein of any one of claims 5 to 15, having an amino acid sequence of SEQ ID NO: 127 to 186.

18. A recombinant fusion protein dimer comprising
(a) a first recombinant fusion protein of any one of claims 5 to 17, and
(b) a second recombinant fusion protein of any one of claims 5 to 17.

19. The recombinant fusion protein dimer of claim 18 comprising any one or any two of SEQ ID NOs 87 to 186 or 188 to 208.

20. The recombinant fusion protein dimer of claim 18 or 19 wherein;
(a) the first recombinant fusion protein comprises a sequence according to SEQ ID NO: 138 (D10-hFc (long linker) S239C+T366Y), and
(b) the second recombinant fusion protein comprises a sequence according to SEQ ID NO: 129 (E06-hFc (long linker) S239C+Y407T);
(a) the first recombinant fusion protein comprises a sequence according to SEQ ID NO: 138 (D10-hFc (long linker) S239C+T366Y), and
(b) the second recombinant fusion protein comprises a sequence according to SEQ ID NO: 130 (E10-hFc (long linker) S239C+Y407T);
(a) the first recombinant fusion protein comprises a sequence according to SEQ ID NO: 138 (D10-hFc (long linker) S239C+T366Y), and
(b) the second recombinant fusion protein comprises a sequence according to SEQ ID NO: 131 (F05-hFc (long linker) S239C+Y407T);
(a) the first recombinant fusion protein comprises a sequence according to SEQ ID NO: 137 (A06-hFc S239C T366Y), and
(b) the second recombinant fusion protein comprises a sequence according to SEQ ID NO: 131 (F05-hFc (long linker) S239C+Y407T);
(a) the first recombinant fusion protein comprises a sequence according to SEQ ID NO: 143 (D10-hFc (short linker) S239C+T366Y), and
(b) the second recombinant fusion protein comprises a sequence according to SEQ ID NO: 134 (E06-hFc (short linker) S239C+Y407T);
(a) the first recombinant fusion protein comprises a sequence according to SEQ ID NO: 143 (D10-hFc (short linker) S239C+T366Y), and
(b) the second recombinant fusion protein comprises a sequence according to SEQ ID NO: 135 (E10-hFc (short linker) S239C Y407T);
(a) the first recombinant fusion protein comprises a sequence according to SEQ ID NO: 143 (D10-hFc (short linker) S239C+T366Y), and
(b) the second recombinant fusion protein comprises a sequence according to SEQ ID NO: 136 (F05-hFc (short linker) S239C+Y407T);
(a) the first recombinant fusion protein comprises a sequence according to SEQ ID NO: 178 (D10-hFc S239C & S442C T366Y), and
(b) the second recombinant fusion protein comprises a sequence according to SEQ ID NO: 169 (E06-hFc S239C & S442C+Y407T);
(a) the first recombinant fusion protein comprises a sequence according to SEQ ID NO: 178 (D10-hFc S239C & S442C+T366Y), and
(b) the second recombinant fusion protein comprises a sequence according to SEQ ID NO: 170 (E10-hFc S239C & S442C+Y407T);
(a) the first recombinant fusion protein comprises a sequence according to SEQ ID NO: 178 (D10-hFc S239C & S442C+T366Y), and
(b) the second recombinant fusion protein comprises a sequence according to SEQ ID NO: 171 (F05-hFc S239C & S442C+Y407T);
(a) the first recombinant fusion protein comprises a sequence according to SEQ ID NO: 177 (A06-hFc S239C & S442C+T366Y), and
(b) the second recombinant fusion protein comprises a sequence according to SEQ ID NO: 171 (F05-hFc S239C & S442C+Y407T);
(a) the first recombinant fusion protein comprises a sequence according to SEQ ID NO: 183 (D10-hFc (short linker) S239C & S442C+T366Y), and
(b) the second recombinant fusion protein comprises a sequence according to SEQ ID NO: 174 (E06-hFc (short linker) S239C & S442C+Y407T);
(a) the first recombinant fusion protein comprises a sequence according to SEQ ID NO: 183 (D10-hFc (short linker) S239C & S442C+T366Y), and
(b) the second recombinant fusion protein comprises a sequence according to SEQ ID NO: 175 (E10-hFc (short linker) S239C & S442C+Y407T); or
(a) the first recombinant fusion protein comprises a sequence according to SEQ ID NO: 183 (D10-hFc (short linker) S239C & S442C+T366Y), and
(b) the second recombinant fusion protein comprises a sequence according to SEQ ID NO: 176 (F05-hFc (short linker) S239C & S442C+Y407T).

21. A target-binding molecule-drug conjugate, comprising
(a) the ALPP and/or ALPPL2 specific antigen binding molecule according to any one of claims 1 to 4, the recombinant fusion protein of any one of claims 5 to 17, or the recombinant fusion protein dimer of any one of claims 18 to 20, and
(b) at least one toxin, cytotoxic or cytostatic agent.

22. The target-binding molecule-drug conjugate of claim 21, wherein the ALPP and/or ALPPL2 specific antigen binding molecule is conjugated to the cytotoxic or cytostatic agent via a linker,

23. The target-binding molecule-drug conjugate of claim 21 or claim 22, wherein (b) is a toxin selected from the group consisting of:
auristatins,
anthracyclines, preferably PNU-derived anthracyclines
maytansinoids,
amanitin derivatives, preferably α-amanitin derivatives
calicheamicins,
tubulysins
duocarmycins
radioisotopes—such as an alpha-emitting radionuclide, such as 227 Th and 225 Ac label
liposomes comprising a toxic payload,
protein toxins
taxanes
pyrrolbenzodiazepines and dimers thereof
indolinobenzodiazepine pseudodimers
spliceosome inhibitors
CDK11 inhibitors
nicotinamide phosphoribosyltransferase inhibitors (NAMPTi)
Pyridinobenzodiazepines and dimers thereof
Cyclopropapyrroloindole (CPI), cyclopropabenzindole (CBI) or cyclopropathienoindole (CTI) and optionally dimers thereof
Irinotecan or exatecan and their derivatives.

24. The target-binding molecule-drug conjugate of any one of claims 21 to 23, wherein the toxin is an auristatin.

25. The target-binding molecule-drug conjugate of any one of claims 21 to 24, wherein the toxin is a monomethyl auristatin.

26. The target-binding molecule-drug conjugate of any one of claims 21 to 24, wherein the toxin is monomethyl auristatin E.

27. The target-binding molecule-drug conjugate of any one of claims 21 to 23 wherein (b) is an a MMAE derivative,
wherein the target-binding molecule-drug conjugate has the structure of formula (VI): 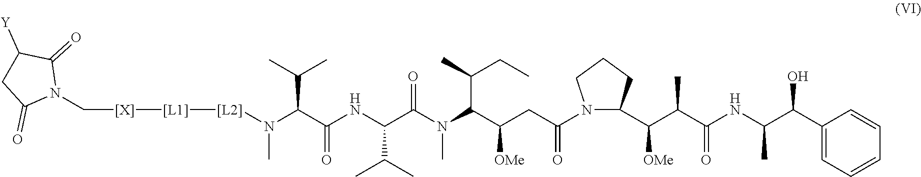
[X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, a dipeptide, a tripeptide, —(CH2)n-, —(CH2CH2O)n-, p-aminobenzyloxycarbonyl (PAB), Val-Cit-PAB, Val-Ala-PAB, Ala-Ala-Asn-PAB, Val-Ala, Asn-Ala, any amino acid, D-amino acids, Phe-Lys-PAB, and combinations thereof; and
Y comprises an ALPP and/or ALPPL2 specific antigen binding molecule or recombinant fusion protein according to any one of claims 1 to 20.

28. The target-binding molecule-drug conjugate of claim 27, wherein (b) is an a MMAE derivative, wherein the target-binding molecule-drug conjugate has the structure of formula (VII):

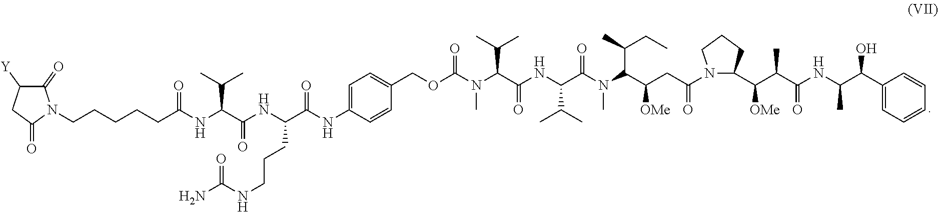

29. The target-binding molecule-drug conjugate of any one of claims 21 to 23, comprising (b) an anthracycline (PNU) derivative,
wherein the target-binding molecule-drug conjugate has the structure of formula (III): 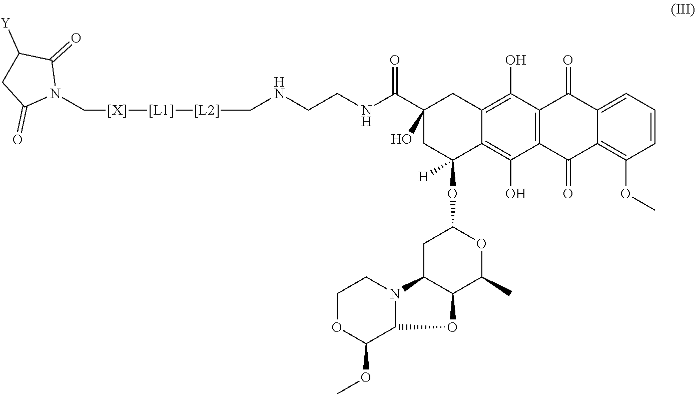
wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH2O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof; and
Y comprises an ALPP and/or ALPPL2 specific antigen binding molecule or recombinant fusion protein according to any one of claims 1 to 20.

30. The target-binding molecule-drug conjugate of claim 29, wherein the target-binding molecule-drug conjugate has a structure selected from:

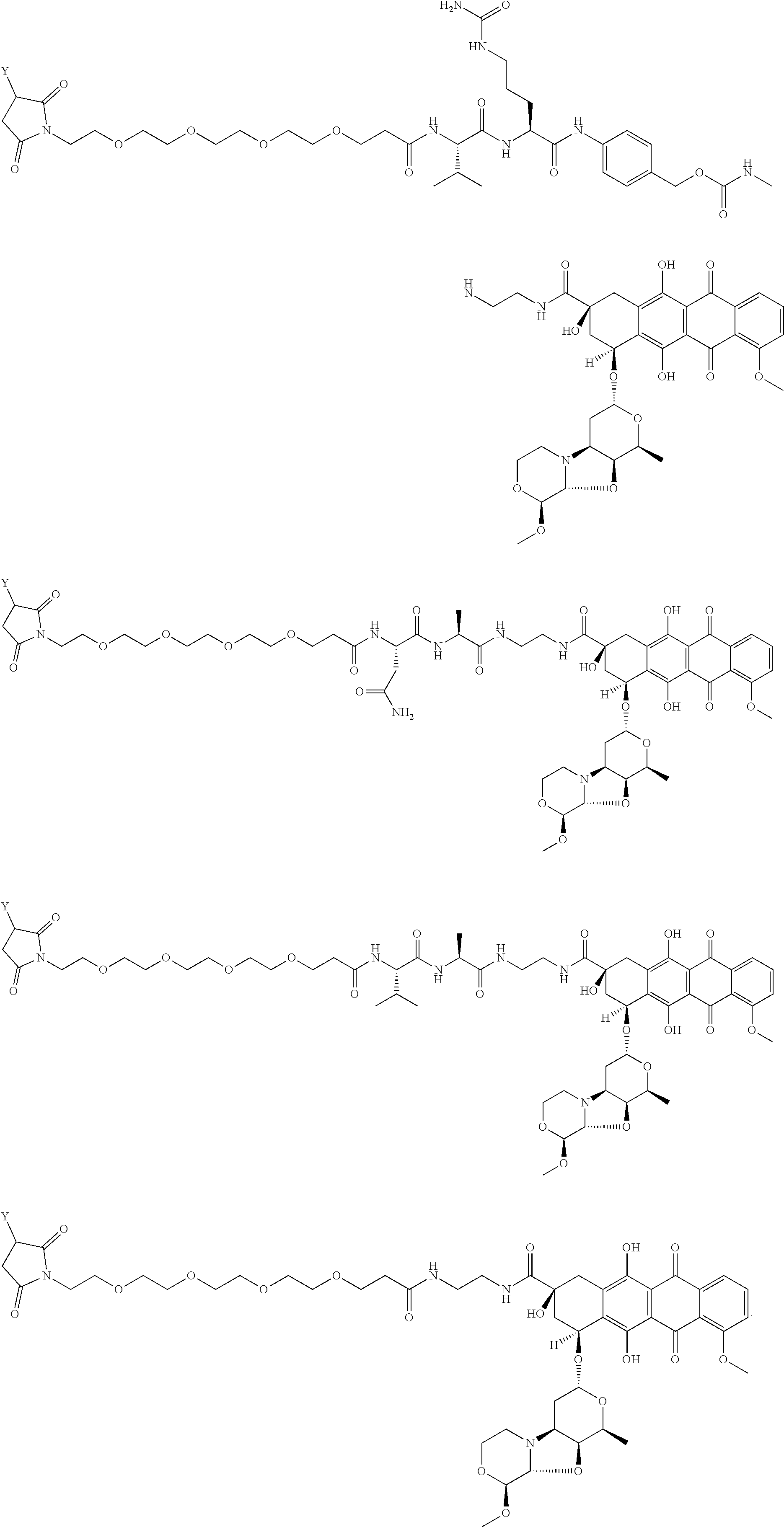

31. The target-binding molecule-drug conjugate of claim 23, wherein the toxin is Exatecan or an Exatecan derivative.

32. An antibody, antibody fragment or antigen-binding molecule that competes for binding to ALPP and/or ALPPL2 with the ALPP and/or ALPPL2-specific antigen binding molecule of claims 1 to 4.

33. An ALPP and/or ALPPL2-specific chimeric antigen receptor (CAR) comprising at least one ALPP and/or ALPPL2-specific antigen binding molecule of claims 1 to 4, fused or conjugated to at least one transmembrane region and at least one intracellular domain.

34. A cell comprising the chimeric antigen receptor according to claim 33, which cell is preferably an engineered T-cell.

35. A nucleic acid sequence comprising a polynucleotide sequence that encodes a specific antigen binding molecule, recombinant fusion protein, recombinant fusion protein dimer or chimeric antigen receptor according to any one of claims 1 to 33.

36. A vector comprising the nucleic acid sequence of claim 35.

37. A cell comprising the vector of claim 36.

38. A pharmaceutical composition comprising the ALPP and/or ALPPL2 specific antigen binding molecule of claims 1 to 4, the recombinant fusion protein of claims 5 to 17, the recombinant fusion protein dimer of claims 18 to 20, the CAR of claim 33, the cell of claim 34 or claim 37, the nucleic acid sequence of claim 35, the vector of claim 36, or the target-binding molecule-drug conjugate of claims 21 to 31.

39. The ALPP and/or ALPPL2 specific antigen binding molecule of claims 1 to 4, the recombinant fusion protein of claims 5 to 17, the recombinant fusion protein dimer of claims 18 to 20, the CAR of claim 33, the cell of claim 34 or claim 37, the nucleic acid sequence of claim 35, the vector of claim 36, or the target-binding molecule-drug conjugate of claims 21 to 31 for use in therapy

40. The ALPP and/or ALPPL2 specific antigen binding molecule of claims 1 to 4, the recombinant fusion protein of claims 5 to 17, the recombinant fusion protein dimer of claims 18 to 20, the CAR of claim 33, the cell of claim 34 or claim 37, the nucleic acid sequence of claim 35, the vector of claim 36, or the target-binding molecule-drug conjugate of claims 21 to 31 for use in the treatment of cancer, optionally wherein the cancer is an ALPP and/or ALPPL2-positive cancer type.

41. The ALPP and/or ALPPL2 specific antigen binding molecule, the recombinant fusion protein, the recombinant fusion protein dimer, the CAR, the cell, the nucleic acid sequence, the vector, or the target-binding molecule-drug conjugate for use of claim 40, wherein the cancer is selected from the group consisting of ovarian cancer, endometrial cancer, cervical cancer, gastric cancer, oesophageal cancer, bladder cancer, lung cancer, pancreatic cancer and testicular cancer.

42. The use of the ALPP and/or ALPPL2 specific antigen binding molecule of claims 1 to 4, the recombinant fusion protein of claims 5 to 17, the recombinant fusion protein dimer of claims 18 to 20, the CAR of claim 33, the cell of claim 34 or claim 37, the nucleic acid sequence of claim 35, the vector of claim 36, or the target-binding molecule-drug conjugate of claims 21 to 31 in the manufacture of a medicament for the treatment of a disease in a patient in need thereof.

43. A method of treatment of a disease in a patient in need of treatment comprising administration to said patient of a therapeutically effective dosage of the ALPP and/or ALPPL2 specific antigen binding molecule of claims 1 to 4, the recombinant fusion protein of claims 5 to 17, the recombinant fusion protein dimer of claims 18 to 20, the CAR of claim 33, the cell of claim 34 or claim 37, the nucleic acid sequence of claim 35, the vector of claim 36, or the target-binding molecule-drug conjugate of claims 21 to 31.

44. The method of treatment of claim 43, wherein the disease to be treated is a cancer, optionally wherein the cancer is an ALPP and/or ALPPL2-positive cancer type.

45. The method of treatment of claim 44, wherein the cancer is selected from the group consisting of ovarian cancer, endometrial cancer, cervical cancer, gastric cancer, oesophageal cancer, bladder cancer, lung cancer, pancreatic cancer and testicular cancer

46. A method for preparing the ALPP and/or ALPPL2 specific antigen binding molecule of claims 1 to 4, the recombinant fusion protein of claims 5 to 17, the recombinant fusion protein dimer of claims 18 to 20, the CAR of claim 33, the cell of claim 34 or claim 37, the nucleic acid sequence of claim 35, the vector of claim 36, or the target-binding molecule-drug conjugate of claims 21 to 31.

47. An engineered ALPP and/or ALPPL2 monomer, wherein the engineered ALPP and/or ALPPL2 monomer is engineered to be capable of forming a covalent link with a further engineered ALPP and/or ALPPL2 monomer.

48. The engineered ALPP and/or ALPPL2 monomer of claim 47, wherein the covalent link is a disulphide bridge and/or the engineered ALPP and/or ALPPL2 monomer comprises an introduced cysteine residue.

49. The engineered ALPP and/or ALPPL2 monomer of claim 48, wherein the engineered ALPP and/or ALPPL2 monomer comprises a G503C substitution in SEQ ID NO: 263, or an equivalent substitution in another ALPP and/or ALPPL2 monomer, or wherein the engineered ALPP monomer comprises a sequence according to SEQ ID NO: 263.

50. An engineered ALPP and/or ALPPL2 homodimer, comprising a first and second engineered ALPP and/or ALPPL2 monomer of claims 47 to 49.

51. A method for generation of ALPP and/or ALPPL2 specific binding molecules comprising;
(a) immunising an animal with the engineered ALPP and/or ALPPL2 homodimer of claim 50,
(b) isolating Peripheral Blood Mononuclear Cells (PBMCs) from the animal,
(c) cloning into vectors RNA or cDNA sequences obtained from the PBMCs,
(d) transforming prokaryotic cells with the above vectors in conditions allowing the production of the specific binding molecules,
(e) selecting the specific binding molecules by subjecting them to antigen-affinity selection,
(f) recovering the specific binding molecules having the desired specificity.

52. A bi-paratopic alkaline phosphatase, placental type (ALPP) and/or alkaline phosphatase, germ cell type (ALPPL2) specific antigen binding molecule.

53. A bi-paratopic target-binding molecule-drug conjugate, comprising (a) the bi-paratopic alkaline phosphatase, placental type (ALPP) and/or alkaline phosphatase, germ cell type (ALPPL2) specific antigen binding molecule of claim 52, and
(b) at least one toxin, cytotoxic or cytostatic agent.

54. The bi-paratopic target-binding molecule-drug conjugate of claim 53, wherein the toxin is MMAE.

55. The bi-paratopic target-binding molecule-drug conjugate of claim 53, wherein (b) is an a MMAE derivative,
wherein the target-binding molecule-drug conjugate has the structure of formula (VI): 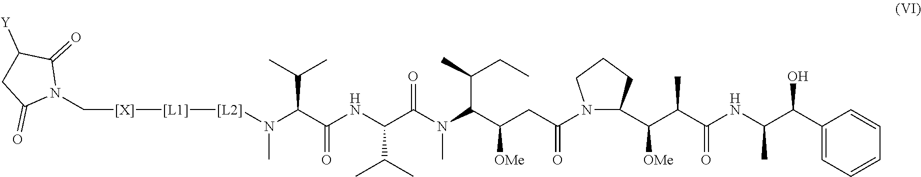
[X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, a dipeptide, a tripeptide, —(CH2)n-, —(CH2CH2O)n-, p-aminobenzyloxycarbonyl (PAB), Val-Cit-PAB, Val-Ala-PAB, Ala-Ala-Asn-PAB, Val-Ala, Asn-Ala, any amino acid, D-amino acids, Phe-Lys-PAB, and combinations thereof; and
Y comprises the bi-paratopic alkaline phosphatase, placental type (ALPP) and/or alkaline phosphatase, germ cell type (ALPPL2) specific antigen binding molecule of claim 52.

56. The bi-paratopic target-binding molecule-drug conjugate of claim 53, comprising (b) an anthracycline (PNU) derivative,
wherein the target-binding molecule-drug conjugate has the structure of formula (III): 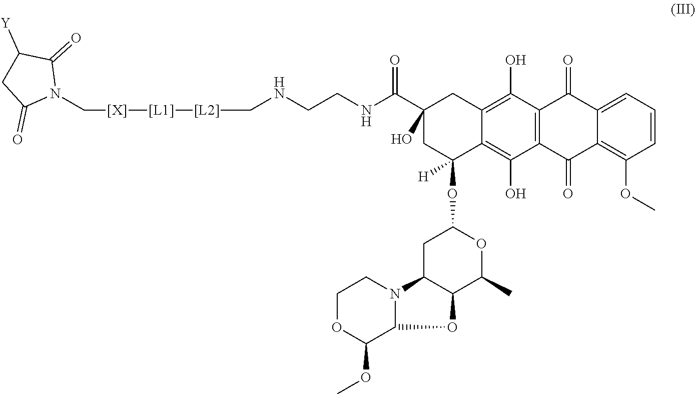
wherein [X] is an optional spacer selected from the group comprising substituted or unsubstituted alkyl groups, substituted or unsubstituted heteroalkyl groups, substituted or unsubstituted aryl groups, substituted or unsubstituted heteroaryl groups, one or more heteroatoms, polyethylene glycol, or a combination thereof;
[L1] and [L2] are optional linkers selected from the group consisting of valine (Val), citrulline (Cit), alanine (Ala), asparagine (Asn), a peptide, —(CH₂)ₙ—, —(CH₂CH2O)ₙ—, p-aminobenzyloxycarbonyl (PAB), Val-Cit-PAB, Val-Ala-PAB, Ala-Ala-Asn-PAB, Val-Ala, Asn-Ala, any amino acid except glycine, and combinations thereof; and
Y comprises the bi-paratopic alkaline phosphatase, placental type (ALPP) and/or alkaline phosphatase, germ cell type (ALPPL2) specific antigen binding molecule of claim 52.

57. The bi-paratopic target-binding molecule-drug conjugate of claim 53, wherein the toxin is Exatecan or an Exatecan derivative.
